(19) United States
(12) Patent Application Publication
(10) Pub. No.: US 2016/0168383 A1
(43) Pub. Date: Jun. 16, 2016

(54) CYANINE COMPOUNDS

(71) Applicants: Greg Hermanson, Loves Park, IL (US); Peter T. Czerney, Weimar (DE); Surbhi Desai, Rockford, IL (US); Matthias S. Wenzel, Jena (DE); Boguslawa R. Dworecki, Rockford, IL (US); Frank G. Lehmann, Jena (DE)

(72) Inventors: Greg Hermanson, Loves Park, IL (US); Peter T. Czerney, Weimar (DE); Surbhi Desai, Rockford, IL (US); Matthias S. Wenzel, Jena (DE); Boguslawa R. Dworecki, Rockford, IL (US); Frank G. Lehmann, Jena (DE)

(21) Appl. No.: 15/042,495

(22) Filed: Feb. 12, 2016

Related U.S. Application Data
(63) Continuation of application No. 13/787,885, filed on Mar. 7, 2013.

(51) Int. Cl. C09B 23/06; G01N 33/53; A61K 49/00; G01N 33/533; C09B 23/08; G01N 33/58

(52) U.S. Cl. CPC C09B 23/06; C09B 23/086; C09B 23/083; G01N 33/582; A61K 49/0032; G01N 33/533; G01N 33/5306

(57) ABSTRACT

Compounds used as labels with properties comparable to known fluorescent compounds. The compounds can be conjugated to proteins and nucleic acids for biological imaging and analysis. Synthesis of the compounds, formation and use of the conjugated compounds, and specific non-limiting examples of each are provided.

This application is a continuation of co-pending U.S. application Ser. No. 13/787,885 filed Mar. 7, 2013, which is expressly incorporated by reference herein in its entirety.

Compounds useful as labels with properties comparable to known fluorescent compounds are disclosed. The compounds can be conjugated to proteins and nucleic acids for biological imaging and analysis. Synthesis of the compounds, formation and use of the conjugated compounds, and specific non-limiting examples of each are disclosed.

Compounds that react with biomolecules (e.g., antigens, antibodies, DNA-segments with the corresponding complimentary species for measuring enzyme kinetics, receptor-ligand interactions, nucleic acid hybridization kinetics in vitro as well as in vivo, etc.), termed labels or dyes, are useful for, e.g., pharmacological characterization of receptors and drugs, binding data, etc. Compounds such as xanthylium salts (U.S. Pat. No. 5,846,737) and/or cyanines (U.S. Pat. No. 5,627,027) are used for such applications, but aggregate and form dimers, especially in aqueous solution, due to planarity of their n-system. Compounds that have insufficient hydrophilicity undergo non-specific interactions with various surfaces, resulting in problems when attempting purify the corresponding conjugate, and an unsatisfactory signal to noise ratio.

Efforts are directed to reducing undesirable properties by introducing substituents that increase the hydrophilicity of the compounds. For example, sulfonic acid function substituents have been introduced into the cyanine chromophore. U.S. Pat. No. 6,083,485 (Licha) and U.S. Pat. Nos. 6,977,305 and 6,974,873 (Molecular Probes) disclose cyanine compounds having one of the common methyl groups in the 3-position of the terminal indole heterocycle substituted by a ω-carboxyalkyl function, and in which the previously present (e.g. in Cy3 or Cy5) N-alkyl or N-ω-carboxyalkyl functions are replaced by N-ω-alkyl sulfonic acid functions. WO 05/044923 discloses cyanine compounds having the common methyl substituent in the 3-position of the terminal indole heterocycle substituted by a N-ω-alkyl sulfonic acid function. In these publications, cyanine compounds having more than two sulfonic acid functional substituents exhibited higher solubility and correspondingly a lower tendency to dimer formation, in comparison to cyanine compounds (Cy3, Cy5) described in U.S. Pat. No. 5,627,027.

The disclosed cyanine compounds are useful as labels in optical, especially fluorescence optical, determination and detection methods. The compounds have high hydrophilicity, high molar absorbance, high photo-stability, and high storage stability. These compounds can be excited by monochromatic (e.g., lasers, laser diodes) or polychromatic (e.g., white light sources) light in the ultraviolet (UV), visible, and near infrared (NIR) spectral region to generate emission of fluorescence light.

Typical application methods are based on the reaction of the compounds with biomolecules such as proteins (e.g., antigens, antibodies, etc.), DNA and/or RNA segments, etc. with the corresponding complimentary species. Thus, among other embodiments, the compounds are useful to measure enzyme kinetics, receptor-ligand interactions, and nucleic acid hybridization kinetics in vitro and/or in vivo.

The compounds are useful for the pharmacological characterization of receptors and/or drugs. Applications include, but are not limited to, uses in medicine, pharmacy, biological sciences, materials sciences, environmental control, detection of organic and inorganic micro samples occurring in nature, etc.

The following nomenclature is used to describe embodiments: 550 Compound 0/X, 550 Compound 1/X, 550 Compound 2/X, 550 Compound 3/X, 550 Compound 4/X, 550 Compound 5/X, 550 Compound 6/X, 650 Compound 0/X, 650 Compound 1/X, 650 Compound 2/X, 650 Compound 3/X, 650 Compound 4/X, 650 Compound 5/X, 650 Compound 6/X, 755 Compound 0/X, 755 Compound 1/X, 755 Compound 2/X, 755 Compound 3/X, 755 Compound 4/X, 755 Compound 5/X, 755 Compound 6/X, where 550, 650, and 755 Compounds comprise a polymethine chain of 3 carbon, 5 carbon, and 7 carbon atoms, respectively; the first number is the length of a ethylene glycol, diethylene glycol, or (poly)ethylene glycol (collectively referred to herein as PEG) on an indole N, e.g. 0 is no PEG on an indole N, 1 is ethylene glycol (PEG₁) on an indole N, 2 is diethylene glycol (PEG₂) on an indole N, 3 is (poly)ethylene glycol (poly=3, PEG₃) on an indole N, 4 is (poly)ethylene glycol (poly=4, PEG₄) on an indole N, 5 is (poly)ethylene glycol (poly=5, PEG₅) on an indole N, and 6 is (poly)ethylene glycol (poly=6, PEG₆) on an indole N; and X is the total number of PEG groups on the compound. For example, 650 Compound 4/4 contains PEG₄ on an indole N and a total of four PEG groups on the compound.

In one embodiment the cyanine compounds have, in an N-position of one heterocycle, an ethylene glycol group or an ethylene glycol polymer (i.e., poly(ethylene) glycol, abbreviated as PEG), and the other heterocycle has, in a N-position, a function for conjugating the compound to a biomolecule, and an ethylene glycol group or an ethylene glycol polymer (i.e., poly(ethylene) glycol, abbreviated as PEG) in another position of the cyanine compound. In one embodiment the cyanine compound has, in any position of the compound, at least one sulfo and/or sulfoalkyl group. In one embodiment the cyanine compound has, in any position of the compound, a sulfonamide and/or carboxamide group comprising an ethylene glycol group or an ethylene glycol polymer (i.e., poly(ethylene) glycol, abbreviated as PEG), either directly or indirectly attached to the compound. Indirect attachment indicates use of a linker, direct attachment indicates lack of such a linker. A linker can be any moiety.

In one embodiment the cyanine compounds have, in an N-position of one heterocycle, an ethylene glycol group or an ethylene glycol polymer (i.e., poly(ethylene) glycol, abbreviated as PEG), and the other heterocycle has, in a N-position, an ethylene glycol group or an ethylene glycol polymer (i.e., poly(ethylene) glycol, abbreviated as PEG) and a function for conjugating the compound to a biomolecule, and an ethylene glycol group or an ethylene glycol polymer (i.e., poly(ethylene) glycol, abbreviated as PEG) in another position of the benzocyanine compound. In one embodiment the cyanine compound has, in any position of the compound, at least one sulfo and/or sulfoalkyl group. In one embodiment the cyanine compound has, in any position of the compound, a sulfonamide and/or carboxamide group comprising an ethylene glycol group or an ethylene glycol polymer (i.e., poly(ethylene) glycol, abbreviated as PEG), either directly or indirectly attached to the compound. Indirect attachment indicates use of a linker, direct attachment indicates lack of such a linker. A linker can be any moiety.

In one embodiment the cyanine compounds have, in an N-position of one heterocycle, an alkyl group, and the other heterocycle has, in a N-position, a function for conjugating the compound to a biomolecule, and an ethylene glycol group or ethylene glycol polymer (i.e., poly(ethylene) glycol, abbreviated as PEG) in another position of the cyanine compound. In one embodiment the cyanine compound has at least one sulfo and/or sulfoalkyl group that is not limited to any particular position or site on the compound. In one embodiment the cyanine compound has, at any position or site on the compound, a sulfonamide and/or carboxamide group comprising an ethylene glycol group or ethylene glycol polymer (i.e., poly(ethylene) glycol, abbreviated as PEG), either directly or indirectly attached to the compound. Indirect attachment uses a linker of any moiety, and direct attachment lacks such a linker.

### BRIEF DESCRIPTION OF THE DRAWINGS

FIG. 1 shows fluorescence plate functional assay results with inventive compounds and commercial dyes according to one embodiment.

FIG. 2 shows results of the functional assay of FIG. 1 expressed as signal-to-background ratio.

FIG. 3 shows fluorescence plate functional assay results with inventive compounds and commercial dyes according to one embodiment.

FIG. 4 shows results of the functional assay of FIG. 3 expressed as signal-to-background ratio.

FIG. 5 shows fluorescence plate functional assay results with inventive compounds and commercial dyes according to one embodiment.

FIG. 6 shows results of the functional assay of FIG. 5 expressed as signal-to-background ratio.

FIG. 7 shows fluorescence plate functional assay results with inventive compounds and commercial dyes according to one embodiment.

FIG. 8 shows results of the functional assay of FIG. 7 expressed as signal-to-background ratio.

FIGS. 9A-9E show results of an immunofluorescence assay with inventive compounds and commercial dyes according to one embodiment.

FIG. 10 shows results of the immunofluorescence assay of FIG. 9 expressed as fluorescence intensity.

FIG. 11 shows results of the immunofluorescence assay of FIG. 9 expressed as signal-to-background ratio.

FIGS. 12A-12E show results of an immunofluorescence assay with inventive compounds and commercial dyes according to one embodiment.

FIG. 13 shows results of the immunofluorescence assay of FIG. 12 expressed as fluorescence intensity.

FIG. 14 shows results of the immunofluorescence assay of FIG. 12 expressed as signal-to-background ratio.

FIGS. 15A-E show results of an immunofluorescence assay with inventive compounds and commercial dyes according to one embodiment.

FIG. 16 shows results of the immunofluorescence assay of FIG. 15 expressed as fluorescence intensity.

FIG. 17 shows results of the immunofluorescence assay of FIG. 15 expressed as signal-to-background ratio.

Unless otherwise noted, reference to general formulas (e.g., I, II, III, IV, V, and VI, each subsequently described), encompasses their respective a, b, c, etc. structures.

In one embodiment the compound is a compound according to general formula Ia with “a” indicating the right N chain terminates in COX:

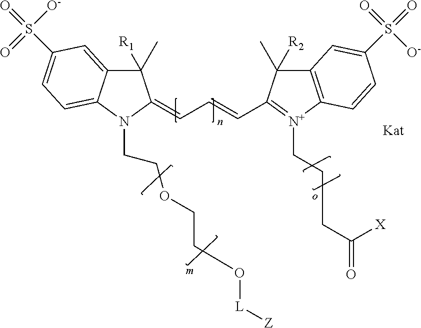

general formula Ib with “b” indicating the right N chain terminates in OH:

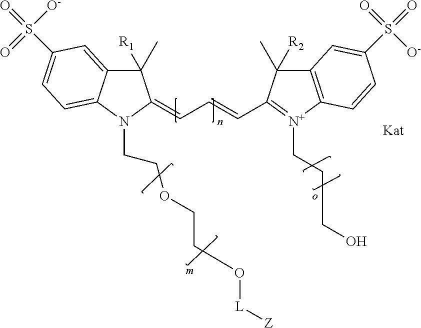

or general formula Ic with “c” indicating ethylene, diethylene, or (poly)ethylene glycol is not required on the left N:

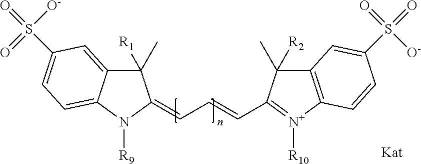

where each of R¹ and R² is the same or different and is independently selected from the group consisting of an aliphatic, heteroaliphatic, sulfoalkyl group, heteroaliphatic with terminal SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group -L-SO₂NH—P-L-Z, and a caboxamide group -L-CONH—P-L-Z; each of R⁹ and R¹⁰ in Formula Ic is the same or different and is independently selected from the group consisting of an alkyl, a sulfoalkyl, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a PEG group P-L-X, L-Z, L-X; where L is selected from the group consisting of a divalent linear (—(CH₂)ₒ—, o=0-15), crossed, or cyclic alkane group that can be substituted by at least one atom selected from the group consisting of oxygen, substituted nitrogen, and/or sulfur; where Z is selected from the group consisting of H, CH₃, alkyl, heteroalkyl, NH₂, —COO⁻, —COOH, —COSH, CO—NH—NH₂, —COF, —COCl, —COBr, —COI, —COO-Su (succinimidyl/sulfo-succinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO⁻, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-O-phosphoramidite, —CONR′-L-CHO, —CONR′-L-maleimide, and —CONR′-L-NH—CO—CH₂—I; R′ and R″ is selected from the group consisting of H, aliphatic group, and heteroaliphatic group, and the biomolecule is a protein, antibody, nucleotide, oligonucleotide, biotin, or hapten; X is selected from the group consisting of —OH, —SH, —NH₂, —NH—NH₂, —F, —Cl, —Br, I, —NHS (hydroxysuccinimidyl/sulfosuccinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group; Kat is a number of Na⁺, K⁺, Ca²⁺, ammonia, or other cation(s) needed to compensate the negative charge brought by the cyanine; m is an integer from 0 to 5 inclusive; o is an integer from 0 to 12 inclusive; and n is an integer from 1 to 3 inclusive; with the proviso that at least one of R¹ and R² contains a PEG group.

In one embodiment one of R9 and R10 is a C1-C6 alkyl, or a C1-C6 alkyl substituted once by hydroxyl, sulfo, carboxy, or amino, and the other of R9 and R10 is an alkyl terminating in a reactive group. In one embodiment the reactive group is selected from X or Z, as defined above.

In one embodiment the PEG group is selected from —C—C—O—C(ethylene glycol with terminal methyl), —C—C—O—C—C—O—C(diethylene glycol with terminal methyl), —C—C—O—C—C—O—C—C—O—C ((poly)ethylene glycol (3) with terminal methyl), —C—C—O—C—C—O—C—C—O—C—C—O—C((poly)ethylene glycol (4) with terminal methyl), —C—C—O—C—C—O—C—C—O—C—C—O—C—C—O—C((poly)ethylene glycol (5) with terminal methyl), or C—C—O—C—C—O—C—C—O—C—C—O—C—C—C—O—C—C—O—C((poly)ethylene glycol (6) with terminal methyl). In one embodiment the PEG group P may be either uncapped, e.g., lack a terminal methyl, or may be capped with an atom or group other than methyl. In one embodiment the PEG group P terminates with a Z group, where Z is selected from H, CH₃, a CH₃ group, an alkyl group, or a heteroalkyl group.

In one embodiment the compound is general formula I, where R1 is sulfoalkyl and R2 is a PEG group; X is —OH, —NHS, —O-TFP, or —NR-L-maleimide; m is 0; o is 3; and n is 1. In one embodiment the compound is general formula I, where R1 is sulfoalkyl and R2 is a PEG group; X is —OH, —NHS, —O-TFP, or —NR-L-maleimide; m is 1; o is 3; and n is 1. In one embodiment the compound is general formula I, where R1 is sulfoalkyl and R2 is a PEG group; X is —OH, —NHS, —O-TFP, or —NR-L-maleimide; m is 2; o is 3; and n is 1. In one embodiment the compound is general formula I, where R1 is sulfoalkyl and R2 is a PEG group; X is —OH, —NHS, —O-TFP, or —NR-L-maleimide; m is 3; o is 3; and n is 1. In one embodiment the compound is general formula I, where R1 is sulfoalkyl and R2 is a PEG group; X is —OH, —NHS, —O-TFP, or —NR-L-maleimide; m is 4; o is 3; and n is 1. In one embodiment the compound is general formula I, where R1 is sulfoalkyl and R2 is a PEG group; X is —OH, —NHS, —O-TFP, or —NR-L-maleimide; m is 5; o is 3; and n is 1.

In one embodiment the compound is general formula I, where R1 is sulfoalkyl and R2 is a PEG group; X is —OH, —NHS, —O-TFP, or —NR-L-maleimide; m is 0; o is 3; and n is 2. In one embodiment the compound is general formula I, where R1 is sulfoalkyl and R2 is a PEG group; X is —OH, —NHS, —O-TFP, or —NR-L-maleimide; m is 1; o is 3; and n is 2. In one embodiment the compound is general formula I, where R1 is sulfoalkyl and R2 is a PEG group; X is —OH, —NHS, —O-TFP, or —NR-L-maleimide; m is 2; o is 3; and n is 2. In one embodiment the compound is general formula I, where R1 is sulfoalkyl and R2 is a PEG group; X is —OH, —NHS, —O-TFP, or —NR-L-maleimide; m is 3; o is 3; and n is 2. In one embodiment the compound is general formula I, where R1 is sulfoalkyl and R2 is a PEG group; X is —OH, —NHS, —O-TFP, or —NR-L-maleimide; m is 4; o is 3; and n is 2. In one embodiment the compound is general formula I, where R1 is sulfoalkyl and R2 is a PEG group; X is —OH, —NHS, —O-TFP, or —NR-L-maleimide; m is 5; o is 3; and n is 2.

In one embodiment the compound is general formula I, where R1 is sulfoalkyl and R2 is a PEG group; X is —OH, —NHS, —O-TFP, or —NR-L-maleimide; m is 0; o is 3; and n is 3. In one embodiment the compound is general formula I, where R1 is sulfoalkyl and R2 is a PEG group; X is —OH, —NHS, —O-TFP, or —NR-L-maleimide; m is 1; o is 3; and n is 3. In one embodiment the compound is general formula I, where R1 is sulfoalkyl and R2 is a PEG group; X is —OH, —NHS, —O-TFP, or —NR-L-maleimide; m is 2; o is 3; and n is 3. In one embodiment the compound is general formula I, where R1 is sulfoalkyl and R2 is a PEG group; X is —OH, —NHS, —O-TFP, or —NR-L-maleimide; m is 3; o is 3; and n is 3. In one embodiment the compound is general formula I, where R1 is sulfoalkyl and R2 is a PEG group; X is —OH, —NHS, —O-TFP, or —NR-L-maleimide; m is 4; o is 3; and n is 3. In one embodiment the compound is general formula I, where R1 is sulfoalkyl and R2 is a PEG group; X is —OH, —NHS, —O-TFP, or —NR-L-maleimide; m is 5; o is 3; and n is 3.

In one embodiment the compound is general formula I, where R1 is methyl and R2 is a PEG group; X is —OH, —NHS, —O-TFP, or —NR-L-maleimide; m is 0; o is 3; and n is 1. In one embodiment the compound is general formula I, where R1 is methyl and R2 is a PEG group; X is —OH, —NHS, —O-TFP, or —NR-L-maleimide; m is 1; o is 3; and n is 1. In one embodiment the compound is general formula I, where R1 is methyl and R2 is a PEG group; X is —OH, —NHS, —O-TFP, or —NR-L-maleimide; m is 2; o is 3; and n is 1. In one embodiment the compound is general formula I, where R1 is methyl and R2 is a PEG group; X is —OH, —NHS, —O-TFP, or —NR-L-maleimide; m is 3; o is 3; and n is 1. In one embodiment the compound is general formula I, where R1 is methyl and R2 is a PEG group; X is —OH, —NHS, —O-TFP, or —NR-L-maleimide; m is 4; o is 3; and n is 1. In one embodiment the compound is general formula I, where R1 is methyl and R2 is a PEG group; X is —OH, —NHS, —O-TFP, or —NR-L-maleimide; m is 5; o is 3; and n is 1.

In one embodiment the compound is general formula I, where R1 is methyl and R2 is a PEG group; X is —OH, —NHS, —O-TFP, or —NR-L-maleimide; m is 0; o is 3; and n is 2. In one embodiment the compound is general formula I, where R1 is methyl and R2 is a PEG group; X is —OH, —NHS, —O-TFP, or —NR-L-maleimide; m is 1; o is 3; and n is 2. In one embodiment the compound is general formula I, where R1 is methyl and R2 is a PEG group; X is —OH, —NHS, —O-TFP, or —NR-L-maleimide; m is 2; o is 3; and n is 2. In one embodiment the compound is general formula I, where R1 is methyl and R2 is a PEG group; X is —OH, —NHS, —O-TFP, or —NR-L-maleimide; m is 3; o is 3; and n is 2. In one embodiment the compound is general formula I, where R1 is methyl and R2 is a PEG group; X is —OH, —NHS, —O-TFP, or —NR-L-maleimide; m is 4; o is 3; and n is 2. In one embodiment the compound is general formula I, where R1 is methyl and R2 is a PEG group; X is —OH, —NHS, —O-TFP, or —NR-L-maleimide; m is 5; o is 3; and n is 2.

In one embodiment the compound is general formula I where R1 is methyl and R2 is a PEG group; X is —OH, —NHS, —O-TFP, or —NR-L-maleimide; m is 0; o is 3; and n is 3. In one embodiment the compound is general formula I where R1 is methyl and R2 is a PEG group; X is —OH, —NHS, —O-TFP, or —NR-L-maleimide; m is 1; o is 3; and n is 3. In one embodiment the compound is general formula I, where R1 is methyl and R2 is a PEG group; X is —OH, —NHS, —O-TFP, or —NR-L-maleimide; m is 2; o is 3; and n is 3. In one embodiment the compound is general formula I where R1 is methyl and R2 is a PEG group; X is —OH, —NHS, —O-TFP, or —NR-L-maleimide; m is 3; o is 3; and n is 3. In one embodiment the compound is general formula I where R1 is methyl and R2 is a PEG group; X is —OH, —NHS, —O-TFP, or —NR-L-maleimide; m is 4; o is 3; and n is 3. In one embodiment the compound is general formula I where R1 is methyl and R2 is a PEG group; X is —OH, —NHS, —O-TFP, or —NR-L-maleimide; m is 5; o is 3; n is 3.

In one embodiment the compound is general formula I, where R1 and R2 are PEG groups; X is —OH, —NHS, —O-TFP, or —NR-L-maleimide; m is 0; o is 3; and n is 1. In one embodiment the compound is general formula I, where R1 and R2 are PEG groups; X is —OH, —NHS, —O-TFP, or —NR-L-maleimide; m is 1; o is 3; and n is 1. In one embodiment the compound is general formula I, where R1 and R2 are PEG groups; X is —OH, —NHS, —O-TFP, or —NR-L-maleimide; m is 2; o is 3; and n is 1. In one embodiment the compound is general formula I, where R1 and R2 are PEG groups; X is —OH, —NHS, —O-TFP, or —NR-L-maleimide; m is 3; o is 3; and n is 1. In one embodiment the compound is general formula I, where R1 and R2 are PEG groups; X is —OH, —NHS, —O-TFP, or —NR-L-maleimide; m is 4; o is 3; and n is 1. In one embodiment the compound is general formula I, where R1 and R2 are PEG groups; X is —OH, —NHS, —O-TFP, or —NR-L-maleimide; m is 5; o is 3; and n is 1.

In one embodiment the compound is general formula I, where R1 and R2 are PEG groups; X is —OH, —NHS, —O-TFP, or —NR-L-maleimide; m is 0; o is 3; and n is 2. In one embodiment the compound is general formula I, where R1 and R2 are PEG groups; X is —OH, —NHS, —O-TFP, or —NR-L-maleimide; m is 1; o is 3; and n is 2. In one embodiment the compound is general formula I, where R1 and R2 are PEG groups; X is —OH, —NHS, —O-TFP, or —NR-L-maleimide; m is 2; o is 3; and n is 2. In one embodiment the compound is general formula I, where R1 and R2 are PEG groups; X is —OH, —NHS, —O-TFP, or —NR-L-maleimide; m is 3; o is 3; and n is 2. In one embodiment the compound is general formula I, where R1 and R2 are PEG groups; X is —OH, —NHS, —O-TFP, or —NR-L-maleimide; m is 4; o is 3; and n is 2. In one embodiment the compound is general formula I, where R1 and R2 are PEG groups; X is —OH, —NHS, —O-TFP, or —NR-L-maleimide; m is 5; o is 3; and n is 2.

In one embodiment the compound is general formula I, where R1 and R2 are PEG groups; X is —OH, —NHS, —O-TFP, or —NR-L-maleimide; m is 0; o is 3; and n is 3. In one embodiment the compound is general formula I, where R1 and R2 are PEG groups; X is —OH, —NHS, —O-TFP, or —NR-L-maleimide; m is 1; o is 3; and n is 3. In one embodiment the compound is general formula I, where R1 and R2 are PEG groups; X is —OH, —NHS, —O-TFP, or —NR-L-maleimide; m is 2; o is 3; and n is 3. In one embodiment the compound is general formula I, where R1 and R2 are PEG groups; X is —OH, —NHS, —O-TFP, or —NR-L-maleimide; m is 3; o is 3; and n is 3. In one embodiment the compound is general formula I, where R1 and R2 are PEG groups; X is —OH, —NHS, —O-TFP, or —NR-L-maleimide; m is 4; o is 3; and n is 3. In one embodiment the compound is general formula I, where R1 and R2 are PEG groups; X is —OH, —NHS, —O-TFP, or —NR-L-maleimide; m is 5; o is 3; and n is 3.

In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 0; o is 3; and n is 1. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 1; o is 3; and n is 1. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 2; o is 3; and n is 1. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 3; o is 3; and n is 1. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 4; o is 3; and n is 1. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 5; o is 3; and n is 1.

In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 0; o is 3; and n is 2. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 1; o is 3; and n is 2. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 2; o is 3; and n is 2. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 3; o is 3; and n is 2. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 4; o is 3; and n is 2. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 5; o is 3; and n is 2.

In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 0; o is 3; and n is 3. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 1; o is 3; and n is 3. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 2; o is 3; and n is 3. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 3; o is 3; and n is 3. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 4; o is 3; and n is 3. In one embodiment the compound is general formula I, where R1 is sulfoalkyl and R2 is a sulfonamide group -L-SO₂NH—P where P is a PEG group; X is —OH, —NHS, —O-TFP, or —NR-L-maleimide; m is 5; o is 3; and n is 3.

In one embodiment the compound is general formula Ic, where R1 is sulfoalkyl and R2 is a PEG group P—Z where P is an ethylene glycol group, and Z is CH₃; R9 is an alkyl; R10 is an alkyl terminating in —OH, —COOH, —NHS, —O-TFP, or —NR-L-maleimide; and n is 1. In one embodiment the compound is general formula Ic, where R1 is sulfoalkyl and R2 is a PEG group P—Z where P is a diethylene glycol group, and Z is CH₃; R9 is an alkyl; R10 is an alkyl terminating in —OH, —COOH, —NHS, —O-TFP, or —NR-L-maleimide; and n is 1. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 3, and Z is CH₃; R9 is alkyl; R10 is alkyl terminating in —OH, —COOH, —NHS, —O-TFP, or —NR-L-maleimide; and n is 1. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 4, and Z is CH₃; R9 is alkyl; R10 is alkyl terminating in —OH, —COOH, —NHS, —O-TFP, or —NR-L-maleimide; and n is 1. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 5, and Z is CH₃; R9 is alkyl; R10 is alkyl terminating in —OH, —COOH, —NHS, —O-TFP, or —NR-L-maleimide; and n is 1. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 6, and Z is CH₃; R9 is alkyl; R10 is alkyl terminating in —OH, —COOH, —NHS, —O-TFP, or —NR-L-maleimide; and n is 1. In one embodiment the alkyl of R9 is ethyl, the alkyl of R10 is pentyl, and R1 is sulfopentyl or sulfopropyl.

In one embodiment the compound is general formula Ic, where R1 is sulfoalkyl and R2 is a PEG group P—Z where P is an ethylene glycol group, and Z is CH₃; R9 is alkyl; R10 is alkyl terminating in —OH, —COOH, —NHS, —O-TFP, or —NR-L-maleimide; and n is 2. In one embodiment the compound is general formula Ic, where R1 is sulfoalkyl and R2 is a PEG group P—Z where P is a diethylene glycol group, and Z is CH₃; R9 is alkyl; R10 is alkyl terminating in —OH, —COOH, —NHS, —O-TFP, or —NR-L-maleimide; and n is 2. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 3, and Z is CH₃; R9 is alkyl; R10 is alkyl terminating in —OH, —COOH, —NHS, —O-TFP, or —NR-L-maleimide; and n is 2. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 4, and Z is CH₃; R9 is alkyl; R10 is alkyl terminating in —OH, —COOH, —NHS, —O-TFP, or —NR-L-maleimide; and n is 2. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 5, and Z is CH₃; R9 is alkyl; R10 is alkyl terminating in —OH, —COOH, —NHS, —O-TFP, or —NR-L-maleimide; and n is 2. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 6, and Z is CH₃; R9 is alkyl; R10 is alkyl terminating in —OH, —COOH, —NHS, —O-TFP, or —NR-L-maleimide; and n is 2. In one embodiment the alkyl of R9 is ethyl, the alkyl of R10 is pentyl, and R1 is sulfopentyl or sulfopropyl.

In one embodiment the compound is general formula Ic, where R1 is sulfoalkyl and R2 is a PEG group P—Z where P is an ethylene glycol group, and Z is CH₃; R9 is alkyl; R10 is alkyl terminating in —OH, —COOH, —NHS, —O-TFP, or —NR-L-maleimide; and n is 3. In one embodiment the compound is general formula Ic, where R1 is sulfoalkyl and R2 is a PEG group P—Z where P is a diethylene glycol group, and Z is CH₃; R9 is alkyl; R10 is alkyl terminating in —OH, —COOH, —NHS, —O-TFP, or —NR-L-maleimide; and n is 3. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 3, and Z is CH₃; R9 is alkyl; R10 is alkyl terminating in —OH, —COOH, —NHS, —O-TFP, or —NR-L-maleimide; and n is 3. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 4, and Z is CH₃; R9 is alkyl; R10 is alkyl terminating in —OH, —COOH, —NHS, —O-TFP, or —NR-L-maleimide; and n is 3. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 5, and Z is CH₃; R9 is alkyl; R10 is alkyl terminating in —OH, —COOH, —NHS, —O-TFP, or —NR-L-maleimide; and n is 3. In one embodiment the compound is general formula Ic, where R1 is sulfoalkyl and R2 is a PEG group P—Z where P is a (poly)ethylene glycol group, where the (poly)ethylene glycol group is (CH₂CH₂O)ₛ, where s is 6, and Z is CH₃; R9 is alkyl; R10 is alkyl terminating in —OH, —COOH, —NHS, —O-TFP, or —NR-L-maleimide; and n is 3. In one embodiment the alkyl of R9 is ethyl, the alkyl of R10 is pentyl, and R1 is sulfopentyl or sulfopropyl.

In one embodiment the compound is general formula Ic, where R2 is sulfoalkyl and R1 is a PEG group P—Z where P is an ethylene glycol group, and Z is CH₃; R9 is alkyl; R10 is alkyl terminating in —OH, —COOH, —NHS, —O-TFP, or —NR-L-maleimide; and n is 1. In one embodiment the compound is general formula Ic, where R2 is sulfoalkyl and R1 is a PEG group P—Z where P is a diethylene glycol group, and Z is CH₃; R9 is alkyl; R10 is alkyl terminating in —OH, —COOH, —NHS, —O-TFP, or —NR-L-maleimide; and n is 1. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 3, and Z is CH₃; R9 is alkyl; R10 is alkyl terminating in —OH, —COOH, —NHS, —O-TFP, or —NR-L-maleimide; and n is 1. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 4, and Z is CH₃; R9 is alkyl; R10 is alkyl terminating in —OH, —COOH, —NHS, —O-TFP, or —NR-L-maleimide; and n is 1. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 5, and Z is CH₃; R9 is alkyl; R10 is alkyl terminating in —OH, —COOH, —NHS, —O-TFP, or —NR-L-maleimide; and n is 1. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 6, and Z is CH₃; R9 is alkyl; R10 is alkyl terminating in —OH, —COOH, —NHS, —O-TFP, or —NR-L-maleimide; and n is 1. In one embodiment the alkyl of R9 is ethyl, the alkyl of R10 is pentyl, and R2 is sulfopentyl or sulfopropyl.

In one embodiment the compound is general formula Ic, where R2 is sulfoalkyl and R1 is a PEG group P—Z where P is an ethylene glycol group, and Z is CH₃; R9 is alkyl; R10 is alkyl terminating in —OH, —COOH, —NHS, —O-TFP, or —NR-L-maleimide; and n is 2. In one embodiment the compound is general formula Ic, where R2 is sulfoalkyl and R1 is a PEG group P—Z where P is a diethylene glycol group, and Z is CH₃; R9 is alkyl; R10 is alkyl terminating in —OH, —COOH, —NHS, —O-TFP, or —NR-L-maleimide; and n is 2. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 3, and Z is CH₃; R9 is alkyl; R10 is alkyl terminating in —OH, —COOH, —NHS, —O-TFP, or —NR-L-maleimide; and n is 2. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 4, and Z is CH₃; R9 is alkyl; R10 is alkyl terminating in —OH, —COOH, —NHS, —O-TFP, or —NR-L-maleimide; and n is 2. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 5, and Z is CH₃; R9 is alkyl; R10 is alkyl terminating in —OH, —COOH, —NHS, —O-TFP, or —NR-L-maleimide; and n is 2. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 6, and Z is CH₃; R9 is alkyl; R10 is alkyl terminating in —OH, —COOH, —NHS, —O-TFP, or —NR-L-maleimide; and n is 2. In one embodiment the alkyl of R9 is ethyl, the alkyl of R10 is pentyl, and R2 is sulfopentyl or sulfopropyl.

In one embodiment the compound is general formula Ic, where R2 is sulfoalkyl and R1 is a PEG group P—Z where P is an ethylene glycol group, and Z is CH₃; R9 is alkyl; R10 is alkyl terminating in —OH, —COOH, —NHS, —O-TFP, or —NR-L-maleimide; and n is 3. In one embodiment the compound is general formula Ic, where R2 is sulfoalkyl and R1 is a PEG group P—Z where P is a diethylene glycol group, and Z is CH₃; R9 is alkyl; R10 is alkyl terminating in —OH, —COOH, —NHS, —O-TFP, or —NR-L-maleimide; and n is 3. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 3, and Z is CH₃; R9 is alkyl; R10 is alkyl terminating in —OH, —COOH, —NHS, —O-TFP, or —NR-L-maleimide; and n is 3. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 4, and Z is CH₃; R9 is alkyl; R10 is alkyl terminating in —OH, —COOH, —NHS, —O-TFP, or —NR-L-maleimide; and n is 3. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 5, and Z is CH₃; R9 is alkyl; R10 is alkyl terminating in —OH, —COOH, —NHS, —O-TFP, or —NR-L-maleimide; and n is 3. In one embodiment the compound is general formula Ic, where R2 is sulfoalkyl and R1 is a PEG group P—Z where P is a (poly)ethylene glycol group, where the (poly)ethylene glycol group is (CH₂CH₂O)ₛ, where s is 6, and Z is CH₃; R9 is alkyl; R10 is alkyl terminating in —OH, —COOH, —NHS, —O-TFP, or —NR-L-maleimide; and n is 3. In one embodiment the alkyl of R9 is ethyl, the alkyl of R10 is pentyl, and R2 is sulfopentyl or sulfopropyl.

In one embodiment the compound is general formula Ic, where R1 and R2 are a PEG group P—Z where P is an ethylene glycol group, and Z is CH₃; R9 is alkyl; R10 is alkyl terminating in —OH, —COOH, —NHS, —O-TFP, or —NR-L-maleimide; and n is 1. In one embodiment the compound is general formula Ic, where R1 and R2 are a PEG group P—Z where P is a diethylene glycol group, and Z is CH₃; R9 is alkyl; R10 is alkyl terminating in —OH, —COOH, —NHS, —O-TFP, or —NR-L-maleimide; and n is 1. In one embodiment the compound is general formula Ic, where R1 and R2 are a PEG group P—Z where P is a (poly)ethylene glycol group, where the (poly)ethylene glycol group is (CH₂CH₂O)ₛ, where s is 3, and Z is CH₃; R9 is alkyl; R10 is alkyl terminating in —OH, —COOH, —NHS, —O-TFP, or —NR-L-maleimide; and n is 1. In one embodiment the compound is general formula Ic, where R1 and R2 are a PEG group P—Z where P is a (poly)ethylene glycol group, where the (poly)ethylene glycol group is (CH₂CH₂O)ₛ, where s is 4, and Z is CH₃; R9 is alkyl; R10 is alkyl terminating in —OH, —COOH, —NHS, —O-TFP, or —NR-L-maleimide; and n is 1. In one embodiment the compound is general formula Ic, where R1 and R2 are a PEG group P—Z where P is a (poly)ethylene glycol group, where the (poly)ethylene glycol group is (CH₂CH₂O)ₛ, where s is 5, and Z is CH₃; R9 is alkyl; R10 is alkyl terminating in —OH, —COOH, —NHS, —O-TFP, or —NR-L-maleimide; and n is 1. In one embodiment the compound is general formula Ic, where R1 and R2 are a PEG group P—Z where P is a (poly)ethylene glycol group, where the (poly)ethylene glycol group is (CH₂CH₂O)ₛ, where s is 6, and Z is CH₃; R9 is alkyl; R10 is alkyl terminating in —OH, —COOH, —NHS, —O-TFP, or —NR-L-maleimide; and n is 1. In one embodiment the alkyl of R9 is ethyl, and the alkyl of R10 is pentyl.

In one embodiment the compound is general formula Ic, where R1 and R2 are a PEG group P—Z where P is an ethylene glycol group, and Z is CH₃; R9 is alkyl; R10 is alkyl terminating in —OH, —COOH, —NHS, —O-TFP, or —NR-L-maleimide; and n is 2. In one embodiment the compound is general formula Ic, where R1 and R2 are a PEG group P—Z where P is a diethylene glycol group, and Z is CH₃; R9 is alkyl; R10 is alkyl terminating in —OH, —COOH, —NHS, —O-TFP, or —NR-L-maleimide; and n is 2. In one embodiment the compound is general formula Ic, where R1 and R2 are a PEG group P—Z where P is a (poly)ethylene glycol group, where the (poly)ethylene glycol group is (CH₂CH₂O)ₛ, where s is 3, and Z is CH₃; R9 is alkyl; R10 is alkyl terminating in —OH, —COOH, —NHS, —O-TFP, or —NR-L-maleimide; and n is 2.

In one embodiment the compound is general formula Ic, where R1 and R2 are a PEG group P—Z where P is a (poly)ethylene glycol group, where the (poly)ethylene glycol group is (CH₂CH₂O)ₛ, where s is 4, and Z is CH₃; R9 is alkyl; R10 is alkyl terminating in —OH, —COOH, —NHS, —O-TFP, or —NR-L-maleimide; and n is 2. In one embodiment the compound is general formula Ic, where R1 and R2 are a PEG group P—Z where P is a (poly)ethylene glycol group, where the (poly)ethylene glycol group is (CH₂CH₂O)ₛ, where s is 5, and Z is CH₃; R9 is alkyl; R10 is alkyl terminating in —OH, —COOH, —NHS, —O-TFP, or —NR-L-maleimide; and n is 2. In one embodiment the compound is general formula Ic, where R1 and R2 are a PEG group P—Z where P is a (poly)ethylene glycol group, where the (poly)ethylene glycol group is (CH₂CH₂O)ₛ, where s is 6, and Z is CH₃; R9 is alkyl; R10 is alkyl terminating in —OH, —COOH, —NHS, —O-TFP, or —NR-L-maleimide; and n is 2. In one embodiment the alkyl of R9 is ethyl, and the alkyl of R10 is pentyl.

In one embodiment the compound is general formula Ic, where R1 and R2 are a PEG group P—Z where P is an ethylene glycol group, and Z is CH₃; R9 is alkyl; R10 is alkyl terminating in —OH, —COOH, —NHS, —O-TFP, or —NR-L-maleimide; and n is 3. In one embodiment the compound is general formula Ic, where R1 and R2 are a PEG group P—Z where P is a diethylene glycol group, and Z is CH₃; R9 is alkyl; R10 is alkyl terminating in —OH, —COOH, —NHS, —O-TFP, or —NR-L-maleimide; and n is 3. In one embodiment the compound is general formula Ic, where R1 and R2 are a PEG group P—Z where P is a (poly)ethylene glycol group, where the (poly)ethylene glycol group is (CH₂CH₂O)ₛ, where s is 3, and Z is CH₃; R9 is alkyl; R10 is alkyl terminating in —OH, —COOH, —NHS, —O-TFP, or —NR-L-maleimide; and n is 3. In one embodiment the compound is general formula Ic, where R1 and R2 are a PEG group P—Z where P is a (poly)ethylene glycol group, where the (poly)ethylene glycol group is (CH₂CH₂O)ₛ, where s is 4, and Z is CH₃; R9 is alkyl; R10 is alkyl terminating in —OH, —COOH, —NHS, —O-TFP, or —NR-L-maleimide; and n is 3. In one embodiment the compound is general formula Ic, where R1 and R2 are a PEG group P—Z where P is a (poly)ethylene glycol group, where the (poly)ethylene glycol group is (CH₂CH₂O)ₛ, where s is 5, and Z is CH₃; R9 is alkyl; R10 is alkyl terminating in —OH, —COOH, —NHS, —O-TFP, or —NR-L-maleimide; and n is 3. In one embodiment the compound is general formula Ic, where R1 and R2 are a PEG group P—Z where P is a (poly)ethylene glycol group, where the (poly)ethylene glycol group is (CH₂CH₂O)ₛ, where s is 6, and Z is CH₃; R9 is alkyl; R10 is alkyl terminating in —OH, —COOH, —NHS, —O-TFP, or —NR-L-maleimide; and n is 3. In one embodiment the alkyl of R9 is ethyl, and the alkyl of R10 is pentyl.

In one embodiment an isolated enantiomeric mixture selected from diastereomer Ia of general formula Ia

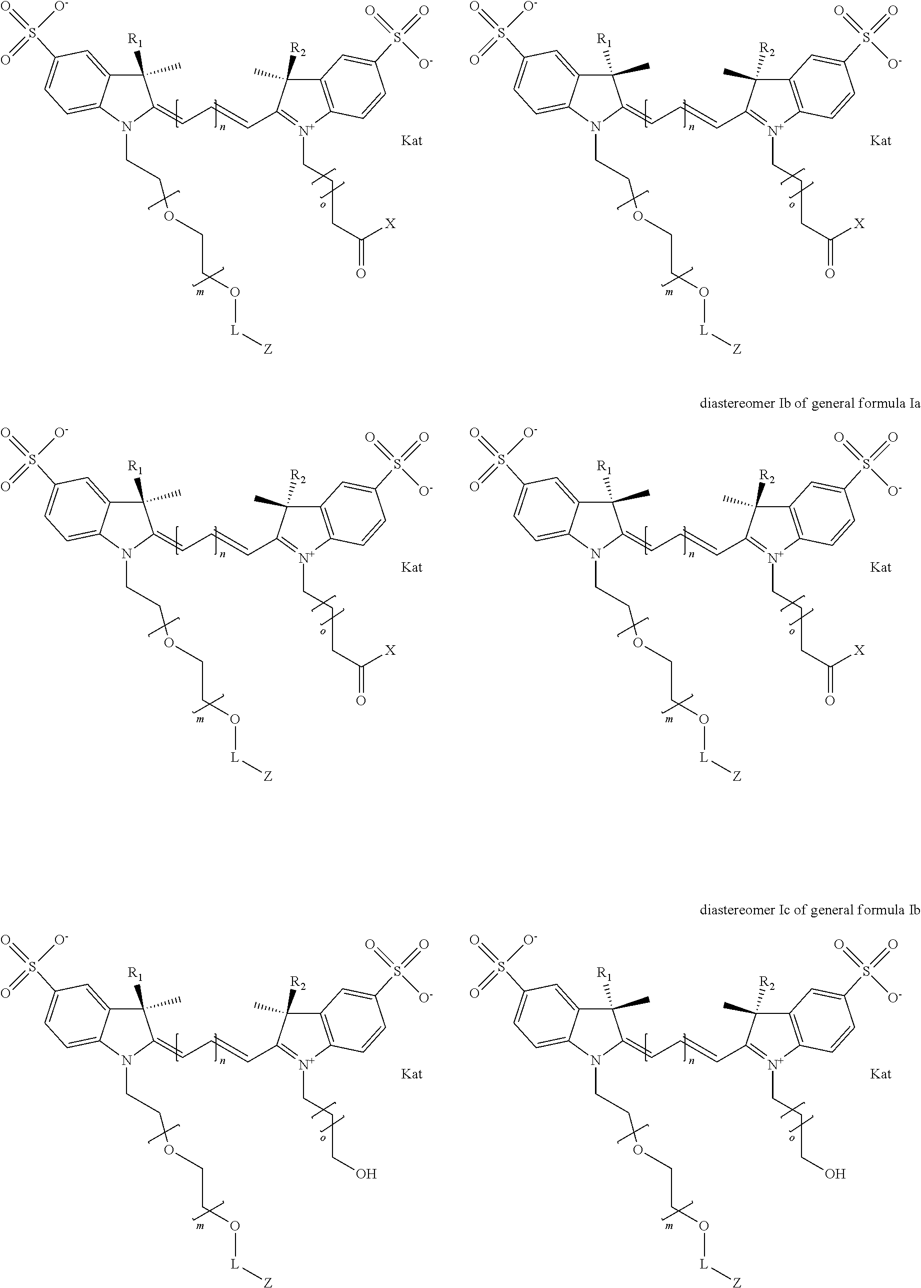

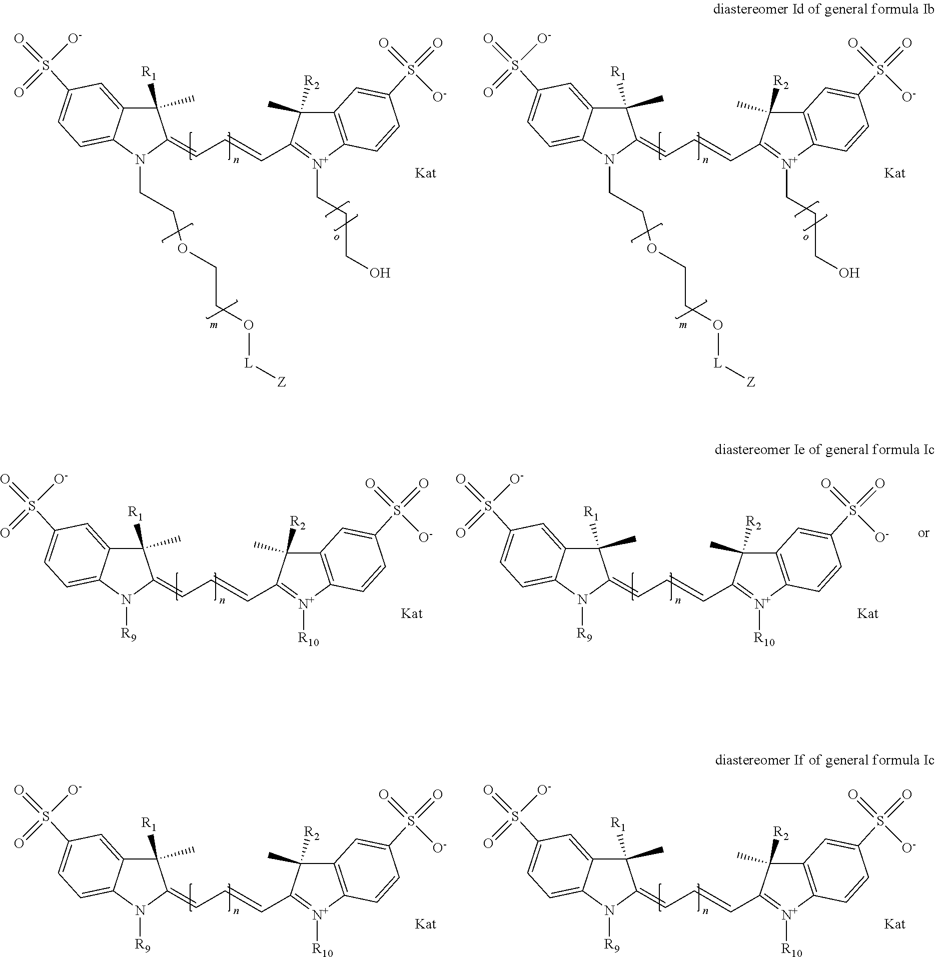

is provided, where each R¹ and R² is the same or different and is independently selected from the group consisting of an aliphatic, heteroaliphatic, sulfoalkyl group, heteroaliphatic with terminal SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group -L-SO₂NH—P-L-Z, and a caboxamide group -L-CONH—P-L-Z; each of R⁹ and R¹⁰ (in Ic) is the same or different and is independently selected from the group consisting of alkyl, sulfoalkyl, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a PEG group P-L-X, L-Z, L-X; where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur; where Z is selected from the group consisting of H, CH₃, an alkyl, a heteroalkyl, NH₂, —COO⁻, —COOH, —COSH, CO—NH—NH₂, —COF, —COCl, —COBr, —COI, —COO-Su (succinimidyl/sulfosuccinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO⁻, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-O-phosphoramidite, —CONR′-L-CHO, —CONR′-L-maleimide, and —CONR′-L-NH—CO—CH₂—I; R′ and R″ is selected from the group consisting of H, aliphatic group, and heteroaliphatic group, and the biomolecule is a protein, antibody, nucleotide, oligonucleotide, biotin, or hapten; X is selected from the group consisting of —OH, —SH, —NH₂, —NH—NH₂, —F, —Cl, —Br, I, —NHS (hydroxysuccinimidyl/sulfo-succinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group; Kat is a number of Na⁺, K⁺, Ca²⁺, ammonia, or other cation(s) needed to compensate the negative charge brought by the cyanine; m is an integer from 0 to 5 inclusive; o is an integer from 0 to 12 inclusive; and n is an integer from 1 to 3 inclusive; with the proviso that at least one of R¹ and R² contains a PEG group.

In one embodiment the compound has general formula IIa with “a” indicating the right N chain terminates in COX:

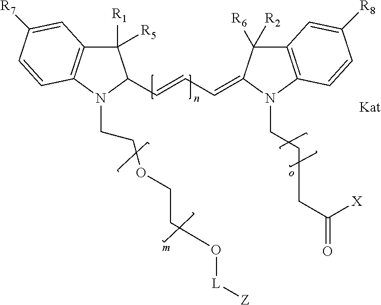

general formula IIb with “b” indicating the right N chain terminates in COH:

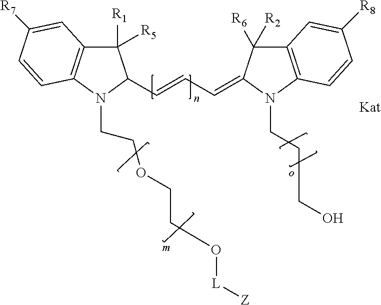

or general formula IIc with “c” indicating ethylene or (poly)ethylene glycol is not required on the left N:

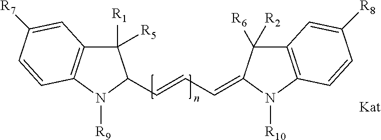

where each R¹, R², R⁵, and R⁶ is the same or different and is independently selected from the group consisting of an aliphatic, heteroaliphatic, sulfoalkyl group, heteroaliphatic with terminal SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group -L-SO₂NH—P-L-Z, and a caboxamide group -L-CONH—P-L-Z; each of R⁷ and R⁸ is the same or different and is independently selected from the group consisting of H, SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group —SO₂NH—P-L-Z, and a caboxamide group —CONH—P-L-Z; each of R⁹ and R¹⁰ in Formula IIc is the same or different and is independently selected from the group consisting of alkyl, sulfoalkyl, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a PEG group P-L-X, L-Z, L-X; where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur; where Z is selected from the group consisting of H, CH₃, an alkyl, a heteroalkyl, NH₂, —COO⁻, —COOH, —COSH, CO—NH—NH₂, —COF, —COCl, —COBr, —COI, —COO-Su (succinimidyl/sulfosuccinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-O-phosphoramidite, —CONR′-L-CHO, —CONR′-L-maleimide, and —CONR′-L-NH—CO—CH₂—I; R′ and R″ is selected from the group consisting of H, aliphatic group, and heteroaliphatic group, and the biomolecule is a protein, antibody, nucleotide, oligonucleotide, biotin, or hapten; X is selected from the group consisting of —OH, —SH, —NH₂, —NH—NH₂, —F, —Cl, —Br, I, —NHS (hydroxysuccinimidyl/sulfo-succinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group; Kat is a number of Na⁺, K⁺, Ca²⁺, ammonia, or other cation(s) needed to compensate the negative charge brought by the cyanine; m is an integer from 0 to 5 inclusive; o is an integer from 0 to 12 inclusive; and n is an integer from 1 to 3 inclusive; with the proviso that at least one of R¹, R², R⁵, R⁶, R⁷, and R⁸ contains a PEG group.

In one embodiment the compound is general formula II where R1, R5, and R6 are methyl and R2 is a PEG group; R7 and R8 are sulfo; X is —OH, —NHS, —O-TFP, or —NR-L-maleimide; m is 0; o is 3; and n is 1. In one embodiment the compound is general formula II where R1, R5, and R6 are methyl and R2 is a PEG group; R7 and R8 are sulfo; X is —OH, —NHS, —O-TFP, or —NR-L-maleimide; m is 1; o is 3; and n is 1. In one embodiment the compound is general formula II where R1, R5, and R6 are methyl and R2 is a PEG group; R7 and R8 are sulfo; X is —OH, —NHS, —O-TFP, or —NR-L-maleimide; m is 2; o is 3; and n is 1. In one embodiment the compound is general formula II where R1, R5, and R6 are methyl and R2 is a PEG group; R7 and R8 are sulfo; X is —OH, —NHS, —O-TFP, or —NR-L-maleimide; m is 3; o is 3; and n is 1. In one embodiment the compound is general formula II where R1, R5, and R6 are methyl and R2 is a PEG group; R7 and R8 are sulfo; X is —OH, —NHS, —O-TFP, or —NR-L-maleimide; m is 4; o is 3; and n is 1. In one embodiment the compound is general formula II where R1, R5, and R6 are methyl and R2 is a PEG group; R7 and R8 are sulfo; X is —OH, —NHS, —O-TFP, or —NR-L-maleimide; m is 5; o is 3; and n is 1.

In one embodiment the compound is general formula II where R1, R5, and R6 are methyl and R2 is a PEG group; R7 and R8 are sulfo; X is —OH, —NHS, —O-TFP, or —NR-L-maleimide; m is 0; o is 3; and n is 2. In one embodiment the compound is general formula II where R1, R5, and R6 are methyl and R2 is a PEG group; R7 and R8 are sulfo; X is —OH, —NHS, —O-TFP, or —NR-L-maleimide; m is 1; o is 3; and n is 2. In one embodiment the compound is general formula II where R1, R5, and R6 are methyl and R2 is a PEG group; R7 and R8 are sulfo; X is —OH, —NHS, —O-TFP, or —NR-L-maleimide; m is 2; o is 3; and n is 2. In one embodiment the compound is general formula II where R1, R5, and R6 are methyl and R2 is a PEG group; R7 and R8 are sulfo; X is —OH, —NHS, —O-TFP, or —NR-L-maleimide; m is 3; o is 3; and n is 2. In one embodiment the compound is general formula II where R1, R5, and R6 are methyl and R2 is a PEG group; R7 and R8 are sulfo; X is —OH, —NHS, —O-TFP, or —NR-L-maleimide; m is 4; o is 3; and n is 2. In one embodiment the compound is general formula II where R1, R5, and R6 are methyl and R2 is a PEG group; R7 and R8 are sulfo; X is —OH, —NHS, —O-TFP, or —NR-L-maleimide; m is 5; o is 3; and n is 2.

In one embodiment the compound is general formula II where R1, R5, and R6 are methyl and R2 is a PEG group; R7 and R8 are sulfo; X is —OH, —NHS, —O-TFP, or —NR-L-maleimide; m is 0; o is 3; and n is 3. In one embodiment the compound is general formula II where R1, R5, and R6 are methyl and R2 is a PEG group; R7 and R8 are sulfo; X is —OH, —NHS, —O-TFP, or —NR-L-maleimide; m is 1; o is 3; and n is 3. In one embodiment the compound is general formula II where R1, R5, and R6 are methyl and R2 is a PEG group; R7 and R8 are sulfo; X is —OH, —NHS, —O-TFP, or —NR-L-maleimide; m is 2; o is 3; and n is 3. In one embodiment the compound is general formula II where R1, R5, and R6 are methyl and R2 is a PEG group; R7 and R8 are sulfo; X is —OH, —NHS, —O-TFP, or —NR-L-maleimide; m is 3; o is 3; and n is 3. In one embodiment the compound is general formula II where R1, R5, and R6 are methyl and R2 is a PEG group; R7 and R8 are sulfo; X is —OH, —NHS, —O-TFP, or —NR-L-maleimide; m is 4; o is 3; and n is 3. In one embodiment the compound is general formula II where R1, R5, and R6 are methyl and R2 is a PEG group; R7 and R8 are sulfo; X is —OH, —NHS, —O-TFP, or —NR-L-maleimide; m is 5; o is 3; and n is 3.

In one embodiment the compound is general formula II where R5 and R6 are methyl; R1 and R2 are PEG groups; R7 and R8 are sulfo; X is —OH, —NHS, —O-TFP, or —NR-L-maleimide; m is 0; o is 3; and n is 1. In one embodiment the compound is general formula II where R5 and R6 are methyl; R1 and R2 are PEG groups; R7 and R8 are sulfo; X is —OH, —NHS, —O-TFP, or —NR-L-maleimide; m is 1; o is 3; and n is 1. In one embodiment the compound is general formula II where R5 and R6 are methyl; R1 and R2 are PEG groups; R7 and R8 are sulfo; X is —OH, —NHS, —O-TFP, or —NR-L-maleimide; m is 2; o is 3; and n is 1. In one embodiment the compound is general formula II where R5 and R6 are methyl; R1 and R2 are PEG groups; R7 and R8 are sulfo; X is —OH, —NHS, —O-TFP, or —NR-L-maleimide; m is 3; o is 3; and n is 1. In one embodiment the compound is general formula II, where R5 and R6 are methyl; R1 and R2 are PEG groups; R7 and R8 are sulfo; X is —OH, —NHS, —O-TFP, or —NR-L-maleimide; m is 4; o is 3; and n is 1. In one embodiment the compound is general formula II where R5 and R6 are methyl; R1 and R2 are PEG groups; R7 and R8 are sulfo; X is —OH, —NHS, —O-TFP, or —NR-L-maleimide; m is 5; o is 3; and n is 1.

In one embodiment the compound is general formula II where R5 and R6 are methyl; R1 and R2 are PEG groups; R7 and R8 are sulfo; X is —OH, —NHS, —O-TFP, or —NR-L-maleimide; m is 0; o is 3; and n is 2. In one embodiment the compound is general formula II where R5 and R6 are methyl; R1 and R2 are PEG groups; R7 and R8 are sulfo; X is —OH, —NHS, —O-TFP, or —NR-L-maleimide; m is 1; o is 3; and n is 2. In one embodiment the compound is general formula II, where R5 and R6 are methyl; R1 and R2 are PEG groups; R7 and R8 are sulfo; X is —OH, —NHS, —O-TFP, or —NR-L-maleimide; m is 2; o is 3; and n is 2. In one embodiment the compound is general formula II, where R5 and R6 are methyl; R1 and R2 are PEG groups; R7 and R8 are sulfo; X is —OH, —NHS, —O-TFP, or —NR-L-maleimide; m is 3; o is 3; and n is 2. In one embodiment the compound is general formula II, where R5 and R6 are methyl; R1 and R2 are PEG groups; R7 and R8 are sulfo; X is —OH, —NHS, —O-TFP, or —NR-L-maleimide; m is 4; o is 3; and n is 2. In one embodiment the compound is general formula II, where R5 and R6 are methyl; R1 and R2 are PEG groups; R7 and R8 are sulfo; X is —OH, —NHS, —O-TFP, or —NR-L-maleimide; m is 5; o is 3; and n is 2.

In one embodiment the compound is general formula II where R5 and R6 are methyl; R1 and R2 are PEG groups; R7 and R8 are sulfo; X is —OH, —NHS, —O-TFP, or —NR-L-maleimide; m is 0; o is 3; and n is 3. In one embodiment the compound is general formula II where R5 and R6 are methyl; R1 and R2 are PEG groups; R7 and R8 are sulfo; X is —OH, —NHS, —O-TFP, or —NR-L-maleimide; m is 1; o is 3; and n is 3. In one embodiment the compound is general formula II where R5 and R6 are methyl; R1 and R2 are PEG groups; R7 and R8 are sulfo; X is —OH, —NHS, —O-TFP, or —NR-L-maleimide; m is 2; o is 3; and n is 3. In one embodiment the compound is general formula II where R5 and R6 are methyl; R1 and R2 are PEG groups; R7 and R8 are sulfo; X is —OH, —NHS, —O-TFP, or —NR-L-maleimide; m is 3; o is 3; and n is 3. In one embodiment the compound is general formula II, where R5 and R6 are methyl; R1 and R2 are PEG groups; R7 and R8 are sulfo; X is —OH, —NHS, —O-TFP, or —NR-L-maleimide; m is 4; o is 3; and n is 3. In one embodiment the compound is general formula II where R5 and R6 are methyl; R1 and R2 are PEG groups; R7 and R8 are sulfo; X is —OH, —NHS, —O-TFP, or —NR-L-maleimide; m is 5; o is 3; and n is 3.

In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 0; o is 3; and n is 1. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 1; o is 3; and n is 1. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 2; o is 3; and n is 1. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 3; o is 3; and n is 1. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 4; o is 3; and n is 1. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 5; o is 3; and n is 1.

In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 0; o is 3; and n is 2. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 1; o is 3; and n is 2. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 2; o is 3; and n is 2. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 3; o is 3; and n is 2. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 4; o is 3; and n is 2. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 5; o is 3; and n is 2.

In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 0; o is 3; and n is 3. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 1; o is 3; and n is 3. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 2; o is 3; and n is 3. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 3; o is 3; and n is 3. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 4; o is 3; and n is 3. In one embodiment the compound is general formula II, where R5 and R6 are methyl; R1 and R2 are PEG groups; R7 is sulfo; R8 is a sulfonamide group -L-SO₂NH—P where P is a PEG group; X is —OH, —NHS, —O-TFP, or —NR-L-maleimide; m is 5; o is 3; and n is 3.

In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 0; o is 3; and n is 1. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 1; o is 3; and n is 1. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 2; o is 3; and n is 1. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 3; o is 3; and n is 1. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 4; o is 3; and n is 1. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 5; o is 3; and n is 1.

In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 0; o is 3; and n is 2. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 1; o is 3; and n is 2. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 2; o is 3; and n is 2. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 3; o is 3; and n is 2. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 4; o is 3; and n is 2. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 5; o is 3; and n is 2.

In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 0; o is 3; and n is 3. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 1; o is 3; and n is 3. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 2; o is 3; and n is 3. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 3; o is 3; and n is 3. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 4; o is 3; and n is 3. In one embodiment the compound is general formula II, where R5 and R6 are methyl; R1 and R2 are PEG groups; R7 is sulfo; R8 is a caboxamide group —CONH—P where P is a PEG group; X is —OH, —NHS, —O-TFP, or —NR-L-maleimide; m is 5; o is 3; and n is 3.

In one embodiment the compound is general formula IIc where R1, R5, and R6 are methyl and R2 is a PEG group P—Z, where P is an ethylene glycol group and Z is CH₃; R7 and R8 are sulfo; R9 is alkyl; R10 is alkyl terminating in —OH, —COOH, —NHS, —O-TFP, or —NR-L-maleimide; and n is 1. In one embodiment the compound is general formula IIc where R1, R5, and R6 are methyl and R2 is a PEG group P—Z, where P is a diethylene glycol group and Z is CH₃; R7 and R8 are sulfo; R9 is alkyl; R10 is alkyl terminating in —OH, —COOH, —NHS, —O-TFP, or —NR-L-maleimide; and n is 1. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are sulfo; R9 is alkyl; R10 is alkyl terminating in —OH, —COOH, —NHS, —O-TFP, or —NR-L-maleimide; and n is 1. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are sulfo; R9 is alkyl; R10 is alkyl terminating in —OH, —COOH, —NHS, —O-TFP, or —NR-L-maleimide; and n is 1. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are sulfo; R9 is alkyl; R10 is alkyl terminating in —OH, —COOH, —NHS, —O-TFP, or —NR-L-maleimide; and n is 1. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are sulfo; R9 is alkyl; R10 is alkyl terminating in —OH, —COOH, —NHS, —O-TFP, or —NR-L-maleimide; and n is 1. In one embodiment the alkyl of R9 is ethyl, and the alkyl of R10 is pentyl.

In one embodiment the compound is general formula IIc where R1, R5, and R6 are methyl and R2 is a PEG group P—Z, where P is an ethylene glycol group and Z is CH₃; R7 and R8 are sulfo; R9 is alkyl; R10 is alkyl terminating in —OH, —COOH, —NHS, —O-TFP, or —NR-L-maleimide; and n is 2. In one embodiment the compound is general formula IIc where R1, R5, and R6 are methyl and R2 is a PEG group P—Z, where P is a diethylene glycol group and Z is CH₃; R7 and R8 are sulfo; R9 is alkyl; R10 is alkyl terminating in —OH, —COOH, —NHS, —O-TFP, or —NR-L-maleimide; and n is 2. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are sulfo; R9 is alkyl; R10 is alkyl terminating in —OH, —COOH, —NHS, —O-TFP, or —NR-L-maleimide; and n is 2. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are sulfo; R9 is alkyl; R10 is alkyl terminating in —OH, —COOH, —NHS, —O-TFP, or —NR-L-maleimide; and n is 2. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are sulfo; R9 is alkyl; R10 is alkyl terminating in —OH, —COOH, —NHS, —O-TFP, or —NR-L-maleimide; and n is 2. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are sulfo; R9 is alkyl; R10 is alkyl terminating in —OH, —COOH, —NHS, —O-TFP, or —NR-L-maleimide; and n is 2. In one embodiment the alkyl of R9 is ethyl, and the alkyl of R10 is pentyl.

In one embodiment the compound is general formula IIc where R1, R5, and R6 are methyl and R2 is a PEG group P—Z, where P is an ethylene glycol group and Z is CH₃; R7 and R8 are sulfo; R9 is alkyl; R10 is alkyl terminating in —OH, —COOH, —NHS, —O-TFP, or —NR-L-maleimide; and n is 3. In one embodiment the compound is general formula IIc where R1, R5, and R6 are methyl and R2 is a PEG group P—Z, where P is a diethylene glycol group and Z is CH₃; R7 and R8 are sulfo; R9 is alkyl; R10 is alkyl terminating in —OH, —COOH, —NHS, —O-TFP, or —NR-L-maleimide; and n is 3. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are sulfo; R9 is alkyl; R10 is alkyl terminating in —OH, —COOH, —NHS, —O-TFP, or —NR-L-maleimide; and n is 3. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are sulfo; R9 is alkyl; R10 is alkyl terminating in —OH, —COOH, —NHS, —O-TFP, or —NR-L-maleimide; and n is 3. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are sulfo; R9 is alkyl; R10 is alkyl terminating in —OH, —COOH, —NHS, —O-TFP, or —NR-L-maleimide; and n is 3. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are sulfo; R9 is alkyl; R10 is alkyl terminating in —OH, —COOH, —NHS, —O-TFP, or —NR-L-maleimide; and n is 3. In one embodiment the alkyl of R9 is ethyl, and the alkyl of R10 is pentyl.

In one embodiment the compound is general formula IIc where R1, R5, and R6 are methyl and R2 is a PEG group P—Z, where P is an ethylene glycol group and Z is CH₃; R7 and R8 are H; R9 is alkyl; R10 is alkyl terminating in —OH, —COOH, —NHS, —O-TFP, or —NR-L-maleimide; and n is 1. In one embodiment the compound is general formula IIc where R1, R5, and R6 are methyl and R2 is a PEG group P—Z, where P is a diethylene glycol group and Z is CH₃; R7 and R8 are H; R9 is alkyl; R10 is alkyl terminating in —OH, —COOH, —NHS, —O-TFP, or —NR-L-maleimide; and n is 1. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are H; R9 is alkyl; R10 is alkyl terminating in —OH, —COOH, —NHS, —O-TFP, or —NR-L-maleimide; and n is 1. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are H; R9 is alkyl; R10 is alkyl terminating in —OH, —COOH, —NHS, —O-TFP, or —NR-L-maleimide; and n is 1. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are H; R9 is alkyl; R10 is alkyl terminating in —OH, —COOH, —NHS, —O-TFP, or —NR-L-maleimide; and n is 1. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are H; R9 is alkyl; R10 is alkyl terminating in —OH, —COOH, —NHS, —O-TFP, or —NR-L-maleimide; and n is 1. In one embodiment the alkyl of R9 is ethyl, and the alkyl of R10 is pentyl.

In one embodiment the compound is general formula IIc where R1, R5, and R6 are methyl and R2 is a PEG group P—Z, where P is an ethylene glycol group and Z is CH₃; R7 and R8 are H; R9 is alkyl; R10 is alkyl terminating in —OH, —COOH, —NHS, —O-TFP, or —NR-L-maleimide; and n is 2. In one embodiment the compound is general formula IIc where R1, R5, and R6 are methyl and R2 is a PEG group P—Z, where P is a diethylene glycol group and Z is CH₃; R7 and R8 are H; R9 is alkyl; R10 is alkyl terminating in —OH, —COOH, —NHS, —O-TFP, or —NR-L-maleimide; and n is 2. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are H; R9 is alkyl; R10 is alkyl terminating in —OH, —COOH, —NHS, —O-TFP, or —NR-L-maleimide; and n is 2. In one embodiment the compound is general formula Ic where R1, R5, and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are H; R9 is alkyl; R10 is alkyl terminating in —OH, —COOH, —NHS, —O-TFP, or —NR-L-maleimide; and n is 2. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are H; R9 is alkyl; R10 is alkyl terminating in —OH, —COOH, —NHS, —O-TFP, or —NR-L-maleimide; and n is 2. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are H; R9 is alkyl; R10 is alkyl terminating in —OH, —COOH, —NHS, —O-TFP, or —NR-L-maleimide; and n is 2. In one embodiment the alkyl of R9 is ethyl, and the alkyl of R10 is pentyl.

In one embodiment the compound is general formula IIc where R1, R5, and R6 are methyl and R2 is a PEG group P—Z, where P is an ethylene glycol group and Z is CH₃; R7 and R8 are H; R9 is alkyl; R10 is alkyl terminating in —OH, —COOH, —NHS, —O-TFP, or —NR-L-maleimide; and n is 3. In one embodiment the compound is general formula IIc where R1, R5, and R6 are methyl and R2 is a PEG group P—Z, where P is a diethylene glycol group and Z is CH₃; R7 and R8 are H; R9 is alkyl; R10 is alkyl terminating in —OH, —COOH, —NHS, —O-TFP, or —NR-L-maleimide; and n is 3. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are H; R9 is alkyl; R10 is alkyl terminating in —OH, —COOH, —NHS, —O-TFP, or —NR-L-maleimide; and n is 3. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are H; R9 is alkyl; R10 is alkyl terminating in —OH, —COOH, —NHS, —O-TFP, or —NR-L-maleimide; and n is 3. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are H; R9 is alkyl; R10 is alkyl terminating in —OH, —COOH, —NHS, —O-TFP, or —NR-L-maleimide; and n is 3. In one embodiment the compound is general formula IIc where R1, R5, and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are H; R9 is alkyl; R10 is alkyl terminating in —OH, —COOH, —NHS, —O-TFP, or —NR-L-maleimide; and n is 3. In one embodiment the alkyl of R9 is ethyl, and the alkyl of R10 is pentyl.

In one embodiment the compound is general formula IIc where R1 is sulfoalkyl, R5 and R6 are methyl and R2 is a PEG group P—Z, where P is an ethylene glycol group and Z is CH₃; R7 and R8 are H; R9 is alkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a diethylene glycol group and Z is CH₃; R7 and R8 are H; R9 is alkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are H; R9 is alkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are H; R9 is alkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are H; R9 is alkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are H; R9 is alkyl; R10 is alkyl terminating in —OH, —COOH, —NHS, —O-TFP, or —NR-L-maleimide; and n is 1. In one embodiment the alkyl of R9 is ethyl, the alkyl of R10 is pentyl, and R1 is sulfopentyl or sulfopropyl.

In one embodiment the compound is general formula IIc where R1 is sulfoalkyl, R5 and R6 are methyl and R2 is a PEG group P—Z, where P is an ethylene glycol group and Z is CH₃; R7 and R8 are H; R9 is alkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a diethylene glycol group and Z is CH₃; R7 and R8 are H; R9 is alkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are H; R9 is alkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are H; R9 is alkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are H; R9 is alkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are H; R9 is alkyl; R10 is alkyl terminating in —OH, —COOH, —NHS, —O-TFP, or —NR-L-maleimide; and n is 2. In one embodiment the alkyl of R9 is ethyl, the alkyl of R10 is pentyl, and R1 is sulfopentyl or sulfopropyl.

In one embodiment the compound is general formula IIc where R1 is sulfoalkyl, R5 and R6 are methyl and R2 is a PEG group P—Z, where P is an ethylene glycol group and Z is CH₃; R7 and R8 are H; R9 is alkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a diethylene glycol group and Z is CH₃; R7 and R8 are H; R9 is alkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are H; R9 is alkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are H; R9 is alkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are H; R9 is alkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are H; R9 is alkyl; R10 is alkyl terminating in —OH, —COOH, —NHS, —O-TFP, or —NR-L-maleimide; and n is 3. In one embodiment the alkyl of R9 is ethyl, the alkyl of R10 is pentyl, and R1 is sulfopentyl or sulfopropyl.

In one embodiment the compound is general formula IIc where R1 is sulfoalkyl, R5 and R6 are methyl and R2 is a PEG group P—Z, where P is an ethylene glycol group and Z is CH₃; R7 and R8 are H; R9 is sulfoalkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a diethylene glycol group and Z is CH₃; R7 and R8 are H; R9 is sulfoalkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are H; R9 is sulfoalkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are H; R9 is sulfoalkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are H; R9 is sulfoalkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are H; R9 is sulfoalkyl; R10 is alkyl terminating in —OH, —COOH, —NHS, —O-TFP, or —NR-L-maleimide; and n is 1. In one embodiment R9 is sulfopentyl or sulfopropyl, the alkyl of R10 is pentyl, and R1 is sulfopentyl or sulfopropyl.

In one embodiment the compound is general formula IIc where R1 is sulfoalkyl, R5 and R6 are methyl and R2 is a PEG group P—Z, where P is an ethylene glycol group and Z is CH₃; R7 and R8 are H; R9 is sulfoalkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a diethylene glycol group and Z is CH₃; R7 and R8 are H; R9 is sulfoalkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are H; R9 is sulfoalkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are H; R9 is sulfoalkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are H; R9 is sulfoalkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are H; R9 is sulfoalkyl; R10 is alkyl terminating in —OH, —COOH, —NHS, —O-TFP, or —NR-L-maleimide; and n is 2. In one embodiment R9 is sulfopentyl or sulfopropyl, the alkyl of R10 is pentyl, and R1 is sulfopentyl or sulfopropyl.

In one embodiment the compound is general formula IIc where R1 is sulfoalkyl, R5 and R6 are methyl and R2 is a PEG group P—Z, where P is an ethylene glycol group and Z is CH₃; R7 and R8 are H; R9 is sulfoalkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a diethylene glycol group and Z is CH₃; R7 and R8 are H; R9 is sulfoalkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ where s is 3, and Z is CH₃; R7 and R8 are H; R9 is sulfoalkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are H; R9 is sulfoalkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are H; R9 is sulfoalkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are H; R9 is sulfoalkyl; R10 is alkyl terminating in —OH, —COOH, —NHS, —O-TFP, or —NR-L-maleimide; and n is 3. In one embodiment R9 is sulfopentyl or sulfopropyl, the alkyl of R10 is pentyl, and R1 is sulfopentyl or sulfopropyl.

In one embodiment the compound is general formula IIc where R1 is sulfoalkyl, R5 and R6 are methyl and R2 is a PEG group P—Z, where P is an ethylene glycol group and Z is CH₃; R7 and R8 are SO₃; R9 is sulfoalkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a diethylene glycol group and Z is CH₃; R7 and R8 are SO₃; R9 is sulfoalkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are SO₃; R9 is sulfoalkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are SO₃; R9 is sulfoalkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are SO₃; R9 is sulfoalkyl; R10 is alkyl terminating in —OH, —COOH, —NHS, —O-TFP, or —NR-L-maleimide; and n is 1. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are SO₃; R9 is sulfoalkyl; R10 is alkyl terminating in —OH, —COOH, —NHS, —O-TFP, or —NR-L-maleimide; and n is 1. In one embodiment R9 is sulfopentyl or sulfopropyl, the alkyl of R10 is pentyl, and R1 is sulfopentyl or sulfopropyl.

In one embodiment the compound is general formula IIc where R1 is sulfoalkyl, R5 and R6 are methyl and R2 is a PEG group P—Z, where P is an ethylene glycol group and Z is CH₃; R7 and R8 are SO₃; R9 is sulfoalkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a diethylene glycol group and Z is CH₃; R7 and R8 are SO₃; R9 is sulfoalkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are SO₃; R9 is sulfoalkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are SO₃; R9 is sulfoalkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are SO₃; R9 is sulfoalkyl; R10 is alkyl terminating in —OH, —COOH, —NHS, —O-TFP, or —NR-L-maleimide; and n is 2. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are SO₃; R9 is sulfoalkyl; R10 is alkyl terminating in —OH, —COOH, —NHS, —O-TFP, or —NR-L-maleimide; and n is 2. In one embodiment R9 is sulfopentyl or sulfopropyl, the alkyl of R10 is pentyl, and R1 is sulfopentyl or sulfopropyl.

In one embodiment the compound is general formula IIc where R1 is sulfoalkyl, R5 and R6 are methyl and R2 is a PEG group P—Z, where P is an ethylene glycol group and Z is CH₃; R7 and R8 are SO₃; R9 is sulfoalkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a diethylene glycol group and Z is CH₃; R7 and R8 are SO₃; R9 is sulfoalkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 3, and Z is CH₃; R7 and R8 are SO₃; R9 is sulfoalkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 4, and Z is CH₃; R7 and R8 are SO₃; R9 is sulfoalkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 5, and Z is CH₃; R7 and R8 are SO₃; R9 is sulfoalkyl; R10 is alkyl terminating in —OH, —COOH, —NHS, —O-TFP, or —NR-L-maleimide; and n is 3. In one embodiment the compound is general formula IIc where R1 is sulfoalkyl, R5 and R6 are methyl and R2 is a PEG group P—Z, where P is a (poly)ethylene glycol group, where the (poly)ethylene glycol group is (CH₂CH₂O)ₛ, where s is 6, and Z is CH₃; R7 and R8 are SO₃; R9 is sulfoalkyl; R10 is alkyl terminating in —OH, —COOH, —NHS, —O-TFP, or —NR-L-maleimide; and n is 3. In one embodiment R9 is sulfopentyl or sulfopropyl, the alkyl of R10 is pentyl, and R1 is sulfopentyl or sulfopropyl.

In one embodiment the compound is general formula IIc where R1 and R2 are a PEG group P—Z, where P is an ethylene glycol group and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 1. In one embodiment the compound is general formula IIc where R1 and R2 are a PEG group P—Z, where P is a diethylene glycol group and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 1. In one embodiment the compound is general formula IIc where R1 and R2 are a PEG group P—Z, where P is a (poly)ethylene glycol group, where the (poly)ethylene glycol group is (CH₂CH₂O)ₛ, where s is 3, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 1. In one embodiment the compound is general formula IIc where R1 and R2 are a PEG group P—Z, where P is a (poly)ethylene glycol group, where the (poly)ethylene glycol group is (CH₂CH₂O)ₛ, where s is 4, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 1. In one embodiment the compound is general formula IIc where R1 and R2 are a PEG group P—Z, where P is a (poly)ethylene glycol group, where the (poly)ethylene glycol group is (CH₂CH₂O)ₛ, where s is 5, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 1. In one embodiment the compound is general formula IIc where R1 and R2 are a PEG group P—Z, where P is a (poly)ethylene glycol group, where the (poly)ethylene glycol group is (CH₂CH₂O)ₛ, where s is 6, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 1. In one embodiment R9 is sulfopentyl or sulfopropyl, and the alkyl of R10 is pentyl.

In one embodiment the compound is general formula IIc where R1 and R2 are a PEG group P—Z, where P is an ethylene glycol group and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 2. In one embodiment the compound is general formula IIc where R1 and R2 are a PEG group P—Z, where P is a diethylene glycol group and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 2. In one embodiment the compound is general formula IIc where R1 and R2 are a PEG group P—Z, where P is a (poly)ethylene glycol group, where the (poly)ethylene glycol group is (CH₂CH₂O)ₛ, where s is 3, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 2. In one embodiment the compound is general formula IIc where R1 and R2 are a PEG group P—Z, where P is a (poly)ethylene glycol group, where the (poly)ethylene glycol group is (CH₂CH₂O)ₛ, where s is 4, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 2. In one embodiment the compound is general formula IIc where R1 and R2 are a PEG group P—Z, where P is a (poly)ethylene glycol group, where the (poly)ethylene glycol group is (CH₂CH₂O)ₛ, where s is 5, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 2. In one embodiment the compound is general formula IIc where R1 and R2 are a PEG group P—Z, where P is a (poly)ethylene glycol group, where the (poly)ethylene glycol group is (CH₂CH₂O)ₛ, where s is 6, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 2. In one embodiment R9 is sulfopentyl or sulfopropyl, and the alkyl of R10 is pentyl.

In one embodiment the compound is general formula IIc where R1 and R2 are a PEG group P—Z, where P is an ethylene glycol group and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 3. In one embodiment the compound is general formula IIc where R1 and R2 are a PEG group P—Z, where P is a diethylene glycol group and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 3. In one embodiment the compound is general formula IIc where R1 and R2 are a PEG group P—Z, where P is a (poly)ethylene glycol group, where the (poly)ethylene glycol group is (CH₂CH₂O)ₛ, where s is 3, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 3. In one embodiment the compound is general formula IIc where R1 and R2 are a PEG group P—Z, where P is a (poly)ethylene glycol group, where the (poly)ethylene glycol group is (CH₂CH₂O)ₛ, where s is 4, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 3. In one embodiment the compound is general formula IIc where R1 and R2 are a PEG group P—Z, where P is a (poly)ethylene glycol group, where the (poly)ethylene glycol group is (CH₂CH₂O)ₛ, where s is 5, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 3. In one embodiment the compound is general formula IIc where R1 and R2 are a PEG group P—Z, where P is a (poly)ethylene glycol group, where the (poly)ethylene glycol group is (CH₂CH₂O)ₛ, where s is 6, and Z is CH₃; R5 and R6 are methyl; R7 and R8 are SO₃; R9 is sulfoalkyl; R10 is alkyl terminating in —OH, —COOH, —NHS, —O-TFP, or —NR-L-maleimide; and n is 3. In one embodiment R9 is sulfopentyl or sulfopropyl, and the alkyl of R10 is pentyl.

In one embodiment the compound is general formula IIc where R1 and R2 are a PEG group P—Z, where P is an ethylene glycol group and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 1. In one embodiment the compound is general formula IIc where R1 and R2 are a PEG group P—Z, where P is a diethylene glycol group and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 1. In one embodiment the compound is general formula IIc where R1 and R2 are a PEG group P—Z, where P is a (poly)ethylene glycol group, where the (poly)ethylene glycol group is (CH₂CH₂O)ₛ, where s is 3, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 1. In one embodiment the compound is general formula IIc where R1 and R2 are a PEG group P—Z, where P is a (poly)ethylene glycol group, where the (poly)ethylene glycol group is (CH₂CH₂O)ₛ, where s is 4, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 1. In one embodiment the compound is general formula IIc where R1 and R2 are a PEG group P—Z, where P is a (poly)ethylene glycol group, where the (poly)ethylene glycol group is (CH₂CH₂O)ₛ, where s is 5, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 1. In one embodiment the compound is general formula IIc where R1 and R2 are a PEG group P—Z, where P is a (poly)ethylene glycol group, where the (poly)ethylene glycol group is (CH₂CH₂O)ₛ, where s is 6, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 1. In one embodiment R9 is sulfopentyl or sulfopropyl, and the alkyl of R10 is pentyl.

In one embodiment the compound is general formula IIc where R1 and R2 are a PEG group P—Z, where P is an ethylene glycol group and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 2. In one embodiment the compound is general formula IIc where R1 and R2 are a PEG group P—Z, where P is a diethylene glycol group and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 2. In one embodiment the compound is general formula IIc where R1 and R2 are a PEG group P—Z, where P is a (poly)ethylene glycol group, where the (poly)ethylene glycol group is (CH₂CH₂O)ₛ, where s is 3, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 2. In one embodiment the compound is general formula IIc where R1 and R2 are a PEG group P—Z, where P is a (poly)ethylene glycol group, where the (poly)ethylene glycol group is (CH₂CH₂O)ₛ, where s is 4, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 2. In one embodiment the compound is general formula IIc where R1 and R2 are a PEG group P—Z, where P is a (poly)ethylene glycol group, where the (poly)ethylene glycol group is (CH₂CH₂O)ₛ, where s is 5, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 2. In one embodiment the compound is general formula IIc where R1 and R2 are a PEG group P—Z, where P is a (poly)ethylene glycol group, where the (poly)ethylene glycol group is (CH₂CH₂O)ₛ, where s is 6, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 2. In one embodiment R9 is sulfopentyl or sulfopropyl, and the alkyl of R10 is pentyl.

In one embodiment the compound is general formula IIc where R1 and R2 are a PEG group P—Z, where P is an ethylene glycol group and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 3. In one embodiment the compound is general formula IIc where R1 and R2 are a PEG group P—Z, where P is a diethylene glycol group and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 3. In one embodiment the compound is general formula IIc where R1 and R2 are a PEG group P—Z, where P is a (poly)ethylene glycol group, where the (poly)ethylene glycol group is (CH₂CH₂O)ₛ, where s is 3, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 3. In one embodiment the compound is general formula IIc where R1 and R2 are a PEG group P—Z, where P is a (poly)ethylene glycol group, where the (poly)ethylene glycol group is (CH₂CH₂O)ₛ, where s is 4, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 3. In one embodiment the compound is general formula IIc where R1 and R2 are a PEG group P—Z, where P is a (poly)ethylene glycol group, where the (poly)ethylene glycol group is (CH₂CH₂O)ₛ, where s is 5, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 3. In one embodiment the compound is general formula IIc where R1 and R2 are a PEG group P—Z, where P is a (poly)ethylene glycol group, where the (poly)ethylene glycol group is (CH₂CH₂O)ₛ, where s is 6, and Z is CH₃; R5 and R6 are methyl; R7 and R8 are H; R9 is sulfoalkyl; R10 is alkyl terminating in —OH, —COOH, —NHS, —O-TFP, or —NR-L-maleimide; and n is 3. In one embodiment R9 is sulfopentyl or sulfopropyl, and the alkyl of R10 is pentyl.

In one embodiment the compound has general formula IIIa with “a” indicating an ethylene or (poly)ethylene glycol at both left N and right N and the right N chain that terminates in COX:

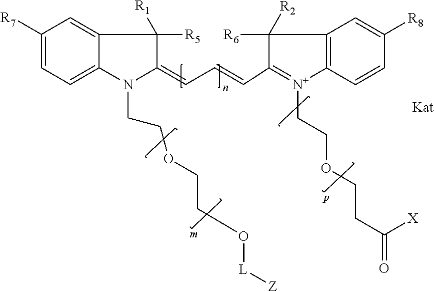

general formula IIIb with “b” indicating an ethylene or (poly)ethylene glycol at both left N and right N and the right N chain that terminates in COH:

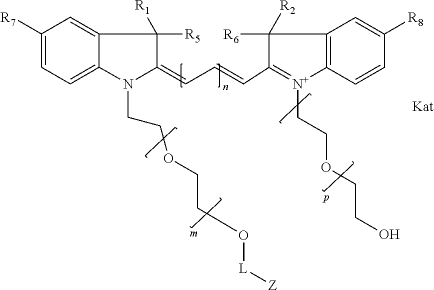

general formula IIIc with “c” indicating an ethylene or (poly)ethylene glycol at only the right N and the right N chain that terminates in COX:

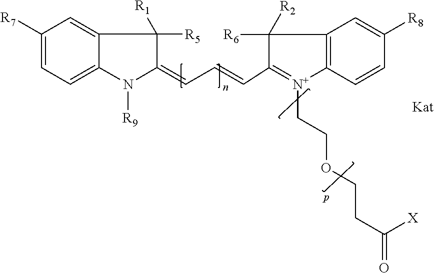

or general formula IIId with “d” indicating an ethylene or (poly)ethylene glycol at only the right N and the right N chain that terminates in COH:

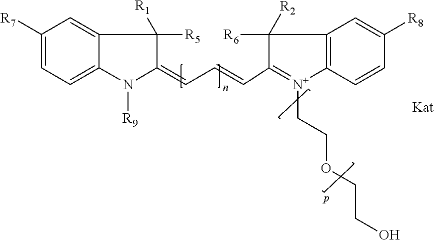

where each R¹, R², R⁵, R⁶, R⁷, and R⁸ is the same or different and is independently selected from the group consisting of an aliphatic, heteroaliphatic, sulfoalkyl group, heteroaliphatic with terminal SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group -L-SO₂NH—P-L-Z, and a caboxamide group -L-CONH—P-L-Z; each of R⁷ and R⁸ is the same or different and is independently selected from the group consisting of H, SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group —SO₂NH—P-L-Z, and a caboxamide group —CONH—P-L-Z; R⁹ is selected from the group consisting of alkyl, sulfoalkyl, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a PEG group P-L-X, L-Z, L-X; where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur; where Z is selected from the group consisting of H, CH₃, alkyl, heteroalkyl, NH₂, —COO⁻, —COOH, —COSH, CO—NH—NH₂, —COF, —COCl, —COBr, —COI, —COO-Su (succinimidyl/sulfosuccinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-O-phosphoramidite, —CONR′-L-CHO, —CONR′-L-maleimide, and —CONR′-L-NH—CO—CH₂—I; R′ and R″ is selected from the group consisting of H, aliphatic group, and heteroaliphatic group, and the biomolecule is a protein, antibody, nucleotide, oligonucleotide, biotin, or hapten; X is selected from the group consisting of —OH, —SH, —NH₂, —NH—NH₂, —F, —Cl, —Br, I, —NHS (hydroxysuccinimidyl/sulfo-succinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group; Kat is a number of Na⁺, K⁺, Ca²⁺, ammonia, or other cation(s) needed to compensate the negative charge brought by the cyanine; m is an integer from 0 to 5 inclusive; p is an integer from 1 to 6 inclusive; and n is an integer from 1 to 3 inclusive; and at least one of R¹, R², R⁵, R⁶, R⁷, and R⁸ contains a PEG group.

In one embodiment the compound is general formula III, where R1, R5, and R6 are methyl; R2 is a PEG group; R7 and R8 are sulfo; X is —OH, —NHS, —O-TFP, or —NR-L-maleimide; m is 0; p is 1; and n is 1. In one embodiment the compound is general formula III, where R1, R5, and R6 are methyl; R2 is a PEG group; R7 and R8 are sulfo; X is —OH, —NHS, —O-TFP, or —NR-L-maleimide; m is 1; p is 2; and n is 1. In one embodiment the compound is general formula III, where R1, R5, and R6 are methyl; R2 is a PEG group; R7 and R8 are sulfo; X is —OH, —NHS, —O-TFP, or —NR-L-maleimide; m is 2; p is 3; and n is 1. In one embodiment the compound is general formula III, where R1, R5, and R6 are methyl; R2 is a PEG group; R7 and R8 are sulfo; X is —OH, —NHS, —O-TFP, or —NR-L-maleimide; m is 3; p is 4; and n is 1. In one embodiment the compound is general formula III, where R1, R5, and R6 are methyl; R2 is a PEG group; R7 and R8 are sulfo; X is —OH, —NHS, —O-TFP, or —NR-L-maleimide; m is 4; p is 5; and n is 1. In one embodiment the compound is general formula III, where R1, R5, and R6 are methyl; R2 is a PEG group; R7 and R8 are sulfo; X is —OH, —NHS, —O-TFP, or —NR-L-maleimide; m is 5; p is 6; and n is 1.

In one embodiment the compound is general formula III, where R1, R5, and R6 are methyl; R2 is a PEG group; R7 and R8 are sulfo; X is —OH, —NHS, —O-TFP, or —NR-L-maleimide; m is 0; p is 1; and n is 2. In one embodiment the compound is general formula III, where R1, R5, and R6 are methyl; R2 is a PEG group; R7 and R8 are sulfo; X is —OH, —NHS, —O-TFP, or —NR-L-maleimide; m is 1; p is 2; and n is 2. In one embodiment the compound is general formula III, where R1, R5, and R6 are methyl; R2 is a PEG group; R7 and R8 are sulfo; X is —OH, —NHS, —O-TFP, or —NR-L-maleimide; m is 2; p is 3; and n is 2. In one embodiment the compound is general formula III, where R1, R5, and R6 are methyl; R2 is a PEG group; R7 and R8 are sulfo; X is —OH, —NHS, —O-TFP, or —NR-L-maleimide; m is 3; p is 4; and n is 2. In one embodiment the compound is general formula III, where R1, R5, and R6 are methyl; R2 is a PEG group; R7 and R8 are sulfo; X is —OH, —NHS, —O-TFP, or —NR-L-maleimide; m is 4; p is 5; and n is 2. In one embodiment the compound is general formula III, where R1, R5, and R6 are methyl; R2 is a PEG group; R7 and R8 are sulfo; X is —OH, —NHS, —O-TFP, or —NR-L-maleimide; m is 5; p is 6; and n is 2.

In one embodiment the compound is general formula III, where R1, R5, and R6 are methyl; R2 is a PEG group; R7 and R8 are sulfo; X is —OH, —NHS, —O-TFP, or —NR-L-maleimide; m is 0; p is 1; and n is 3. In one embodiment the compound is general formula III, where R1, R5, and R6 are methyl; R2 is a PEG group; R7 and R8 are sulfo; X is —OH, —NHS, —O-TFP, or —NR-L-maleimide; m is 1; p is 2; and n is 3. In one embodiment the compound is general formula III, where R1, R5, and R6 are methyl; R2 is a PEG group; R7 and R8 are sulfo; X is —OH, —NHS, —O-TFP, or —NR-L-maleimide; m is 2; p is 3; and n is 3. In one embodiment the compound is general formula III, where R1, R5, and R6 are methyl; R2 is a PEG group; R7 and R8 are sulfo; X is —OH, —NHS, —O-TFP, or —NR-L-maleimide; m is 3; p is 4; and n is 3. In one embodiment the compound is general formula III, where R1, R5, and R6 are methyl; R2 is a PEG group; R7 and R8 are sulfo; X is —OH, —NHS, —O-TFP, or —NR-L-maleimide; m is 4; p is 5; and n is 3. In one embodiment the compound is general formula III, where R1, R5, and R6 are methyl; R2 is a PEG group; R7 and R8 are sulfo; X is —OH, —NHS, —O-TFP, or —NR-L-maleimide; m is 5; p is 6; and n is 3.

In one embodiment the compound is general formula III where R5 and R6 are methyl; R1 and R2 are a PEG group; R7 and R8 are sulfo; X is —OH, —NHS, —O-TFP, or —NR-L-maleimide; m is 0; p is 1; and n is 1. In one embodiment the compound is general formula III where R5 and R6 are methyl; R1 and R2 are a PEG group; R7 and R8 are sulfo; X is —OH, —NHS, —O-TFP, or —NR-L-maleimide; m is 1; p is 2; and n is 1. In one embodiment the compound is general formula III where R5 and R6 are methyl; R1 and R2 are a PEG group; R7 and R8 are sulfo; X is —OH, —NHS, —O-TFP, or —NR-L-maleimide; m is 2; p is 3; and n is 1. In one embodiment the compound is general formula III where R5 and R6 are methyl; R1 and R2 are a PEG group; R7 and R8 are sulfo; X is —OH, —NHS, —O-TFP, or —NR-L-maleimide; m is 3; p is 4; and n is 1. In one embodiment the compound is general formula III where R5 and R6 are methyl; R1 and R2 are a PEG group; R7 and R8 are sulfo; X is —OH, —NHS, —O-TFP, or —NR-L-maleimide; m is 4; p is 5; n is 1. In one embodiment the compound is general formula III where R5 and R6 are methyl; R1 and R2 are a PEG group; R7 and R8 are sulfo; X is —OH, —NHS, —O-TFP, or —NR-L-maleimide; m is 5; p is 6; n is 1.

In one embodiment the compound is general formula III, where R5 and R6 are methyl; R1 and R2 are a PEG group; R7 and R8 are sulfo; X is —OH, —NHS, —O-TFP, or —NR-L-maleimide; m is 0; p is 1; and n is 2. In one embodiment the compound is general formula III, where R5 and R6 are methyl; R1 and R2 are a PEG group; R7 and R8 are sulfo; X is —OH, —NHS, —O-TFP, or —NR-L-maleimide; m is 1; p is 2; and n is 2. In one embodiment the compound is general formula III, where R5 and R6 are methyl; R1 and R2 are a PEG group; R7 and R8 are sulfo; X is —OH, —NHS, —O-TFP, or —NR-L-maleimide; m is 2; p is 3; and n is 2. In one embodiment the compound is general formula III, where R5 and R6 are methyl; R1 and R2 are a PEG group; R7 and R8 are sulfo; X is —OH, —NHS, —O-TFP, or —NR-L-maleimide; m is 3; p is 4; and n is 2. In one embodiment the compound is general formula III, where R5 and R6 are methyl; R1 and R2 are a PEG group; R7 and R8 are sulfo; X is —OH, —NHS, —O-TFP, or —NR-L-maleimide; m is 4; p is 5; and n is 2. In one embodiment the compound is general formula III, where R5 and R6 are methyl; R1 and R2 are a PEG group; R7 and R8 are sulfo; X is —OH, —NHS, —O-TFP, or —NR-L-maleimide; m is 5; p is 6; and n is 2.

In one embodiment the compound is general formula III, where R5 and R6 are methyl; R1 and R2 are a PEG group; R7 and R8 are sulfo; X is —OH, —NHS, —O-TFP, or —NR-L-maleimide; m is 0; p is 1; and n is 3. In one embodiment the compound is general formula III, where R5 and R6 are methyl; R1 and R2 are a PEG group; R7 and R8 are sulfo; X is —OH, —NHS, —O-TFP, or —NR-L-maleimide; m is 1; p is 2; and n is 3. In one embodiment the compound is general formula III, where R5 and R6 are methyl; R1 and R2 are a PEG group; R7 and R8 are sulfo; X is —OH, —NHS, —O-TFP, or —NR-L-maleimide; m is 2; p is 3; and n is 3. In one embodiment the compound is general formula III, where R5 and R6 are methyl; R1 and R2 are a PEG group; R7 and R8 are sulfo; X is —OH, —NHS, —O-TFP, or —NR-L-maleimide; m is 3; p is 4; and n is 3. In one embodiment the compound is general formula III, where R5 and R6 are methyl; R1 and R2 are a PEG group; R7 and R8 are sulfo; X is —OH, —NHS, —O-TFP, or —NR-L-maleimide; m is 4; p is 5; and n is 3. In one embodiment the compound is general formula III, where R5 and R6 are methyl; R1 and R2 are a PEG group; R7 and R8 are sulfo; X is —OH, —NHS, —O-TFP, or —NR-L-maleimide; m is 5; p is 6; and n is 3.

In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 0; p is 1; and n is 1. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 1; p is 2; and n is 1. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 2; p is 3; and n is 1. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 3; p is 4; and n is 1. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 4; p is 5; and n is 1. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 5; p is 6; and n is 1.

In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 0; p is 1; and n is 2. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 1; p is 2; and n is 2. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 2; p is 3; and n is 2. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 3; p is 4; and n is 2. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 4; p is 5; and n is 2. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 5; p is 6; and n is 2.

In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 0; p is 1; and n is 3. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 1; p is 2; and n is 3. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 2; p is 3; and n is 3. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 3; p is 4; and n is 3. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 4; p is 5; and n is 3. In one embodiment the compound is general formula III, where R5 and R6 are methyl; R1 and R2 are a PEG group; R7 is sulfo; R8 is a sulfonamide group —SO₂NH—P where P is a PEG group; X is —OH, —NHS, —O-TFP, or —NR-L-maleimide; m is 5; p is 6; and n is 3.

In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 0; p is 1; and n is 1. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 1; p is 2; and n is 1. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 2; p is 3; and n is 1. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 3; p is 4; and n is 1. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 4; p is 5; and n is 1. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 5; p is 6; and n is 1.

In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 0; p is 1; and n is 2. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 1; p is 2; and n is 2. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 2; p is 3; and n is 2. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 3; p is 4; and n is 2. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 4; p is 5; and n is 2. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 5; p is 6; and n is 2.

In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 0; p is 1; and n is 3. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 1; p is 2; and n is 3. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 2; p is 3; and n is 3. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 3; p is 4; and n is 3. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 4; p is 5; and n is 3. In one embodiment the compound is general formula III, where R5 and R6 are methyl; R1 and R2 are a PEG group; R7 is sulfo; R8 is a caboxamide group —CONH—P where P is a PEG group; X is —OH, —NHS, —O-TFP, or —NR-L-maleimide; m is 5; p is 6; and n is 3.

In one embodiment the compound is general formula IIIc or IIId where R1 and R2 are a PEG group P—Z, where P is an ethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 1; and n is 1. In one embodiment the compound is general formula IIIc or IIId where R1 and R2 are a PEG group P—Z, where P is a diethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 2; and n is 1. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 3, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 3; and n is 1. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 4, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 4; and n is 1. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 5, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 5; and n is 1. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 6, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 6; and n is 1. In one embodiment R9 is ethyl, sulfopentyl or sulfopropyl.

In one embodiment the compound is general formula IIIc or IIId where R1 and R2 are a PEG group P—Z, where P is an ethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 1; and n is 2. In one embodiment the compound is general formula IIIc or IIId where R1 and R2 are a PEG group P—Z, where P is a diethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 2; and n is 2. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 3, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 3; and n is 2. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 4, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 4; and n is 2. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 5, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 5; and n is 2. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 6, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 6; and n is 2. In one embodiment R9 is ethyl, sulfopentyl or sulfopropyl.

In one embodiment the compound is general formula IIIc or IIId where R1 and R2 are a PEG group P—Z, where P is an ethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 1; and n is 3. In one embodiment the compound is general formula IIIc or IIId where R1 and R2 are a PEG group P—Z, where P is a diethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 2; and n is 3. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 3, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 3; and n is 3. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 4, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 4; and n is 3. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 5, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 5; and n is 3. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 6, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 6; and n is 3. In one embodiment R9 is ethyl, sulfopentyl or sulfopropyl.

In one embodiment the compound is general formula IIIc or IIId where R1 and R2 are a PEG group P—Z, where P is an ethylene glycol group and Z is CH₃; R5 and R6 are methyl; R7 and R8 are H; R9 is alkyl or sulfoalkyl; X is —OH, —COOH, —NHS, —O-TFP, or —NR-L-maleimide; p is 1; and n is 1. In one embodiment the compound is general formula IIIc or IIId where R1 and R2 are a PEG group P—Z, where P is a diethylene glycol group and Z is CH₃; R5 and R6 are methyl; R7 and R8 are H; R9 is alkyl or sulfoalkyl; X is —OH, —COOH, —NHS, —O-TFP, or —NR-L-maleimide; p is 2; and n is 1. In one embodiment the compound is general formula IIIc or IIId where R1 and R2 are a PEG group P—Z, where P is a (poly)ethylene glycol group, where the (poly)ethylene glycol group is (CH₂CH₂O)ₛ where s is 3, and Z is CH₃; R5 and R6 are methyl; R7 and R8 are H; R9 is alkyl or sulfoalkyl; X is —OH, —COOH, —NHS, —O-TFP, or —NR-L-maleimide; p is 3; and n is 1. In one embodiment the compound is general formula IIIc or IIId where R1 and R2 are a PEG group P—Z, where P is a (poly)ethylene glycol group, where the (poly)ethylene glycol group is (CH₂CH₂O)ₛ where s is 4, and Z is CH₃; R5 and R6 are methyl; R7 and R8 are H; R9 is alkyl or sulfoalkyl; X is —OH, —COOH, —NHS, —O-TFP, or —NR-L-maleimide; p is 4; and n is 1. In one embodiment the compound is general formula IIIc or IIId where R1 and R2 are a PEG group P—Z, where P is a (poly)ethylene glycol group, where the (poly)ethylene glycol group is (CH₂CH₂O)ₛ, where s is 5, and Z is CH₃; R5 and R6 are methyl; R7 and R8 are H; R9 is alkyl or sulfoalkyl; X is —OH, —COOH, —NHS, —O-TFP, or —NR-L-maleimide; p is 5; and n is 1. In one embodiment the compound is general formula IIIc or IIId where R1 and R2 are a PEG group P—Z, where P is a (poly)ethylene glycol group, where the (poly)ethylene glycol group is (CH₂CH₂O)ₛ where s is 6, and Z is CH₃; R5 and R6 are methyl; R7 and R8 are H; R9 is alkyl or sulfoalkyl; X is —OH, —COOH, —NHS, —O-TFP, or —NR-L-maleimide; p is 6; and n is 1. In one embodiment R9 is ethyl, sulfopentyl or sulfopropyl.

In one embodiment the compound is general formula IIIc or IIId where R1 and R2 are a PEG group P—Z, where P is an ethylene glycol group and Z is CH₃; R5 and R6 are methyl; R7 and R8 are H; R9 is alkyl or sulfoalkyl; X is —OH, —COOH, —NHS, —O-TFP, or —NR-L-maleimide; p is 1; and n is 2. In one embodiment the compound is general formula IIIc or IIId where R1 and R2 are a PEG group P—Z, where P is a diethylene glycol group and Z is CH₃; R5 and R6 are methyl; R7 and R8 are H; R9 is alkyl or sulfoalkyl; X is —OH, —COOH, —NHS, —O-TFP, or —NR-L-maleimide; p is 2; and n is 2. In one embodiment the compound is general formula IIIc or IIId where R1 and R2 are a PEG group P—Z, where P is a (poly)ethylene glycol group, where the (poly)ethylene glycol group is (CH₂CH₂O)ₛ where s is 3, and Z is CH₃; R5 and R6 are methyl; R7 and R8 are H; R9 is alkyl or sulfoalkyl; X is —OH, —COOH, —NHS, —O-TFP, or —NR-L-maleimide; p is 3; and n is 2. In one embodiment the compound is general formula IIIc or IIId where R1 and R2 are a PEG group P—Z, where P is a (poly)ethylene glycol group, where the (poly)ethylene glycol group is (CH₂CH₂O)ₛ where s is 4, and Z is CH₃; R5 and R6 are methyl; R7 and R8 are H; R9 is alkyl or sulfoalkyl; X is —OH, —COOH, —NHS, —O-TFP, or —NR-L-maleimide; p is 4; and n is 2. In one embodiment the compound is general formula IIIc or IIId where R1 and R2 are a PEG group P—Z, where P is a (poly)ethylene glycol group, where the (poly)ethylene glycol group is (CH₂CH₂O)ₛ where s is 5, and Z is CH₃; R5 and R6 are methyl; R7 and R8 are H; R9 is alkyl or sulfoalkyl; X is —OH, —COOH, —NHS, —O-TFP, or —NR-L-maleimide; p is 5; and n is 2. In one embodiment the compound is general formula IIIc or IIId where R1 and R2 are a PEG group P—Z, where P is a (poly)ethylene glycol group, where the (poly)ethylene glycol group is (CH₂CH₂O)ₛ where s is 6, and Z is CH₃; R5 and R6 are methyl; R7 and R8 are H; R9 is alkyl or sulfoalkyl; X is —OH, —COOH, —NHS, —O-TFP, or —NR-L-maleimide; p is 6; and n is 2. In one embodiment R9 is ethyl, sulfopentyl or sulfopropyl.

In one embodiment the compound is general formula IIIc or IIId where R1 and R2 are a PEG group P—Z, where P is an ethylene glycol group and Z is CH₃; R5 and R6 are methyl; R7 and R8 are H; R9 is alkyl or sulfoalkyl; X is —OH, —COOH, —NHS, —O-TFP, or —NR-L-maleimide; p is 1; and n is 3. In one embodiment the compound is general formula IIIc or IIId where R1 and R2 are a PEG group P—Z, where P is a diethylene glycol group and Z is CH₃; R5 and R6 are methyl; R7 and R8 are H; R9 is alkyl or sulfoalkyl; X is —OH, —COOH, —NHS, —O-TFP, or —NR-L-maleimide; p is 2; and n is 3. In one embodiment the compound is general formula IIIc or IIId where R1 and R2 are a PEG group P—Z, where P is a (poly)ethylene glycol group, where the (poly)ethylene glycol group is (CH₂CH₂O)ₛ where s is 3, and Z is CH₃; R5 and R6 are methyl; R7 and R8 are H; R9 is alkyl or sulfoalkyl; X is —OH, —COOH, —NHS, —O-TFP, or —NR-L-maleimide; p is 3; and n is 3. In one embodiment the compound is general formula IIIc or IIId where R1 and R2 are a PEG group P—Z, where P is a (poly)ethylene glycol group, where the (poly)ethylene glycol group is (CH₂CH₂O)ₛ where s is 4, and Z is CH₃; R5 and R6 are methyl; R7 and R8 are H; R9 is alkyl or sulfoalkyl; X is —OH, —COOH, —NHS, —O-TFP, or —NR-L-maleimide; p is 4; and n is 3. In one embodiment the compound is general formula IIIc or IIId where R1 and R2 are a PEG group P—Z, where P is a (poly)ethylene glycol group, where the (poly)ethylene glycol group is (CH₂CH₂O)ₛ where s is 5, and Z is CH₃; R5 and R6 are methyl; R7 and R8 are H; R9 is alkyl or sulfoalkyl; X is —OH, —COOH, —NHS, —O-TFP, or —NR-L-maleimide; p is 5; and n is 3. In one embodiment the compound is general formula IIIc or IIId where R1 and R2 are a PEG group P—Z, where P is a (poly)ethylene glycol group, where the (poly)ethylene glycol group is (CH₂CH₂O)ₛ where s is 6, and Z is CH₃; R5 and R6 are methyl; R7 and R8 are H; R9 is alkyl or sulfoalkyl; X is —OH, —COOH, —NHS, —O-TFP, or —NR-L-maleimide; p is 6; and n is 3. In one embodiment R9 is ethyl, sulfopentyl or sulfopropyl.

In one embodiment the compound is general formula IIIc or IIId where R1 is alkyl or sulfoalkyl; R2 is a PEG group P—Z, where P is an ethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 1; and n is 1. In one embodiment the compound is general formula IIIc or IIId where R1 is alkyl or sulfoalkyl; R2 is a PEG group P—Z, where P is a diethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 2; and n is 1. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ, where s is 3 and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 3; and n is 1. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ, where s is 4, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 4; and n is 1. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ where s is 5, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 5; and n is 1. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ where s is 6, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 6; and n is 1. In one embodiment each of R1 and R9 are independently selected from methyl, ethyl, sulfopentyl or sulfopropyl.

In one embodiment the compound is general formula IIIc or IIId where R1 is alkyl or sulfoalkyl; R2 is a PEG group P—Z, where P is an ethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 1; and n is 2. In one embodiment the compound is general formula IIIc or IIId where R1 is alkyl or sulfoalkyl; R2 is a PEG group P—Z, where P is a diethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 2; and n is 2. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ where s is 3, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 3; and n is 2. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ where s is 4, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 4; and n is 2. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ where s is 5, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 5; and n is 2. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ, where s is 6, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 6; and n is 2. In one embodiment each of R1 and R9 are independently selected from methyl, ethyl, sulfopentyl or sulfopropyl.

In one embodiment the compound is general formula IIIc or IIId where R1 is alkyl or sulfoalkyl; R2 is a PEG group P—Z, where P is an ethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 1; and n is 3. In one embodiment the compound is general formula IIIc or IIId where R1 is alkyl or sulfoalkyl; R2 is a PEG group P—Z, where P is a diethylene glycol group and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 2; and n is 3. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ, where s is 3, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 3; and n is 3. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ where s is 4, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 4; and n is 3. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ where s is 5, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 5; and n is 3. In one embodiment the compound is general formula IIIc or IIId where R1 is alkyl or sulfoalkyl; R2 is a PEG group P—Z, where P is a (poly)ethylene glycol group, where the (poly)ethylene glycol group is (CH₂CH₂O)ₛ where s is 6, and Z is CH₃; R5 and R6 are methyl; R7 and R8 are SO₃; R9 is alkyl or sulfoalkyl; X is —OH, —COOH, —NHS, —O-TFP, or —NR-L-maleimide; p is 6; and n is 3. In one embodiment each of R1 and R9 are independently selected from methyl, ethyl, sulfopentyl or sulfopropyl.

In one embodiment the compound is 550 Compound 1/2

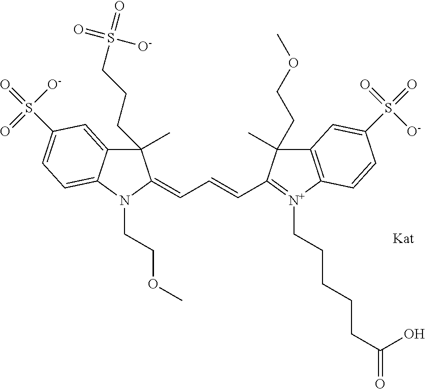 [SMILES: COCCN1/C(=C/C=C/C2=[N+](CCCCCC(=O)O)c3ccc(S(=O)(=O)[O-])cc3C2(C)CCOC)C(C)(CCCS(=O)(=O)[O-])c2cc(S(=O)(=O)[O-])ccc21]

550 Compound 1/2 (1-(5-carboxypentyl)-3-(2-methoxyethyl)-2-((1E,3E)-3-(1-(2-methoxyethyl)-3-methyl-5-sulfonato-3-(3-sulfonatopropyl)indolin-2-ylidene)prop-1-enyl)-3-methyl-3H-indolium-5-sulfonate) contains an ethylene glycol on the indole N of the left heterocycle, i.e., methylated ethylene glycol, as shown in the structure above, and the ethylene glycol can be represented in abbreviated format as —[C—C—O]₁—, which is used throughout. The methyl group on the ethylene glycol prevents the terminal —OH from oxidation. Oxidation is known to occur, over time, on an unprotected terminus of an ethylene glycol group, diethylene glycol group, or (poly)ethylene glycol group, collectively referred to herein as an unprotected PEG terminus. Adding a methyl ether provides this protection, and prevents reaction with electrophilic reactive groups.

In embodiments, e.g., for functional assays, the inventive compounds are activated. Activation of the compound adds a chemical moiety such that the compound is in a form that can be conjugated to a biological moiety. Examples of chemical moieties for activation are described below with reference to activation of 550 Compound 1, but one skilled in the art appreciates that activation is not limited to these examples. One non-limiting example of an activated compound is the NHS-ester of 550 Compound 1/2, shown below:

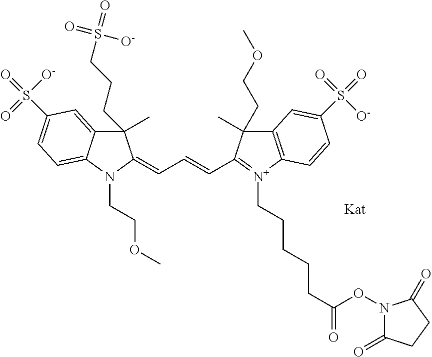 [SMILES: COCCN1/C(=C/C=C/C2=[N+](CCCCCC(=O)ON3C(=O)CCC3=O)c3ccc(S(=O)(=O)[O-])cc3C2(C)CCOC)C(C)(CCCS(=O)(=O)[O-])c2cc(S(=O)(=O)[O-])ccc21]

In one embodiment the compound is a NHS-ester of 550 Compound 1/2 where, according to general formula I, o is 1, shown below:

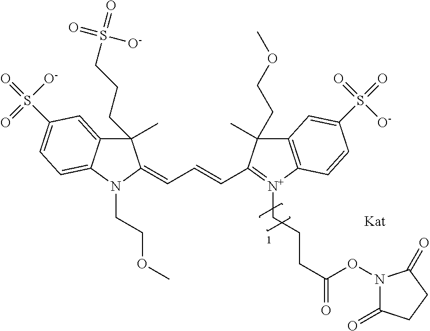

In one embodiment the compound is an NHS-ester of 550 Compound 1/2 where, according to general formula I, o is 5, shown below:

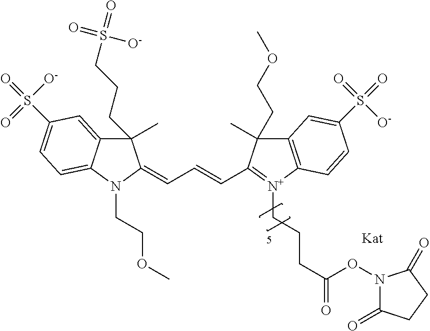

One non-limiting example of a NHS-ester of 550 Compound 1/3, according to general formula III, where m=1 and p=1, is shown below:

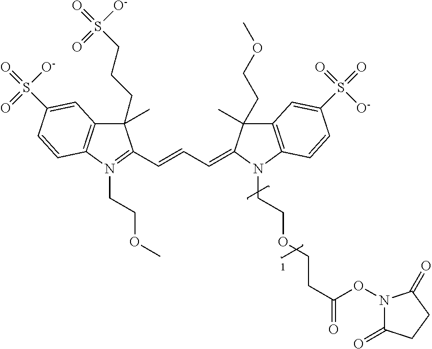

One non-limiting example of a NHS-ester of 550 Compound 1/3, according to general formula III, where m=1 and p=2, is shown below:

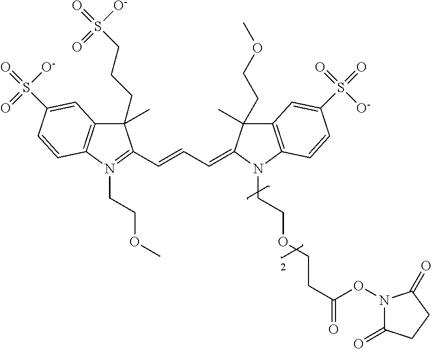

One non-limiting example of a NHS-ester of 550 Compound 1/3, according to general formula III, where m=1 and p=3, is shown below:

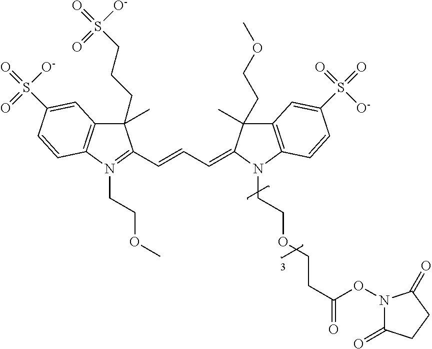

One non-limiting example of a NHS-ester of 550 Compound 1/3, according to general formula III, where m=1 and p=4, is shown below:

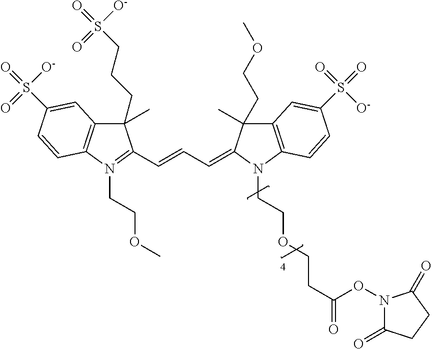

One non-limiting example of a NHS-ester of 550 Compound 1/3, according to general formula III, where m=1 and p=5, is shown below:

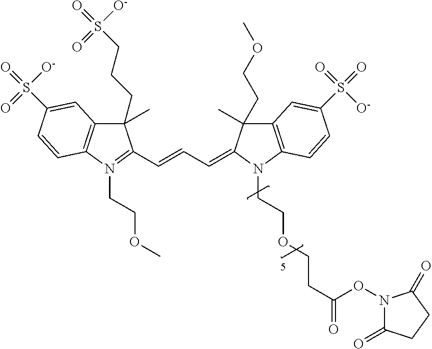

One non-limiting example of a NHS-ester of 550 Compound 1/3, according to general formula III, where m=1 and p=6, is shown below:

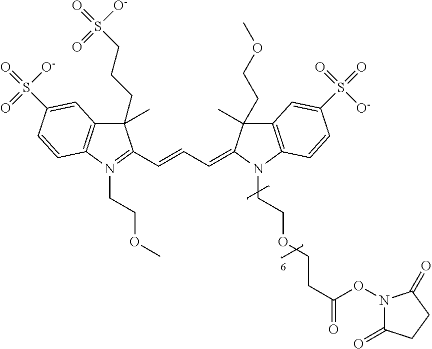

One non-limiting example of a NHS-ester of 550 Compound 2/3, according to general formula III, where m=2 and p=1, is shown below:

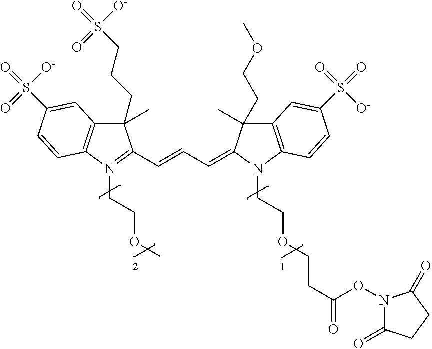

One non-limiting example of a NHS-ester of 550 Compound 2/3, according to general formula III, where m=2 and p=2, is shown below:

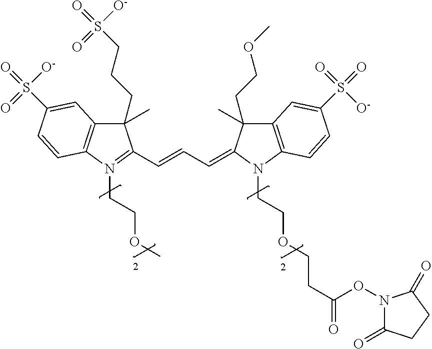

One non-limiting example of a NHS-ester of 550 Compound 2/3, according to general formula III, where m=2 and p=3, is shown below:

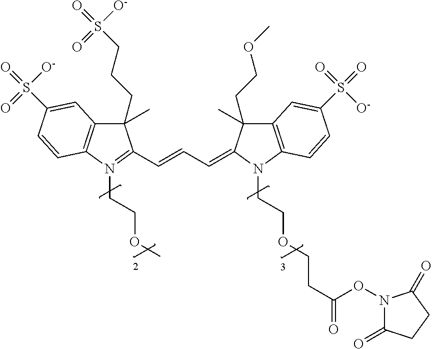

One non-limiting example of a NHS-ester of 550 Compound 3/3, according to general formula III, where m=3 and p=1, is shown below:

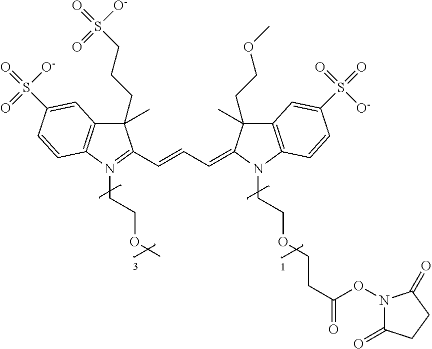

One non-limiting example of a NHS-ester of 550 Compound 3/3, according to general formula III, where m=3 and p=2, is shown below:

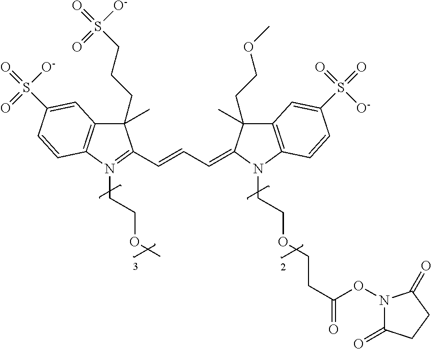

One non-limiting example of a NHS-ester of 550 Compound 3/3, according to general formula III, where m=3 and p=3, is shown below:

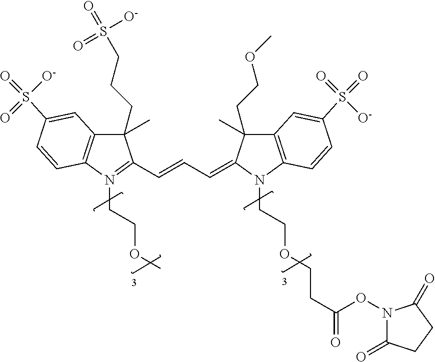

One non-limiting example of a NHS-ester of 550 Compound 4/3, according to general formula III, where m=4 and p=1, is shown below:

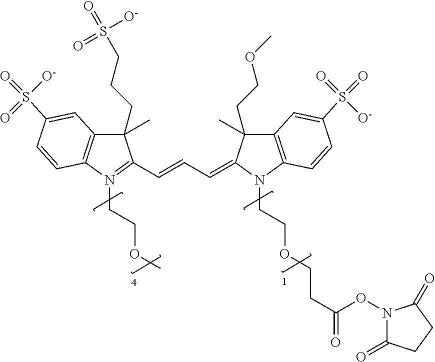

One non-limiting example of a NHS-ester of 550 Compound 5/3, according to general formula III, where m=5 and p=1, is shown below:

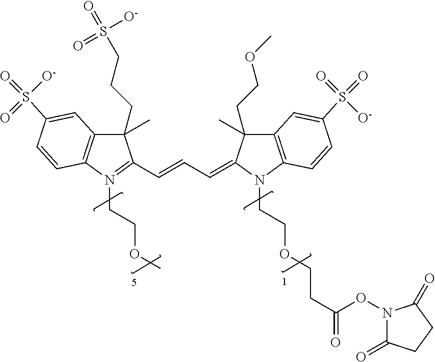

One non-limiting example of a NHS-ester of 550 Compound 6/3, according to general formula III, where m=6 and p=1, is shown below:

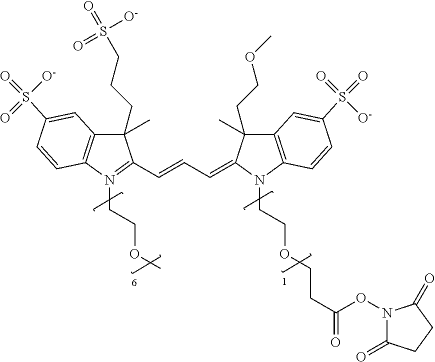

One non-limiting example of an activated 550 Compound 1/2 is a tetrafluorophenyl (TFP)-ester form of 550 Compound 1, shown below:

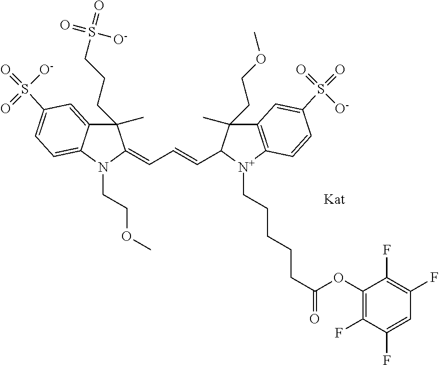 [SMILES: COCCN1/C(=C/C=C/C2[NH+](CCCCCC(=O)Oc3c(F)c(F)cc(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 550 Compound 1/2 is a sulfotetrafluorophenyl (STP)-ester form of 550 Compound 1, shown below:

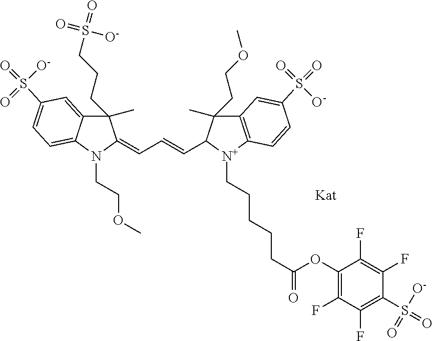 [SMILES: COCCN1/C(=C/C=C/C2[NH+](CCCCCC(=O)Oc3c(F)c(F)c(S(=O)(=O)[O-])c(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 550 Compound 1/2 is a hydrazide form of 550 Compound 1, shown below:

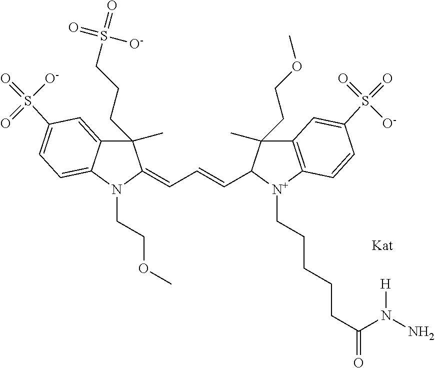 [SMILES: COCCN1/C(=C/C=C/C2[NH+](CCCCCC(=O)NN)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 550 Compound 1/2 is a maleimide form of 550 Compound 1, shown below:

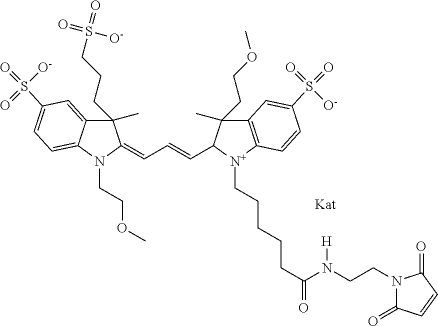 [SMILES: COCCN1/C(=C/C=C/C2[NH+](CCCCCC(=O)NCCN3C(=O)C=CC3=O)c3ccc(S(=O)(=O)[O-])cc3C2(C)CCOC)C(C)(CCCS(=O)(=O)[O-])c2cc(S(=O)(=O)[O-])ccc21]

In one embodiment the compound is 550 Compound 2/2

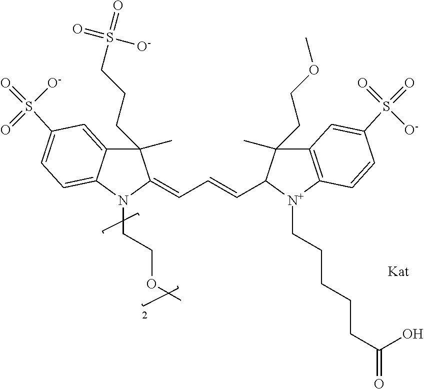

550 Compound 2/2 (1-(5-carboxypentyl)-2-((1E,3E)-3-(1-(2-(2-methoxyethoxy)ethyl)-3-methyl-5-sulfonato-3-(3-sulfonatopropyl)indolin-2-ylidene)prop-1-enyl)-3-(2-methoxyethyl)-3-methyl-3H-indolium-5-sulfonate) contains a diethylene glycol on the indole N of the left heterocycle. 550 Compound 2/2, with the diethylene glycol shown in abbreviated notation used throughout, represents the following structure.

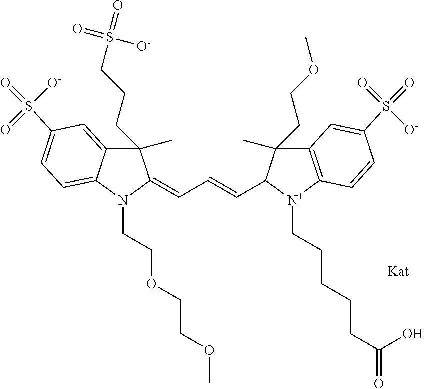 [SMILES: COCCOCCN1/C(=C/C=C/C2[NH+](CCCCCC(=O)O)c3ccc(S(=O)(=O)[O-])cc3C2(C)CCOC)C(C)(CCCS(=O)(=O)[O-])c2cc(S(=O)(=O)[O-])ccc21]

The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 550 Compound 2/2 is activated as described above, one non-limiting example of which is the NHS-ester form of 550 Compound 2/2, shown below.

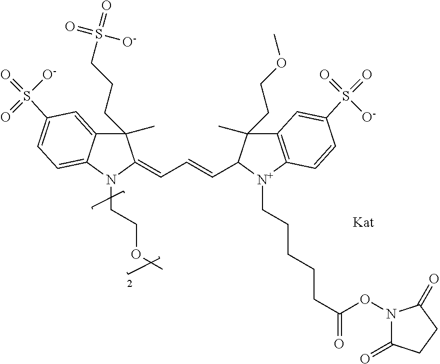

In one embodiment the compound is 550 Compound 3/2

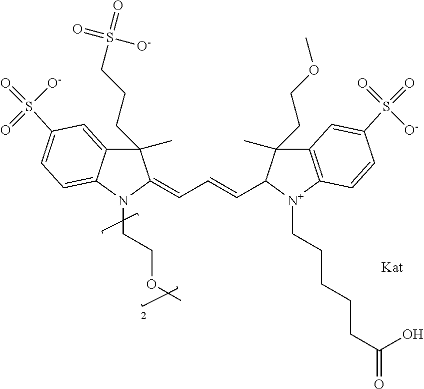

550 Compound 3/2 (1-(5-carboxypentyl)-2-((1E,3E)-3-(1-(2-(2-(2-methoxyethoxy)ethoxy)ethyl)-3-methyl-5-sulfonato-3-(3-sulfonatopropyl)indolin-2-ylidene)prop-1-enyl)-3-(2-methoxyethyl)-3-methyl-3H-indolium-5-sulfonate) contains a (poly)ethylene glycol on the indole N of the left heterocycle. 550 Compound 3/2, with the (poly)ethylene glycol shown in abbreviated notation used throughout, represents the following structure.

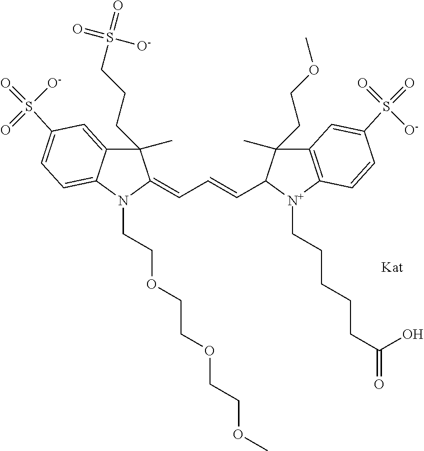 [SMILES: COCCOCCOCCN1/C(=C/C=C/C2[NH+](CCCCCC(=O)O)c3ccc(S(=O)(=O)[O-])cc3C2(C)CCOC)C(C)(CCCS(=O)(=O)[O-])c2cc(S(=O)(=O)[O-])ccc21]

The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 550 Compound 3/2 is activated as described above.

In one embodiment the compound is 550 Compound 4/2

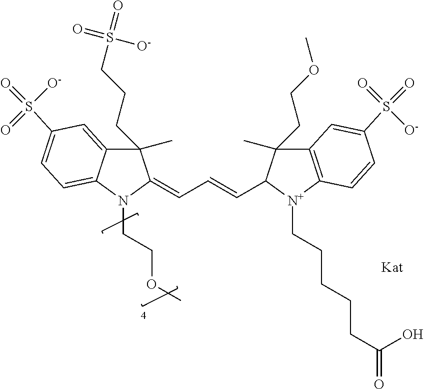

550 Compound 4/2 (1-(5-carboxypentyl)-3-(2-methoxyethyl)-3-methyl-2-((1E,3E)-3-(3-methyl-5-sulfonato-3-(3-sulfonatopropyl)-1-(2,5,8,11-tetraoxatridecan-13-yl)indolin-2-ylidene)prop-1-enyl)-3H-indolium-5-sulfonate) contains a (poly)ethylene glycol on the indole N of the left heterocycle. 550 Compound 4/2, with the (poly)ethylene glycol shown in abbreviated notation used throughout, represents the following structure.

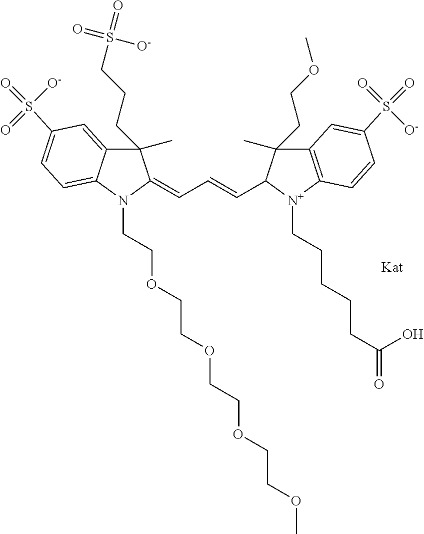 [SMILES: COCCOCCOCCOCCN1/C(=C/C=C/C2[NH+](CCCCCC(=O)O)c3ccc(S(=O)(=O)[O-])cc3C2(C)CCOC)C(C)(CCCS(=O)(=O)[O-])c2cc(S(=O)(=O)[O-])ccc21]

The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 550 Compound 4/2 is activated as described above.

In one embodiment the compound is 550 Compound 5/2

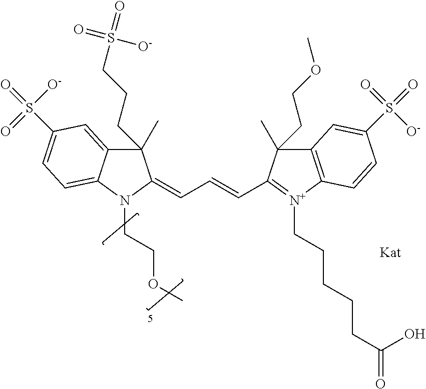

550 Compound 5/2 (2-((1E,3E)-3-(1-(2,5,8,11,14-pentaoxahexadecan-16-yl)-3-methyl-5-sulfonato-3-(3-sulfonatopropyl)indolin-2-ylidene)prop-1-enyl)-1-(5-carboxypentyl)-3-(2-methoxyethyl)-3-methyl-3H-indolium-5-sulfonate) contains a (poly)ethylene glycol on the indole N of the left heterocycle. 550 Compound 5/2, with the (poly)ethylene glycol shown in abbreviated notation used throughout, represents the following structure.

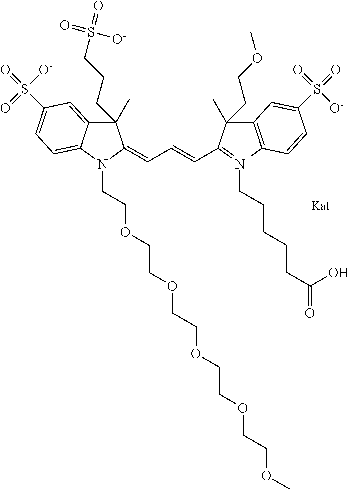 [SMILES: COCCOCCOCCOCCOCCN1/C(=C/C=C/C2=[N+](CCCCCC(=O)O)c3ccc(S(=O)(=O)[O-])cc3C2(C)CCOC)C(C)(CCCS(=O)(=O)[O-])c2cc(S(=O)(=O)[O-])ccc21]

The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 550 Compound 5/2 is activated as described above.

In one embodiment the compound is 550 Compound 6/2

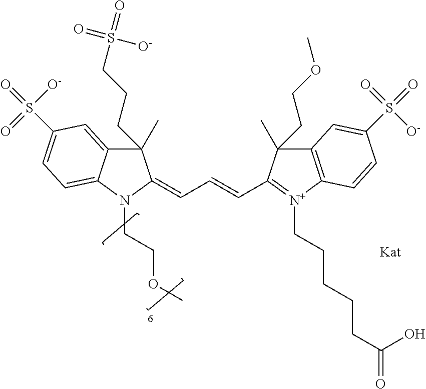

550 Compound 6/2 (1-(5-carboxypentyl)-3-(2-methoxyethyl)-3-methyl-2-((1E,3E)-3-(3-methyl-1-(2,5,8,11,14,17-hexaoxanonadecan-19-yl)-5-sulfonato-3-(3-sulfonatopropyl)indolin-2-ylidene)prop-1-enyl)-3H-indolium-5-sulfonate) contains a (poly)ethylene glycol on the indole N of the left heterocycle. 550 Compound 6/2, with the (poly)ethylene glycol shown in abbreviated notation used throughout, represents the following structure.

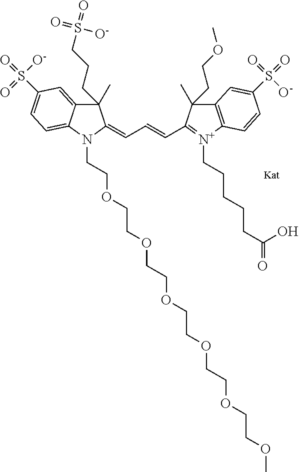 [SMILES: COCCOCCOCCOCCOCCOCCN1/C(=C/C=C/C2=[N+](CCCCCC(=O)O)c3ccc(S(=O)(=O)[O-])cc3C2(C)CCOC)C(C)(CCCS(=O)(=O)[O-])c2cc(S(=O)(=O)[O-])ccc21]

The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 550 Compound 6/2 is activated as described above.

In one embodiment the compound is 550 Compound 0/1

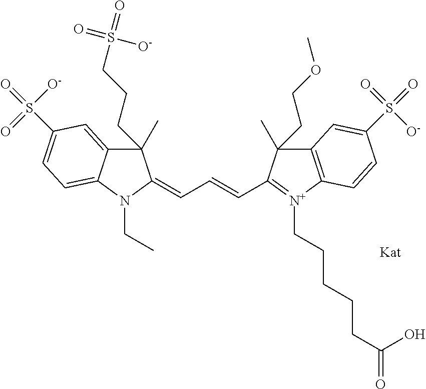 [SMILES: CCN1/C(=C/C=C/C2=[N+](CCCCCC(=O)O)c3ccc(S(=O)(=O)[O-])cc3C2(C)CCOC)C(C)(CCCS(=O)(=O)[O-])c2cc(S(=O)(=O)[O-])ccc21]

550 Compound 0/1 (1-(5-carboxypentyl)-2-((1E,3E)-3-(1-ethyl-3-methyl-5-sulfonato-3-(3-sulfonatopropyl)indolin-2-ylidene)prop-1-enyl)-3-(2-methoxyethyl)-3-methyl-3H-indolium-5-sulfonate) contains ethylene glycol at R2, sulfoalkyl at R1, ethyl at R9, and carboxyalkyl at R10.

One non-limiting example of an activated compound is the NHS-ester of 550 Compound 0/1, shown below:

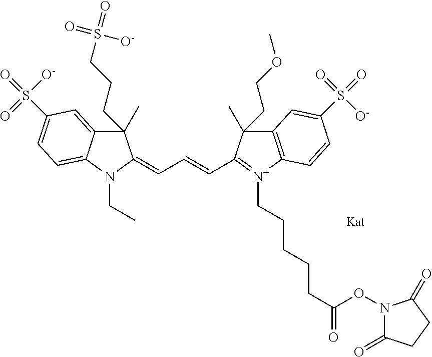 [SMILES: CCN1/C(=C/C=C/C2=[N+](CCCCCC(=O)ON3C(=O)CCC3=O)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 550 Compound 0/1 is a tetrafluorophenyl (TFP)-ester form, shown below:

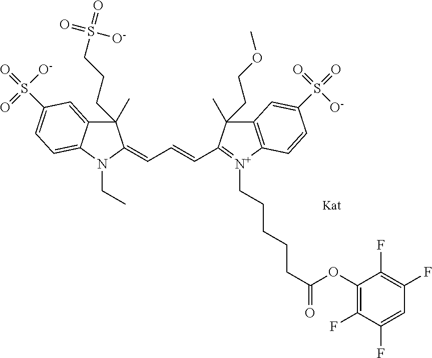 [SMILES: CCN1/C(=C/C=C/C2=[N+](CCCCCC(=O)Oc3c(F)c(F)cc(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 550 Compound 0/1 is a sulfotetrafluorophenyl (STP)-ester form, shown below:

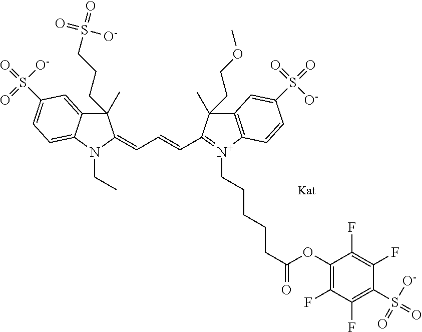 [SMILES: CCN1/C(=C/C=C/C2=[N+](CCCCCC(=O)Oc3c(F)c(F)c(S(=O)(=O)[O-])c(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 550 Compound 0/1 is a hydrazide form, shown below:

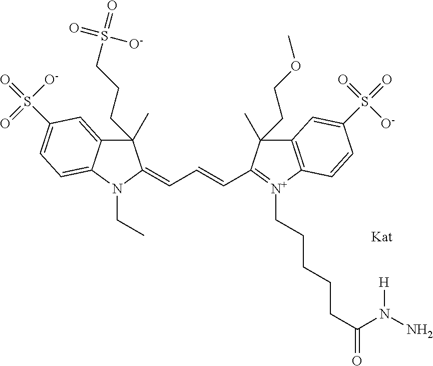 [SMILES: CCN1/C(=C/C=C/C2=[N+](CCCCCC(=O)NN)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 550 Compound 0/1 is a maleimide form, shown below:

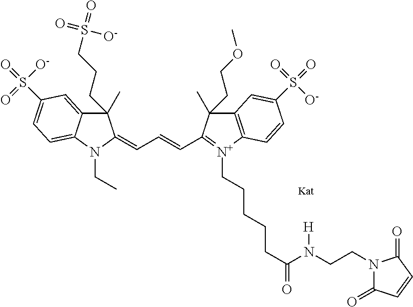 [SMILES: CCN1/C(=C/C=C/C2=[N+](CCCCCC(=O)NCCN3C(=O)C=CC3=O)c3ccc(S(=O)(=O)[O-])cc3C2(C)CCOC)C(C)(CCCS(=O)(=O)[O-])c2cc(S(=O)(=O)[O-])ccc21]

In one embodiment the compound is 550 Compound 0/1

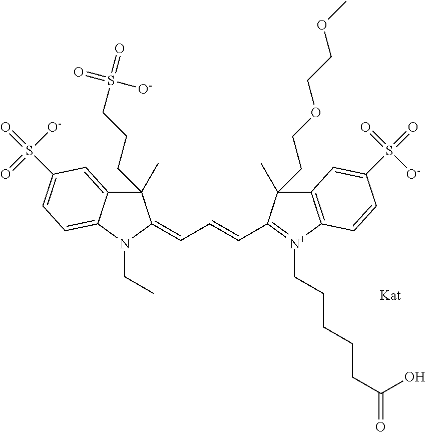 [SMILES: CCN1/C(=C/C=C/C2=[N+](CCCCCC(=O)O)c3ccc(S(=O)(=O)[O-])cc3C2(C)CCOCCOC)C(C)(CCCS(=O)(=O)[O-])c2cc(S(=O)(=O)[O-])ccc21]

550 Compound 0/1 (1-(5-carboxypentyl)-2-((1E,3E)-3-(1-ethyl-3-methyl-5-sulfonato-3-(3-sulfonatopropyl)indolin-2-ylidene)prop-1-enyl)-3-(2-(2-methoxyethoxy)ethyl)-3-methyl-3H-indolium-5-sulfonate) contains a diethylene glycol at R2, sulfoalkyl at R1, ethyl at R9, and carboxyalkyl at R10.

In one embodiment the compound is 550 Compound 0/1

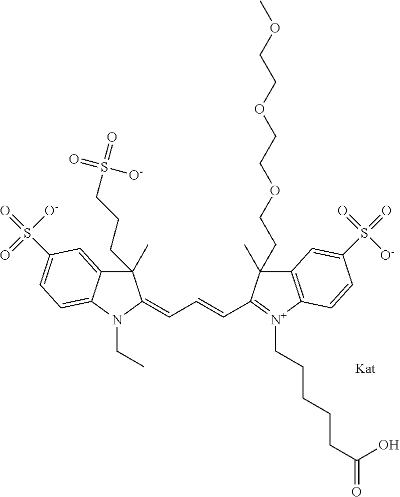 [SMILES: CCN1/C(=C/C=C/C2=[N+](CCCCCC(=O)O)c3ccc(S(=O)(=O)[O-])cc3C2(C)CCOCCOCCOC)C(C)(CCCS(=O)(=O)[O-])c2cc(S(=O)(=O)[O-])ccc21]

550 Compound 0/1 (1-(5-carboxypentyl)-2-((1E,3E)-3-(1-ethyl-3-methyl-5-sulfonato-3-(3-sulfonatopropyl)indolin-2-ylidene)prop-1-enyl)-3-(2-(2-(2-methoxyethoxy)ethoxy)ethyl)-3-methyl-3H-indolium-5-sulfonate) contains a (poly)ethylene glycol (PEG₃) at R2, sulfoalkyl at R1, ethyl at R9, and carboxyalkyl at R10.

In one embodiment the compound is 550 Compound 0/1

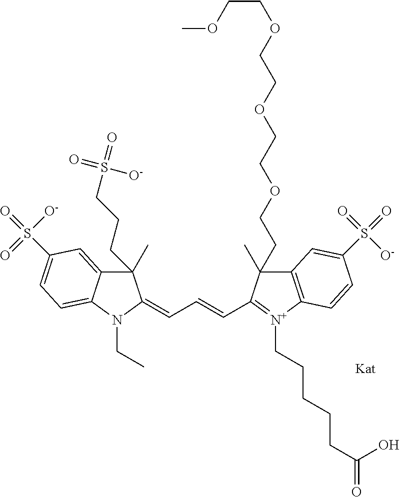 [SMILES: CCN1/C(=C/C=C/C2=[N+](CCCCCC(=O)O)c3ccc(S(=O)(=O)[O-])cc3C2(C)CCOCCOCCOCCOC)C(C)(CCCS(=O)(=O)[O-])c2cc(S(=O)(=O)[O-])ccc21]

550 Compound 0/1 (1-(5-carboxypentyl)-2-((1E,3E)-3-(1-ethyl-3-methyl-5-sulfonato-3-(3-sulfonatopropyl)indolin-2-ylidene)prop-1-enyl)-3-methyl-3-(2,5,8,11-tetraoxatridecan-13-yl)-3H-indolium-5-sulfonate) contains a (poly)ethylene glycol (PEG₄) at R2, sulfoalkyl at R1, ethyl at R9, and carboxyalkyl at R10.

In one embodiment the compound is 550 Compound 0/1

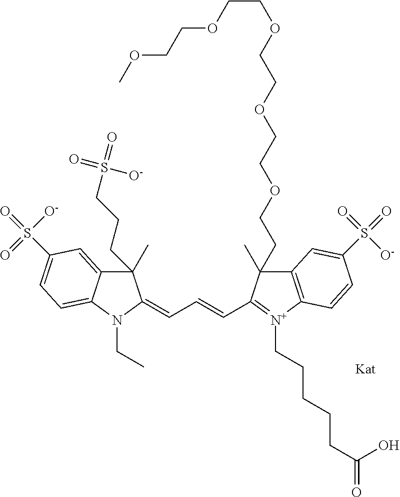 [SMILES: CCN1/C(=C/C=C/C2=[N+](CCCCCC(=O)O)c3ccc(S(=O)(=O)[O-])cc3C2(C)CCOCCOCCOCCOCCOC)C(C)(CCCS(=O)(=O)[O-])c2cc(S(=O)(=O)[O-])ccc21]

550 Compound 0/1 (1-(5-carboxypentyl)-2-((1E,3E)-3-(1-ethyl-3-methyl-5-sulfonato-3-(3-sulfonatopropyl)indolin-2-ylidene)prop-1-enyl)-3-(2,5,8,11,14-pentaoxahexadecan-16-yl)-3-methyl-3H-indolium-5-sulfonate) contains a (poly)ethylene glycol (PEG₅) at R2, sulfoalkyl at R1, ethyl at R9, and carboxyalkyl at R10.

In one embodiment the compound is 550 Compound 0/1

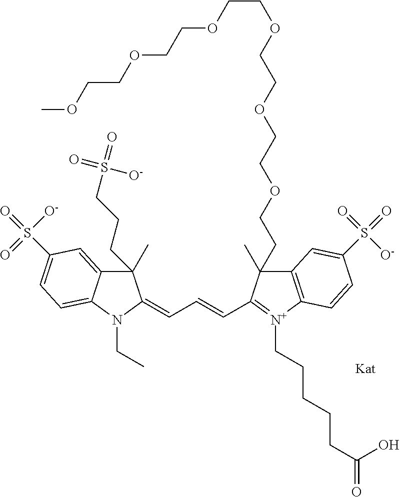 [SMILES: CCN1/C(=C/C=C/C2=[N+](CCCCCC(=O)O)c3ccc(S(=O)(=O)[O-])cc3C2(C)CCOCCOCCOCCOCCOCCOC)C(C)(CCCS(=O)(=O)[O-])c2cc(S(=O)(=O)[O-])ccc21]

550 Compound 0/1 (1-(5-carboxypentyl)-2-((1E,3E)-3-(1-ethyl-3-methyl-5-sulfonato-3-(3-sulfonatopropyl)indolin-2-ylidene)prop-1-enyl)-3-methyl-3-(2,5,8,11,14,17-hexaoxanonadecan-19-yl)-3H-indolium-5-sulfonate) contains a (poly)ethylene glycol (PEG₆) at R2, sulfoalkyl at R1, ethyl at R9, and carboxyalkyl at R10.

In one embodiment the compound is 550 Compound 0/1

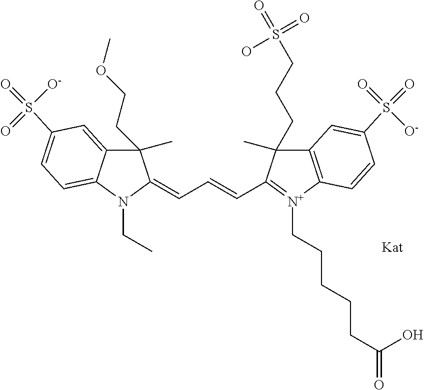 [SMILES: CCN1/C(=C/C=C/C2=[N+](CCCCCC(=O)O)c3ccc(S(=O)(=O)[O-])cc3C2(C)CCCS(=O)(=O)O)C(C)(CCOC)c2cc(S(=O)(=O)[O-])ccc21]

550 Compound 0/1 (1-(5-carboxypentyl)-2-((1E,3E)-3-(1-ethyl-3-(2-methoxyethyl)-3-methyl-5-sulfonatoindolin-2-ylidene)prop-1-enyl)-3-methyl-3-(3-sulfopropyl)-3H-indolium-5-sulfonate) contains an ethylene glycol at R1, sulfoalkyl at R2, ethyl at R9, and carboxyalkyl at R10.

One non-limiting example of an activated compound is the NHS-ester of 550 Compound 0/1, shown below:

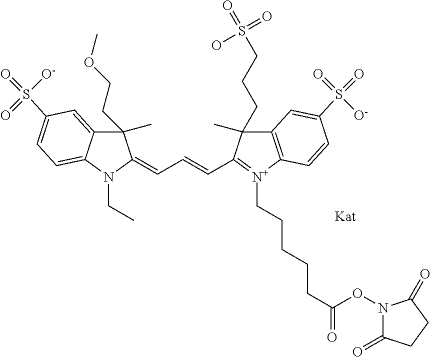 [SMILES: CCN1/C(=C/C=C/C2=[N+](CCCCCC(=O)ON3C(=O)CCC3=O)c3ccc(S(=O)(=O)[O-])cc3C2(C)CCCS(=O)(=O)O)C(C)(CCOC)c2cc(S(=O)(=O)[O-])ccc21]

In one embodiment the compound is 550 Compound 0/1

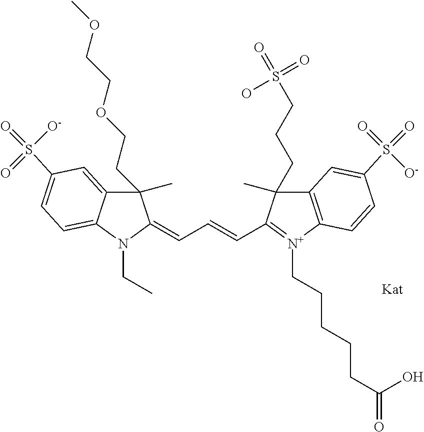 [SMILES: CCN1/C(=C/C=C/C2=[N+](CCCCCC(=O)O)c3ccc(S(=O)(=O)[O-])cc3C2(C)CCCS(=O)(=O)O)C(C)(CCOCCOC)c2cc(S(=O)(=O)[O-])ccc21]

550 Compound 0/1 (1-(5-carboxypentyl)-2-((1E,3E)-3-(1-ethyl-3-(2-(2-methoxyethoxy)ethyl)-3-methyl-5-sulfonatoindolin-2-ylidene)prop-1-enyl)-3-methyl-3-(3-sulfopropyl)-3H-indolium-5-sulfonate) contains a diethylene glycol at R1, sulfoalkyl at R2, ethyl at R9, and carboxyalkyl at R10.

In one embodiment the compound is 550 Compound 0/1

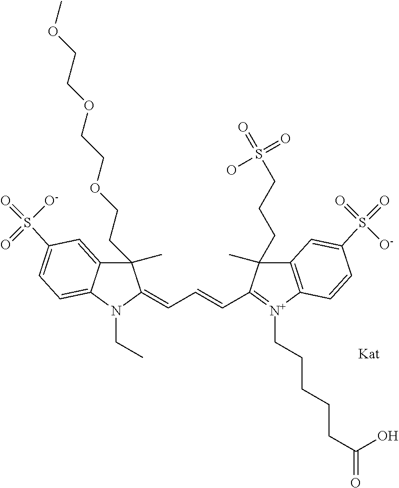 [SMILES: CCN1/C(=C/C=C/C2=[N+](CCCCCC(=O)O)c3ccc(S(=O)(=O)[O-])cc3C2(C)CCCS(=O)(=O)O)C(C)(CCOCCOCCOC)c2cc(S(=O)(=O)[O-])ccc21]

550 Compound 0/1 (1-(5-carboxypentyl)-2-((1E,3E)-3-(1-ethyl-3-(2-(2-(2-methoxyethoxy)ethoxy)ethyl)-3-methyl-5-sulfonatoindolin-2-ylidene)prop-1-enyl)-3-methyl-3-(3-sulfopropyl)-3H-indolium-5-sulfonate) contains a (poly)ethylene glycol (PEG₃) at R1, sulfoalkyl at R2, ethyl at R9, and carboxyalkyl at R10.

In one embodiment the compound is 550 Compound 0/1

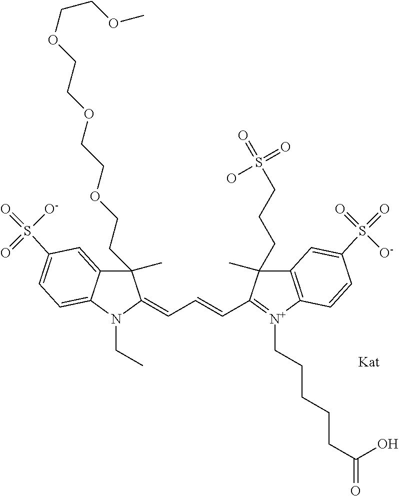 [SMILES: CCN1/C(=C/C=C/C2=[N+](CCCCCC(=O)O)c3ccc(S(=O)(=O)[O-])cc3C2(C)CCCS(=O)(=O)O)C(C)(CCOCCOCCOCCOC)c2cc(S(=O)(=O)[O-])ccc21]

550 Compound 0/1 (1-(5-carboxypentyl)-2-((1E,3E)-3-(1-ethyl-3-methyl-5-sulfonato-3-(2,5,8,11-tetraoxatridecan-13-yl)indolin-2-ylidene)prop-1-enyl)-3-methyl-3-(3-sulfopropyl)-3H-indolium-5-sulfonate) contains a (poly)ethylene glycol (PEG₄) at R1, sulfoalkyl at R2, ethyl at R9, and carboxyalkyl at R10.

In one embodiment the compound is 550 Compound 0/1

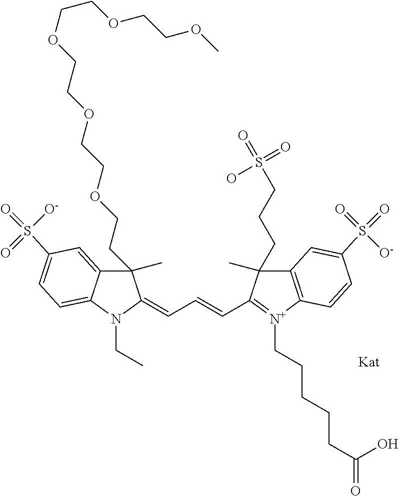 [SMILES: CCN1/C(=C/C=C/C2=[N+](CCCCCC(=O)O)c3ccc(S(=O)(=O)[O-])cc3C2(C)CCCS(=O)(=O)O)C(C)(CCOCCOCCOCCOCCOC)c2cc(S(=O)(=O)[O-])ccc21]

550 Compound 0/1 (1-(5-carboxypentyl)-2-((1E,3E)-3-(1-ethyl-3-(2,5,8,11,14-pentaoxahexadecan-16-yl)-3-methyl-5-sulfonatoindolin-2-ylidene)prop-1-enyl)-3-methyl-3-(3-sulfopropyl)-3H-indolium-5-sulfonate) contains a (poly)ethylene glycol (PEG₅) at R1, sulfoalkyl at R2, ethyl at R9, and carboxyalkyl at R10.

In one embodiment the compound is 550 Compound 0/1

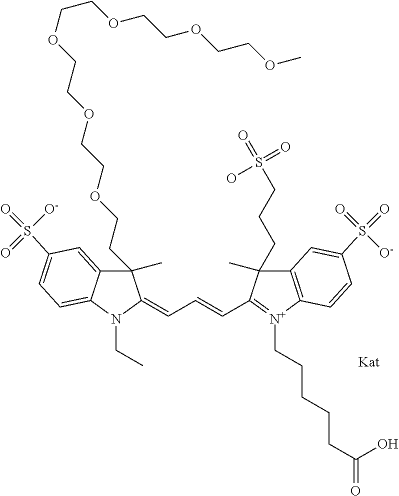 [SMILES: CCN1/C(=C/C=C/C2=[N+](CCCCCC(=O)O)c3ccc(S(=O)(=O)[O-])cc3C2(C)CCCS(=O)(=O)O)C(C)(CCOCCOCCOCCOCCOCCOC)c2cc(S(=O)(=O)[O-])ccc21]

550 Compound 0/1 (1-(5-carboxypentyl)-2-((1E,3E)-3-(1-ethyl-3-methyl-3-(2, 5,8,11,14,17-hexaoxanonadecan-19-yl)-5-sulfonatoindolin-2-ylidene)prop-1-enyl)-3-methyl-3-(3-sulfopropyl)-3H-indolium-5-sulfonate) contains a (poly)ethylene glycol (PEG₆) at R1, sulfoalkyl at R2, ethyl at R9, and carboxyalkyl at R10.

In one embodiment the compound is 550 Compound 0/2

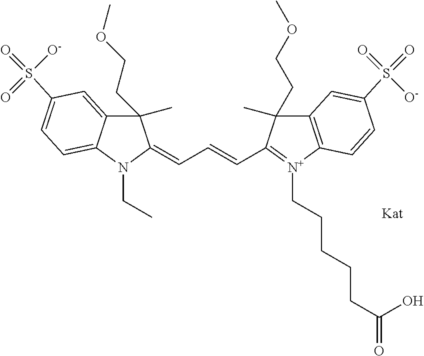 [SMILES: CCN1/C(=C/C=C/C2=[N+](CCCCCC(=O)O)c3ccc(S(=O)(=O)[O-])cc3C2(C)CCOC)C(C)(CCOC)c2cc(S(=O)(=O)[O-])ccc21]

550 Compound 0/2 (1-(5-carboxypentyl)-2-((1E,3E)-3-(1-ethyl-3-(2-methoxyethyl)-3-methyl-5-sulfonatoindolin-2-ylidene)prop-1-enyl)-3-(2-methoxyethyl)-3-methyl-3H-indolium-5-sulfonate) contains ethylene glycol at R1 and R2, ethyl at R9, and carboxyalkyl at R10.

One non-limiting example of an activated compound is the NHS-ester of 550 Compound 0/2, shown below:

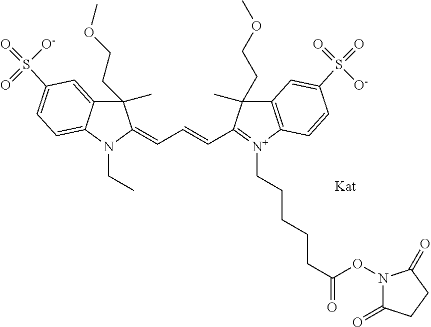 [SMILES: CCN1/C(=C/C=C/C2=[N+](CCCCCC(=O)ON3C(=O)CCC3=O)c3ccc(S(=O)(=O)[O-])cc3C2(C)CCOC)C(C)(CCOC)c2cc(S(=O)(=O)[O-])ccc21]

In one embodiment the compound is 550 Compound 0/2

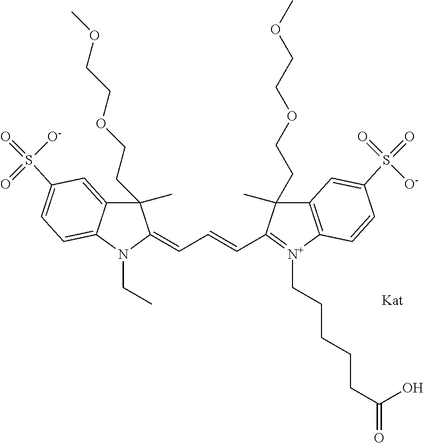 [SMILES: CCN1/C(=C/C=C/C2=[N+](CCCCCC(=O)O)c3ccc(S(=O)(=O)[O-])cc3C2(C)CCOCCOC)C(C)(CCOCCOC)c2cc(S(=O)(=O)[O-])ccc21]

550 Compound 0/2 (1-(5-carboxypentyl)-2-((1E,3E)-3-(1-ethyl-3-(2-(2-methoxyethoxy)ethyl)-3-methyl-5-sulfonatoindolin-2-ylidene)prop-1-enyl)-3-(2-(2-methoxyethoxy)ethyl)-3-methyl-3H-indolium-5-sulfonate) contains a diethylene glycol at R1 and R2, ethyl at R9, and carboxyalkyl at R10.

In one embodiment the compound is 550 Compound 0/2

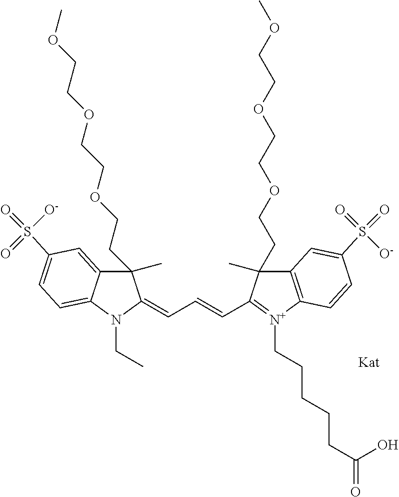 [SMILES: CCN1/C(=C/C=C/C2=[N+](CCCCCC(=O)O)c3ccc(S(=O)(=O)[O-])cc3C2(C)CCOCCOCCOC)C(C)(CCOCCOCCOC)c2cc(S(=O)(=O)[O-])ccc21]

550 Compound 0/2 (1-(5-carboxypentyl)-2-((1E,3E)-3-(1-ethyl-3-(2-(2-(2-methoxyethoxy)ethoxy)ethyl)-3-methyl-5-sulfonatoindolin-2-ylidene)prop-1-enyl)-3-(2-(2-(2-methoxyethoxy)ethoxy)ethyl)-3-methyl-3H-indolium-5-sulfonate) contains a (poly)ethylene glycol (PEG₃) at R1 and R2, ethyl at R9, and carboxyalkyl at R10.

In one embodiment the compound is 550 Compound 0/2

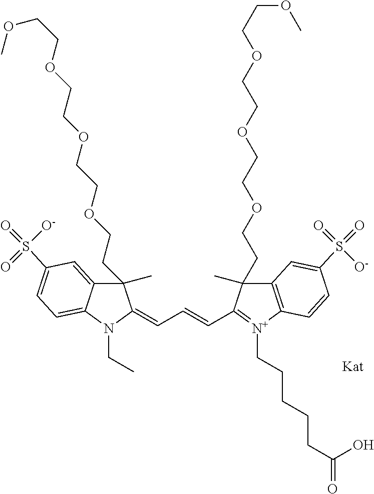 [SMILES: CCN1/C(=C/C=C/C2=[N+](CCCCCC(=O)O)c3ccc(S(=O)(=O)[O-])cc3C2(C)CCOCCOCCOCCOC)C(C)(CCOCCOCCOCCOC)c2cc(S(=O)(=O)[O-])ccc21]

550 Compound 0/2 (1-(5-carboxypentyl)-2-((1E,3E)-3-(1-ethyl-3-methyl-5-sulfonato-3-(2,5,8,11-tetraoxatridecan-13-yl)indolin-2-ylidene)prop-1-enyl)-3-methyl-3-(2,5,8,11-tetraoxatridecan-13-yl)-3H-indolium-5-sulfonate) contains a (poly)ethylene glycol (PEG₄) at R1 and R2, ethyl at R9, and carboxyalkyl at R10.

In one embodiment the compound is 550 Compound 0/2

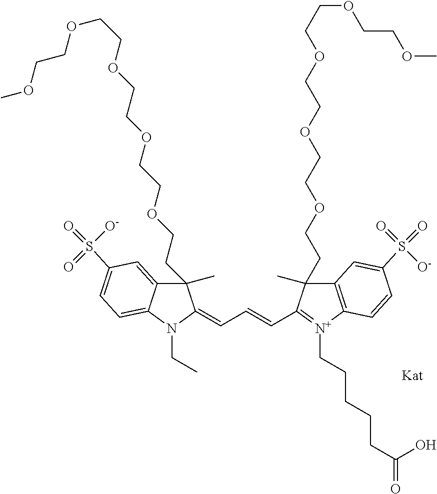

550 Compound 0/2 (1-(5-carboxypentyl)-2-((1E,3E)-3-(1-ethyl-3-(2,5,8,11,14-pentaoxahexadecan-16-yl)-3-methyl-5-sulfonatoindolin-2-ylidene)prop-1-enyl)-3-(2,5,8,11,14-pentaoxahexadecan-16-yl)-3-methyl-3H-indolium-5-sulfonate) contains a (poly)ethylene glycol (PEG₅) at R1 and R2, ethyl at R9, and carboxyalkyl at R10.

In one embodiment the compound is 550 Compound 0/2

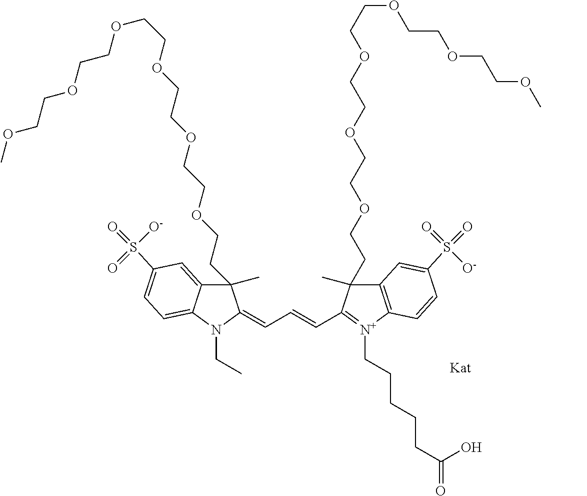

550 Compound 0/2 (1-(5-carboxypentyl)-2-((1E,3E)-3-(1-ethyl-3-methyl-3-(2,5,8,11,14,17-hexaoxanonadecan-19-yl)-5-sulfonatoindolin-2-ylidene)prop-1-enyl)-3-methyl-3-(2,5,8,11,14,17-hexaoxanonadecan-19-yl)-3H-indolium-5-sulfonate) contains a (poly)ethylene glycol (PEG₆) at R1 and R2, ethyl at R9, and carboxyalkyl at R10.

In one embodiment the compound is 550 Compound 0/1

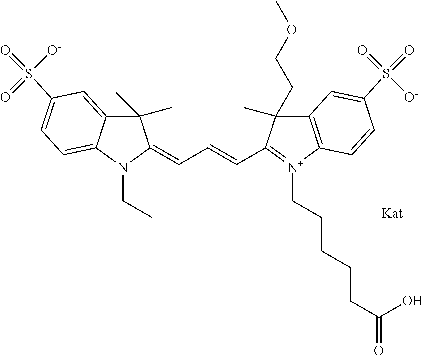 [SMILES: CCN1/C(=C/C=C/C2=[N+](CCCCCC(=O)O)c3ccc(S(=O)(=O)[O-])cc3C2(C)CCOC)C(C)(C)c2cc(S(=O)(=O)[O-])ccc21]

550 Compound 0/1 (1-(5-carboxypentyl)-2-((1E,3E)-3-(1-ethyl-3, 3-dimethyl-5-sulfonatoindolin-2-ylidene)prop-1-enyl)-3-(2-methoxyethyl)-3-methyl-3H-indolium-5-sulfonate) contains ethylene glycol at R1, methyl at R2, ethyl at R9, and carboxyalkyl at R10.

One non-limiting example of an activated compound is the NHS-ester of 550 Compound 0/1, shown below:

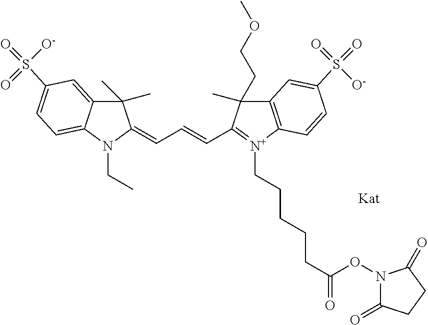 [SMILES: CCN1/C(=C/C=C/C2=[N+](CCCCCC(=O)ON3C(=O)CCC3=O)c3ccc(S(=O)(=O)[O-])cc3C2(C)CCOC)C(C)(C)c2cc(S(=O)(=O)[O-])ccc21]

In one embodiment the compound is 550 Compound 0/1

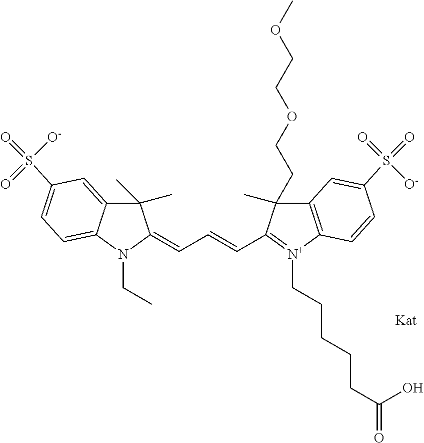 [SMILES: CCN1/C(=C/C=C/C2=[N+](CCCCCC(=O)O)c3ccc(S(=O)(=O)[O-])cc3C2(C)CCOCCOC)C(C)(C)c2cc(S(=O)(=O)[O-])ccc21]

550 Compound 0/1 (1-(5-carboxypentyl)-2-((1E,3E)-3-(1-ethyl-3, 3-dimethyl-5-sulfonatoindolin-2-ylidene)prop-1-enyl)-3-(2-(2-methoxyethoxy)ethyl)-3-methyl-3H-indolium-5-sulfonate) contains diethylene glycol at R1, methyl at R2, ethyl at R9, and carboxyalkyl at R10.

In one embodiment the compound is 550 Compound 0/1

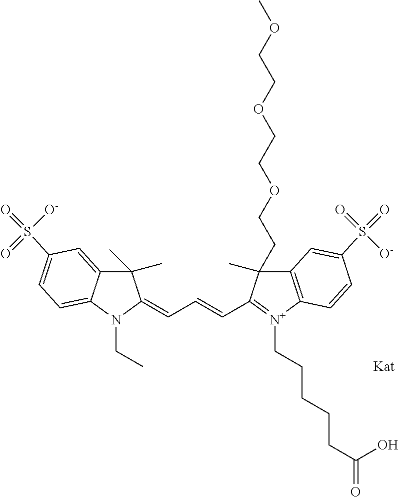 [SMILES: CCN1/C(=C/C=C/C2=[N+](CCCCCC(=O)O)c3ccc(S(=O)(=O)[O-])cc3C2(C)CCOCCOCCOC)C(C)(C)c2cc(S(=O)(=O)[O-])ccc21]

550 Compound 0/1 (1-(5-carboxypentyl)-2-((1E,3E)-3-(1-ethyl-3,3-dimethyl-5-sulfonatoindolin-2-ylidene)prop-1-enyl)-3-(2-(2-(2-methoxyethoxy)ethoxy)ethyl)-3-methyl-3H-indolium-5-sulfonate) contains a (poly)ethylene glycol (PEG₃) at R1, methyl at R2, ethyl at R9, and carboxyalkyl at R10.

In one embodiment the compound is 550 Compound 0/1

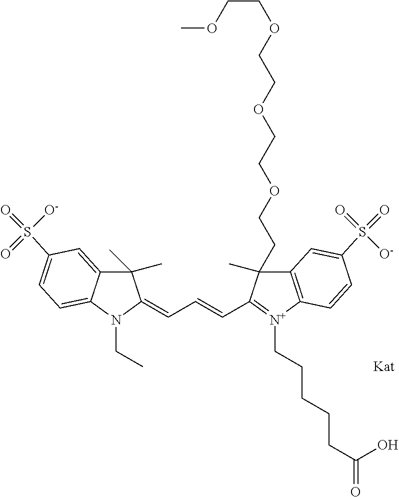 [SMILES: CCN1/C(=C/C=C/C2=[N+](CCCCCC(=O)O)c3ccc(S(=O)(=O)[O-])cc3C2(C)CCOCCOCCOCCOC)C(C)(C)c2cc(S(=O)(=O)[O-])ccc21]

550 Compound 0/1 (1-(5-carboxypentyl)-2-((1E,3E)-3-(1-ethyl-3,3-dimethyl-5-sulfonatoindolin-2-ylidene)prop-1-enyl)-3-methyl-3-(2,5,8,11-tetraoxatridecan-13-yl)-3H-indolium-5-sulfonate) contains a (poly)ethylene glycol (PEG₄) at R1, methyl at R2, ethyl at R9, and carboxyalkyl at R10.

In one embodiment the compound is 550 Compound 0/1

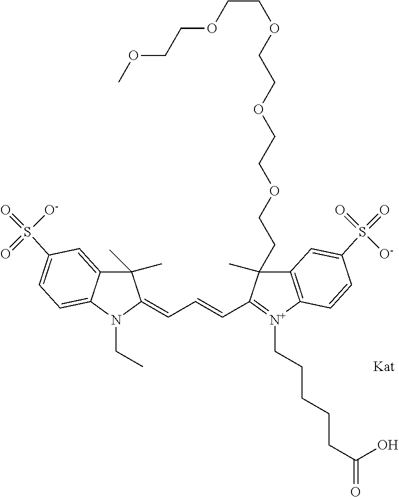 [SMILES: CCN1/C(=C/C=C/C2=[N+](CCCCCC(=O)O)c3ccc(S(=O)(=O)[O-])cc3C2(C)CCOCCOCCOCCOCCOC)C(C)(C)c2cc(S(=O)(=O)[O-])ccc21]

550 Compound 0/1 (1-(5-carboxypentyl)-2-((1E,3E)-3-(1-ethyl-3,3-dimethyl-5-sulfonatoindolin-2-ylidene)prop-1-enyl)-3-(2,5,8,11,14-pentaoxahexadecan-16-yl)-3-methyl-3H-indolium-5-sulfonate) contains a (poly)ethylene glycol (PEG₅) at R1, methyl at R2, ethyl at R9, and carboxyalkyl at R10.

In one embodiment the compound is 550 Compound 0/1

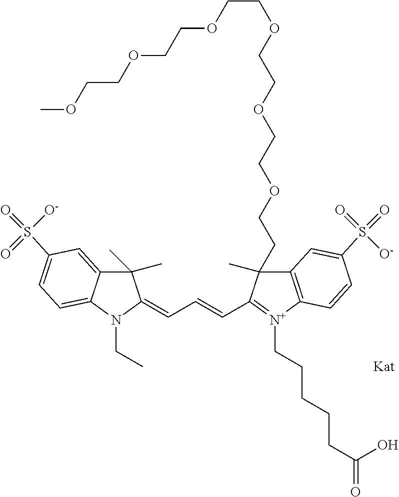 [SMILES: CCN1/C(=C/C=C/C2=[N+](CCCCCC(=O)O)c3ccc(S(=O)(=O)[O-])cc3C2(C)CCOCCOCCOCCOCCOCCOC)C(C)(C)c2cc(S(=O)(=O)[O-])ccc21]

550 Compound 0/1 (1-(5-carboxypentyl)-2-((1E,3E)-3-(1-ethyl-3,3-dimethyl-5-sulfonatoindolin-2-ylidene)prop-1-enyl)-3-methyl-3-(2,5,8,11,14,17-hexaoxanonadecan-19-yl)-3H-indolium-5-sulfonate) contains a (poly)ethylene glycol (PEG₆) at R1, methyl at R2, ethyl at R9, and carboxyalkyl at R10.

In one embodiment the compound is 550 Compound 0/1

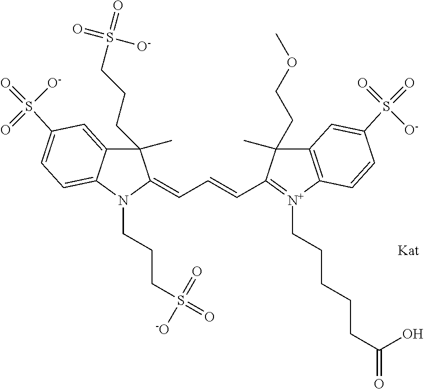

550 Compound 0/1 (3,3′-((E)-2-((E)-3-(1-(5-carboxypentyl)-3-(2-methoxyethyl)-3-methyl-5-sulfonato-3H-indolium-2-yl)allylidene)-3-methyl-5-sulfonatoindoline-1,3-diyl)dipropane-1-sulfonate) contains ethylene glycol at R2, a sulfoalkyl at R1, a sulfoalkyl at R9, and carboxyalkyl at R10.

One non-limiting example of an activated compound is the NHS-ester of 550 Compound 0/1, shown below:

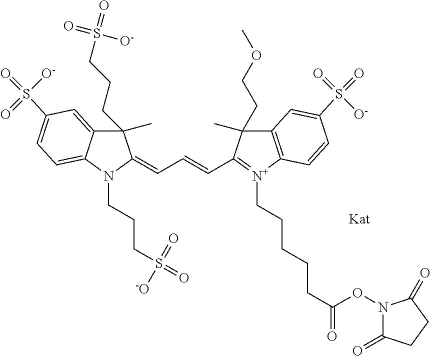

In one embodiment the compound is 550 Compound 0/1

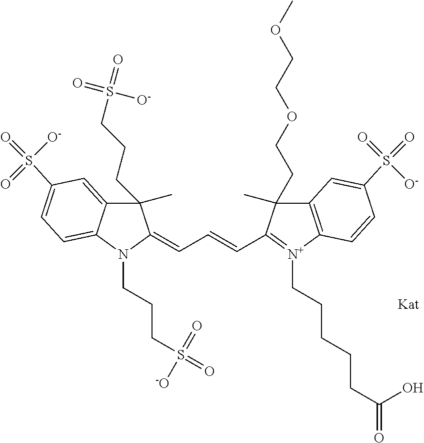

550 Compound 0/1 (3,3′-((E)-2-((E)-3-(1-(5-carboxypentyl)-3-(2-(2-methoxyethoxy)ethyl)-3-methyl-5-sulfonato-3H-indolium-2-yl)allylidene)-3-methyl-5-sulfonatoindoline-1,3-diyl)dipropane-1-sulfonate) contains diethylene glycol at R2, sulfoalkyl at R1, sulfoalkyl at R9, and carboxyalkyl at R10.

In one embodiment the compound is 550 Compound 0/1

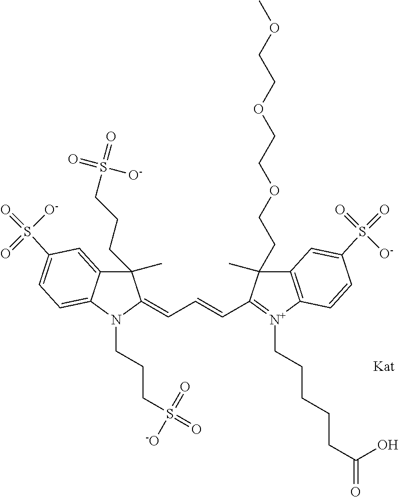

550 Compound 0/1 (3,3′-((E)-2-((E)-3-(1-(5-carboxypentyl)-3-(2-(2-(2-methoxyethoxy)ethoxy)ethyl)-3-methyl-5-sulfonato-3H-indolium-2-yl)allylidene)-3-methyl-5-sulfonatoindoline-1,3-diyl)dipropane-1-sulfonate) contains a (poly)ethylene glycol (PEG₃) at R2, sulfoalkyl at R1, sulfoalkyl at R9, and carboxyalkyl at R10.

In one embodiment the compound is 550 Compound 0/1

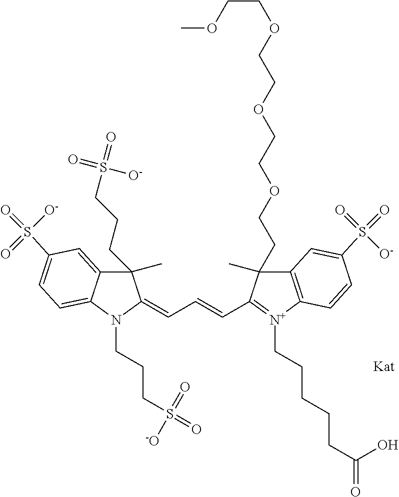

550 Compound 0/1 (3,3′-((E)-2-((E)-3-(1-(5-carboxypentyl)-3-methyl-5-sulfonato-3-(2,5,8,11-tetraoxatridecan-13-yl)-3H-indolium-2-yl)allylidene)-3-methyl-5-sulfonatoindoline-1,3-diyl)dipropane-1-sulfonate) contains a (poly)ethylene glycol (PEG₄) at R2, sulfoalkyl at R1, sulfoalkyl at R9, and carboxyalkyl at R10.

In one embodiment the compound is 550 Compound 0/1

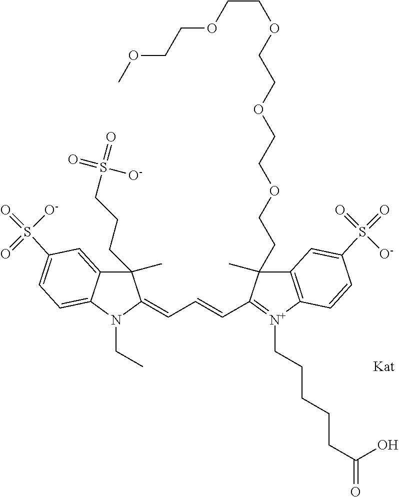 [SMILES: CCN1/C(=C/C=C/C2=[N+](CCCCCC(=O)O)c3ccc(S(=O)(=O)[O-])cc3C2(C)CCOCCOCCOCCOCCOC)C(C)(CCCS(=O)(=O)[O-])c2cc(S(=O)(=O)[O-])ccc21]

550 Compound 0/1 (1-(5-carboxypentyl)-2-((1E,3E)-3-(1-ethyl-3-methyl-5-sulfonato-3-(3-sulfonatopropyl)indolin-2-ylidene)prop-1-enyl)-3-(2,5,8,11,14-pentaoxahexadecan-16-yl)-3-methyl-3H-indolium-5-sulfonate) contains a (poly)ethylene glycol (PEG₅) at R2, sulfoalkyl at R1, ethyl at R9, and carboxyalkyl at R10.

In one embodiment the compound is 550 Compound 0/1

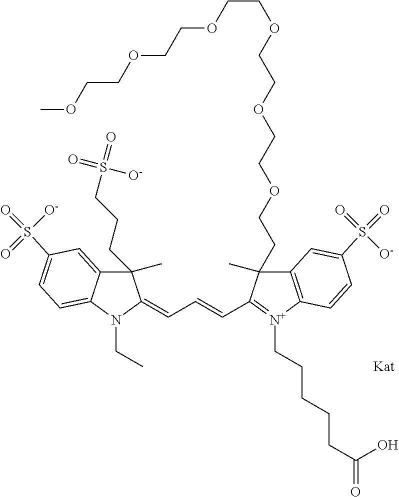 [SMILES: CCN1/C(=C/C=C/C2=[N+](CCCCCC(=O)O)c3ccc(S(=O)(=O)[O-])cc3C2(C)CCOCCOCCOCCOCCOCCOC)C(C)(CCCS(=O)(=O)[O-])c2cc(S(=O)(=O)[O-])ccc21]

550 Compound 0/1 (1-(5-carboxypentyl)-2-((1E,3E)-3-(1-ethyl-3-methyl-5-sulfonato-3-(3-sulfonatopropyl)indolin-2-ylidene)prop-1-enyl)-3-methyl-3-(2,5,8,11,14,17-hexaoxanonadecan-19-yl)-3H-indolium-5-sulfonate) contains a (poly)ethylene glycol (PEG₆) at R2, sulfoalkyl at R1, ethyl at R9, and carboxyalkyl at R10.

In one embodiment the compound is 550 Compound 0/2

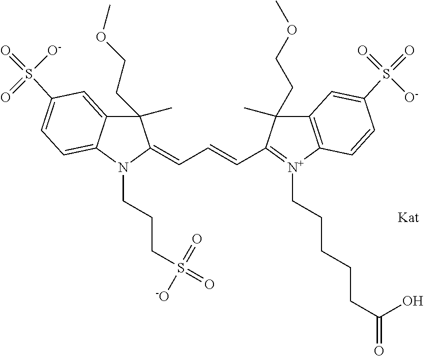

550 Compound 0/2 (1-(5-carboxypentyl)-3-(2-methoxyethyl)-2-((1E,3E)-3-(3-(2-methoxyethyl)-3-methyl-5-sulfonato-1-(3-sulfonatopropyl)indolin-2-ylidene)prop-1-enyl)-3-methyl-3H-indolium-5-sulfonate) contains ethylene glycol at R1 and R2, sulfoalkyl at R9, and carboxyalkyl at R10.

One non-limiting example of an activated compound is the NHS-ester of 550 Compound 0/2, shown below:

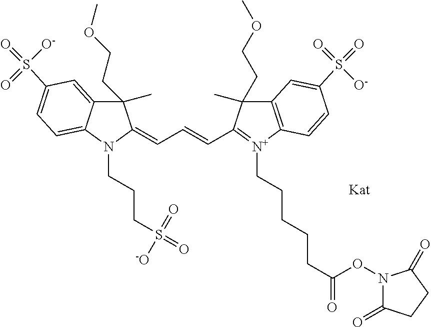

In one embodiment the compound is 550 Compound 0/2

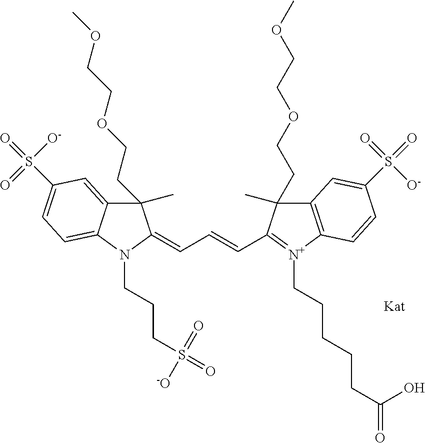

550 Compound 0/2 (1-(5-carboxypentyl)-3-(2-(2-methoxyethoxy)ethyl)-2-((1E,3E)-3-(3-(2-(2-methoxyethoxy)ethyl)-3-methyl-5-sulfonato-1-(3-sulfonatopropyl)indolin-2-ylidene)prop-1-enyl)-3-methyl-3H-indolium-5-sulfonate) contains a diethylene glycol at R1 and R2, sulfoalkyl at R9, and carboxyalkyl at R10.

In one embodiment the compound is 550 Compound 0/2

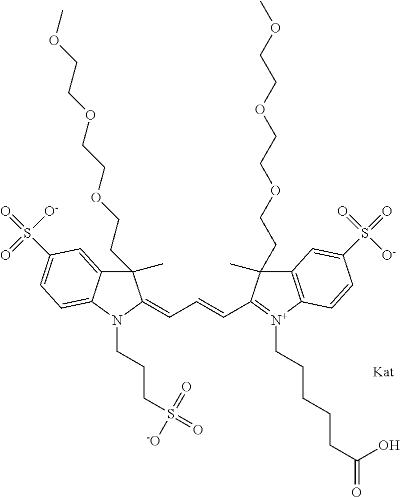

550 Compound 0/2 (1-(5-carboxypentyl)-3-(2-(2-(2-methoxyethoxy)ethoxy)ethyl)-2-((1E,3E)-3-(3-(2-(2-(2-methoxyethoxy)ethoxy)ethyl)-3-methyl-5-sulfonato-1-(3-sulfonatopropyl)indolin-2-ylidene)prop-1-enyl)-3-methyl-3H-indolium-5-sulfonate) contains a (poly)ethylene glycol (PEG₃) at R1 and R2, sulfoalkyl at R9, and carboxyalkyl at R10.

In one embodiment the compound is 550 Compound 0/2

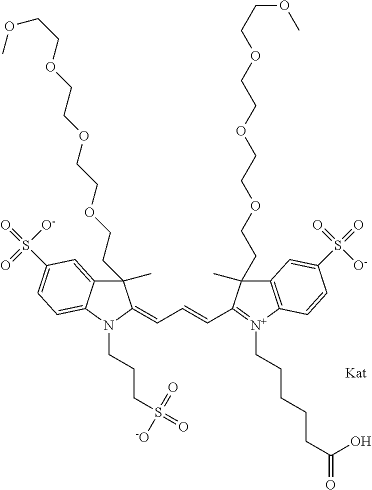

550 Compound 0/2 (1-(5-carboxypentyl)-3-methyl-2-((1E,3E)-3-(3-methyl-5-sulfonato-1-(3-sulfonatopropyl)-3-(2,5,8,11-tetraoxatridecan-13-yl)indolin-2-ylidene)prop-1-enyl)-3-(2,5,8,11-tetraoxatridecan-13-yl)-3H-indolium-5-sulfonate) contains a (poly)ethylene glycol (PEG₄) at R1 and R2, sulfoalkyl at R9, and carboxyalkyl at R10.

In one embodiment the compound is 550 Compound 0/2

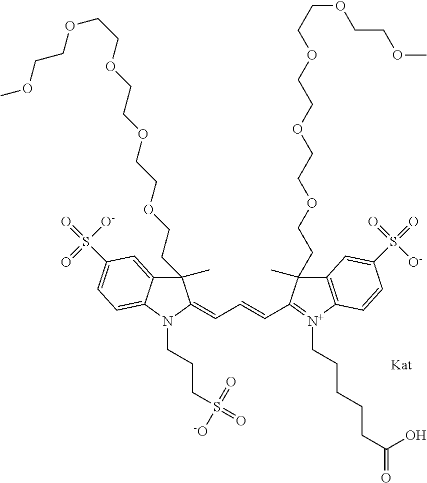

550 Compound 0/2 (2-((1E,3E)-3-(3-(2,5,8,11,14-pentaoxahexadecan-16-yl)-3-methyl-5-sulfonato-1-(3-sulfonatopropyl)indolin-2-ylidene)prop-1-enyl)-1-(5-carboxypentyl)-3-(2,5,8,11,14-pentaoxahexadecan-16-yl)-3-methyl-3H-indolium-5-sulfonate) contains a (poly)ethylene glycol (PEG₅) at R1 and R2, ethyl at R9, and carboxyalkyl at R10.

In one embodiment the compound is 550 Compound 0/2

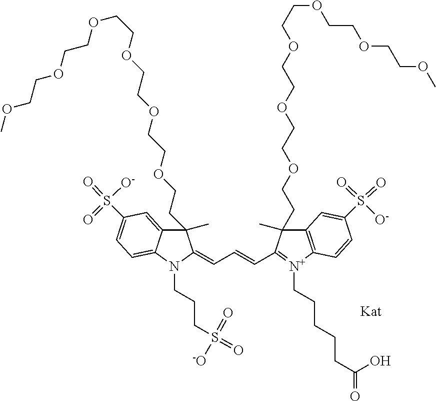

550 Compound 0/2 (1-(5-carboxypentyl)-3-methyl-2-((1E,3E)-3-(3-methyl-3-(2,5,8,11,14,17-hexaoxanonadecan-19-yl)-5-sulfonato-1-(3-sulfonatopropyl)indolin-2-ylidene)prop-1-enyl)-3-(2,5,8,11,14,17-hexaoxanonadecan-19-yl)-3H-indolium-5-sulfonate) contains a (poly)ethylene glycol (PEG₆) at R1 and R2, ethyl at R9, and carboxyalkyl at R10.

In embodiments, the degree of sulfonation is varied to, e.g., vary the compound's degree of hydrophilicity or hydrophobicity. One non-limiting example is a monosulfonate form of 550 Compound 0/1, shown below, but it is understood that the single sulfo group can be at any of the described positions:

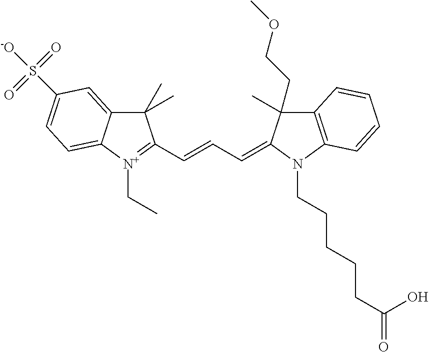

One non-limiting example is a disulfonate form of 550 Compound 0/1, shown below, but it is understood that each of the two sulfos can be at any of the described positions:

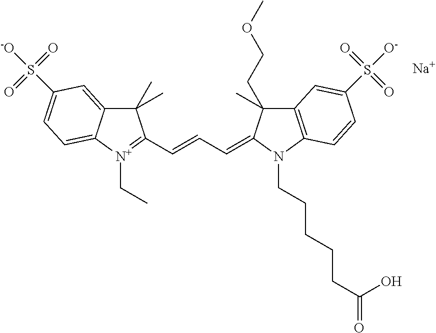

One non-limiting example is a trisulfonate form of 550 Compound 0/1, shown below, but it is understood that each of the three sulfos can be at any of the described positions:

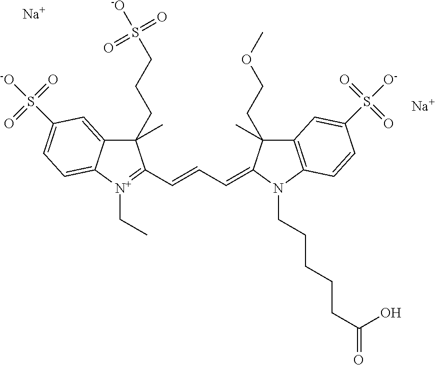

One non-limiting example is a tetrasulfonate form of 550 Compound 0/1, shown below, but it is understood that each of the four sulfo can be at any of the described positions:

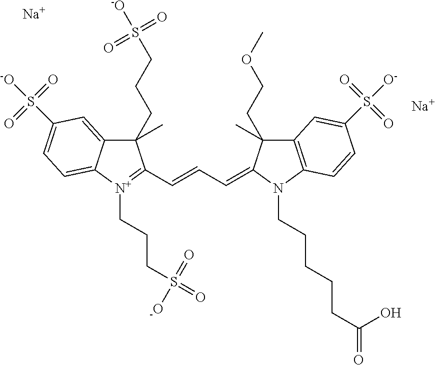

In embodiments, the compound contains one or more substitutions of the polymethine linker. In one embodiment the compound has general formula IVa

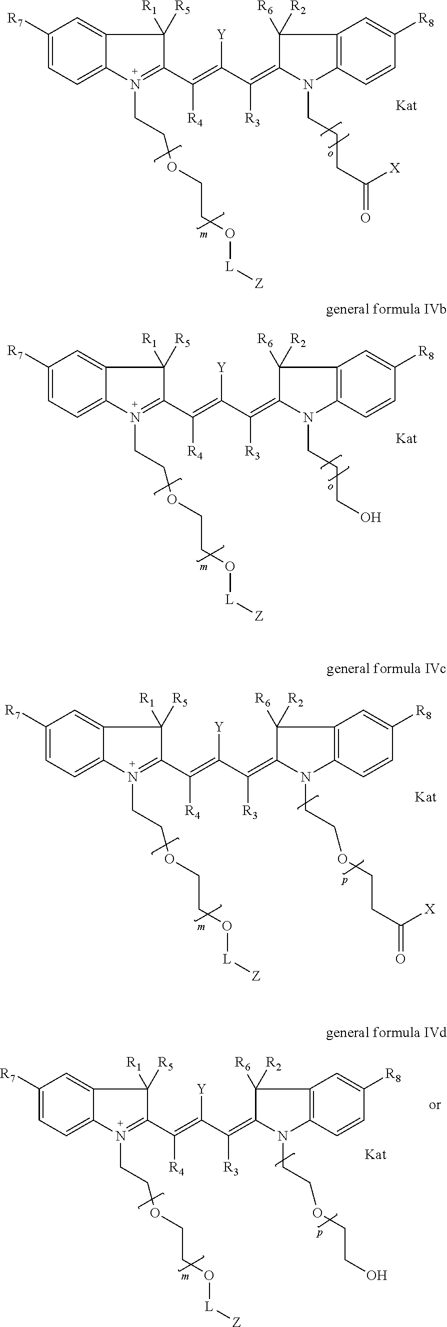

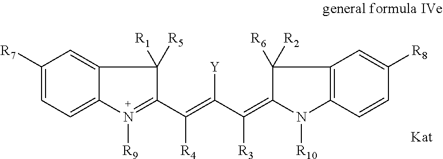

where each of R¹, R², R⁵, R⁶, R⁷, and R⁸ is the same or different and is independently selected from the group consisting of an aliphatic, heteroaliphatic, sulfoalkyl group, heteroaliphatic with terminal SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group -L-SO₂NH—P-L-Z, and a caboxamide group -L-CONH—P-L-Z; each of R⁷ and R⁸ is the same or different and is independently selected from either H, SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group —SO₂NH—P-L-Z, or a caboxamide group —CONH—P-L-Z; each of R⁹ and R¹⁰ is the same or different and is independently selected from the group consisting of alkyl, sulfoalkyl, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a PEG group P-L-X, L-Z, L-X; where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur; where Z is selected from the group consisting of H, CH₃, an alkyl, a heteroalkyl, NH₂, —COO, —COOH, —COSH, CO—NH—NH₂, —COF, —COCl, —COBr, —COI, —COO-Su (succinimidyl/sulfo-succinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO⁻, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′—L-CO—NH—NH₂, —CONR′-L-OH, —CONR′-L-O-phosphoramidite, —CONR′-L-CHO, —CONR′-L-maleimide, and —CONR′-L-NH—CO—CH₂—I; R′ and R″ is selected from the group consisting of H, aliphatic group, and heteroaliphatic group, and the biomolecule is a protein, antibody, nucleotide, oligonucleotide, biotin, or hapten; X is selected from the group consisting of —OH, —SH, —NH₂, —NH—NH₂, —F, —Cl, —Br, I, —NHS hydroxysuccinimidyl/sulfosuccinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group; Kat is a number of Na⁺, K⁺, Ca²⁺, ammonia, or other cation(s) needed to compensate the negative charge brought by the cyanine; m is an integer from 0 to 5 inclusive; p is an integer from 1 to 6 inclusive; each of R3 and R4 is the same or different and is independently hydrogen, an aliphatic group, a heteroaliphatic group, or a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive; or R3 and R4 together form a cyclic structure where R3 and R4 are joined using a divalent structural element selected from the group consisting of —(CH₂)_{q}—, —(CH₂)_{q}O(CH₂)_{q′}—, —(CH₂)_{q}S(CH₂)_{q′}—, —(CH₂)_{q}CH═CH—, —OCH═CH— where each of q and q′ is the same or different and is a integer from 2 to 6 inclusive; and Y is selected from the group consisting of hydrogen, alkyl, sulfoalkyl, fluorine, chlorine, bromine, a substituted or unsubstituted aryl-, phenoxy- or phenylmercapto function; and Y is selected from the group consisting of hydrogen, alkyl, sulfoalkyl, fluorine, chlorine, bromine, and a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive; with the proviso that at least one of R¹, R², R³, R⁴, R⁵, R⁶, R⁷, and R⁸ contains a PEG group.

In one embodiment the compound of general formula IV wherein each of R3 and R4 is the same or different and is independently hydrogen, an aliphatic group, or a heteroaliphatic group, or R3 and R4 together form a cyclic structure where R3 and R4 are directly joined or joined using a divalent structural element selected from the group consisting of —(CH₂)_{q}— and CH═CH, where q is an integer from 1 to 2 inclusive, to result in a 3-, 4-, or 5-membered ring.

One non-limiting example is a substituted polymethine form of 550 Compound 1/2, shown below:

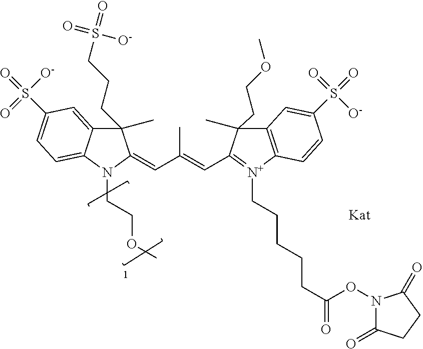

One non-limiting example is a substituted polymethine form of 550 Compound 2/2, shown below:

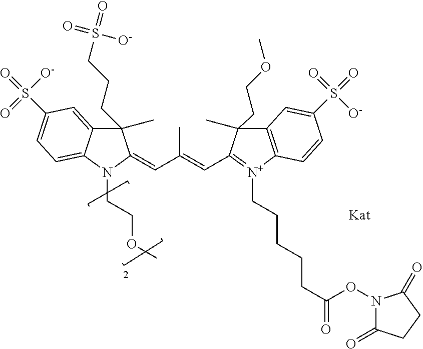

One non-limiting example is a substituted polymethine form of 550 Compound 3/2, shown below:

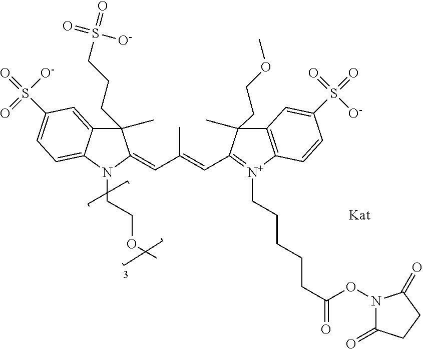

One non-limiting example is a substituted polymethine form of 550 Compound 4/2, shown below:

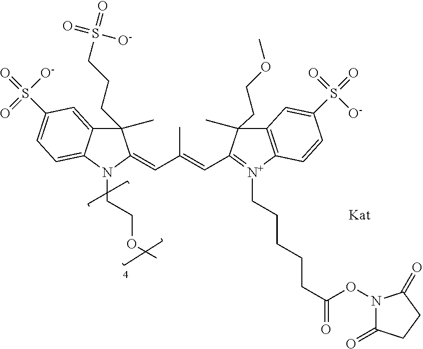

One non-limiting example is a substituted polymethine form of 550 Compound 5/2, shown below:

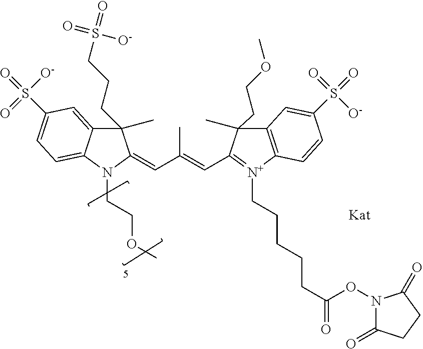

One non-limiting example is a substituted polymethine form of 550 Compound 6/2, shown below:

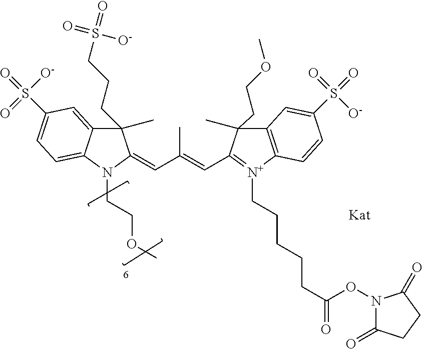

One non-limiting example is a substituted polymethine form of 550 having an ethylene glycol, diethylene glycol, or (poly)ethylene glycol as described for general formula IV, such as the compound

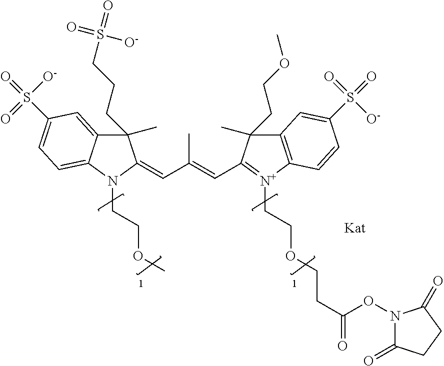

One non-limiting example is a substituted polymethine form of 550 Compound 0/1, shown below:

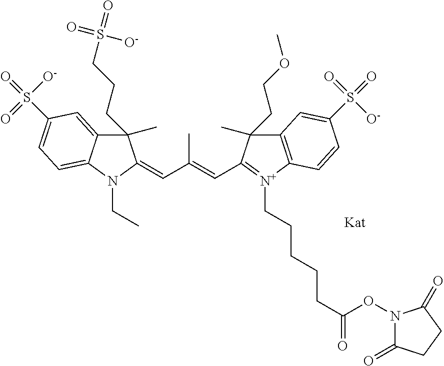 [SMILES: CCN1/C(=C/C(C)=C/C2=[N+](CCCCCC(=O)ON3C(=O)CCC3=O)c3ccc(S(=O)(=O)[O-])cc3C2(C)CCOC)C(C)(CCCS(=O)(=O)[O-])c2cc(S(=O)(=O)[O-])ccc21]

One non-limiting example is a substituted polymethine form of 550 Compound 0/1, shown below:

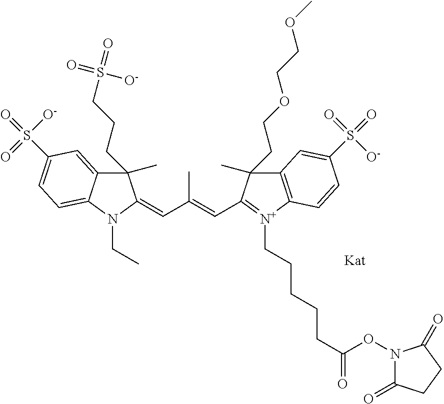 [SMILES: CCN1/C(=C/C(C)=C/C2=[N+](CCCCCC(=O)ON3C(=O)CCC3=O)c3ccc(S(=O)(=O)[O-])cc3C2(C)CCOCCOC)C(C)(CCCS(=O)(=O)[O-])c2cc(S(=O)(=O)[O-])ccc21]

One non-limiting example is a substituted polymethine form of 550 Compound 0/1, shown below:

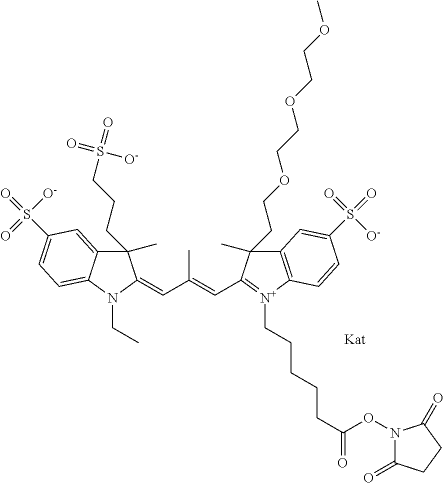 [SMILES: CCN1/C(=C/C(C)=C/C2=[N+](CCCCCC(=O)ON3C(=O)CCC3=O)c3ccc(S(=O)(=O)[O-])cc3C2(C)CCOCCOCCOC)C(C)(CCCS(=O)(=O)[O-])c2cc(S(=O)(=O)[O-])ccc21]

One non-limiting example is a substituted polymethine form of 550 Compound 0/1, shown below:

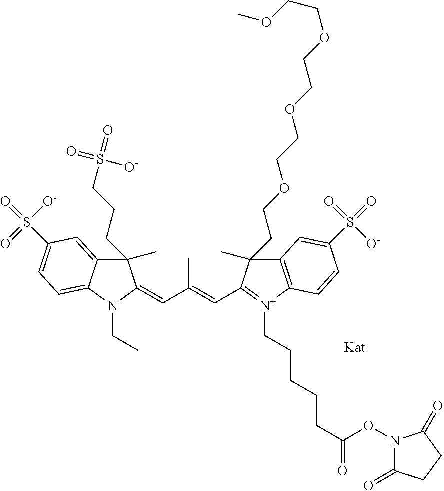 [SMILES: CCN1/C(=C/C(C)=C/C2=[N+](CCCCCC(=O)ON3C(=O)CCC3=O)c3ccc(S(=O)(=O)[O-])cc3C2(C)CCOCCOCCOCCOC)C(C)(CCCS(=O)(=O)[O-])c2cc(S(=O)(=O)[O-])ccc21]

One non-limiting example is a substituted polymethine form of 550 Compound 0/1, shown below:

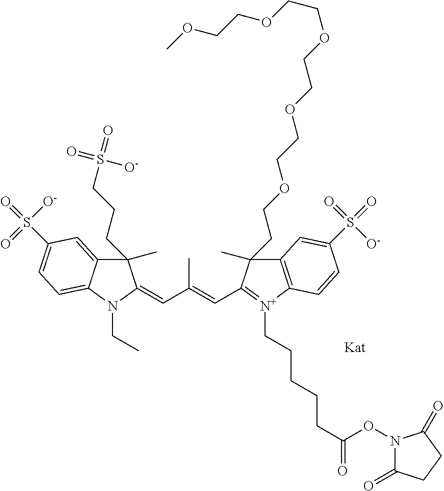 [SMILES: CCN1/C(=C/C(C)=C/C2=[N+](CCCCCC(=O)ON3C(=O)CCC3=O)c3ccc(S(=O)(=O)[O-])cc3C2(C)CCOCCOCCOCCOCCOC)C(C)(CCCS(=O)(=O)[O-])c2cc(S(=O)(=O)[O-])ccc21]

One non-limiting example is a substituted polymethine form of 550 Compound 0/1, shown below:

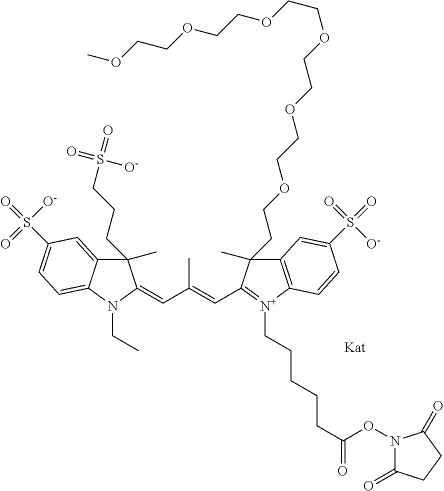 [SMILES: CCN1/C(=C/C(C)=C/C2=[N+](CCCCCC(=O)ON3C(=O)CCC3=O)c3ccc(S(=O)(=O)[O-])cc3C2(C)CCOCCOCCOCCOCCOCCOC)C(C)(CCCS(=O)(=O)[O-])c2cc(S(=O)(=O)[O-])ccc21]

In various embodiments, an ethylene glycol group, diethylene glycol group, and/or a (poly)ethylene glycol group, which will collectively be referred to as a PEG group, unless specifically defined, may be present at position(s) in addition to such groups being present on the N atom(s) of the indole structure.

One non-limiting example of an additionally PEG-substituted compound is a 550 Compound 1/2 according to general formula II where R1 is an ethylene glycol group terminating with a methyl group, shown below:

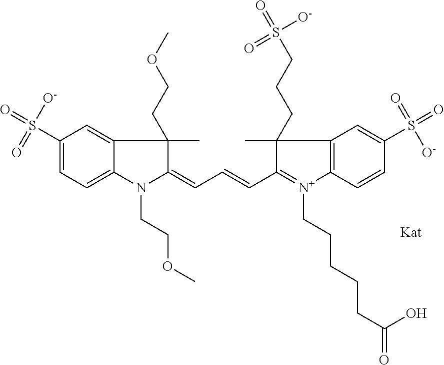

One non-limiting example of an additionally PEG-substituted compound is a 550 Compound 1/2 according to general formula II where R1 is a diethylene glycol group terminating with a methyl group, shown below:

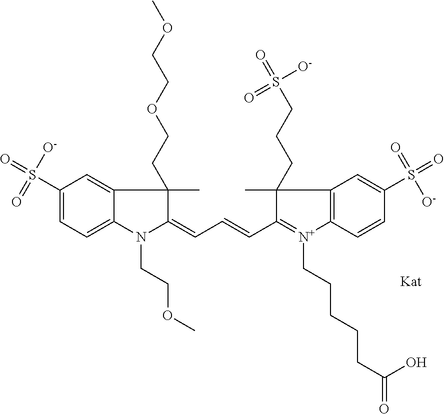

One non-limiting example of an additionally PEG-substituted compound is a 550 Compound 1/2 according to general formula II where R1 is a (poly)ethylene glycol (3) group terminating with a methyl group, shown below:

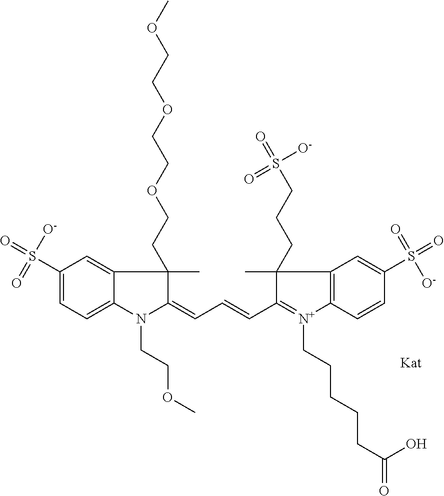

One non-limiting example of an additionally PEG-substituted compound is a 550 Compound 1/2 according to general formula II where R1 is a (poly)ethylene glycol (4) group terminating with a methyl group, shown below:

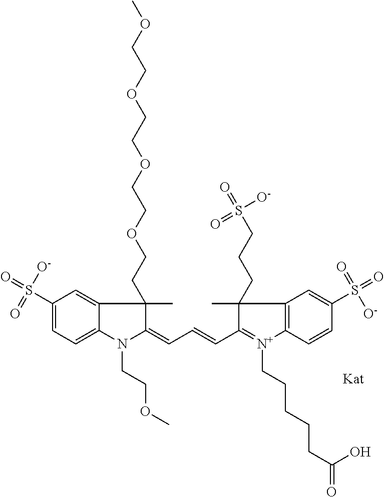

One non-limiting example of an additionally PEG-substituted compound is a 550 Compound 1/2 according to general formula II where R1 is a (poly)ethylene glycol (5) group terminating with a methyl group, shown below:

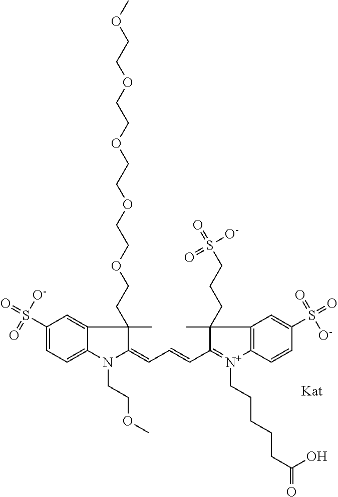

One non-limiting example of an additionally PEG-substituted compound is a 550 Compound 1/2 according to general formula II where R1 is a (poly)ethylene glycol (6) group terminating with a methyl group, shown below:

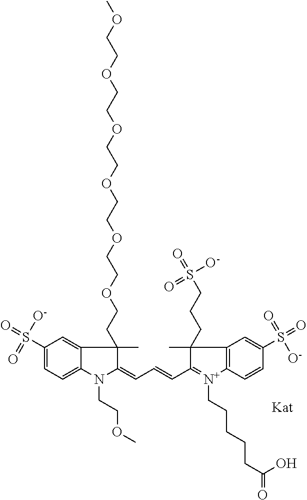

One non-limiting example of an additionally PEG-substituted compound is a 550 Compound 1/2 according to general formula II where R1 is a sulfonamide group -L-SO₂NH—P—Z where Z is a methyl group, shown below:

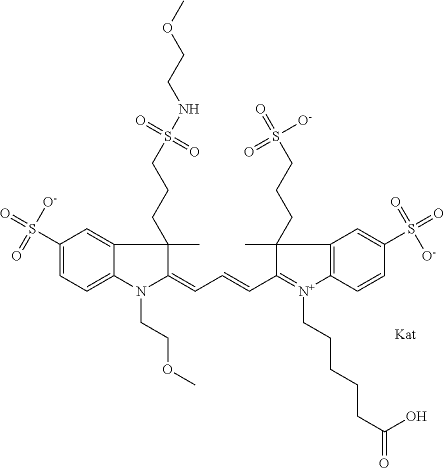

One non-limiting example of an additionally PEG-substituted compound is a 550 Compound 1/2 according to general formula II where R1 is a carboxamide group -L-CONH—P—Z where Z is a methyl group, shown below:

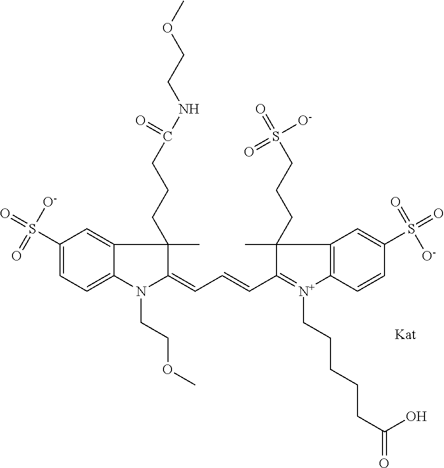

One non-limiting example of an additionally PEG-substituted compound is a 550 Compound 1/2 according to general formula II where R2 is an ethylene glycol group terminating with a methyl group, shown below:

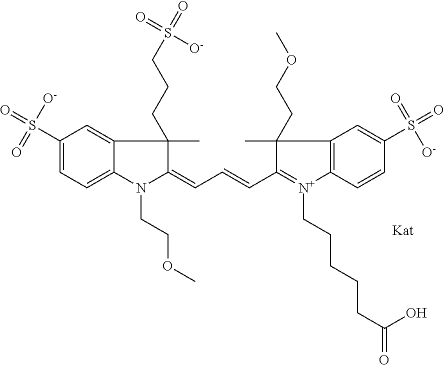

One non-limiting example of an additionally PEG-substituted compound is a 550 Compound 1/2 according to general formula II where R2 is a diethylene glycol group terminating with a methyl group, shown below:

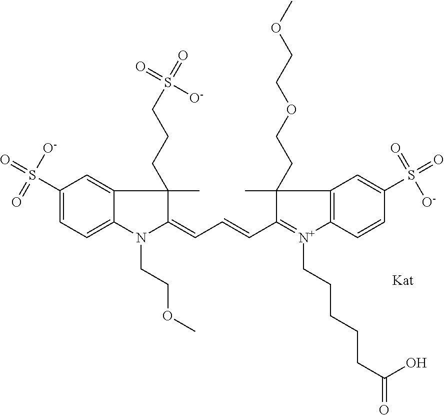

One non-limiting example of an additionally PEG-substituted compound is a 550 Compound 1/2 according to general formula II where R2 is a (poly)ethylene glycol (3) group terminating with a methyl group, shown below:

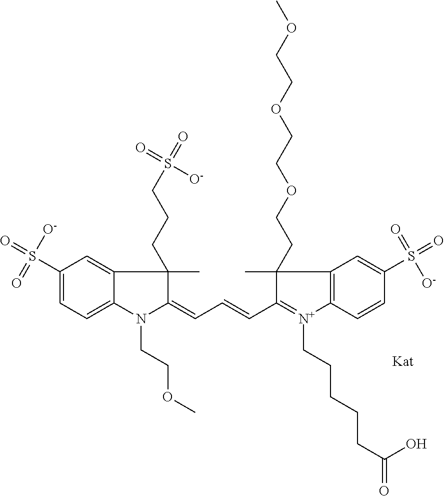

One non-limiting example of an additionally PEG-substituted compound is a 550 Compound 1/2 according to general formula II where R2 is a (poly)ethylene glycol (4) group terminating with a methyl group, shown below:

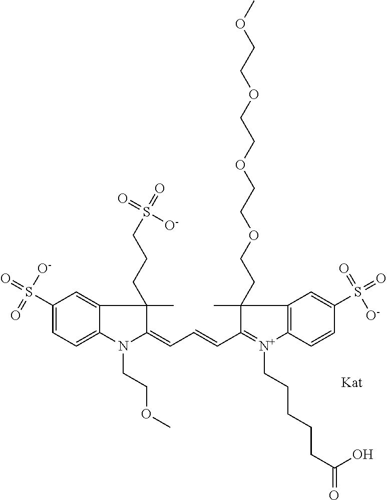

One non-limiting example of an additionally PEG-substituted compound is a 550 Compound 1/2 according to general formula II where R2 is a (poly)ethylene glycol (5) group terminating with a methyl group, shown below:

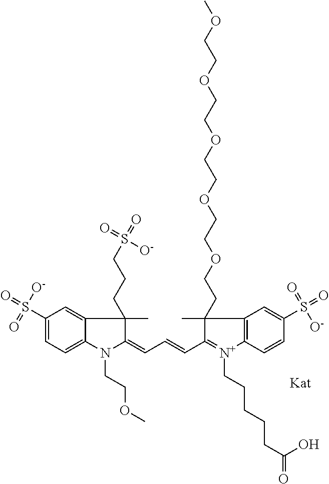

One non-limiting example of an additionally PEG-substituted compound is a 550 Compound 1/2 according to general formula II where R2 is a (poly)ethylene glycol (6) group terminating with a methyl group, shown below:

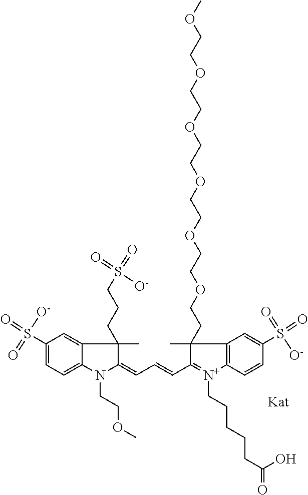

One non-limiting example of an additionally PEG-substituted compound is a 550 Compound 1/2 according to general formula II where R2 is a sulfonamide group -L-SO₂NH—P—Z where Z is a methyl group, shown below:

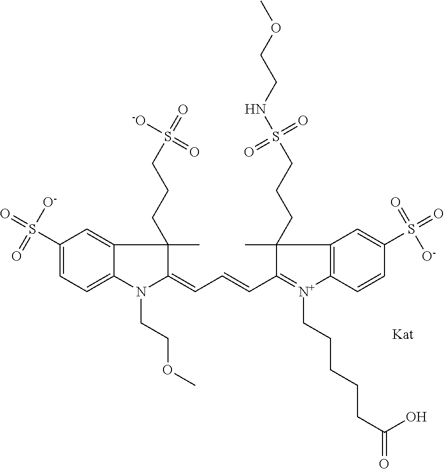

One non-limiting example of an additionally PEG-substituted compound is a 550 Compound 1/2 according to general formula II where R2 is a carboxamide group -L-CONH—P—Z where Z is a methyl group, shown below:

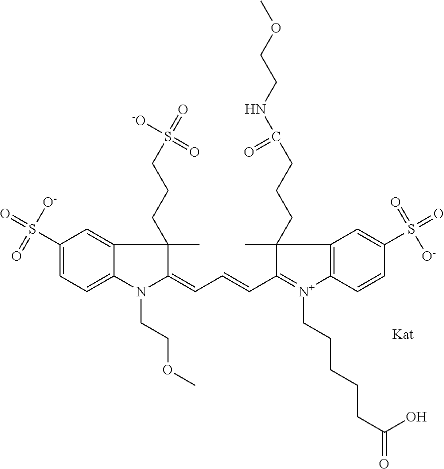

One non-limiting example of an additionally PEG-substituted compound is a 550 Compound 1/3 according to general formula II where both R1 and R2 are an ethylene glycol group terminating with a methyl group, shown below:

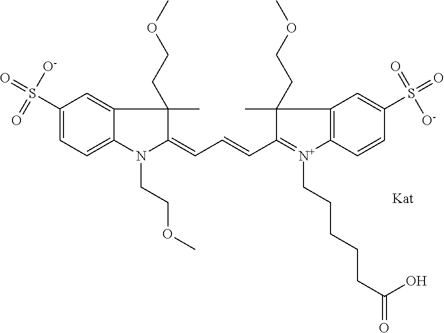

One non-limiting example of an additionally PEG-substituted compound is a 550 Compound 1/3 according to general formula II where both R1 and R2 are a diethylene glycol group terminating with a methyl group, shown below:

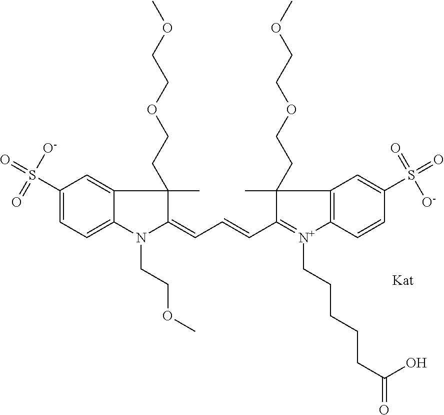

One non-limiting example of an additionally PEG-substituted compound is a 550 Compound 1/3 according to general formula II where both R1 and R2 are a (poly)ethylene glycol (3) group terminating with a methyl group, shown below:

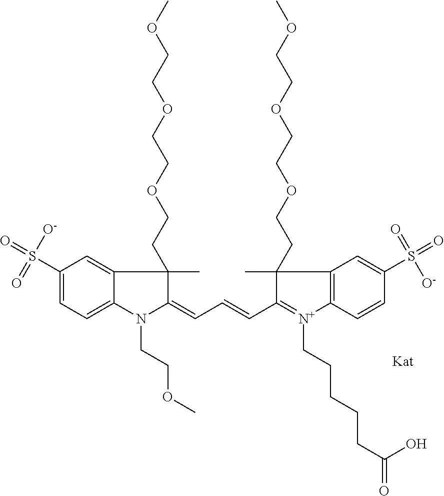

One non-limiting example of an additionally PEG-substituted compound is a 550 Compound 1/3 according to general formula II where both R1 and R2 are a (poly)ethylene glycol (4) group terminating with a methyl group, shown below:

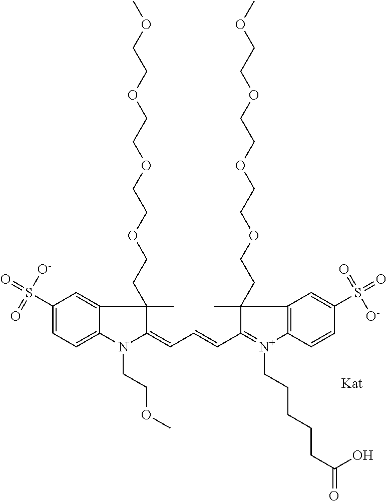

One non-limiting example of an additionally PEG-substituted compound is a 550 Compound 4/4 according to general formula III where both R1 and R2 are a (poly)ethylene glycol (4) group terminating with a methyl group, and R7 and R8 are sulfo, shown below:

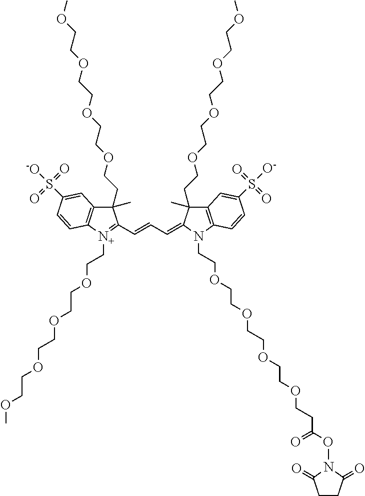

One non-limiting example of an additionally PEG-substituted compound is a 550 Compound 4/4 according to general formula III where both R1 and R2 are a (poly)ethylene glycol (4) group terminating with a methyl group, and R7 and R8 are H, shown below:

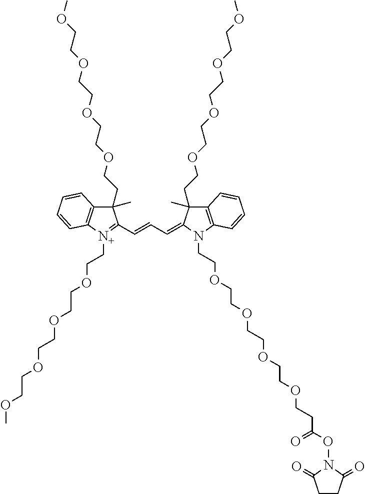

One non-limiting example of an additionally PEG-substituted compound is a 550 Compound 1/3 according to general formula II where both R1 and R2 are a (poly)ethylene glycol (5) group terminating with a methyl group, shown below:

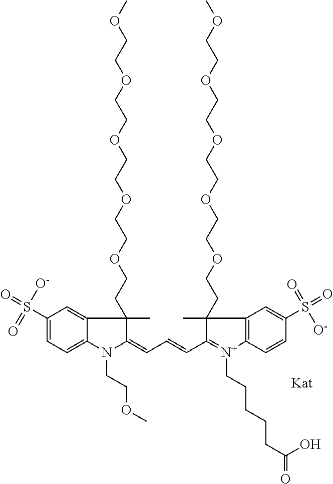

One non-limiting example of an additionally PEG-substituted compound is a 550 Compound 1/3 according to general formula II where both R1 and R2 are a (poly)ethylene glycol (6) group terminating with a methyl group, shown below:

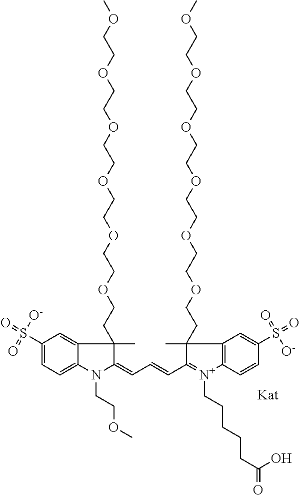

One non-limiting example of an additionally PEG-substituted compound is a 550 Compound 1/2 according to general formula II where R8 is an ethylene glycol group terminating with a methyl group, shown below:

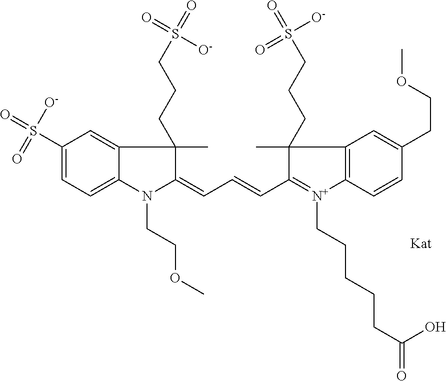

One non-limiting example of an additionally PEG-substituted compound is a 550 Compound 1/2 according to general formula II where R8 is a sulfonamide group —SO₂NH—P—Z where Z is a methyl group, shown below:

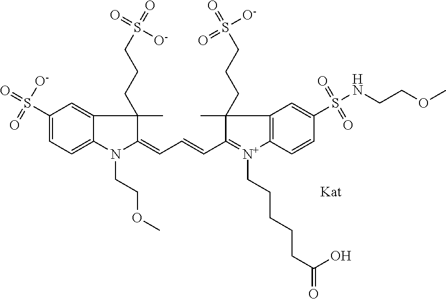

One non-limiting example of an additionally PEG-substituted compound is a 550 Compound 1/2 according to general formula II where R8 is a carboxamide group —CONH—P—Z where Z is a methyl group, shown below:

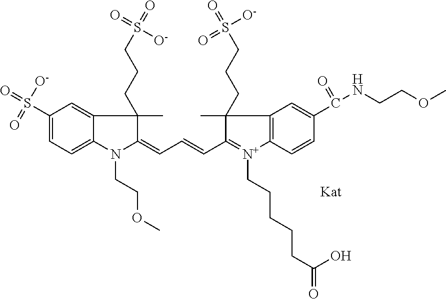

One non-limiting example of an additionally PEG-substituted compound is a 550 Compound 1/2 according to general formula II where R7 is an ethylene glycol group terminating with a methyl group, shown below:

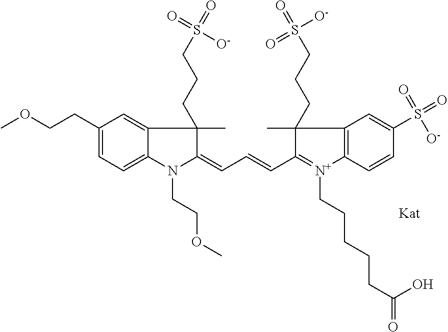

One non-limiting example of an additionally PEG-substituted compound is a 550 Compound 1/2 according to general formula II where R7 is a sulfonamide group —SO₂NH—P—Z where Z is a methyl group, shown below:

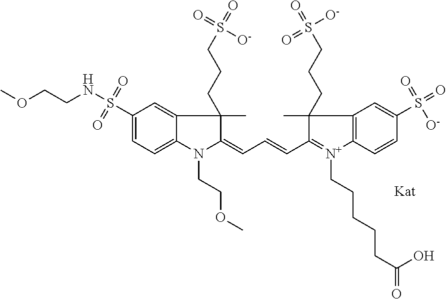

One non-limiting example of an additionally PEG-substituted compound is a 550 Compound 1/2 according to general formula II where R7 is a carboxamide group —CONH—P—Z where Z is a methyl group, shown below:

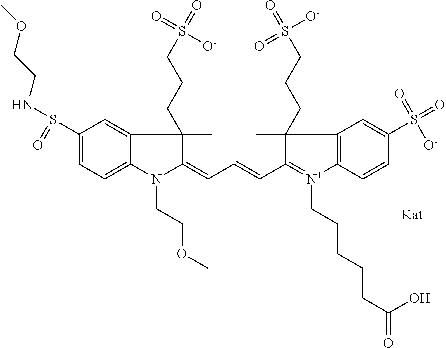

One non-limiting example of an additionally PEG-substituted compound is a 550 Compound 1/3 according to general formula II where both R7 and R8 are an ethylene glycol group terminating with a methyl group, shown below:

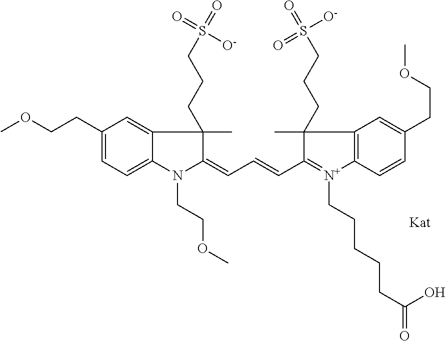

One non-limiting example of an additionally PEG-substituted compound is a 550 Compound 1/3 according to general formula II where both R7 and R8 are a sulfonamide group —SO₂NH—P—Z where Z is a methyl group, shown below:

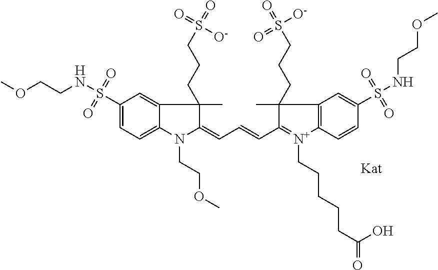

One non-limiting example of an additionally PEG-substituted compound is a 550 Compound 1/3 according to general formula II where both R7 and R8 are a carboxamide group —CONH—P—Z where Z is a methyl group, shown below:

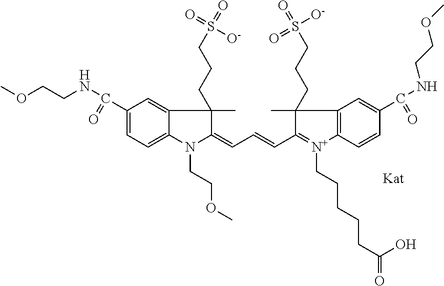

In one embodiment the compound is 650 Compound 1/2

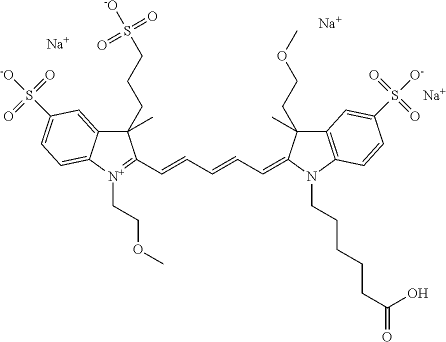

650 Compound 1/2 ((2-((1E,3E,5E)-5-(1-(5-carboxypentyl)-3-(2-methoxyethyl)-3-methyl-5-sulfonatoindolin-2-ylidene)penta-1,3-dienyl)-1-(2-methoxyethyl)-3-methyl-3-(3-sulfonatopropyl)-3H-indolium-5-sulfonate tri sodium salt) contains ethylene glycol on the indole N of the left heterocycle, i.e., a methylated ethylene glycol. The methyl group on the ethylene glycol prevents the terminal —OH from oxidation. Oxidation is known to occur, over time, on an unprotected PEG terminus (i.e., an unprotected ethylene glycol group, diethylene glycol group, or (poly)ethylene glycol group). Adding a methyl ether provides this protection, and prevents reaction with electrophilic reactive groups.

In embodiments, e.g., for functional assays, the inventive compounds are activated. Activation of the compound adds a chemical moiety such that the compound is in a form that can be conjugated to a biological moiety. Examples of chemical moieties for activation are described below with reference to activation of 650 Compound 1, but one skilled in the art appreciates that activation is not limited to these examples. One non-limiting example of an activated compound is the NHS-ester of 650 Compound 1/2, shown below:

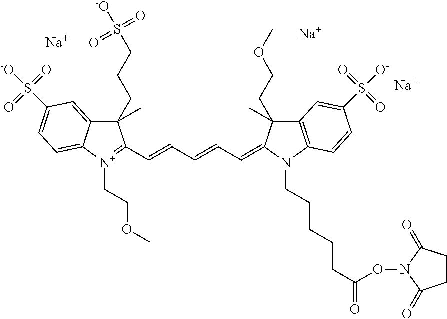

In one embodiment the compound is a NHS-ester of 650 Compound 1/2 where, according to general formula I, o is 1, shown below:

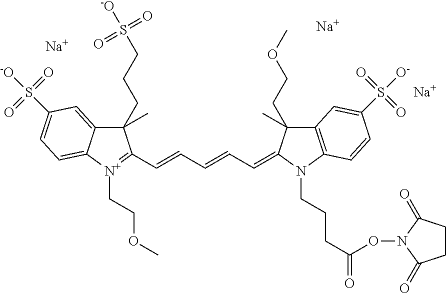

In one embodiment the compound is a NHS-ester of 650 Compound 1/2 where, according to general formula I, o is 5, shown below:

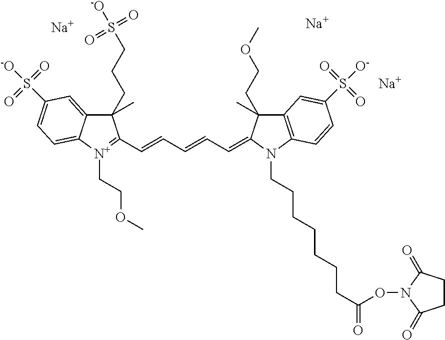

One non-limiting example of a NHS-ester of 650 Compound 1/3, according to formula III, where m=1 and p=1, is shown below:

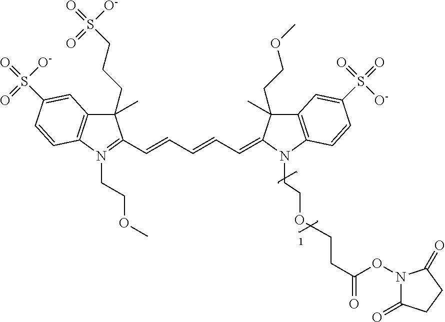

One non-limiting example of a NHS-ester of 650 Compound 1/3, according to general formula III, where m=1 and p=2, is shown below:

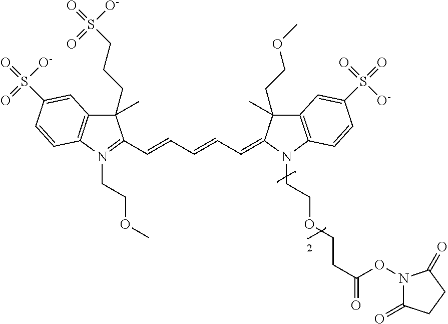

One non-limiting example of a NHS-ester of 650 Compound 1/3, according to general formula III, where m=1 and p=3, is shown below:

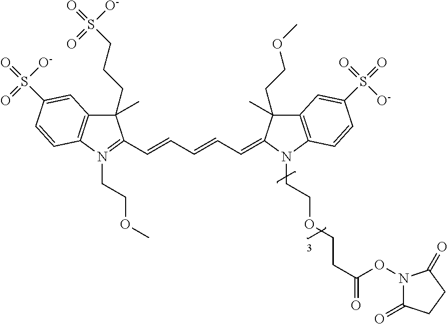

One non-limiting example of a NHS-ester of 650 Compound 1/3, according to general formula III, where m=1 and p=4, is shown below:

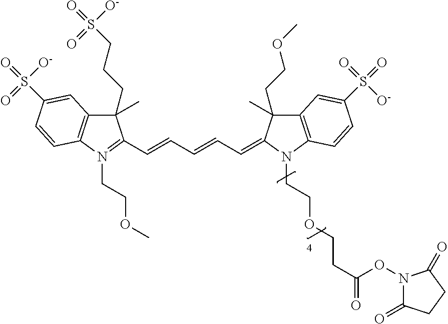

One non-limiting example of a NHS-ester of 650 Compound 1/3, according to general formula III, where m=1 and p=6, is shown below:

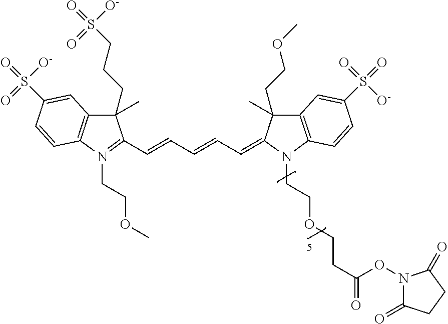

One non-limiting example of a NHS-ester of 650 Compound 1/3, according to general formula III, where m=1 and p=6, is shown below:

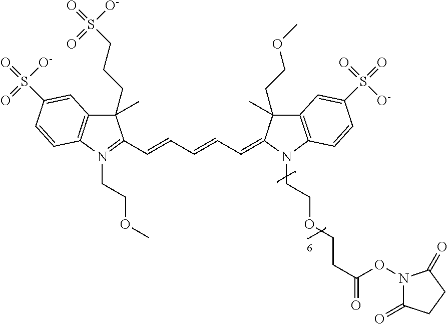

One non-limiting example of a NHS-ester of 650 Compound 2/3, according to general formula III, where m=2 and p=1, is shown below:

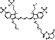

One non-limiting example of a NHS-ester of 650 Compound 2/3, according to general formula III, where m=2 and p=2, is shown below:

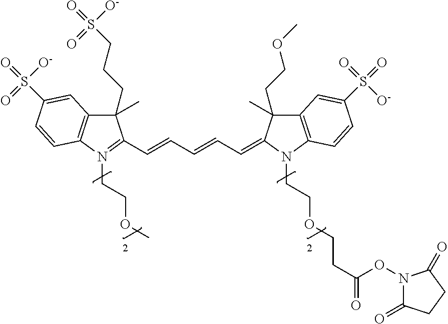

One non-limiting example of a NHS-ester of 650 Compound 2/3, according to general formula III, where m=2 and p=3, is shown below:

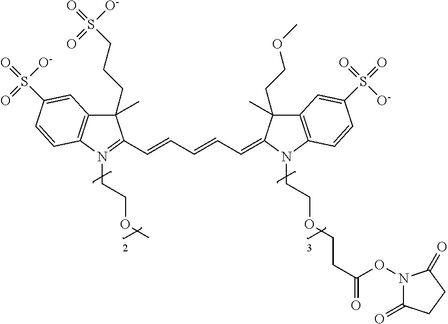

One non-limiting example of a NHS-ester of 650 Compound 3/3, according to general formula III, where m=3 and p=1, is shown below:

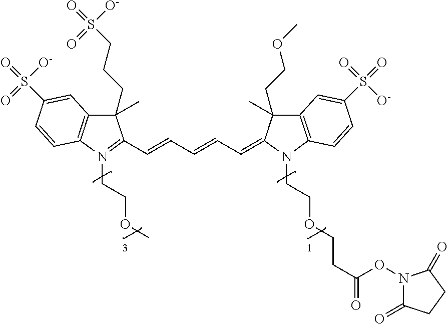

One non-limiting example of a NHS-ester of 650 Compound 3/3, according to general formula III, where m=3 and p=2, is shown below:

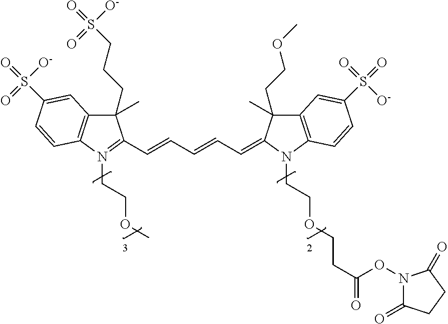

One non-limiting example of a NHS-ester of 650 Compound 3/3, according to general formula III, where m=3 and p=3, is shown below:

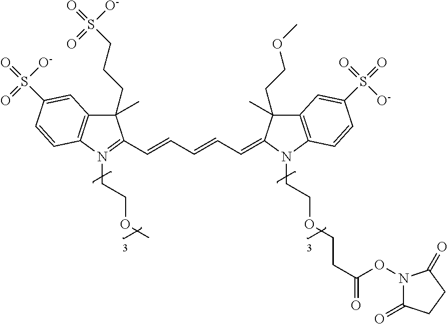

One non-limiting example of a NHS-ester of 650 Compound 4/3, according to general formula III, where m=4 and p=1, is shown below:

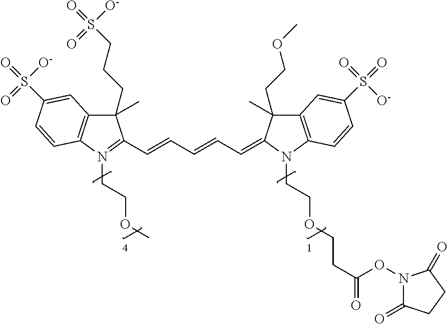

One non-limiting example of a NHS-ester of 650 Compound 5/3, according to general formula III, where m=5 and p=1, is shown below:

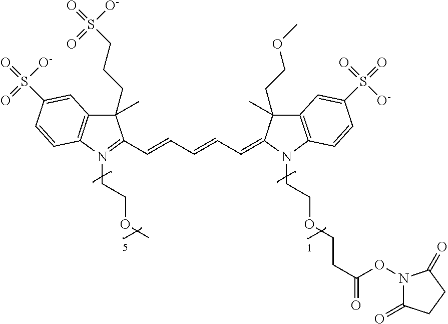

One non-limiting example of a NHS-ester of 650 Compound 6/3, according to general formula III, where m=6 and p=1, is shown below:

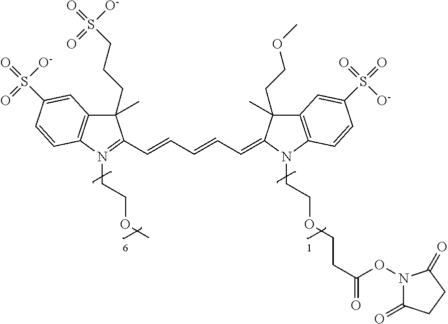

One non-limiting example of an activated 650 Compound 1/2 is the tetrafluorophenyl (TFP)-ester of 650 Compound 1/2, shown below:

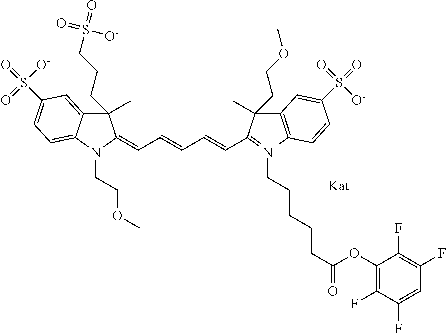 [SMILES: COCCN1/C(=C/C=C/C=C/C2=[N+](CCCCCC(=O)Oc3c(F)c(F)cc(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 650 Compound 1/2 is the sulfotetrafluorophenyl (STP)-ester of 650 Compound 1/2, shown below:

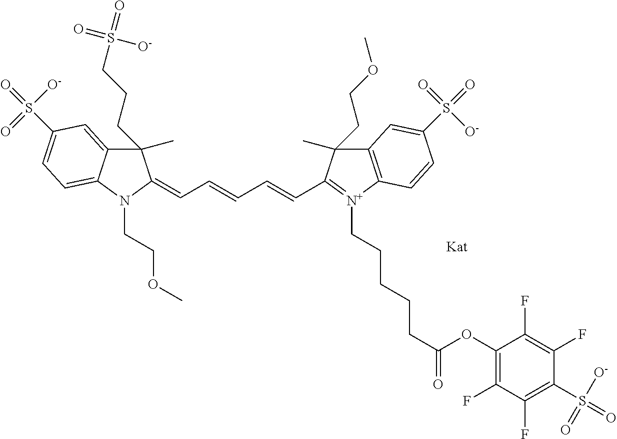 [SMILES: COCCN1/C(=C/C=C/C=C/C2=[N+](CCCCCC(=O)Oc3c(F)c(F)c(S(=O)(=O)[O-])c(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 650 Compound 1/2 is the hydrazide of 650 Compound 1, shown below:

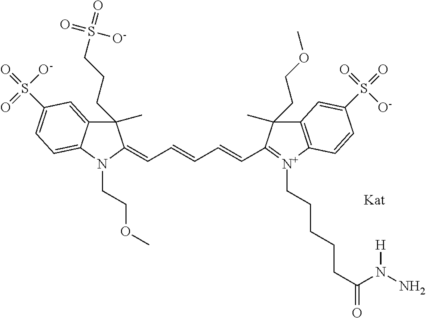 [SMILES: COCCN1/C(=C/C=C/C=C/C2=[N+](CCCCCC(=O)NN)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 650 Compound 1/2 is the maleimide of 650 Compound 1, shown below:

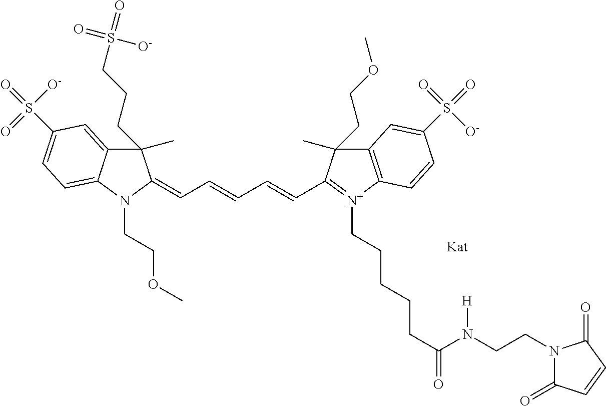 [SMILES: COCCN1/C(=C/C=C/C=C/C2=[N+](CCCCCC(=O)NCCN3C(=O)C=CC3=O)c3ccc(S(=O)(=O)[O-])cc3C2(C)CCOC)C(C)(CCCS(=O)(=O)[O-])c2cc(S(=O)(=O)[O-])ccc21]

In one embodiment the compound is 650 Compound 2/2

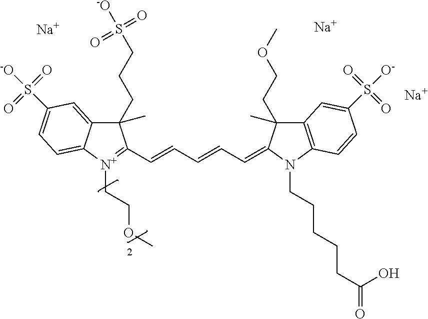

650 Compound 2/2 (2-((1E,3E,5E)-5-(1-(5-carboxypentyl)-3-(2-methoxyethyl)-3-methyl-5-sulfonatoindolin-2-ylidene)penta-1,3-dienyl)-1-(2-(2-methoxyethoxy)ethyl)-3-methyl-3-(3-sulfonatopropyl)-3H-indolium-5-sulfonate tri sodium salt.) contains a (poly)ethylene glycol on the indole N of the left heterocycle. The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 650 Compound 2/2 is activated as described above, one non-limiting example of which is the NHS-ester form of 650 Compound 2/2, shown below.

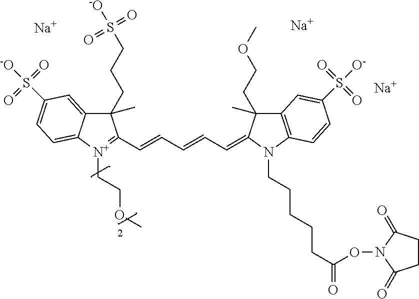

In one embodiment the compound is 650 Compound 3/2

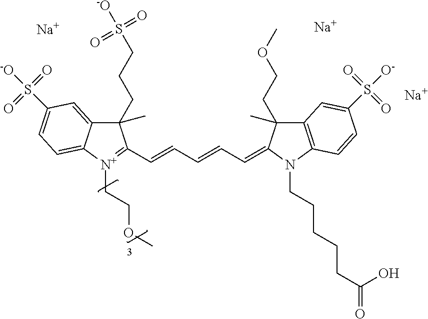

650 Compound 3/2 (2-((1E,3E,5E)-5-(1-(5-carboxypentyl)-3-(2-methoxyethyl)-3-methyl-5-sulfonatoindolin-2-ylidene)penta-1,3-dienyl)-1-(2-(2-(2-methoxyethoxy)ethoxy)ethyl)-3-methyl-3-(3-sulfonatopropyl)-3H-indolium-5-sulfonate tri sodium salt) contains a (poly)ethylene glycol on the indole N of the left heterocycle. The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 650 Compound 3/2 is activated as described above, one non-limiting example of which is the NHS-ester form of 650 Compound 3/2, shown below.

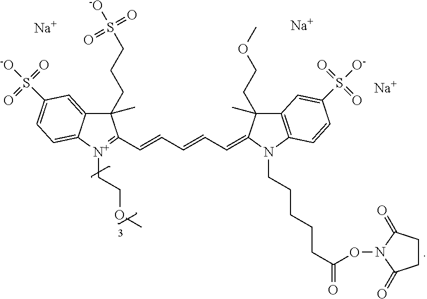

In one embodiment the compound is 650 Compound 4/2

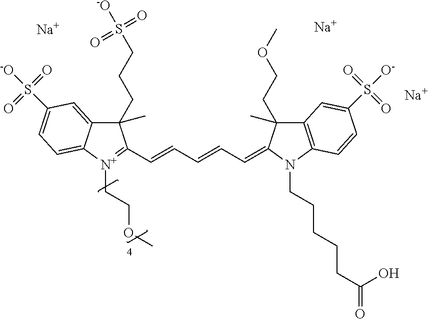

650 Compound 4/2 (2-((1E,3E,5E)-5-(1-(5-carboxypentyl)-3-(2-methoxyethyl)-3-methyl-5-sulfonatoindolin-2-ylidene)penta-1,3-dienyl)-3-methyl-3-(3-sulfonatopropyl)-1-(2,5,8,11-tetraoxatridecan-13-yl)-3H-indolium-5-sulfonate) contains a (poly)ethylene glycol on the indole N of the left heterocycle. The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 650 Compound 4/2 is activated as described above.

In one embodiment the compound is 650 Compound 5/2

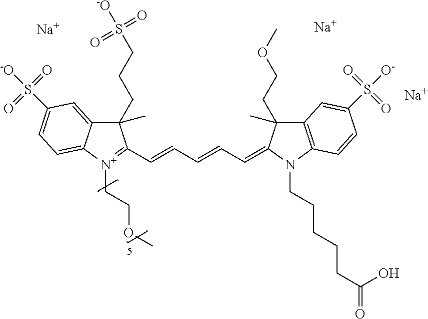

650 Compound 5/2 (2-((1E,3E,5E)-5-(1-(5-carboxypentyl)-3-(2-methoxyethyl)-3-methyl-5-sulfonatoindolin-2-ylidene)penta-1,3-dienyl)-1-(2,5,8,11,14-pentaoxahexadecan-16-yl)-3-methyl-3-(3-sulfonatopropyl)-3H-indolium-5-sulfonate) contains a (poly)ethylene glycol on the indole N of the left heterocycle. The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 650 Compound 5/2 is activated as described above.

In one embodiment the compound is 650 Compound 6/2

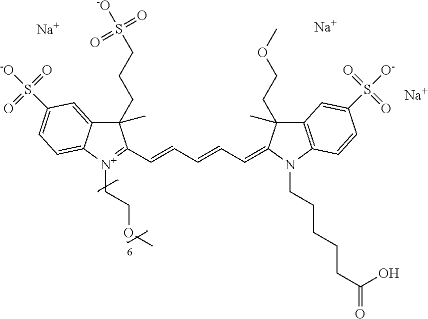

650 Compound 6/2 (2-((1E,3E,5E)-5-(1-(5-carboxypentyl)-3-(2-methoxyethyl)-3-methyl-5-sulfonatoindolin-2-ylidene)penta-1,3-dienyl)-3-methyl-1-(2,5,8,11,14,17-hexaoxanonadecan-19-yl)-3-(3-sulfonatopropyl)-3H-indolium-5-sulfonate) contains a (poly)ethylene glycol on the indole N of the left heterocycle. The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 650 Compound 6/2 is activated as described above.

In embodiments, the compound contains one or more substitutions of the polymethine linker. In one embodiment the compound has general formula Va

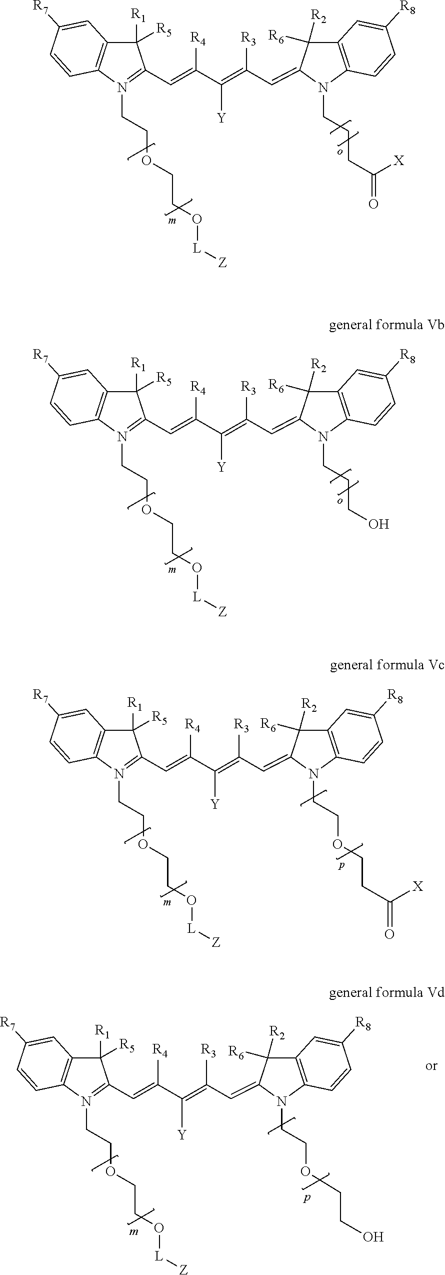

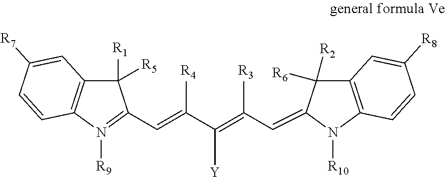

where each of R¹, R², R⁵, and R⁶ is the same or different and is independently selected from the group consisting of an aliphatic, heteroaliphatic, sulfoalkyl group, heteroaliphatic with terminal SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group -L-SO₂NH—P-L-Z, and a caboxamide group -L-CONH—P-L-Z; each of R⁷ and R⁸ is the same or different and is independently selected from either H, SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group —SO₂NH—P-L-Z, or a caboxamide group —CONH—P-L-Z; each of R⁹ and R¹⁰ is the same or different and is independently selected from the group consisting of an alkyl, a sulfoalkyl, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a PEG group P-L-X, L-Z, L-X; where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur; where Z is selected from the group consisting of H, CH₃, an alkyl, a heteroalkyl, NH₂, —COO, —COOH, —COSH, CO—NH—NH₂, —COF, —COCl, —COBr, —COI, —COO-Su (succinimidyl/sulfo-succinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO⁻, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-O-phosphoramidite, —CONR′-L-CHO, —CONR′-L-maleimide, and —CONR′-L-NH—CO—CH₂—I; R′ and R″ is selected from the group consisting of H, aliphatic group, and heteroaliphatic group, and the biomolecule is a protein, antibody, nucleotide, oligonucleotide, biotin, or hapten; X is selected from the group consisting of —OH, —SH, —NH₂, —NH—NH₂, —F, —Cl, —Br, I, —NHS (hydroxysuccinimidyl/sulfosuccinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group; Kat is a number of Na⁺, K⁺, Ca²⁺, ammonia, or other cation(s) needed to compensate the negative charge brought by the cyanine; m is an integer from 0 to 5 inclusive; p is an integer from 1 to 6 inclusive; each of R3 and R4 is the same or different and is independently hydrogen, an aliphatic group, a heteroaliphatic group, or a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive; or R3 and R4 together form a cyclic structure where R3 and R4 are joined using a divalent structural element selected from the group consisting of —(CH₂)_{q}—, —(CH₂)_{q}O(CH₂)_{q′}—, —(CH₂)_{q}S(CH₂)_{q′}—, —(CH₂)_{q}CH═CH—, —OCH═CH— where each of q and q′ is the same or different and is a integer from 2 to 6 inclusive; and Y is selected from the group consisting of hydrogen, alkyl, sulfoalkyl, fluorine, chlorine, bromine, and a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive; with the proviso that at least one of R¹, R², R³, R⁴, R⁵, R⁶, R⁷, and R⁸ contains a PEG group.

In one embodiment the compound of general formula V wherein each of R3 and R4 is the same or different and is independently hydrogen, an aliphatic group, or a heteroaliphatic group, or R3 and R4 together form a cyclic structure where R3 and R4 are directly joined or joined using a divalent structural element selected from the group consisting of —(CH₂)_{q}— and CH═CH, where q is an integer from 1 to 2 inclusive, to result in a 3-, 4-, or 5-membered ring.

In one embodiment an isolated enantiomeric mixture selected from diastereomer Ia of general formula Va

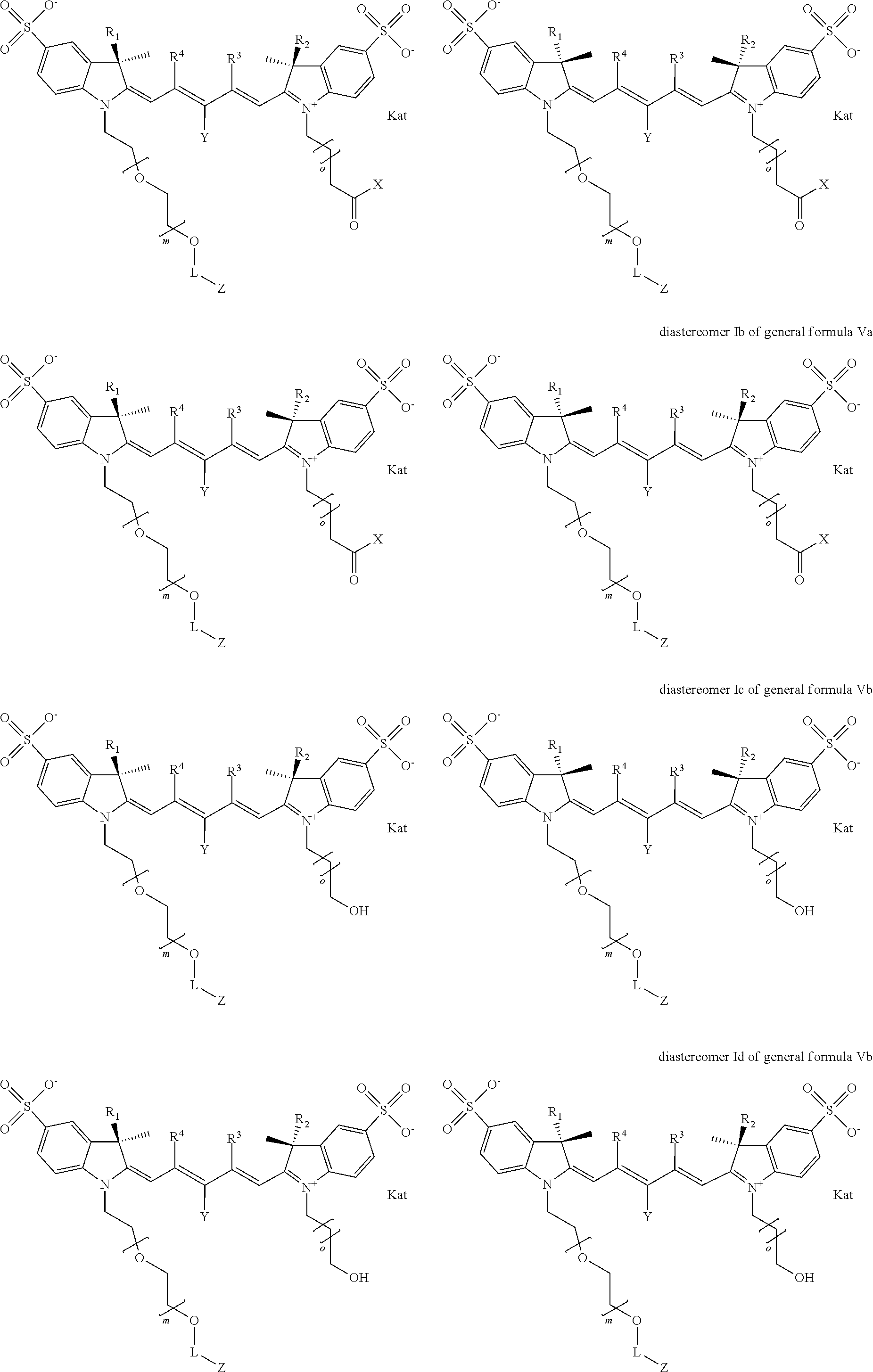

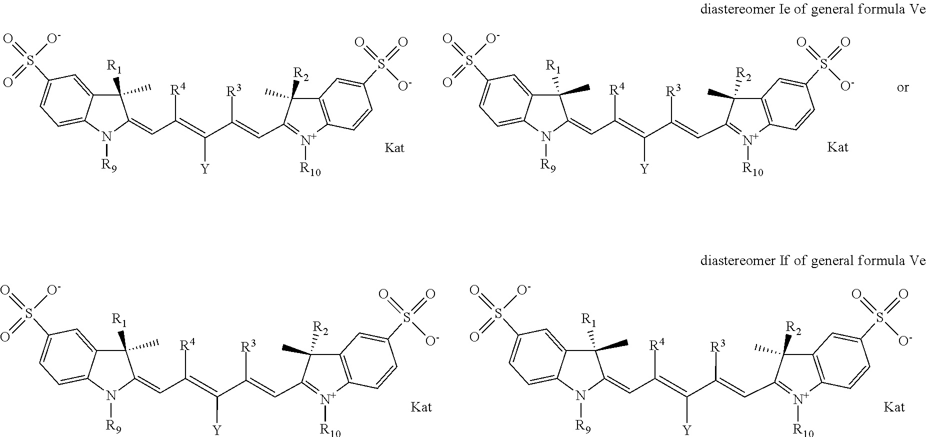

is provided, where each of R¹, R², R⁵, and R⁶ is the same or different and is independently selected from the group consisting of an aliphatic, heteroaliphatic, sulfoalkyl group, heteroaliphatic with terminal SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group -L-SO₂NH—P-L-Z, and a caboxamide group -L-CONH—P-L-Z; each of R⁷ and R⁸ is the same or different and is independently selected from either H, SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group —SO₂NH—P-L-Z, or a caboxamide group —CONH—P-L-Z; where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur; where Z is selected from the group consisting of H, CH₃, an alkyl, a heteroalkyl, NH₂, —COO⁻, —COOH, —COSH, CO—NH—NH₂, —COF, —COCl, —COBr, —COI, —COO-Su (succinimidyl/sulfo-succinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-O-phosphoramidite, —CONR′-L-CHO, —CONR′-L-maleimide, and —CONR′-L-NH—CO—CH₂—I; R′ and R″ is selected from the group consisting of H, aliphatic group, and heteroaliphatic group, and the biomolecule is a protein, antibody, nucleotide, oligonucleotide, biotin, or hapten; X is selected from the group consisting of —OH, —SH, —NH₂, —NH—NH₂, —F, —Cl, —Br, I, —NHS (hydroxysuccinimidyl/sulfosuccinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group; Kat is a number of Na⁺, K⁺, Ca²⁺, ammonia, or other cation(s) needed to compensate the negative charge brought by the cyanine; m is an integer from 0 to 5 inclusive; o is an integer from 0 to 12 inclusive; each of R3 and R4 is the same or different and is independently hydrogen, an aliphatic group, a heteroaliphatic group, or a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive; or R3 and R4 together form a cyclic structure where R3 and R4 are joined using a divalent structural element selected from the group consisting of —(CH₂)_{q}—, —(CH₂)_{q}O(CH₂)_{q′}—, —(CH₂)_{q}S(CH₂)_{q′}—, —(CH₂)_{q}CH═CH—, —OCH═CH— where each of q and q′ is the same or different and is a integer from 2 to 6 inclusive; and Y is selected from the group consisting of hydrogen, alkyl, sulfoalkyl, fluorine, chlorine, bromine, and a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive; with the proviso that at least one of R¹, R², R³, and R⁴ contains a PEG group.

In one embodiment the compound of general formula V wherein each of R3 and R4 is the same or different and is independently hydrogen, an aliphatic group, or a heteroaliphatic group, or R3 and R4 together form a cyclic structure where R3 and R4 are directly joined or joined using a divalent structural element selected from the group consisting of —(CH₂)_{q}— and CH═CH, where q is an integer from 1 to 2 inclusive, to result in a 3-, 4-, or 5-membered ring.

One non-limiting example is a substituted polymethine form of 650 Compound 1/2, shown below:

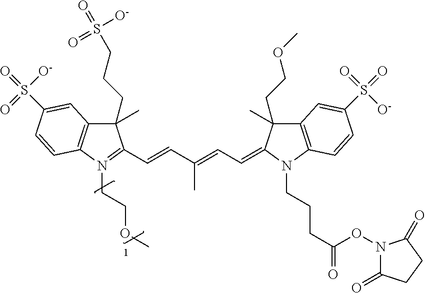

One non-limiting example is a substituted polymethine form of 650 Compound 2/2, shown below:

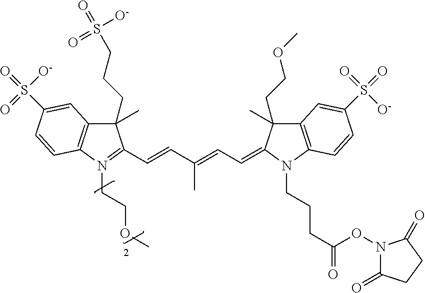

One non-limiting example is a substituted polymethine form of 650 Compound 3/2, shown below:

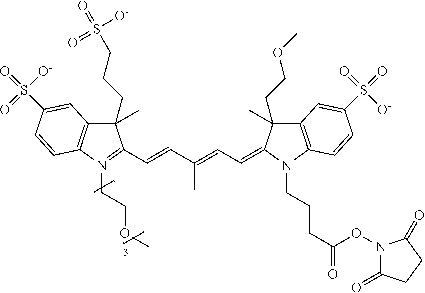

One non-limiting example is a substituted polymethine form of 650 Compound 4/2, shown below:

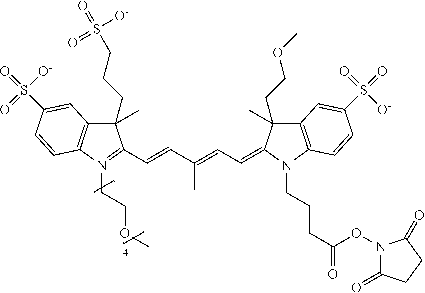

One non-limiting example is a substituted polymethine form of 650 Compound 5/2, shown below:

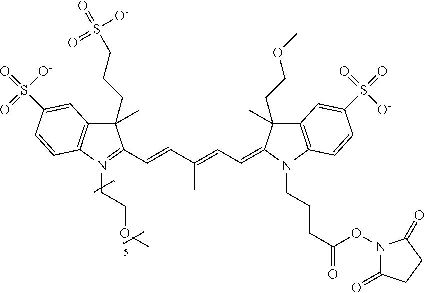

One non-limiting example is a substituted polymethine form of 650 Compound 6/2, shown below:

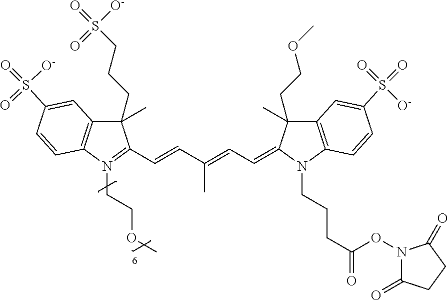

One non-limiting example is a substituted polymethine form of 650 Compound 0/1, shown below:

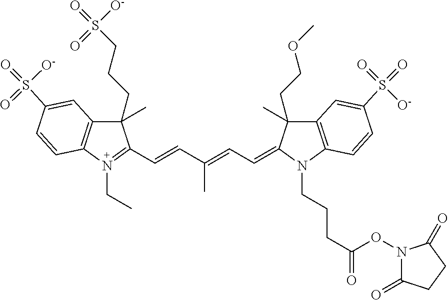

One non-limiting example is a substituted polymethine form of 650 Compound 0/1, shown below:

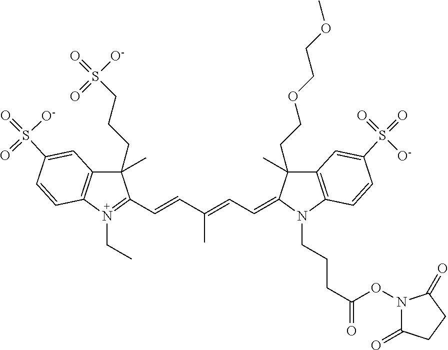

One non-limiting example is a substituted polymethine form of 650 Compound 0/1, shown below:

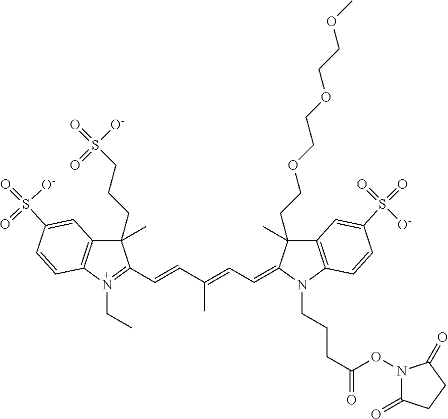

One non-limiting example is a substituted polymethine form of 650 Compound 0/1, shown below:

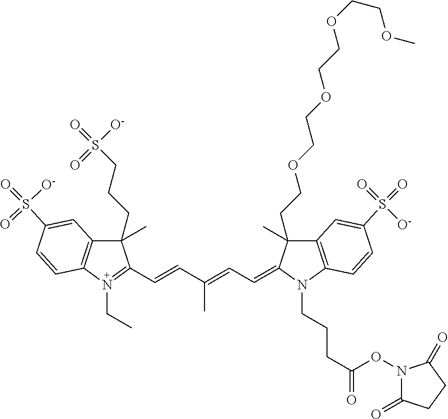

One non-limiting example is a substituted polymethine form of 650 Compound 0/1, shown below:

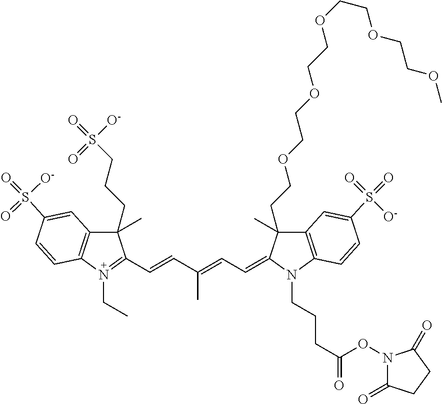

One non-limiting example is a substituted polymethine form of 650 Compound 0/1, shown below:

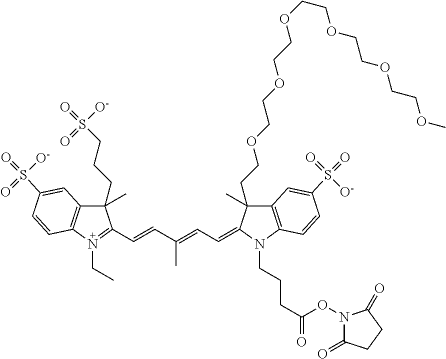

One non-limiting example is a substituted polymethine form of 650 Compound 1/3 having an ethylene glycol, diethylene glycol, or (poly)ethylene glycol as described for general formula V, such as the compound shown below:

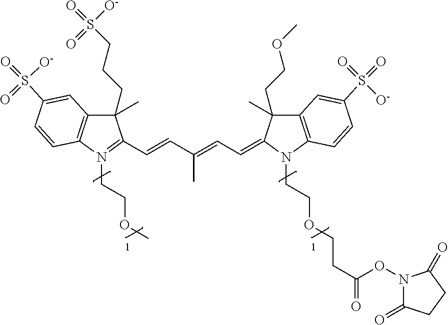

One non-limiting example is a substituted polymethine form of 650 Compound 1/2, shown below:

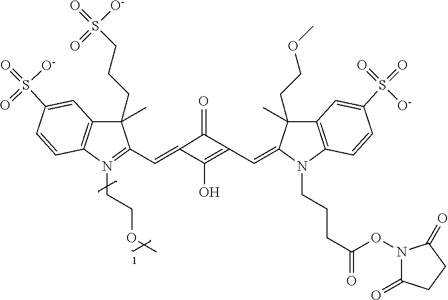

One non-limiting example is a substituted polymethine form of 650 Compound 2/2, shown below:

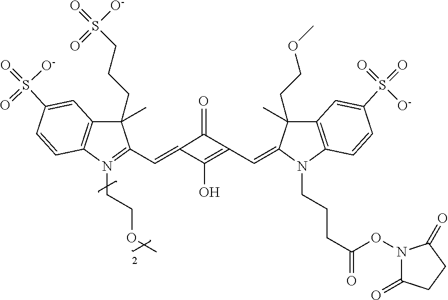

One non-limiting example is a substituted polymethine form of 650 Compound 3/2, shown below:

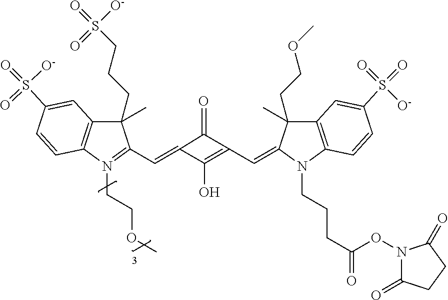

One non-limiting example is a substituted polymethine form of 650 Compound 4/2, shown below:

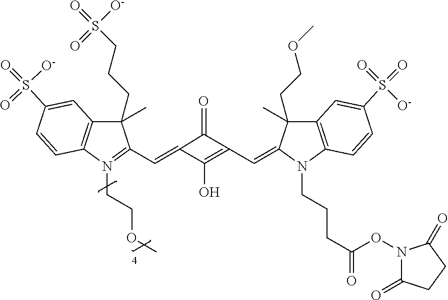

One non-limiting example is a substituted polymethine form of 650 Compound 5/2, shown below:

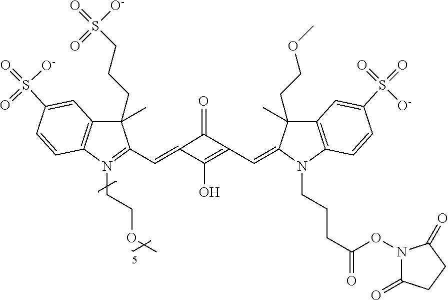

One non-limiting example is a substituted polymethine form of 650 Compound 6/2, shown below:

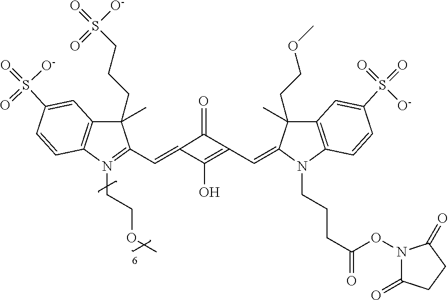

One non-limiting example is a substituted polymethine form of 650 Compound 0/1, shown below:

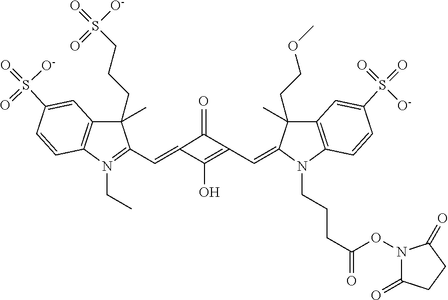

One non-limiting example is a substituted polymethine form of 650 Compound 0/1, shown below:

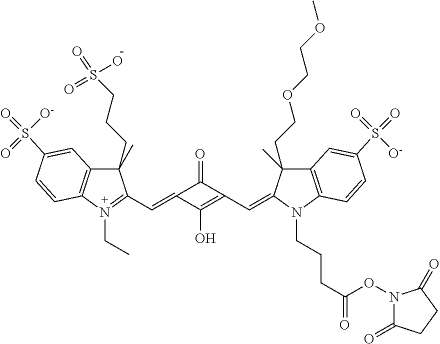

One non-limiting example is a substituted polymethine form of 650 Compound 0/1, shown below:

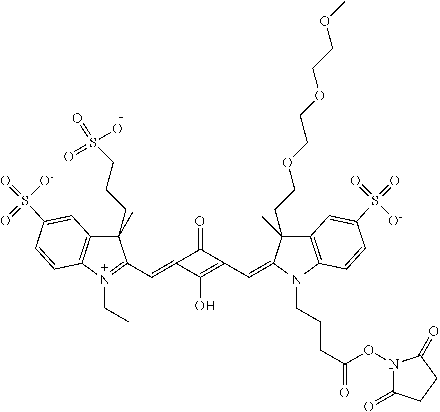

One non-limiting example is a substituted polymethine form of 650 Compound 0/1, shown below:

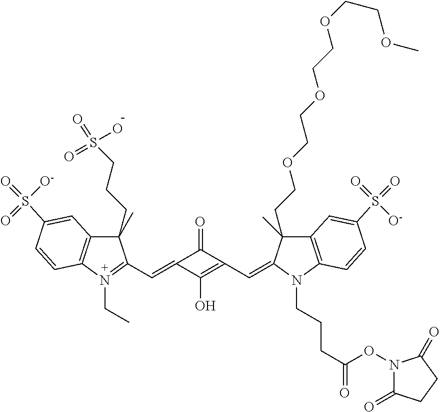

One non-limiting example is a substituted polymethine form of 650 Compound 0/1, shown below:

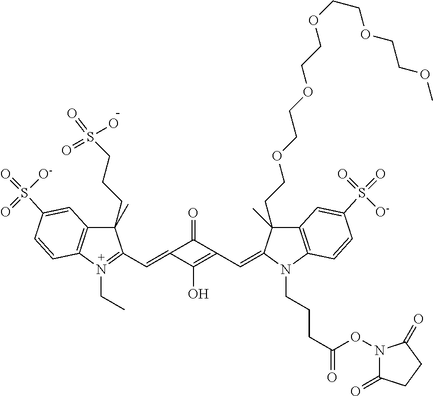

One non-limiting example is a substituted polymethine form of 650 Compound 0/1, shown below:

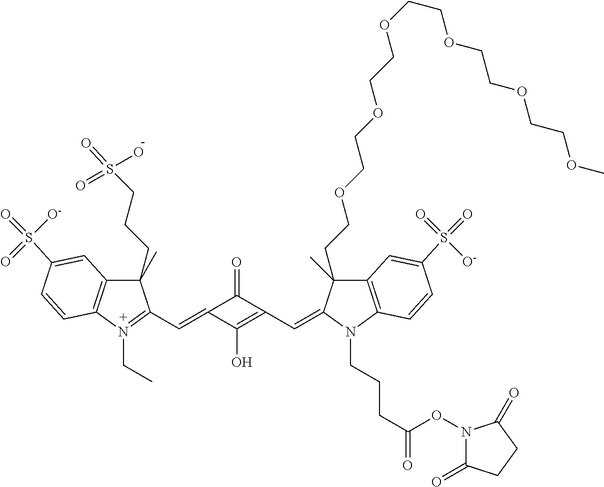

One non-limiting example is a substituted polymethine form of 650 Compound 1/3 having an ethylene glycol, diethylene glycol, or (poly)ethylene glycol as described for general formula V, such as the compound shown below:

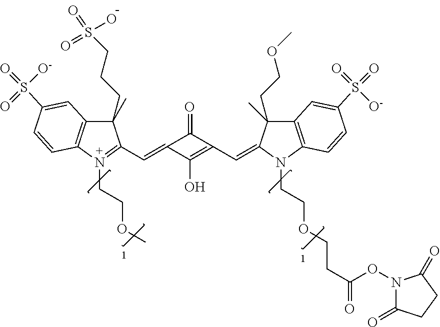

In one embodiment the compound is 650 Compound 0/1

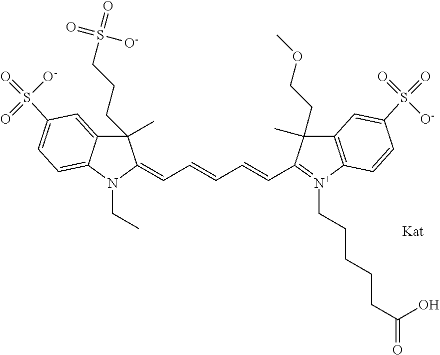 [SMILES: CCN1/C(=C/C=C/C=C/C2=[N+](CCCCCC(=O)O)c3ccc(S(=O)(=O)[O-])cc3C2(C)CCOC)C(C)(CCCS(=O)(=O)[O-])c2cc(S(=O)(=O)[O-])ccc21]

650 Compound 0/1 (1-(5-carboxypentyl)-2-((1E,3E,5E)-5-(1-ethyl-3-methyl-5-sulfonato-3-(3-sulfonatopropyl)indolin-2-ylidene)penta-1,3-dienyl)-3-(2-methoxyethyl)-3-methyl-3H-indolium-5-sulfonate) contains ethylene glycol at R2, a sulfoalkyl at R1, ethyl at R9, and carboxyalkyl at R10.

One non-limiting example of an activated compound is the NHS-ester of 650 Compound 0/1, shown below:

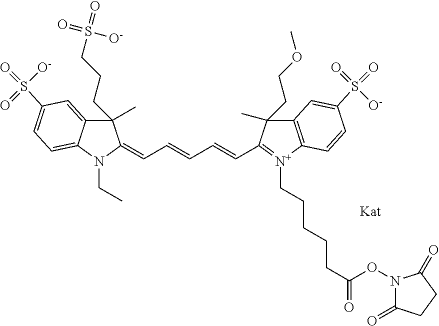 [SMILES: CCN1/C(=C/C=C/C=C/C2=[N+](CCCCCC(=O)ON3C(=O)CCC3=O)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 650 Compound 0/1 is a tetrafluorophenyl (TFP)-ester form, shown below:

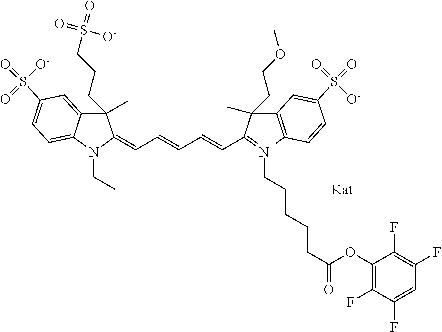 [SMILES: CCN1/C(=C/C=C/C=C/C2=[N+](CCCCCC(=O)Oc3c(F)c(F)cc(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 650 Compound 0/1 is a sulfotetrafluorophenyl (STP)-ester form, shown below:

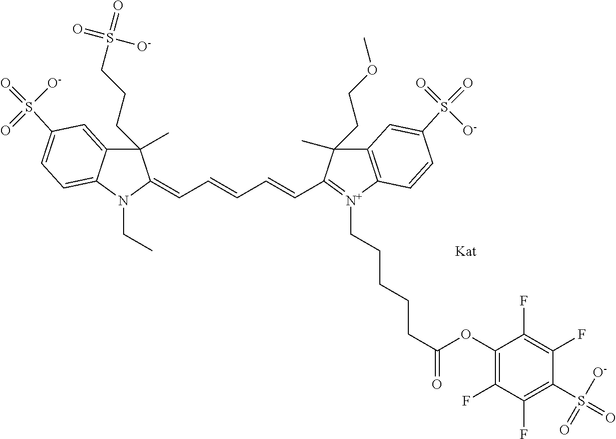 [SMILES: CCN1/C(=C/C=C/C=C/C2=[N+](CCCCCC(=O)Oc3c(F)c(F)c(S(=O)(=O)[O-])c(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 650 Compound 0/1 is a hydrazide form, shown below:

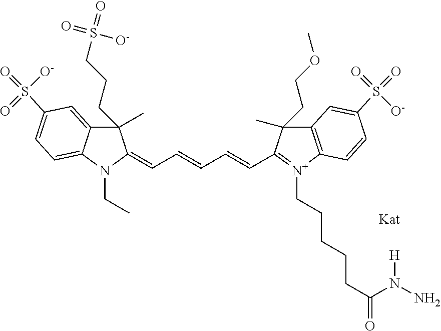 [SMILES: CCN1/C(=C/C=C/C=C/C2=[N+](CCCCCC(=O)NN)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 650 Compound 0/1 is a maleimide form, shown below:

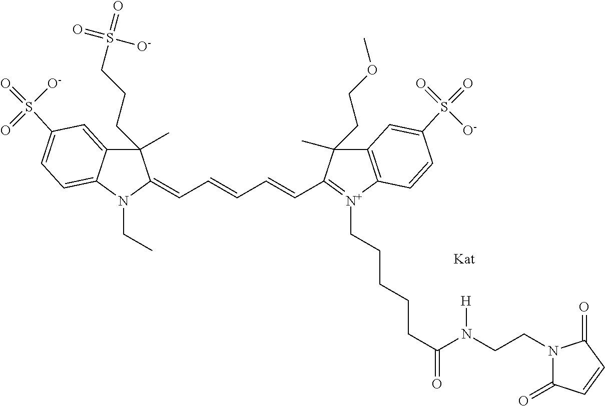 [SMILES: CCN1/C(=C/C=C/C=C/C2=[N+](CCCCCC(=O)NCCN3C(=O)C=CC3=O)c3ccc(S(=O)(=O)[O-])cc3C2(C)CCOC)C(C)(CCCS(=O)(=O)[O-])c2cc(S(=O)(=O)[O-])ccc21]

In one embodiment the compound is 650 Compound 0/1

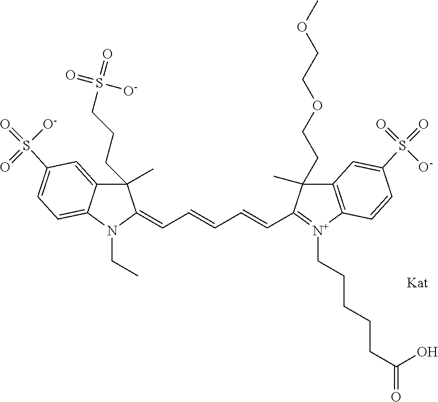 [SMILES: CCN1/C(=C/C=C/C=C/C2=[N+](CCCCCC(=O)O)c3ccc(S(=O)(=O)[O-])cc3C2(C)CCOCCOC)C(C)(CCCS(=O)(=O)[O-])c2cc(S(=O)(=O)[O-])ccc21]

650 Compound 0/1 (1-(5-carboxypentyl)-2-((1E,3E,5E)-5-(1-ethyl-3-methyl-5-sulfonato-3-(3-sulfonatopropyl)indolin-2-ylidene)penta-1,3-dienyl)-3-(2-(2-(2-methoxyethoxy)ethyl)-3-methyl-3H-indolium-5-sulfonate) contains a diethylene glycol at R2, sulfoalkyl at R1, ethyl at R9, and carboxyalkyl at R10.

In one embodiment the compound is 650 Compound 0/1

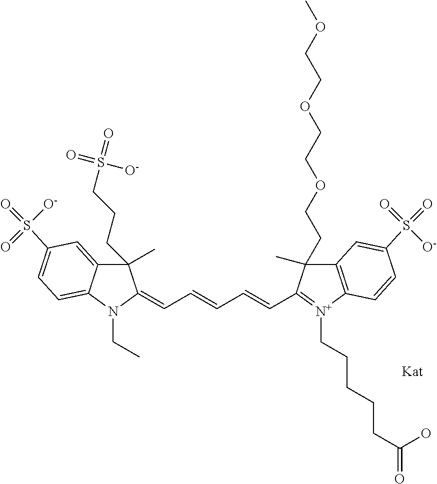 [SMILES: CCN1/C(=C/C=C/C=C/C2=[N+](CCCCCC(=O)O)c3ccc(S(=O)(=O)[O-])cc3C2(C)CCOCCOCCOC)C(C)(CCCS(=O)(=O)[O-])c2cc(S(=O)(=O)[O-])ccc21]

650 Compound 0/1 (1-(5-carboxypentyl)-2-((1E,3E,5E)-5-(1-ethyl-3-methyl-5-sulfonato-3-(3-sulfonatopropyl)indolin-2-ylidene)penta-1,3-dienyl)-3-(2-(2-(2-methoxyethoxy)ethoxy)ethyl)-3-methyl-3H-indolium-5-sulfonate) contains a (poly)ethylene glycol (PEG₃) at R2, sulfoalkyl at R1, ethyl at R9, and carboxyalkyl at R10.

In one embodiment the compound is 650 Compound 0/1

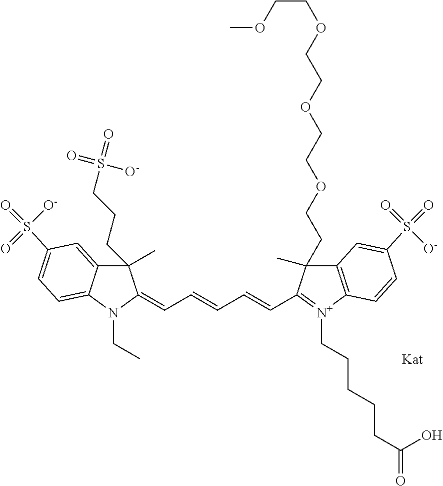 [SMILES: CCN1/C(=C/C=C/C=C/C2=[N+](CCCCCC(=O)O)c3ccc(S(=O)(=O)[O-])cc3C2(C)CCOCCOCCOCCOC)C(C)(CCCS(=O)(=O)[O-])c2cc(S(=O)(=O)[O-])ccc21]

650 Compound 0/1 (1-(5-carboxypentyl)-2-((1E,3E,5E)-5-(1-ethyl-3-methyl-5-sulfonato-3-(3-sulfonatopropyl)indolin-2-ylidene)penta-1,3-dienyl)-3-methyl-3-(2,5,8,11-tetraoxatridecan-13-yl)-3H-indolium-5-sulfonate) contains a (poly)ethylene glycol (PEG₄) at R2, sulfoalkyl at R1, ethyl at R9, and carboxyalkyl at R10.

In one embodiment the compound is 650 Compound 0/1

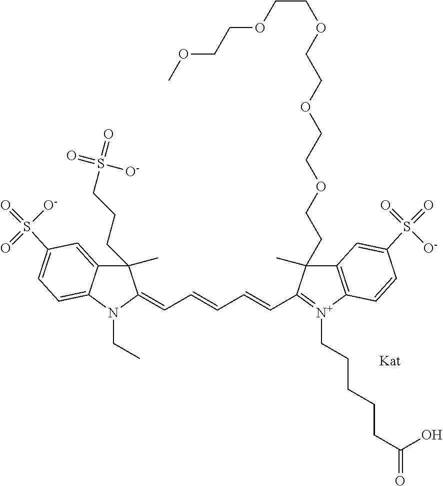 [SMILES: CCN1/C(=C/C=C/C=C/C2=[N+](CCCCCC(=O)O)c3ccc(S(=O)(=O)[O-])cc3C2(C)CCOCCOCCOCCOCCOC)C(C)(CCCS(=O)(=O)[O-])c2cc(S(=O)(=O)[O-])ccc21]

650 Compound 0/1 (1-(5-carboxypentyl)-2-((1E,3E,5E)-5-(1-ethyl-3-methyl-5-sulfonato-3-(3-sulfonatopropyl)indolin-2-ylidene)penta-1,3-dienyl)-3-(2,5,8,11,14-pentaoxahexadecan-16-yl)-3-methyl-3H-indolium-5-sulfonate) contains a (poly)ethylene glycol (PEG₅) at R2, sulfoalkyl at R1, ethyl at R9, and carboxyalkyl at R10.

In one embodiment the compound is 650 Compound 0/1

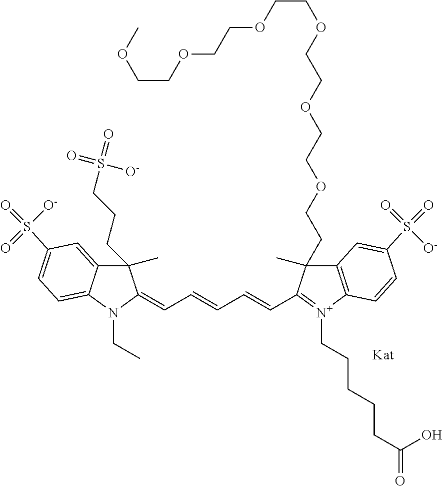 [SMILES: CCN1/C(=C/C=C/C=C/C2=[N+](CCCCCC(=O)O)c3ccc(S(=O)(=O)[O-])cc3C2(C)CCOCCOCCOCCOCCOCCOC)C(C)(CCCS(=O)(=O)[O-])c2cc(S(=O)(=O)[O-])ccc21]

650 Compound 0/1 (1-(5-carboxypentyl)-2-((1E,3E,5E)-5-(1-ethyl-3-methyl-5-sulfonato-3-(3-sulfonatopropyl)indolin-2-ylidene)penta-1,3-dienyl)-3-methyl-3-(2, 5,8,11,14,17-hexaoxanonadecan-19-yl)-3H-indolium-5-sulfonate) contains a (poly)ethylene glycol (PEG₆) at R2, sulfoalkyl at R1, ethyl at R9, and carboxyalkyl at R10.

In one embodiment the compound is 650 Compound 0/1

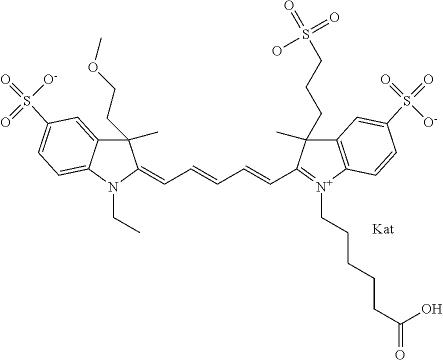 [SMILES: CCN1/C(=C/C=C/C=C/C2=[N+](CCCCCC(=O)O)c3ccc(S(=O)(=O)[O-])cc3C2(C)CCCS(=O)(=O)O)C(C)(CCOC)c2cc(S(=O)(=O)[O-])ccc21]

650 Compound 0/1 (1-(5-carboxypentyl)-2-((1E,3E,5E)-5-(1-ethyl-3-(2-methoxyethyl)-3-methyl-5-sulfonatoindolin-2-ylidene)penta-1,3-dienyl)-3-methyl-3-(3-sulfopropyl)-3H-indolium-5-sulfonate) contains ethylene glycol at R1, a sulfoalkyl at R2, ethyl at R9, and carboxyalkyl at R10.

One non-limiting example of an activated compound is the NHS-ester of 650 Compound 0/1, shown below:

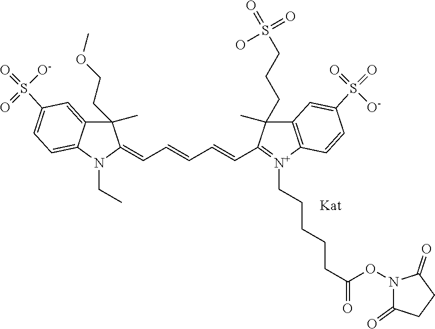 [SMILES: CCN1/C(=C/C=C/C=C/C2=[N+](CCCCCC(=O)ON3C(=O)CCC3=O)c3ccc(S(=O)(=O)[O-])cc3C2(C)CCCS(=O)(=O)O)C(C)(CCOC)c2cc(S(=O)(=O)[O-])ccc21]

In one embodiment the compound is 650 Compound 0/1

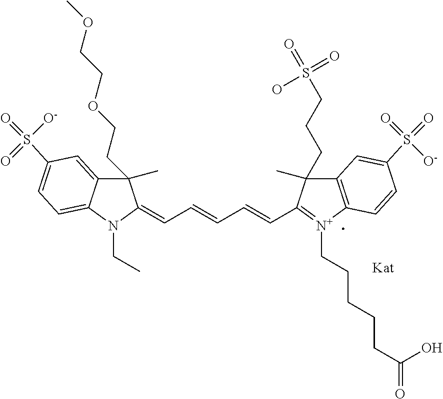

650 Compound 0/1 (1-(5-carboxypentyl)-2-((1E,3E,5E)-5-(1-ethyl-3-(2-(2-methoxyethoxy)ethyl)-3-methyl-5-sulfonatoindolin-2-ylidene)penta-1,3-dienyl)-3-methyl-3-(3-sulfopropyl)-3H-indolium-5-sulfonate) contains a diethylene glycol at R1, sulfoalkyl at R2, ethyl at R9, and carboxyalkyl at R10.

In one embodiment the compound is 650 Compound 0/1

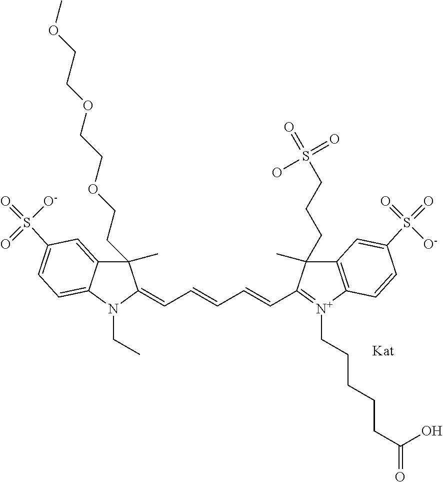 [SMILES: CCN1/C(=C/C=C/C=C/C2=[N+](CCCCCC(=O)O)c3ccc(S(=O)(=O)[O-])cc3C2(C)CCCS(=O)(=O)O)C(C)(CCOCCOCCOC)c2cc(S(=O)(=O)[O-])ccc21]

650 Compound 0/1 (1-(5-carboxypentyl)-2-((1E,3E,5E)-5-(1-ethyl-3-(2-(2-(2-methoxyethoxy)ethoxy)ethyl)-3-methyl-5-sulfonatoindolin-2-ylidene)penta-1,3-dienyl)-3-methyl-3-(3-sulfopropyl)-3H-indolium-5-sulfonate) contains a (poly)ethylene glycol (PEG₃) at R1, sulfoalkyl at R2, ethyl at R9, and carboxyalkyl at R10.

In one embodiment the compound is 650 Compound 0/1

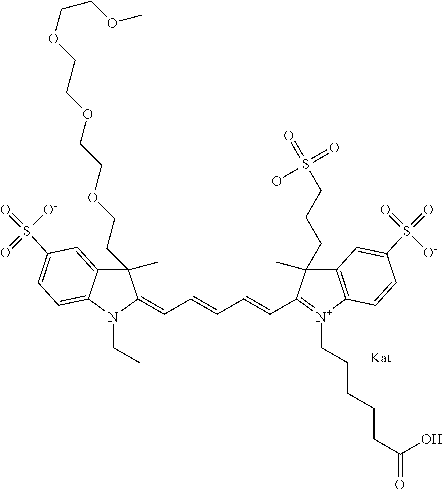 [SMILES: CCN1/C(=C/C=C/C=C/C2=[N+](CCCCCC(=O)O)c3ccc(S(=O)(=O)[O-])cc3C2(C)CCCS(=O)(=O)O)C(C)(CCOCCOCCOCCOC)c2cc(S(=O)(=O)[O-])ccc21]

650 Compound 0/1 (1-(5-carboxypentyl)-2-((1E,3E,5E)-5-(1-ethyl-3-methyl-5-sulfonato-3-(2,5,8,11-tetraoxatridecan-13-yl)indolin-2-ylidene)penta-1,3-dienyl)-3-methyl-3-(3-sulfopropyl)-3H-indolium-5-sulfonate) contains a (poly)ethylene glycol (PEG₄) at R1, sulfoalkyl at R2, ethyl at R9, and carboxyalkyl at R10.

In one embodiment the compound is 650 Compound 0/1

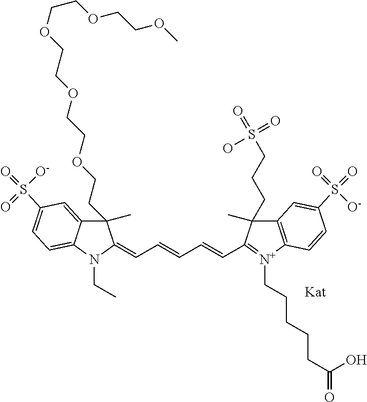 [SMILES: CCN1/C(=C/C=C/C=C/C2=[N+](CCCCCC(=O)O)c3ccc(S(=O)(=O)[O-])cc3C2(C)CCCS(=O)(=O)O)C(C)(CCOCCOCCOCCOCCOC)c2cc(S(=O)(=O)[O-])ccc21]

650 Compound 0/1 (1-(5-carboxypentyl)-2-((1E,3E,5E)-5-(1-ethyl-3-(2,5,8,11,14-pentaoxahexadecan-16-yl)-3-methyl-5-sulfonatoindolin-2-ylidene)penta-1,3-dienyl)-3-methyl-3-(3-sulfopropyl)-3H-indolium-5-sulfonate) contains a (poly)ethylene glycol (PEG₅) at R1, sulfoalkyl at R2, ethyl at R9, and carboxyalkyl at R10.

In one embodiment the compound is 650 Compound 0/1

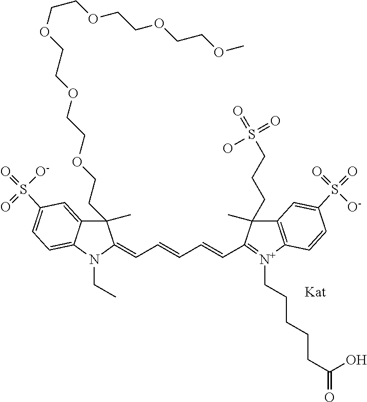 [SMILES: CCN1/C(=C/C=C/C=C/C2=[N+](CCCCCC(=O)O)c3ccc(S(=O)(=O)[O-])cc3C2(C)CCCS(=O)(=O)O)C(C)(CCOCCOCCOCCOCCOCCOC)c2cc(S(=O)(=O)[O-])ccc21]

650 Compound 0/1 (1-(5-carboxypentyl)-2-((1E,3E,5E)-5-(1-ethyl-3-methyl-3-(2,5,8,11,14,17-hexaoxanonadecan-19-yl)-5-sulfonatoindolin-2-ylidene)penta-1,3-dienyl)-3-methyl-3-(3-sulfopropyl)-3H-indolium-5-sulfonate) contains a (poly)ethylene glycol (PEG₆) at R1, sulfoalkyl at R2, ethyl at R9, and carboxyalkyl at R10.

In one embodiment the compound is 650 Compound 0/2

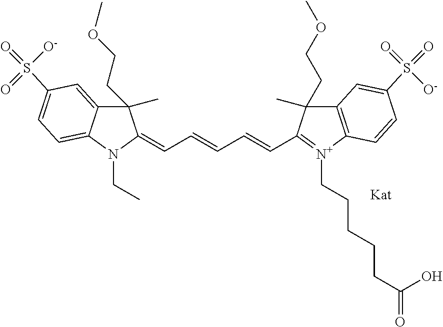 [SMILES: CCN1/C(=C/C=C/C=C/C2=[N+](CCCCCC(=O)O)c3ccc(S(=O)(=O)[O-])cc3C2(C)CCOC)C(C)(CCOC)c2cc(S(=O)(=O)[O-])ccc21]

650 Compound 0/2 (1-(5-carboxypentyl)-2-((1E,3E,5E)-5-(1-ethyl-3-(2-methoxyethyl)-3-methyl-5-sulfonatoindolin-2-ylidene)penta-1,3-dienyl)-3-(2-methoxyethyl)-3-methyl-3H-indolium-5-sulfonate) contains ethylene glycol at R1 and R2, ethyl at R9, and carboxyalkyl at R10.

One non-limiting example of an activated compound is the NHS-ester of 650 Compound 0/2, shown below:

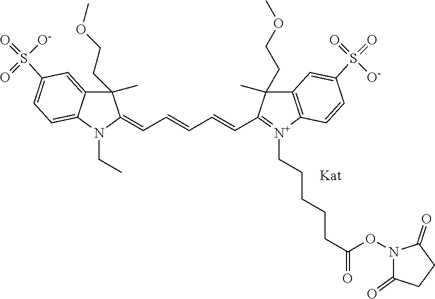 [SMILES: CCN1/C(=C/C=C/C=C/C2=[N+](CCCCCC(=O)ON3C(=O)CCC3=O)c3ccc(S(=O)(=O)[O-])cc3C2(C)CCOC)C(C)(CCOC)c2cc(S(=O)(=O)[O-])ccc21]

In one embodiment the compound is 650 Compound 0/2

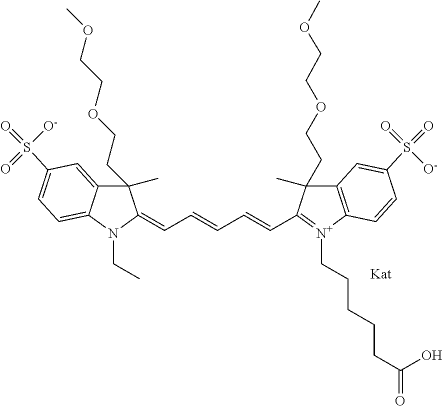 [SMILES: CCN1/C(=C/C=C/C=C/C2=[N+](CCCCCC(=O)O)c3ccc(S(=O)(=O)[O-])cc3C2(C)CCOCCOC)C(C)(CCOCCOC)c2cc(S(=O)(=O)[O-])ccc21]

650 Compound 0/2 (1-(5-carboxypentyl)-2-((1E,3E,5E)-5-(1-ethyl-3-(2-(2-methoxyethoxy)ethyl)-3-methyl-5-sulfonatoindolin-2-ylidene)penta-1,3-dienyl)-3-(2-(2-methoxyethoxy)ethyl)-3-methyl-3H-indolium-5-sulfonate) contains a diethylene glycol at R1 and R2, ethyl at R9, and carboxyalkyl at R10.

In one embodiment the compound is 650 Compound 0/2

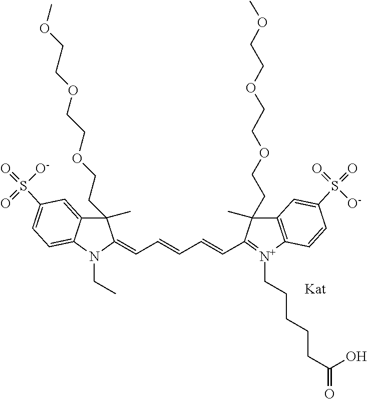 [SMILES: CCN1/C(=C/C=C/C=C/C2=[N+](CCCCCC(=O)O)c3ccc(S(=O)(=O)[O-])cc3C2(C)CCOCCOCCOC)C(C)(CCOCCOCCOC)c2cc(S(=O)(=O)[O-])ccc21]

650 Compound 0/2 (1-(5-carboxypentyl)-2-((1E,3E,5E)-5-(1-ethyl-3-(2-(2-(2-methoxyethoxy)ethoxy)ethyl)-3-methyl-5-sulfonatoindolin-2-ylidene)penta-1,3-dienyl)-3-(2-(2-(2-methoxyethoxy)ethoxy)ethyl)-3-methyl-3H-indolium-5-sulfonate) contains a (poly)ethylene glycol (PEG₃) at R1 and R2, ethyl at R9, and carboxyalkyl at R10.

In one embodiment the compound is 650 Compound 0/2

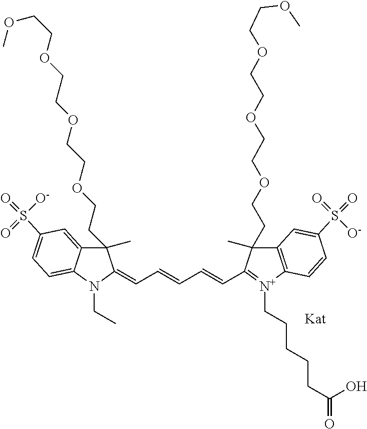 [SMILES: CCN1/C(=C/C=C/C=C/C2=[N+](CCCCCC(=O)O)c3ccc(S(=O)(=O)[O-])cc3C2(C)CCOCCOCCOCCOC)C(C)(CCOCCOCCOCCOC)c2cc(S(=O)(=O)[O-])ccc21]

650 Compound 0/2 (1-(5-carboxypentyl)-2-((1E,3E,5E)-5-(1-ethyl-3-methyl-5-sulfonato-3-(2,5,8,11-tetraoxatridecan-13-yl)indolin-2-ylidene)penta-1,3-dienyl)-3-methyl-3-(2,5,8,11-tetraoxatridecan-13-yl)-3H-indolium-5-sulfonate) contains a (poly)ethylene glycol (PEG₄) at R1 and R2, ethyl at R9, and carboxyalkyl at R10.

In one embodiment the compound is 650 Compound 0/2

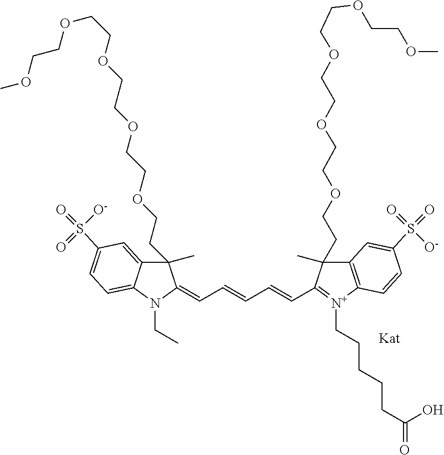

650 Compound 0/2 (1-(5-carboxypentyl)-2-((1E,3E,5E)-5-(1-ethyl-3-(2,5,8,11,14-pentaoxahexadecan-16-yl)-3-methyl-5-sulfonatoindolin-2-ylidene)penta-1, 3-dienyl)-3-(2,5,8,11,14-pentaoxahexadecan-16-yl)-3-methyl-3H-indolium-5-sulfonate) contains a (poly)ethylene glycol (PEG₅) at R1 and R2, ethyl at R9, and carboxyalkyl at R10.

In one embodiment the compound is 650 Compound 0/2

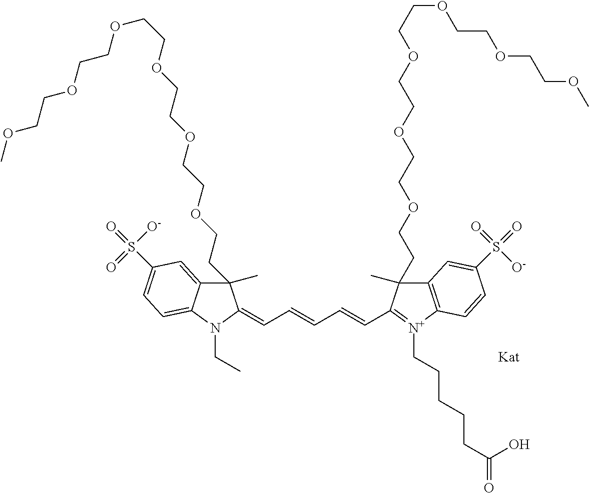

650 Compound 0/2 (1-(5-carboxypentyl)-2-((1E,3E,5E)-5-(1-ethyl-3-methyl-3-(2,5,8,11,14,17-hexaoxanonadecan-19-yl)-5-sulfonatoindolin-2-ylidene)penta-1, 3-dienyl)-3-methyl-3-(2,5,8,11,14,17-hexaoxanonadecan-19-yl)-3H-indolium-5-sulfonate) contains a (poly)ethylene glycol (PEG₆) at R1 and R2, ethyl at R9, and carboxyalkyl at R10.

In one embodiment the compound is 650 Compound 0/1

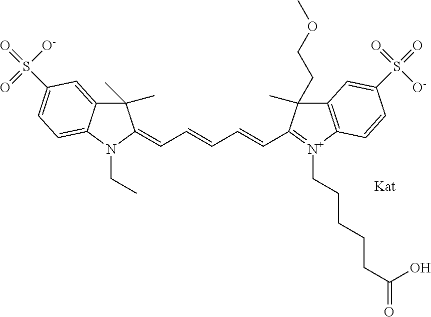 [SMILES: CCN1/C(=C/C=C/C=C/C2=[N+](CCCCCC(=O)O)c3ccc(S(=O)(=O)[O-])cc3C2(C)CCOC)C(C)(C)c2cc(S(=O)(=O)[O-])ccc21]

650 Compound 0/1 (1-(5-carboxypentyl)-2-((1E,3E,5E)-5-(1-ethyl-3,3-dimethyl-5-sulfonatoindolin-2-ylidene)penta-1,3-dienyl)-3-(2-methoxyethyl)-3-methyl-3H-indolium-5-sulfonate) contains ethylene glycol at R2, methyl at R1, ethyl at R9, and carboxyalkyl at R10.

One non-limiting example of an activated compound is the NHS-ester of 650 Compound 0/1, shown below:

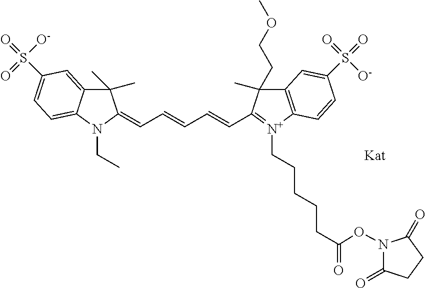 [SMILES: CCN1/C(=C/C=C/C=C/C2=[N+](CCCCCC(=O)ON3C(=O)CCC3=O)c3ccc(S(=O)(=O)[O-])cc3C2(C)CCOC)C(C)(C)c2cc(S(=O)(=O)[O-])ccc21]

In one embodiment the compound is 650 Compound 0/1

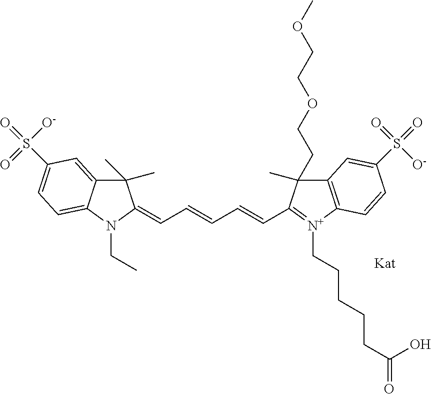 [SMILES: CCN1/C(=C/C=C/C=C/C2=[N+](CCCCCC(=O)O)c3ccc(S(=O)(=O)[O-])cc3C2(C)CCOCCOC)C(C)(C)c2cc(S(=O)(=O)[O-])ccc21]

650 Compound 0/1 (1-(5-carboxypentyl)-2-((1E,3E,5E)-5-(1-ethyl-3,3-dimethyl-5-sulfonatoindolin-2-ylidene)penta-1,3-dienyl)-3-(2-(2-methoxyethoxy)ethyl)-3-methyl-3H-indolium-5-sulfonate) contains a diethylene glycol at R2, methyl at R1, ethyl at R9, and carboxyalkyl at R10.

In one embodiment the compound is 650 Compound 0/1

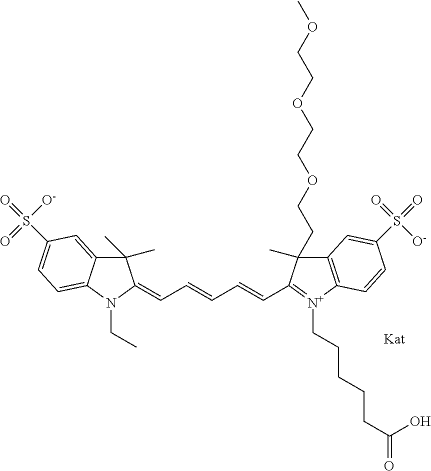 [SMILES: CCN1/C(=C/C=C/C=C/C2=[N+](CCCCCC(=O)O)c3ccc(S(=O)(=O)[O-])cc3C2(C)CCOCCOCCOC)C(C)(C)c2cc(S(=O)(=O)[O-])ccc21]

650 Compound 0/1 (1-(5-carboxypentyl)-2-((1E,3E,5E)-5-(1-ethyl-3,3-dimethyl-5-sulfonatoindolin-2-ylidene)penta-1,3-dienyl)-3-(2-(2-(2-methoxyethoxy)ethoxy)ethyl)-3-methyl-3H-indolium-5-sulfonate) contains a (poly)ethylene glycol (PEG₃) at R2, methyl at R1, ethyl at R9, and carboxyalkyl at R10.

In one embodiment the compound is 650 Compound 0/1

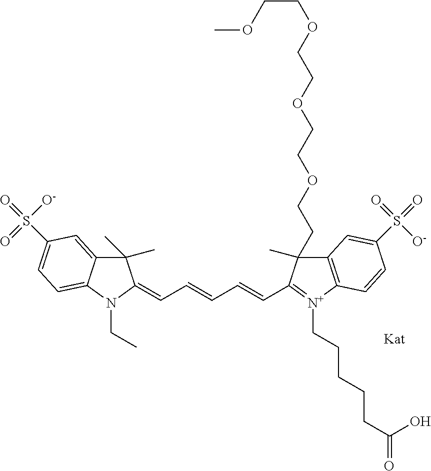 [SMILES: CCN1/C(=C/C=C/C=C/C2=[N+](CCCCCC(=O)O)c3ccc(S(=O)(=O)[O-])cc3C2(C)CCOCCOCCOCCOC)C(C)(C)c2cc(S(=O)(=O)[O-])ccc21]

650 Compound 0/1 (1-(5-carboxypentyl)-2-((1E,3E,5E)-5-(1-ethyl-3,3-dimethyl-5-sulfonatoindolin-2-ylidene)penta-1,3-dienyl)-3-methyl-3-(2,5,8,11-tetraoxatridecan-13-yl)-3H-indolium-5-sulfonate) contains a (poly)ethylene glycol (PEG₄) at R2, methyl at R1, ethyl at R9, and carboxyalkyl at R10.

In one embodiment the compound is 650 Compound 0/1

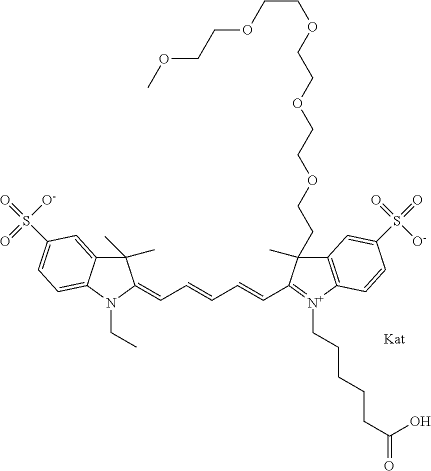 [SMILES: CCN1/C(=C/C=C/C=C/C2=[N+](CCCCCC(=O)O)c3ccc(S(=O)(=O)[O-])cc3C2(C)CCOCCOCCOCCOCCOC)C(C)(C)c2cc(S(=O)(=O)[O-])ccc21]

650 Compound 0/1 (1-(5-carboxypentyl)-2-((1E,3E,5E)-5-(1-ethyl-3,3-dimethyl-5-sulfonatoindolin-2-ylidene)penta-1,3-dienyl)-3-(2,5,8,11,14-pentaoxahexadecan-16-yl)-3-methyl-3H-indolium-5-sulfonate) contains a (poly)ethylene glycol (PEG₅) at R2, methyl at R1, ethyl at R9, and carboxyalkyl at R10.

In one embodiment the compound is 650 Compound 0/1

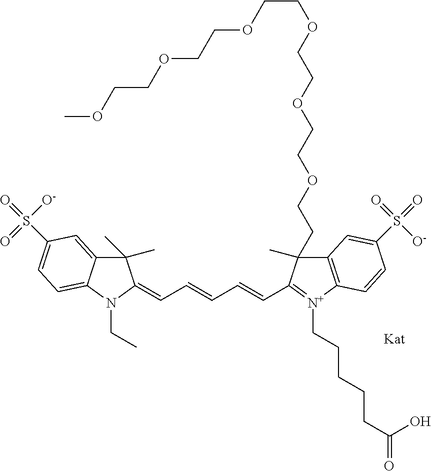 [SMILES: CCN1/C(=C/C=C/C=C/C2=[N+](CCCCCC(=O)O)c3ccc(S(=O)(=O)[O-])cc3C2(C)CCOCCOCCOCCOCCOCCOC)C(C)(C)c2cc(S(=O)(=O)[O-])ccc21]

650 Compound 0/1 (1-(5-carboxypentyl)-2-((1E,3E,5E)-5-(1-ethyl-3,3-dimethyl-5-sulfonatoindolin-2-ylidene)penta-1,3-dienyl)-3-methyl-3-(2,5,8,11,14,17-hexaoxanonadecan-19-yl)-3H-indolium-5-sulfonate) contains a (poly)ethylene glycol (PEG₆) at R2, methyl at R1, ethyl at R9, and carboxyalkyl at R10.

In one embodiment the compound is 650 Compound 0/1

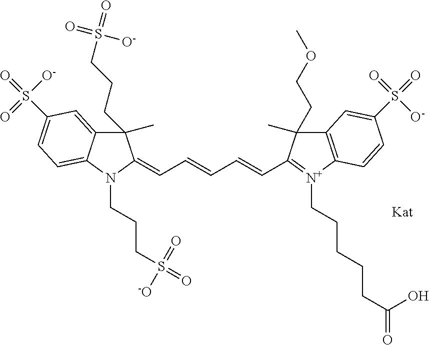

650 Compound 0/1 (3,3′-((E)-2-((2E,4E)-5-(1-(5-carboxypentyl)-3-(2-methoxyethyl)-3-methyl-5-sulfonato-3H-indolium-2-yl)penta-2,4-dienylidene)-3-methyl-5-sulfonatoindoline-1,3-diyl)dipropane-1-sulfonate) contains ethylene glycol at R2, sulfoalkyl at R1, a sulfoalkyl at R9, and carboxyalkyl at R10.

One non-limiting example of an activated compound is the NHS-ester of 650 Compound 0/1, shown below:

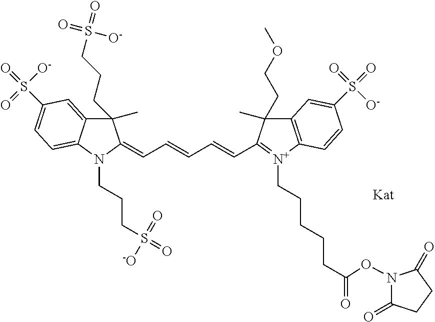

In one embodiment the compound is 650 Compound 0/1

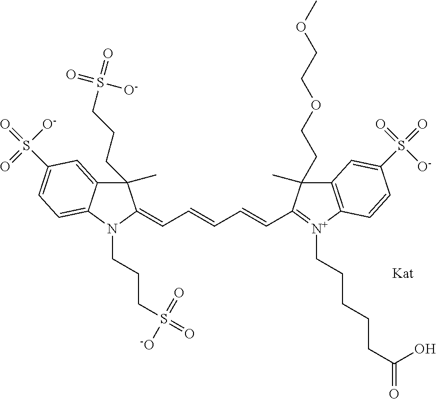

650 Compound 0/1 (3,3′-((E)-2-((2E,4E)-5-(1-(5-carboxypentyl)-3-(2-(2-methoxyethoxy)ethyl)-3-methyl-5-sulfonato-3H-indolium-2-yl)penta-2,4-dienylidene)-3-methyl-5-sulfonatoindoline-1,3-diyl)dipropane-1-sulfonate) contains a diethylene glycol at R2, sulfoalkyl at R1, a sulfoalkyl at R9, and carboxyalkyl at R10.

In one embodiment the compound is 650 Compound 0/1

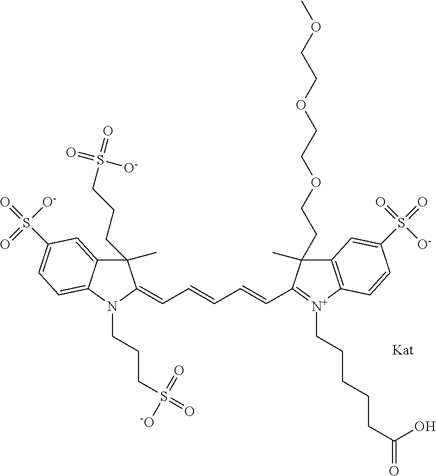

650 Compound 0/1 (3,3′-((E)-2-((2E,4E)-5-(1-(5-carboxypentyl)-3-(2-(2-(2-methoxyethoxy)ethoxy)ethyl)-3-methyl-5-sulfonato-3H-indolium-2-yl)penta-2,4-dienylidene)-3-methyl-5-sulfonatoindoline-1,3-diyl)dipropane-1-sulfonate) contains a (poly)ethylene glycol (PEG₃) at R2, sulfoalkyl at R1, sulfoalkyl at R9, and carboxyalkyl at R10.

In one embodiment the compound is 650 Compound 0/1

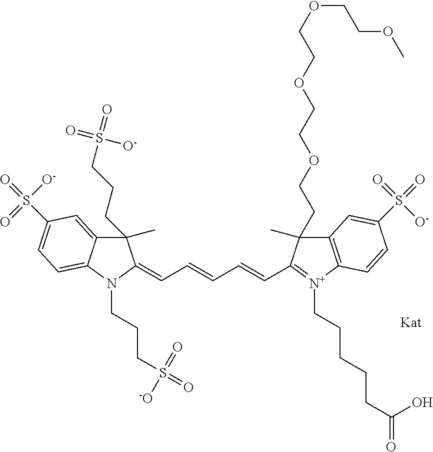

650 Compound 0/1 (3, 3′-((E)-2-((2E,4E)-5-(1-(5-carboxypentyl)-3-methyl-5-sulfonato-3-(2,5,8,11-tetraoxatridecan-13-yl)-3H-indolium-2-yl)penta-2,4-dienylidene)-3-methyl-5-sulfonatoindoline-1,3-diyl)dipropane-1-sulfonate) contains a (poly)ethylene glycol (PEG₄) at R2, sulfoalkyl at R1, sulfoalkyl at R9, and carboxyalkyl at R10.

In one embodiment the compound is 650 Compound 0/1

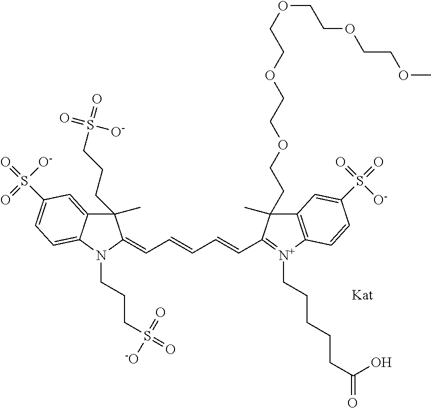

650 Compound 0/1 (3,3′-((E)-2-((2E,4E)-5-(1-(5-carboxypentyl)-3-(2,5,8,11,14-pentaoxahexadecan-16-yl)-3-methyl-5-sulfonato-3H-indolium-2-yl)penta-2,4-dienylidene)-3-methyl-5-sulfonatoindoline-1,3-diyl)dipropane-1-sulfonate) contains a (poly)ethylene glycol (PEG₅) at R2, sulfoalkyl at R1, ethyl at R9, and carboxyalkyl at R10.

In one embodiment the compound is 650 Compound 0/1

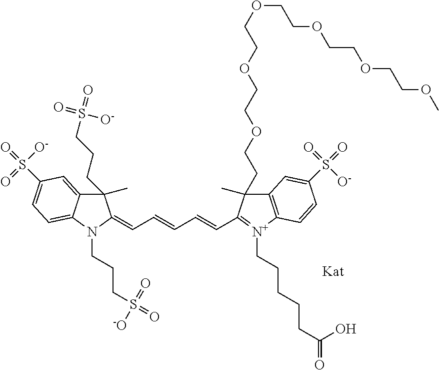

650 Compound 0/1 (3,3′-((E)-2-((2E,4E)-5-(1-(5-carboxypentyl)-3-methyl-3-(2,5,8,11,14,17-hexaoxanonadecan-19-yl)-5-sulfonato-3H-indolium-2-yl)penta-2,4-dienylidene)-3-methyl-5-sulfonatoindoline-1,3-diyl)dipropane-1-sulfonate) contains a (poly)ethylene glycol (PEG₆) at R2, sulfoalkyl at R1, ethyl at R9, and carboxyalkyl at R10.

In one embodiment the compound is 650 Compound 0/2

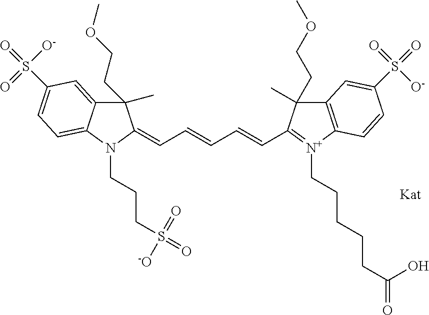

650 Compound 0/2 (1-(5-carboxypentyl)-3-(2-methoxyethyl)-2-((1E,3E,5E)-5-(3-(2-methoxyethyl)-3-methyl-5-sulfonato-1-(3-sulfonatopropyl)indolin-2-ylidene)penta-1,3-dienyl)-3-methyl-3H-indolium-5-sulfonate) contains ethylene glycol at R1 and R2, sulfoalkyl at R9, and carboxyalkyl at R10.

One non-limiting example of an activated compound is the NHS-ester of 650 Compound 0/2, shown below:

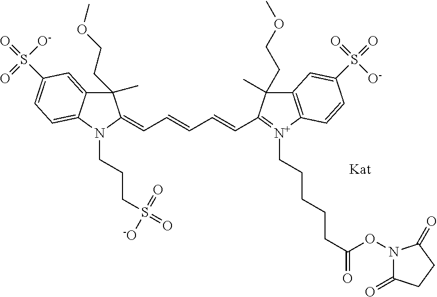

In one embodiment the compound is 650 Compound 0/2

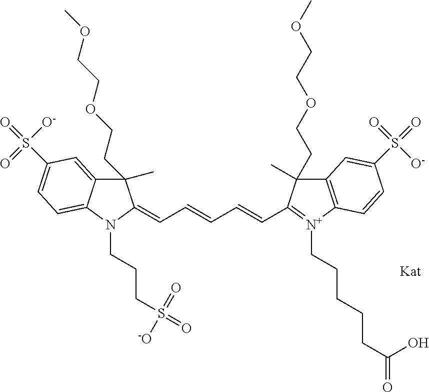

650 Compound 0/2 (1-(5-carboxypentyl)-3-(2-(2-methoxyethoxy)ethyl)-2-((1E,3E,5E)-5-(3-(2-(2-methoxyethoxy)ethyl)-3-methyl-5-sulfonato-1-(3-sulfonatopropyl)indolin-2-ylidene)penta-1,3-dienyl)-3-methyl-3H-indolium-5-sulfonate) contains a diethylene glycol at R1 and R2, sulfoalkyl at R9, and carboxyalkyl at R10.

In one embodiment the compound is 650 Compound 0/2

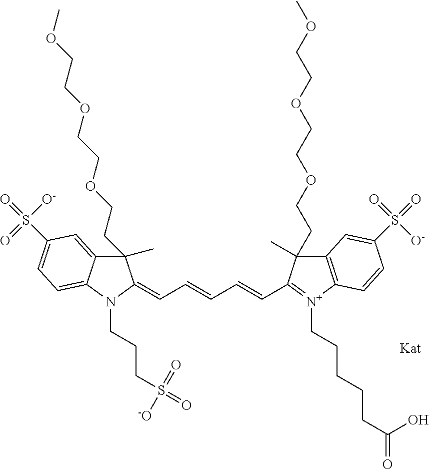

650 Compound 0/2 (1-(5-carboxypentyl)-3-(2-(2-(2-methoxyethoxy)ethoxy)ethyl)-2-((1E,3E,5E)-5-(3-(2-(2-(2-methoxyethoxy)ethoxy)ethyl)-3-methyl-5-sulfonato-1-(3-sulfonatopropyl)indolin-2-ylidene)penta-1,3-dienyl)-3-methyl-3H-indolium-5-sulfonate) contains a (poly)ethylene glycol (PEG₃) at R1 and R2, sulfoalkyl at R9, and carboxyalkyl at R10.

In one embodiment the compound is 650 Compound 0/2

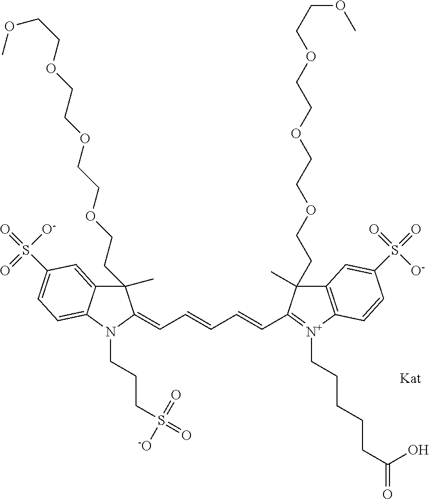

650 Compound 0/2 (1-(5-carboxypentyl)-3-methyl-2-((1E,3E,5E)-5-(3-methyl-5-sulfonato-1-(3-sulfonatopropyl)-3-(2,5,8,11-tetraoxatridecan-13-yl)indolin-2-ylidene)penta-1,3-dienyl)-3-(2,5,8,11-tetraoxatridecan-13-yl)-3H-indolium-5-sulfonate) contains a (poly)ethylene glycol (PEG₄) at R1 and R2, sulfoalkyl at R9, and carboxyalkyl at R10.

In one embodiment the compound is 650 Compound 0/2

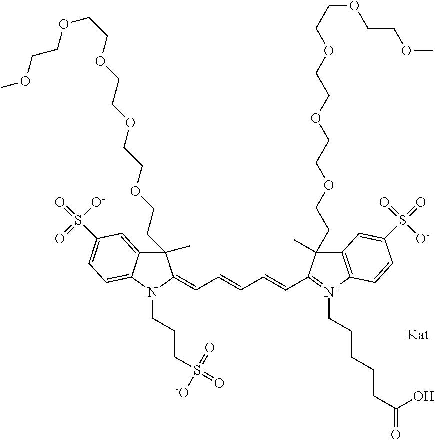

650 Compound 0/2 (2-((1E,3E,5E)-5-(3-(2,5,8,11,14-pentaoxahexadecan-16-yl)-3-methyl-5-sulfonato-1-(3-sulfonatopropyl)indolin-2-ylidene)penta-1,3-dienyl)-1-(5-carboxypentyl)-3-(2,5,8,11,14-pentaoxahexadecan-16-yl)-3-methyl-3H-indolium-5-sulfonate) contains a (poly)ethylene glycol (PEG₅) at R1 and R2, ethyl at R9, and carboxyalkyl at R10.

In one embodiment the compound is 650 Compound 0/2

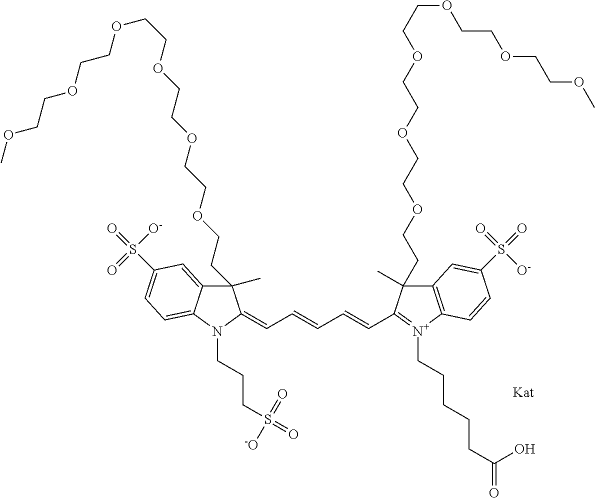

650 Compound 0/2 (1-(5-carboxypentyl)-3-methyl-2-((1E,3E,5E)-5-(3-methyl-3-(2,5,8,11,14,17-hexaoxanonadecan-19-yl)-5-sulfonato-1-(3-sulfonatopropyl)indolin-2-ylidene)penta-1,3-dienyl)-3-(2,5,8,11,14,17-hexaoxanonadecan-19-yl)-3H-indolium-5-sulfonate) contains a (poly)ethylene glycol (PEG₆) at R1 and R2, ethyl at R9, and carboxyalkyl at R10.

In embodiments, the degree of sulfonation is varied to, e.g., vary the compound's degree of hydrophilicity or hydrophobicity. One non-limiting example is a monosulfonate form of 650 Compound 1/2, shown below, but it is understood that the single sulfo group can be at any of the described positions:

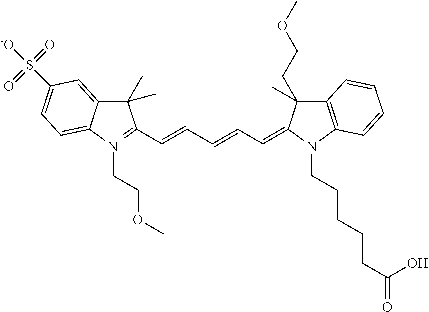

One non-limiting example is a disulfonate form of 650 Compound 1/2, shown below, but it is understood that each of the two sulfo groups can be at any of the described positions:

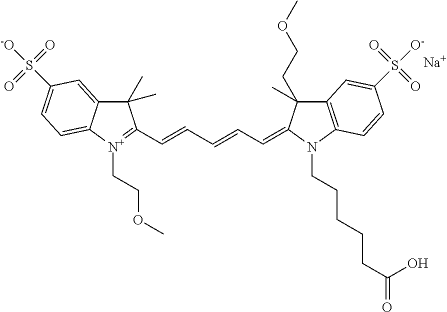

One non-limiting example is a trisulfonate form of 650 Compound 1/2, shown below, but it is understood that each of the three sulfo groups can be at any of the described positions:

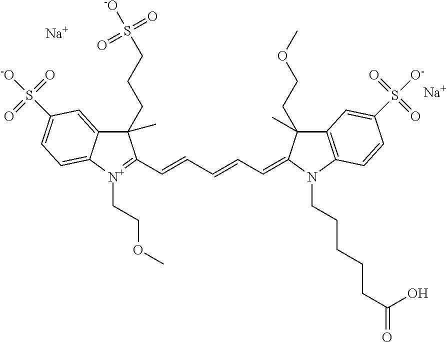

One non-limiting example is a tetrasulfonate form of 650 Compound 1/2, shown below, but it is understood that each of the four sulfo groups can be at any of the described positions:

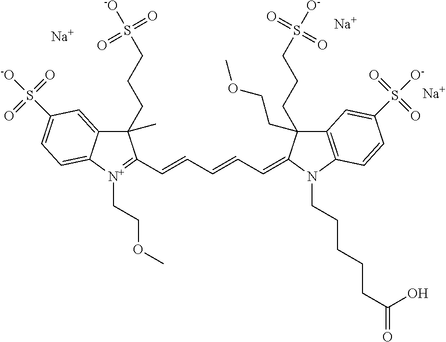

One non-limiting example is a monosulfonate form of 650 Compound 0/1, shown below, but it is understood that the single sulfo group can be at any of the described positions:

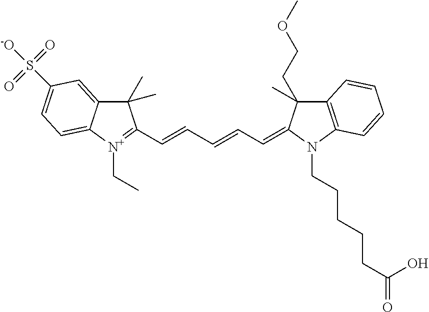

One non-limiting example is a disulfonate form of 650 Compound 0/1, shown below, but it is understood that each of the two sulfo groups can be at any of the described positions:

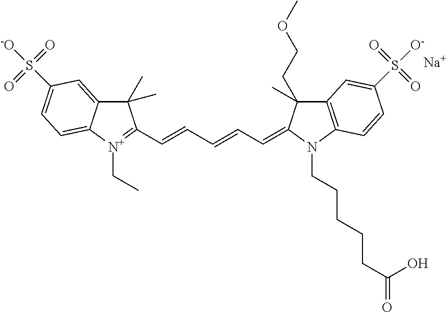

One non-limiting example is a trisulfonate form of 650 Compound 0/1, shown below, but it is understood that each of the three sulfo groups can be at any of the described positions:

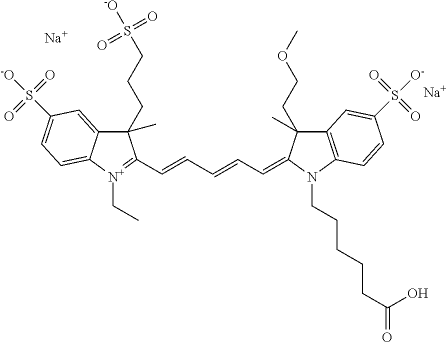

One non-limiting example is a tetrasulfonate form of 650 Compound 0/1, shown below, but it is understood that each of the four sulfo groups can be at any of the described positions:

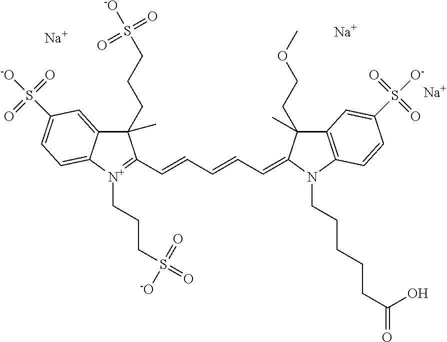

In various embodiments, an ethylene glycol group, diethylene glycol group, and/or a (poly)ethylene glycol group, which will collectively be referred to as a PEG group, unless specifically defined, may be present at position(s) in addition to such groups being present on the N atom(s) of the indole structure.

One non-limiting example of an additionally PEG-substituted compound is a 650 Compound 1/2 according to general formula II where R1 is an ethylene glycol group terminating with a methyl group, shown below:

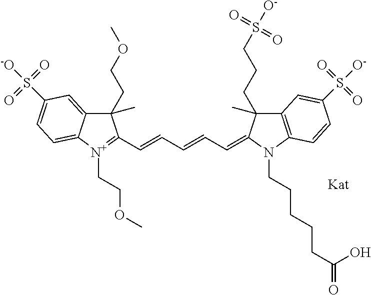

One non-limiting example of an additionally PEG-substituted compound is a 650 Compound 1/2 according to general formula II where R1 is a diethylene glycol group terminating with a methyl group, shown below:

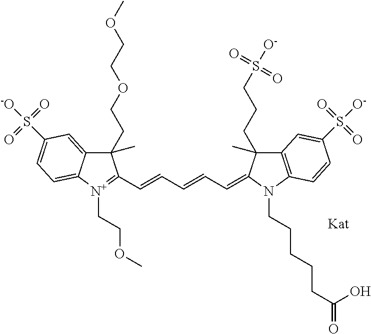

One non-limiting example of an additionally PEG-substituted compound is a 650 Compound 1/2 according to general formula II where R1 is a (poly)ethylene glycol (3) group terminating with a methyl group, shown below:

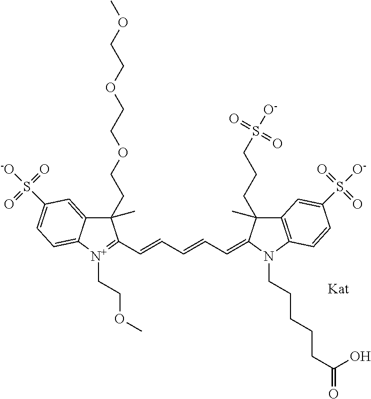

One non-limiting example of an additionally PEG-substituted compound is a 650 Compound 1/2 according to general formula II where R1 is a (poly)ethylene glycol (4) group terminating with a methyl group, shown below:

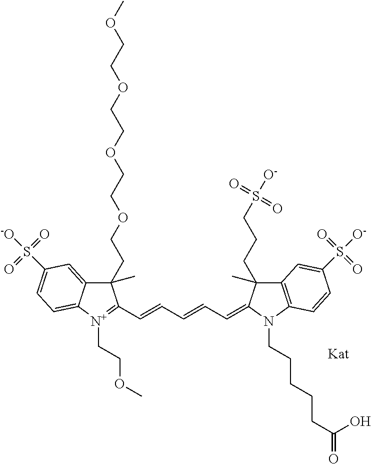

One non-limiting example of an additionally PEG-substituted compound is a 650 Compound 1/2 according to general formula II where R1 is a (poly)ethylene glycol (5) group terminating with a methyl group, shown below:

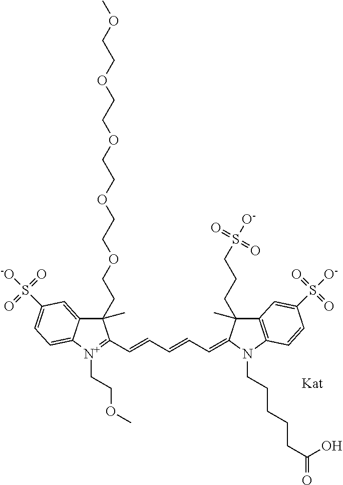

One non-limiting example of an additionally PEG-substituted compound is a 650 Compound 1/2 according to general formula II where R1 is a (poly)ethylene glycol (6) group terminating with a methyl group, shown below:

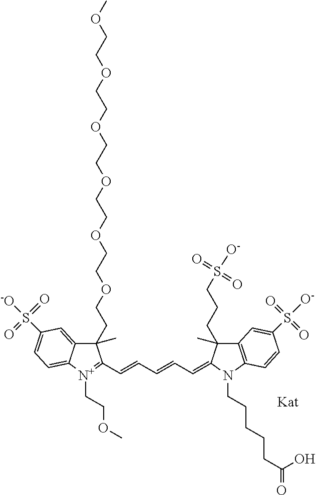

One non-limiting example of an additionally PEG-substituted compound is a 650 Compound 1/2 according to general formula II where R1 is a sulfonamide group -L-SO₂NH—P—Z where Z is a methyl group, shown below:

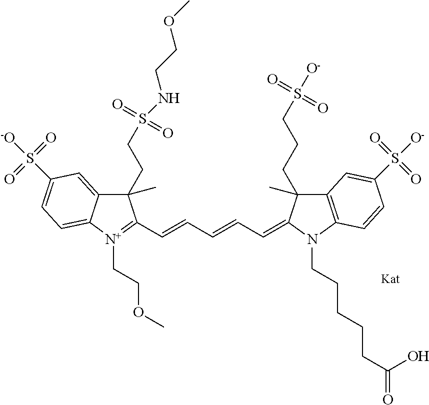

One non-limiting example of an additionally PEG-substituted compound is a 650 Compound 1/2 according to general formula II where R1 is a carboxamide group -L-CONH—P—Z where Z is a methyl group, shown below:

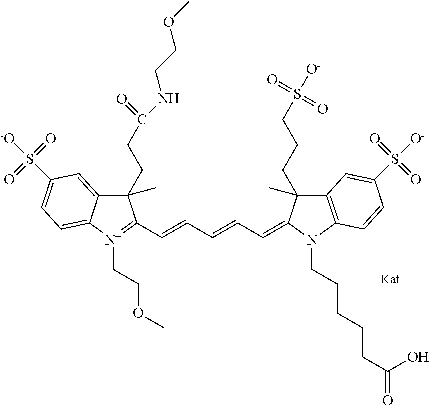

One non-limiting example of an additionally PEG-substituted compound is a 650 Compound 1/2 according to general formula II where R2 is an ethylene glycol group terminating with a methyl group, shown below:

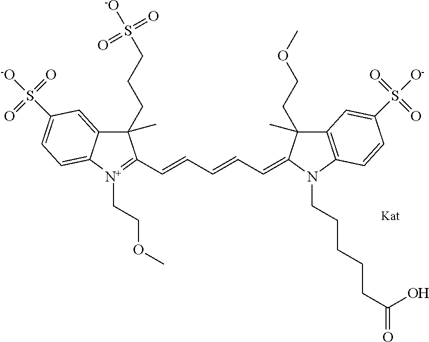

One non-limiting example of an additionally PEG-substituted compound is a 650 Compound 1/2 according to general formula II where R2 is a diethylene glycol group terminating with a methyl group, shown below:

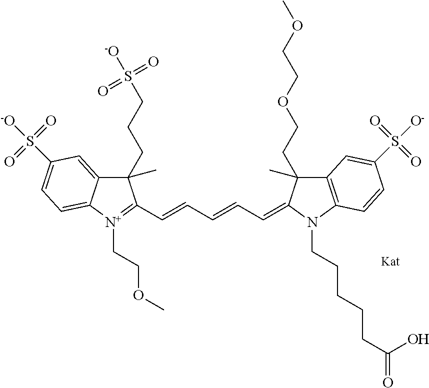

One non-limiting example of an additionally PEG-substituted compound is a 650 Compound 1/2 according to general formula II where R2 is a (poly)ethylene glycol (3) group terminating with a methyl group, shown below:

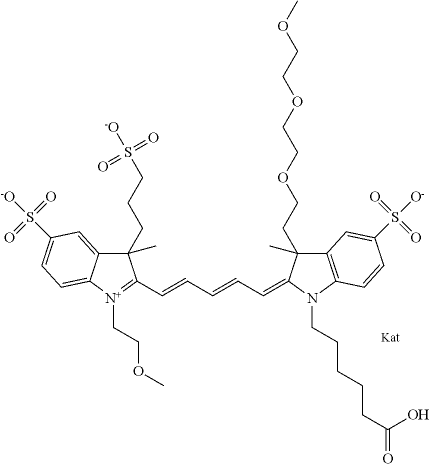

One non-limiting example of an additionally PEG-substituted compound is a 650 Compound 1/2 according to general formula II where R2 is a (poly)ethylene glycol (4) group terminating with a methyl group, shown below:

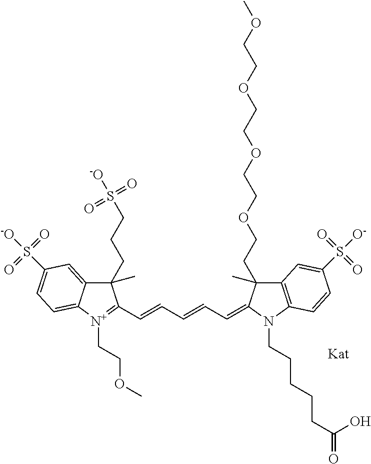

One non-limiting example of an additionally PEG-substituted compound is a 650 Compound 1/2 according to general formula II where R2 is a (poly)ethylene glycol (5) group terminating with a methyl group, shown below:

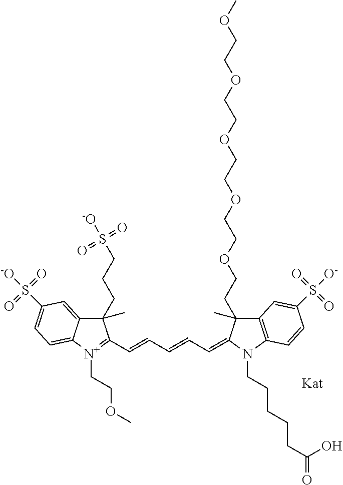

One non-limiting example of an additionally PEG-substituted compound is a 650 Compound 1/2 according to general formula II where R2 is a (poly)ethylene glycol (6) group terminating with a methyl group, shown below:

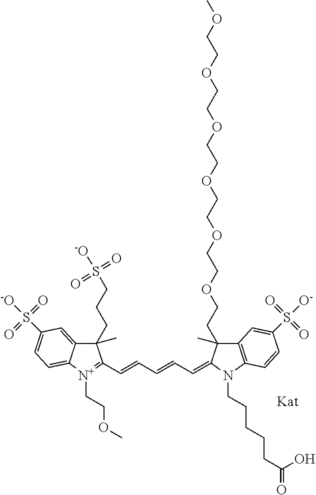

One non-limiting example of an additionally PEG-substituted compound is a 650 Compound 1/2 according to general formula II where R2 is a sulfonamide group -L-SO₂NH—P—Z where Z is a methyl group, shown below:

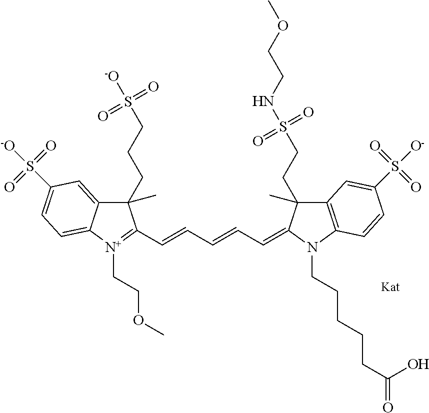

One non-limiting example of an additionally PEG-substituted compound is a 650 Compound 1/2 according to general formula II where R2 is a carboxamide group -L-CONH—P—Z where Z is a methyl group, shown below:

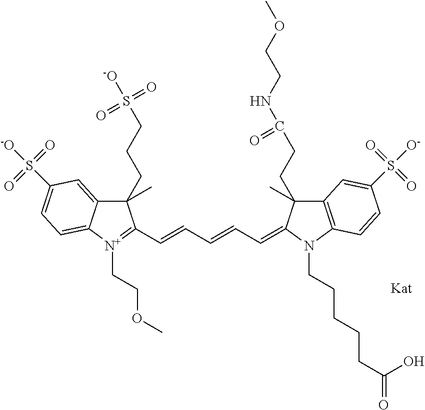

One non-limiting example of an additionally PEG-substituted compound is a 650 Compound 1/3 according to general formula II where both R1 and R2 are an ethylene glycol group terminating with a methyl group, shown below:

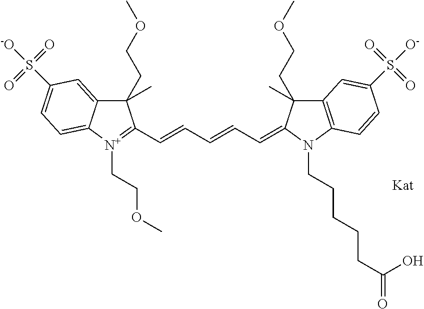

One non-limiting example of an additionally PEG-substituted compound is a 650 Compound 1/3 according to general formula II where both R1 and R2 are a diethylene glycol group terminating with a methyl group, shown below:

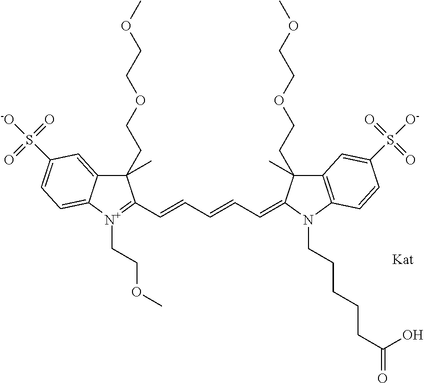

One non-limiting example of an additionally PEG-substituted compound is a 650 Compound 1/3 according to general formula II where both R1 and R2 are a (poly)ethylene glycol (3) group terminating with a methyl group, shown below:

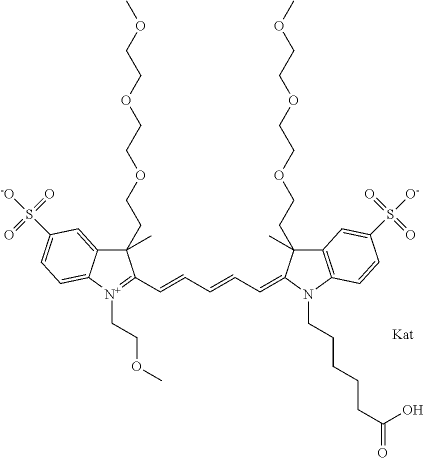

One non-limiting example of an additionally PEG-substituted compound is a 650 Compound 1/3 according to general formula II where both R1 and R2 are a (poly)ethylene glycol (4) group terminating with a methyl group, shown below:

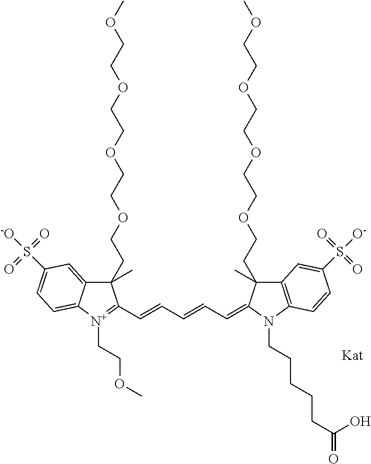

One non-limiting example of an additionally PEG-substituted compound is a 650 Compound 4/4 according to general formula II where both R1 and R2 are a (poly)ethylene glycol (4) group terminating with a methyl group, and R7 and R8 are sulfo, shown below:

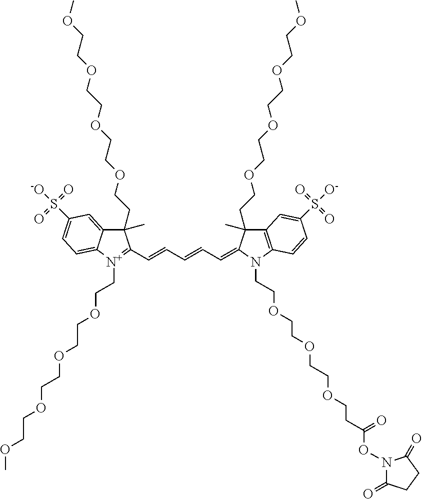

One non-limiting example of an additionally PEG-substituted compound is a 650 Compound 4/4 according to general formula III (V19-03005) where both R1 and R2 are a (poly)ethylene glycol (4) group terminating with a methyl group, and R7 and R8 are sulfo, shown below:

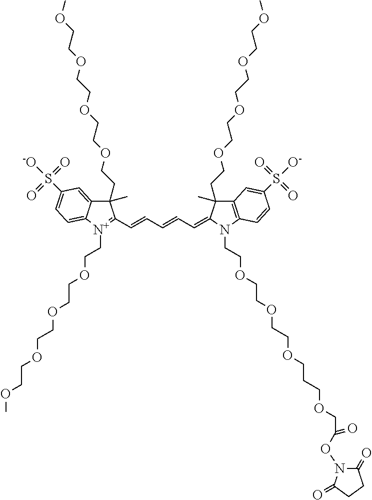

One non-limiting example of an additionally PEG-substituted compound is a 650 Compound 4/4 according to general formula III where both R1 and R2 are a (poly)ethylene glycol (4) group terminating with a methyl group, and R7 and R8 are H, shown below:

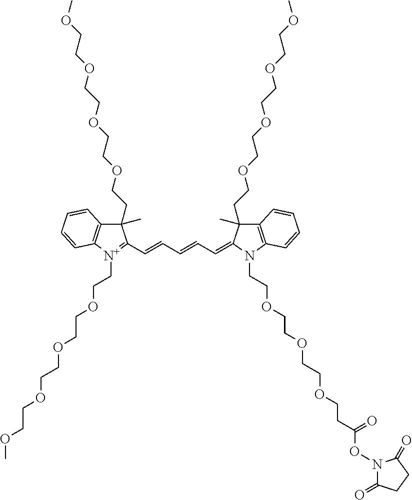

One non-limiting example of an additionally PEG-substituted compound is a 650 Compound 4/4 according to general formula III where both R1 and R2 are a (poly)ethylene glycol (4) group terminating with a methyl group, and R7 and R8 are H, shown below:

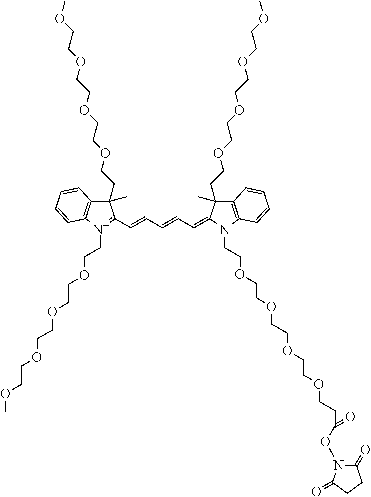

One non-limiting example of an additionally PEG-substituted compound is a 650 Compound 1/3 according to general formula II where both R1 and R2 are a (poly)ethylene glycol (5) group terminating with a methyl group, shown below:

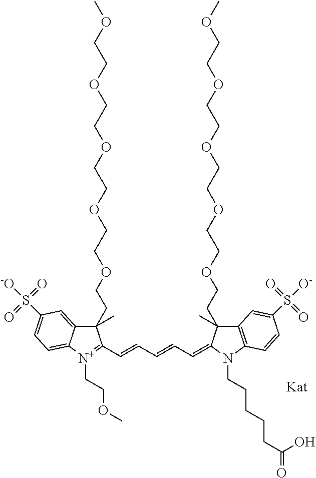

One non-limiting example of an additionally PEG-substituted compound is a 650 Compound 1/3 according to general formula II where both R1 and R2 are a (poly)ethylene glycol (6) group terminating with a methyl group, shown below:

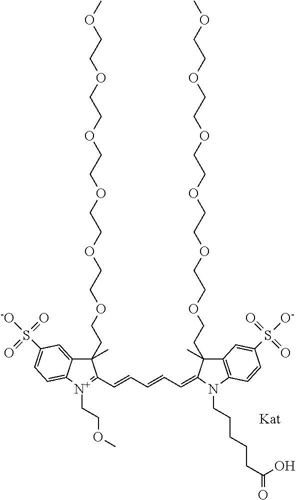

One non-limiting example of an additionally PEG-substituted compound is a 650 Compound 1/3 according to general formula II where both R1 and R2 are a sulfonamide group -L-SO₂NH—P—Z where Z is a methyl group, shown below:

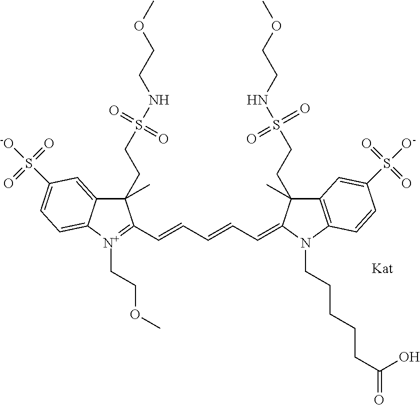

One non-limiting example of an additionally PEG-substituted compound is a 650 Compound 1/3 according to general formula II where both R1 and R2 are a carboxamide group -L-CONH—P—Z where Z is a methyl group, shown below:

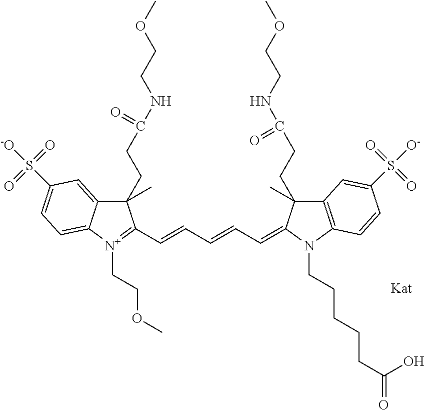

One non-limiting example of an additionally PEG-substituted compound is a 650 Compound 1/2 according to general formula II where R8 is an ethylene glycol group terminating with a methyl group, shown below:

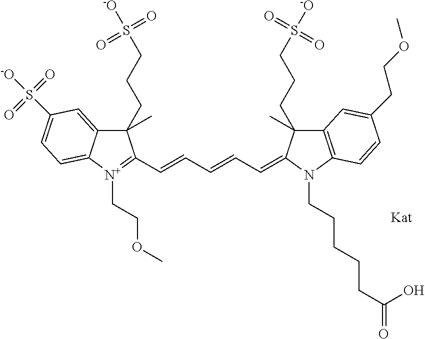

One non-limiting example of an additionally PEG-substituted compound is a 650 Compound 1/2 according to general formula II where R8 is a sulfonamide group —SO₂NH—P—Z where Z is a methyl group, shown below:

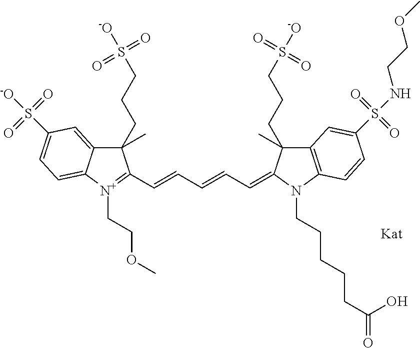

One non-limiting example of an additionally PEG-substituted compound is a 650 Compound 1/2 according to general formula II where R8 is a carboxamide group —CONH—P—Z where Z is a methyl group, shown below:

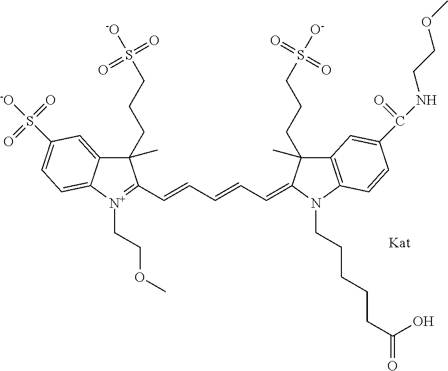

One non-limiting example of an additionally PEG-substituted compound is a 650 Compound 1/2 according to general formula II where R7 is an ethylene glycol group terminating with a methyl group, shown below:

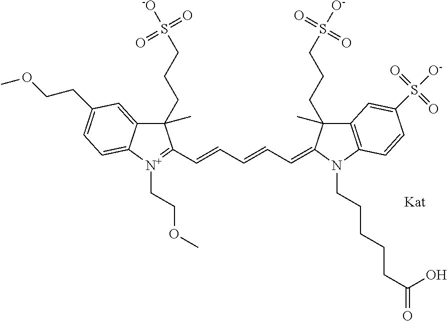

One non-limiting example of an additionally PEG-substituted compound is a 650 Compound 1/2 according to general formula II where R7 is a sulfonamide group —SO₂NH—P—Z where Z is a methyl group, shown below:

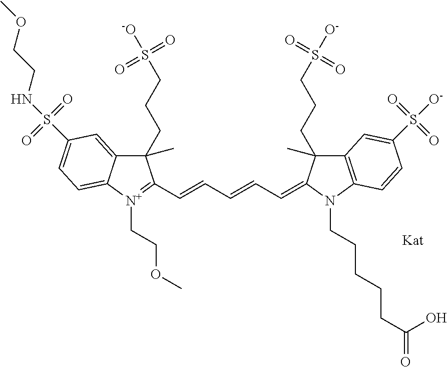

One non-limiting example of an additionally PEG-substituted compound is a 650 Compound 1/2 according to general formula II where R7 is a carboxamide group —CONH—P—Z where Z is a methyl group, shown below:

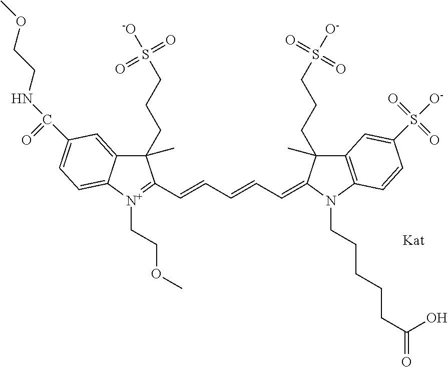

One non-limiting example of an additionally PEG-substituted compound is a 650 Compound 1/3 according to general formula II where both R7 and R8 are an ethylene glycol group terminating with a methyl group, shown below:

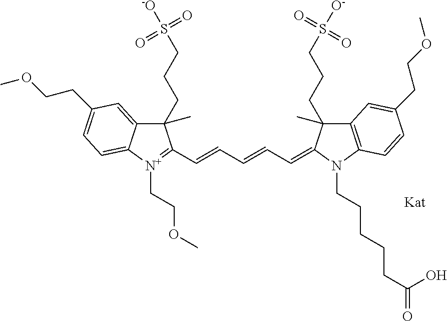

One non-limiting example of an additionally PEG-substituted compound is a 650 Compound 1/3 according to general formula II where both R7 and R8 are a sulfonamide group —SO₂NH—P—Z where Z is a methyl group, shown below:

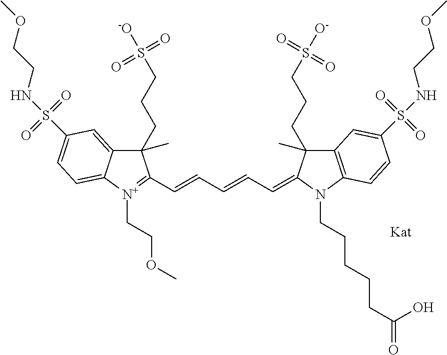

One non-limiting example of an additionally PEG-substituted compound is a 650 Compound 1/3 according to general formula II where both R7 and R8 are a carboxamide group —CONH—P—Z where Z is a methyl group, shown below:

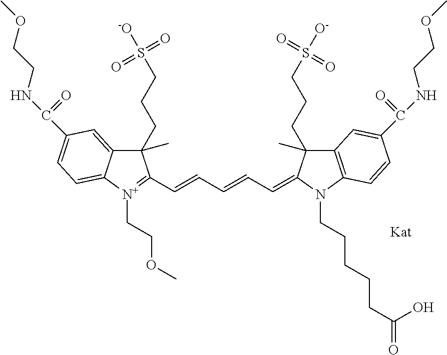

In one embodiment the compound is 755 Compound 1/2

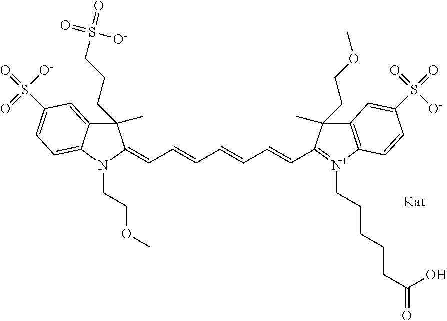 [SMILES: COCCN1/C(=C/C=C/C=C/C=C/C2=[N+](CCCCCC(=O)O)c3ccc(S(=O)(=O)[O-])cc3C2(C)CCOC)C(C)(CCCS(=O)(=O)[O-])c2cc(S(=O)(=O)[O-])ccc21]

755 Compound 1/2 (1-(5-carboxypentyl)-3-(2-methoxyethyl)-2-((1E,3E,5E,7E)-7-(1-(2-methoxyethyl)-3-methyl-5-sulfonato-3-(3-sulfonatopropyl)indolin-2-ylidene)hepta-1,3,5-trienyl)-3-methyl-3H-indolium-5-sulfonate tri sodium salt) contains an ethylene glycol on the indole N of the left heterocycle, i.e., a methylated ethylene glycol. The methyl group on the ethylene glycol prevents the terminal —OH from oxidation. Oxidation is known to occur, over time, on an unprotected PEG terminus (i.e., an unprotected terminus of an ethylene glycol group, diethylene glycol group, or (poly)ethylene glycol group). Adding a methyl ether provides this protection, and prevents reaction with electrophilic reactive groups.

In embodiments, e.g., for functional assays, the inventive compounds are activated. Activation of the compound adds a chemical moiety such that the compound is in a form that can be conjugated to a biological moiety. Examples of chemical moieties for activation are described below with reference to activation of 755 Compound 1/2, but one skilled in the art appreciates that activation is not limited to these examples. One non-limiting example of an activated compound is the NHS-ester of 755 Compound 1/2, shown below:

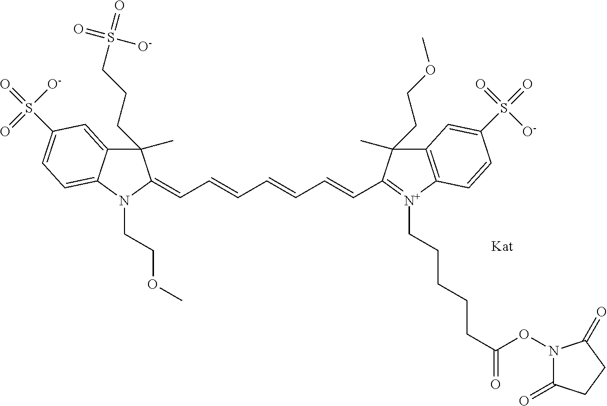 [SMILES: COCCN1/C(=C/C=C/C=C/C=C/C2=[N+](CCCCCC(=O)ON3C(=O)CCC3=O)c3ccc(S(=O)(=O)[O-])cc3C2(C)CCOC)C(C)(CCCS(=O)(=O)[O-])c2cc(S(=O)(=O)[O-])ccc21]

In one embodiment the compound is an NHS-ester of 755 Compound 1/2 where, according to general formula I, o is 1, shown below:

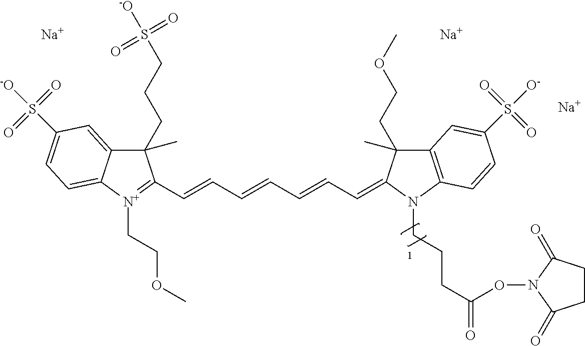

In one embodiment the compound is an NHS-ester of 755 Compound 1/2 where, according to general formula I, o is 5, shown below:

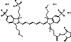

One non-limiting example of a NHS-ester of 755 Compound 1/3, according to general formula III, where m=1 and p=1, is shown below:

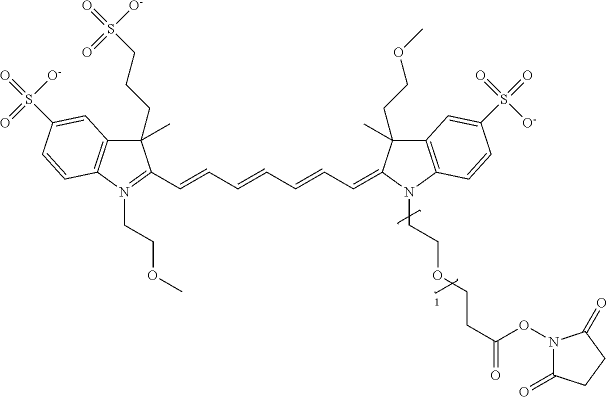

One non-limiting example of a NHS-ester of 755 Compound 1/3, according to general formula III, where m=1 and p=2, is shown below:

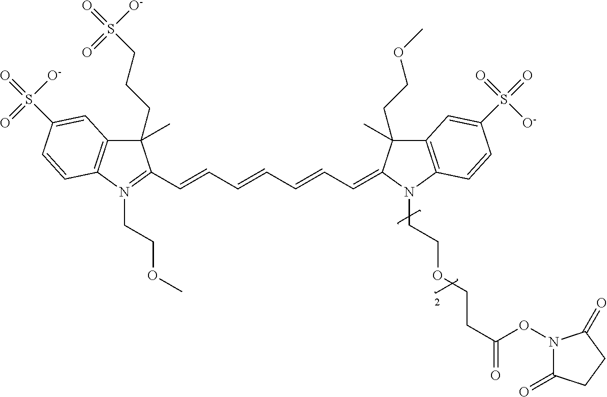

One non-limiting example of a NHS-ester of 755 Compound 1/3, according to general formula III, where m=1 and p=3, is shown below:

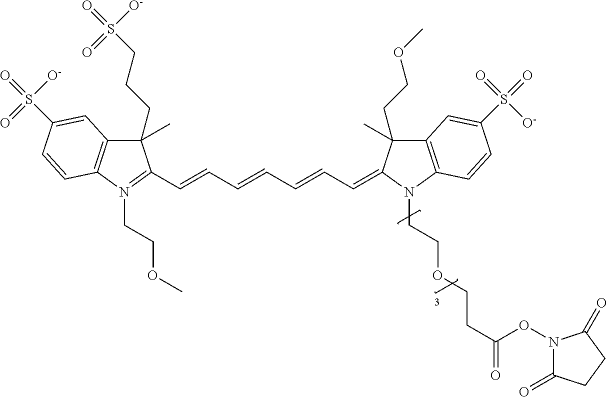

One non-limiting example of a NHS-ester of 755 Compound 1/3, according to general formula III, where m=1 and p=4, is shown below:

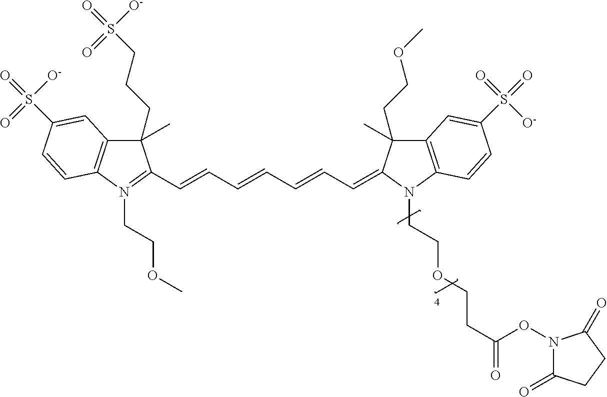

One non-limiting example of a NHS-ester of 755 Compound 1/3, according to general formula III, where m=1 and p=5, is shown below:

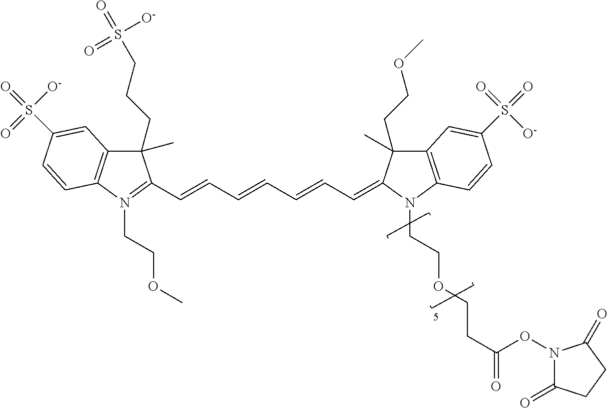

One non-limiting example of a NHS-ester of 755 Compound 1/3, according to general formula III, where m=1 and p=6, is shown below:

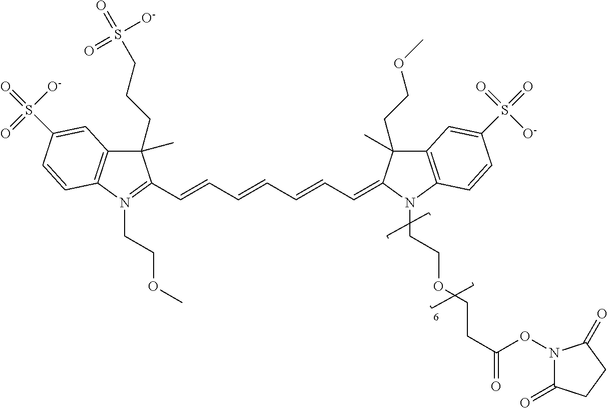

One non-limiting example of a NHS-ester of 755 Compound 2/3, according to general formula III, where m=2 and p=1, is shown below:

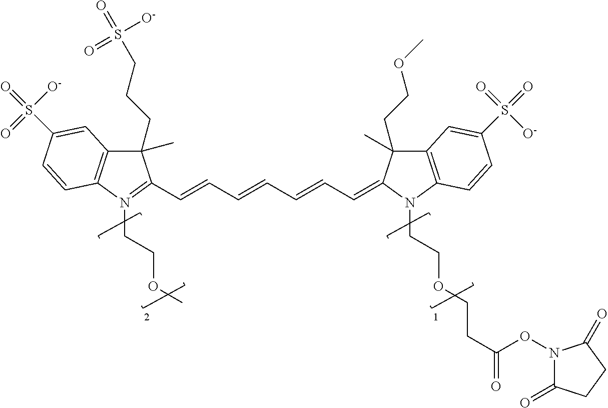

One non-limiting example of a NHS-ester of 755 Compound 2/3, according to general formula III, where m=2 and p=2, is shown below:

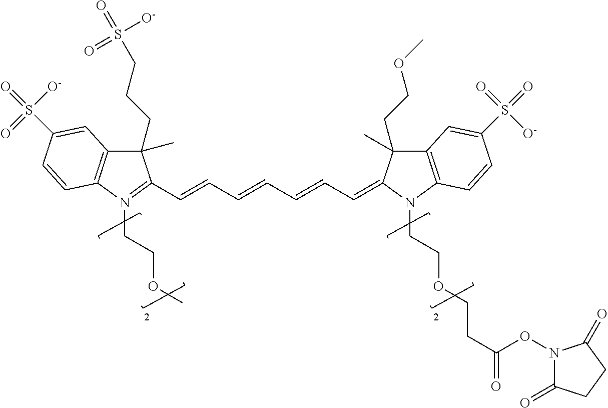

One non-limiting example of a NHS-ester of 755 Compound 2/3, according to general formula III, where m=2 and p=3, is shown below:

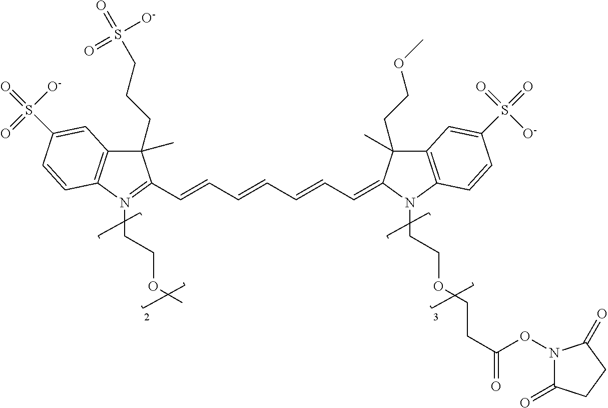

One non-limiting example of a NHS-ester of 755 Compound 3/3, according to general formula III, where m=3 and p=1, is shown below:

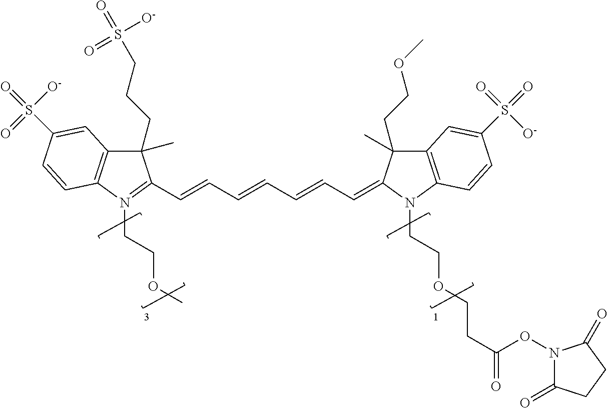

One non-limiting example of a NHS-ester of 755 Compound 3/3, according to general formula III, where m=3 and p=2, is shown below:

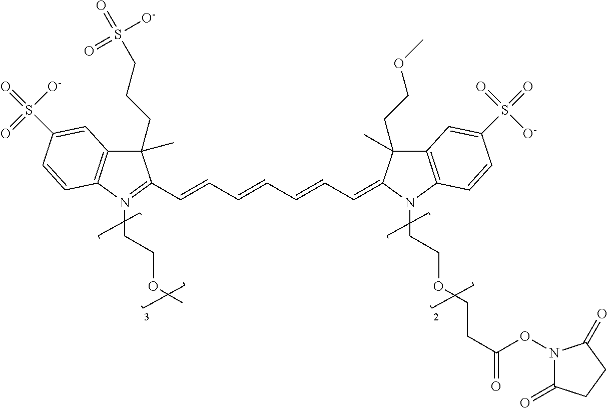

One non-limiting example of a NHS-ester of 755 Compound 3/3, according to general formula III, where m=3 and p=3, is shown below:

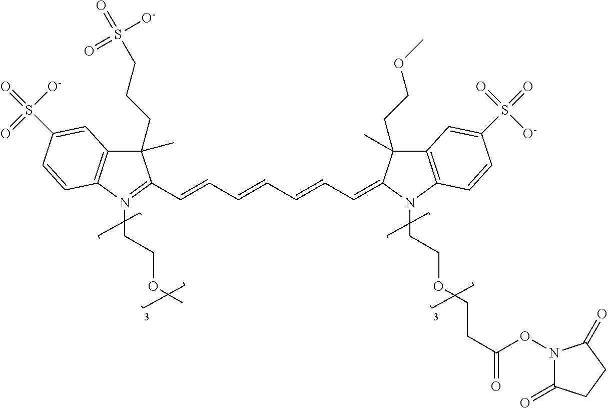

One non-limiting example of a NHS-ester of 755 Compound 4/3, according to general formula III, where m=4 and p=1, is shown below:

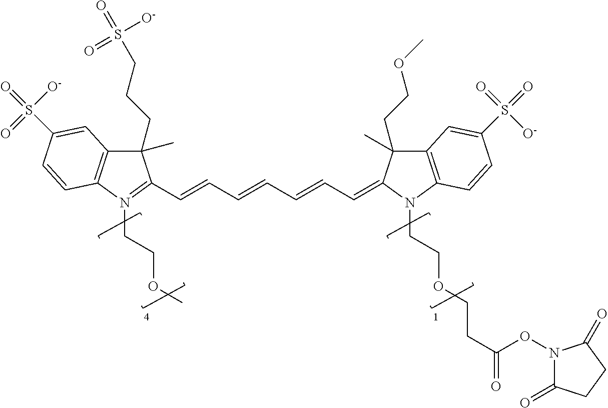

One non-limiting example of a NHS-ester of 755 Compound 5/3, according to general formula III, where m=5 and p=1, is shown below:

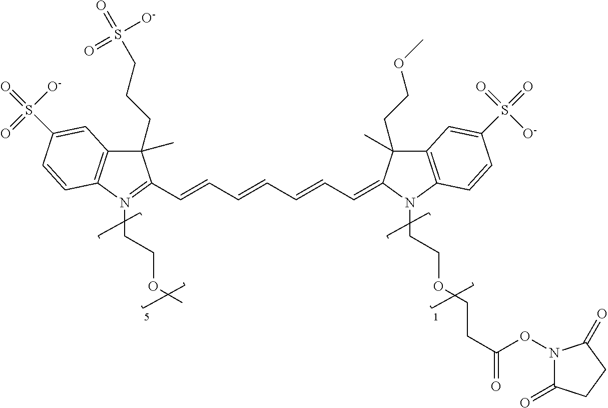

One non-limiting example of a NHS-ester of 755 Compound 6/3, according to general formula III, where m=6 and p=1, is shown below:

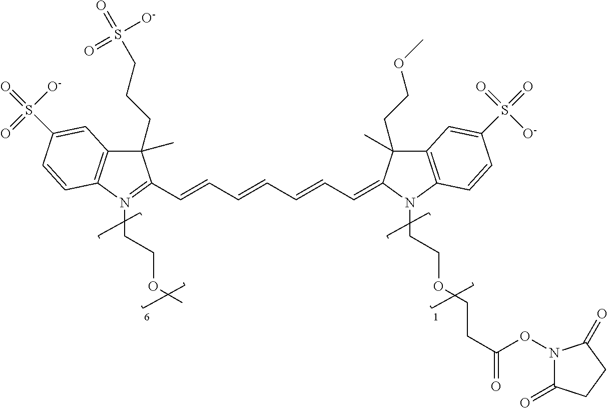

One non-limiting example of an activated 755 Compound 1/2 is a tetrafluorophenyl (TFP)-ester form of 755 Compound 1/2, shown below:

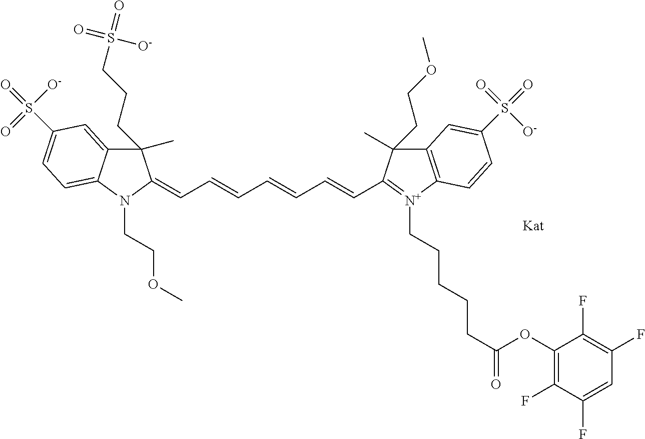 [SMILES: COCCN1/C(=C/C=C/C=C/C=C/C2=[N+](CCCCCC(=O)Oc3c(F)c(F)cc(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 755 Compound 1/2 is a sulfotetrafluorophenyl (STP)-ester form of 755 Compound 1/2, shown below:

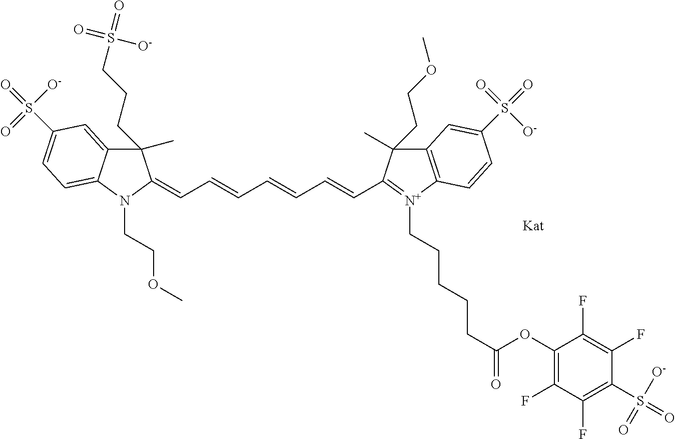 [SMILES: COCCN1/C(=C/C=C/C=C/C=C/C2=[N+](CCCCCC(=O)Oc3c(F)c(F)c(S(=O)(=O)[O-])c(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 755 Compound 1/2 is a hydrazide form of 755 Compound 1/2, shown below:

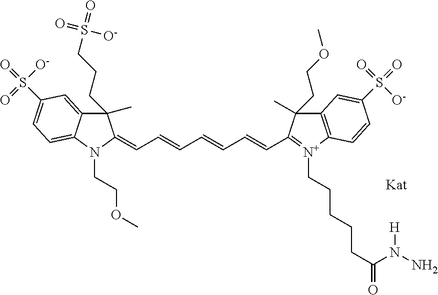 [SMILES: COCCN1/C(=C/C=C/C=C/C=C/C2=[N+](CCCCCC(=O)NN)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 755 Compound 1/2 is a maleimide form of 755 Compound 1/2, shown below:

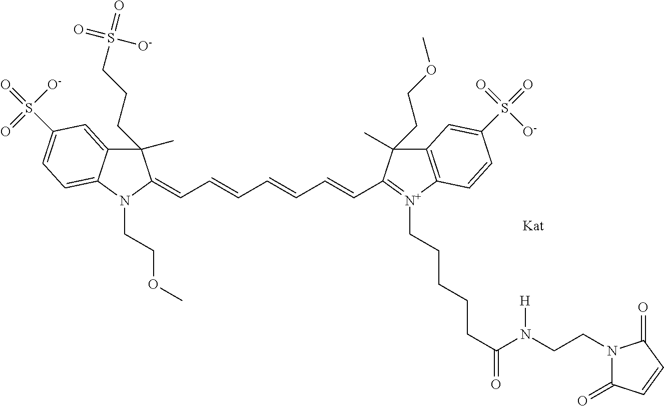 [SMILES: COCCN1/C(=C/C=C/C=C/C=C/C2=[N+](CCCCCC(=O)NCCN3C(=O)C=CC3=O)c3ccc(S(=O)(=O)[O-])cc3C2(C)CCOC)C(C)(CCCS(=O)(=O)[O-])c2cc(S(=O)(=O)[O-])ccc21]

In one embodiment the compound is 755 Compound 2/2

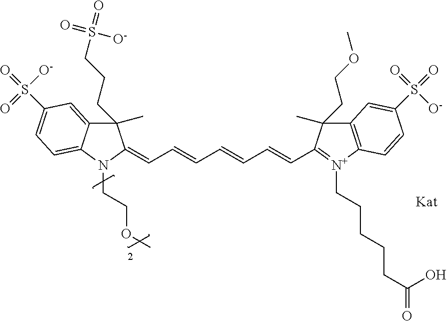

755 Compound 2/2 (1-(5-carboxypentyl)-2-((1E,3E,5E,7E)-7-(1-(2-(2-methoxyethoxy)ethyl)-3-methyl-5-sulfonato-3-(3-sulfonatopropyl)indolin-2-ylidene)hepta-1,3,5-trienyl)-3-(2-methoxyethyl)-3-methyl-3H-indolium-5-sulfonate) contains a (poly)ethylene glycol on the indole N of the left heterocycle. The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 755 Compound 2/2 is activated as described above, one non-limiting example of which is the NHS-ester form of 755 Compound 2/2, shown below.

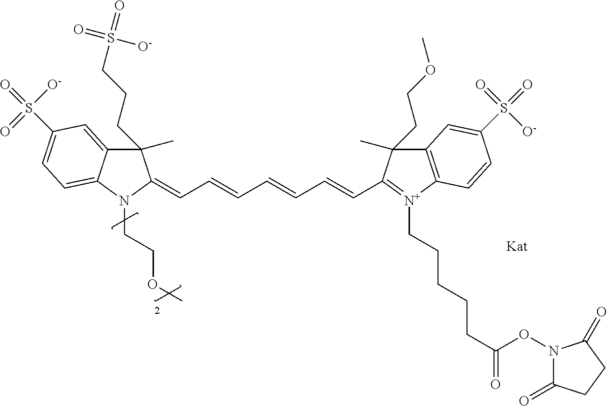

In one embodiment the compound is 755 Compound 3/2

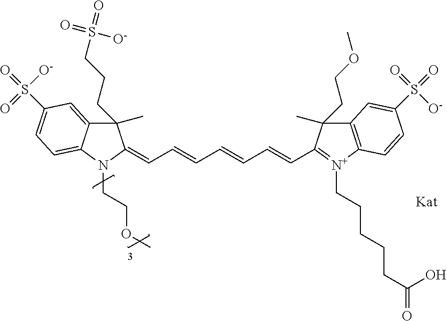

755 Compound 3/2 (1-(5-carboxypentyl)-2-((1E,3E,5E,7E)-7-(1-(2-(2-(2-methoxyethoxy)ethoxy)ethyl)-3-methyl-5-sulfonato-3-(3-sulfonatopropyl)indolin-2-ylidene)hepta-1,3,5-trienyl)-3-(2-methoxyethyl)-3-methyl-3H-indolium-5-sulfonate) contains a (poly)ethylene glycol on the indole N of the left heterocycle. The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 755 Compound 3/2 is activated as described above, one non-limiting example of which is the NHS-ester form of 755 Compound 3/2, shown below.

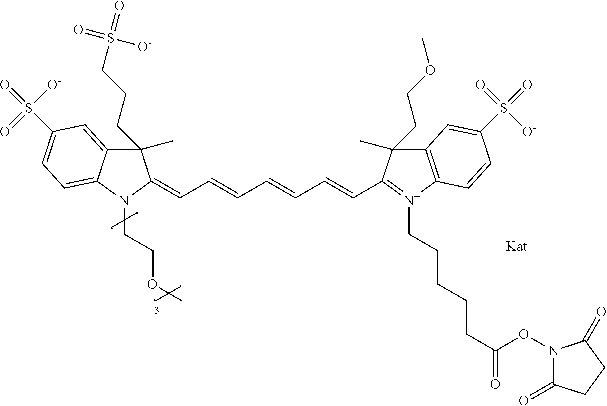

In one embodiment the compound is 755 Compound 4/2

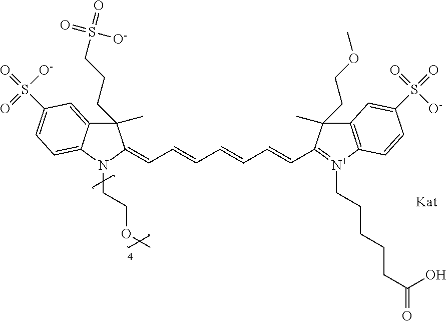

755 Compound 4/2 (1-(5-carboxypentyl)-3-(2-methoxyethyl)-3-methyl-2-((1E,3E,5E,7E)-7-(3-methyl-5-sulfonato-3-(3-sulfonatopropyl)-1-(2,5,8,11-tetraoxatridecan-13-yl)indolin-2-ylidene)hepta-1,3,5-trienyl)-3H-indolium-5-sulfonate) contains a (poly)ethylene glycol on the indole N of the left heterocycle. The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 755 Compound 4/2 is activated as described above.

In one embodiment the compound is 755 Compound 5/2

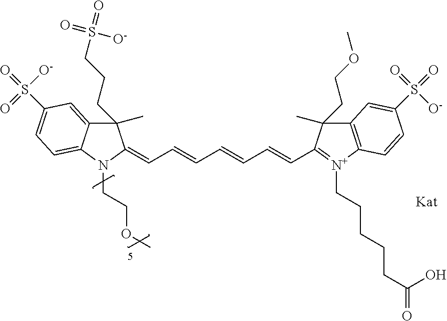

755 Compound 5/2 (2-((1E,3E,5E,7E)-7-(1-(2,5,8,11,14-pentaoxahexadecan-16-yl)-3-methyl-5-sulfonato-3-(3-sulfonatopropyl)indolin-2-ylidene)hepta-1,3,5-trienyl)-1-(5-carboxypentyl)-3-(2-methoxyethyl)-3-methyl-3H-indolium-5-sulfonate) contains a (poly)ethylene glycol on the indole N of the left heterocycle. The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 755 Compound 5/2 is activated as described above.

In one embodiment the compound is 755 Compound 6/2

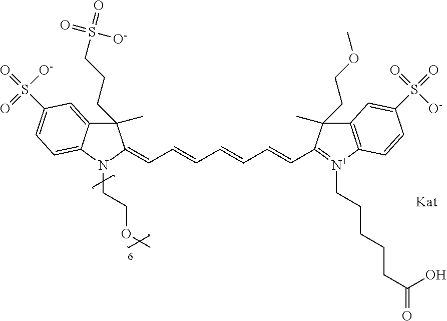

755 Compound 6/2 (1-(5-carboxypentyl)-3-(2-methoxyethyl)-3-methyl-2-((1E,3E,5E,7E)-7-(3-methyl-1-(2,5,8,11,14,17-hexaoxanonadecan-19-yl)-5-sulfonato-3-(3-sulfonatopropyl)indolin-2-ylidene)hepta-1,3,5-trienyl)-3H-indolium-5-sulfonate) contains a (poly)ethylene glycol on the indole N of the left heterocycle. The methyl group on the ethylene glycol prevents the terminal —OH from oxidation. Oxidation is known to occur, over time, on an unprotected PEG terminus. Adding a methyl ether provides this protection, and prevents reaction with electrophilic reactive groups. For functional assays, 755 Compound 6/2 is activated as described above.

In embodiments, the compound contains one or more substitutions of the polymethine linker. In one embodiment the compound has general formula VIa

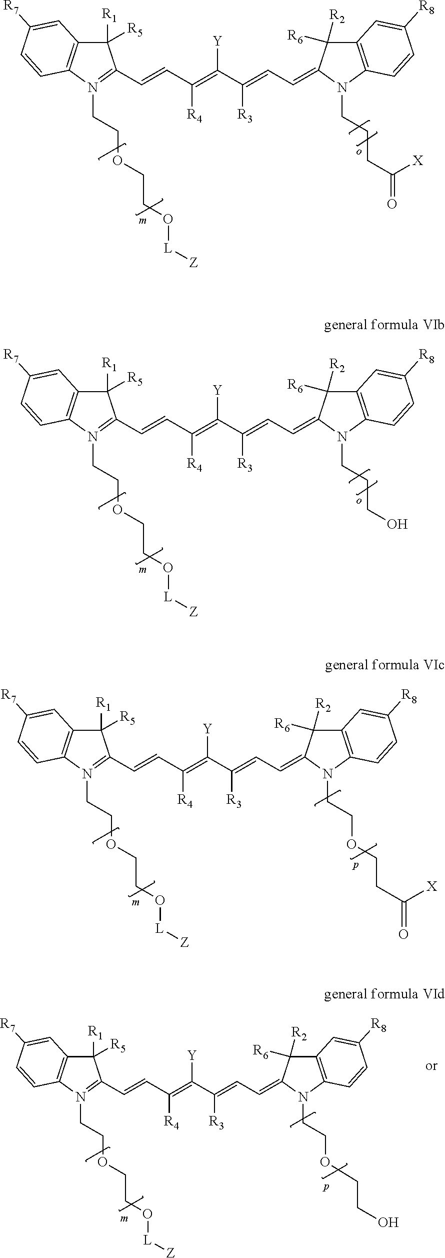

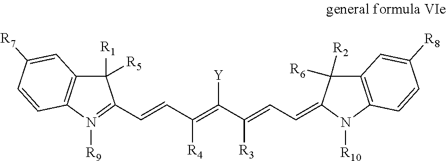

where each of R¹, R², R⁵, and R⁶ is the same or different and is independently selected from the group consisting of an aliphatic, heteroaliphatic, sulfoalkyl, heteroaliphatic with terminal SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group -L-SO₂NH—P-L-Z, and caboxamide group -L-CONH—P-L-Z; each of R⁷ and R⁸ is the same or different and is independently selected from either H, SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, sulfonamide group —SO₂NH—P-L-Z, or caboxamide group —CONH—P-L-Z; each of R⁹ and R¹⁰ is the same or different and is independently selected from the group consisting of alkyl, sulfoalkyl, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a PEG group P-L-X, L-Z, L-X; where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur; where Z is selected from the group consisting of H, CH₃, an alkyl, a heteroalkyl, NH₂, —COO, —COOH, —COSH, CO—NH—NH₂, —COF, —COCl, —COBr, —COI, —COO-Su (succinimidyl/sulfo-succinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO⁻, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-O-phosphoramidite, —CONR′-L-CHO, —CONR′-L-maleimide, and —CONR′-L-NH—CO—CH₂—I; R′ and R″ is selected from the group consisting of H, aliphatic group, and heteroaliphatic group, and the biomolecule is a protein, antibody, nucleotide, oligonucleotide, biotin, or hapten; X is selected from the group consisting of —OH, —SH, —NH₂, —NH—NH₂, —F, —Cl, —Br, I, —NHS (hydroxysuccinimidyl/sulfosuccinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group; Kat is a number of Na⁺, K⁺, Ca²⁺, ammonia, or other cation(s) needed to compensate the negative charge brought by the cyanine; m is an integer from 0 to 5 inclusive; p is an integer from 1 to 6 inclusive; each of R3 and R4 is the same or different and is independently hydrogen, an aliphatic group, a heteroaliphatic group, or a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive; or R3 and R4 together form a cyclic structure where R3 and R4 are joined using a divalent structural element selected from the group consisting of —(CH₂)_{q}—, —(CH₂)_{q}O(CH₂)_{q′}—, —(CH₂)_{q}S(CH₂)_{q′}—, —(CH₂)_{q}CH═CH—, —OCH═CH— where each of q and q′ is the same or different and is a integer from 2 to 6 inclusive; and Y is selected from the group consisting of hydrogen, alkyl, sulfoalkyl, fluorine, chlorine, bromine, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, and an oxygen-containing group OR^{PM}, where R^{PM} is selected from the group consisting of hydrogen, a substituted or unsubstituted alkyl group, a substituted or unsubstituted heteroalkyl group, a substituted or unsubstituted cyclic alkyl group, a substituted or unsubstituted heterocyclic alkyl group, a substituted or unsubstituted aryl group, and a substituted or unsubstituted heteroaryl group, where the group can be substituted one or more times with at least one of hydroxyl, sulfo, carboxy, and/or amino; with the proviso that at least one of R¹, R², R³, R⁴, R⁵, R⁶, R⁷, and R⁸ contains a PEG group.

In one embodiment the compound of general formula IV wherein each of R3 and R4 is the same or different and is independently hydrogen, an aliphatic group, or a heteroaliphatic group, or R3 and R4 together form a cyclic structure where R3 and R4 are directly joined or joined using a divalent structural element selected from the group consisting of —(CH₂)_{q}— and CH═CH, where q is an integer from 1 to 2 inclusive, to result in a 3-, 4-, or 5-membered ring.

In one embodiment the compound of general formula IV wherein R3 and R4 together form a cyclic structure where R3 and R4 are joined using a divalent structural element of —(CH₂)_{q}—, where q is 3, to result in a 6-membered ring, and Y is OR^{PM}, where R^{PM} is a substituted 6-membered aryl group, where the substituted group is a sulfo group.

One non-limiting example is a substituted polymethine form of 755 Compound 1/2, shown below:

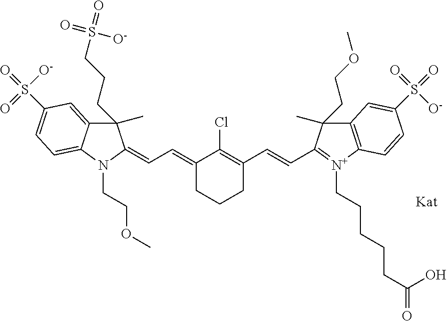 [SMILES: COCCN1/C(=C/C=C2\CCCC(/C=C/C3=[N+](CCCCCC(=O)O)c4ccc(S(=O)(=O)[O-])cc4C3(C)CCOC)=C2Cl)C(C)(CCCS(=O)(=O)[O-])c2cc(S(=O)(=O)[O-])ccc21]

One non-limiting example is a substituted polymethine form of 755 Compound 2/2, shown below:

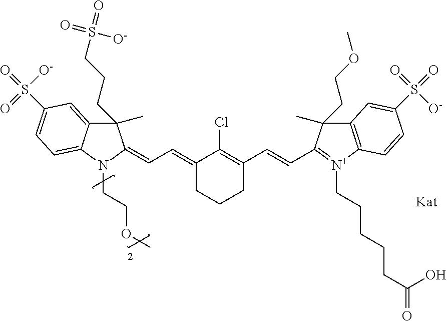

One non-limiting example is a substituted polymethine form of 755 Compound 3/2, shown below:

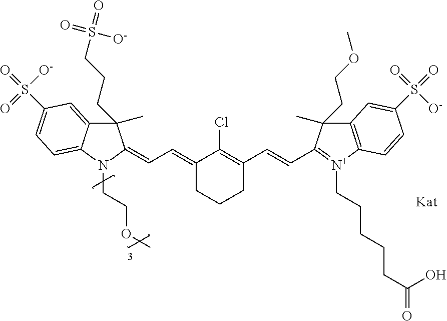

One non-limiting example is a substituted polymethine form of 755 Compound 4/2, shown below:

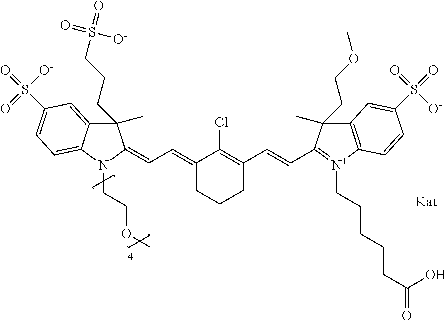

One non-limiting example is a substituted polymethine form of 755 Compound 5/2, shown below:

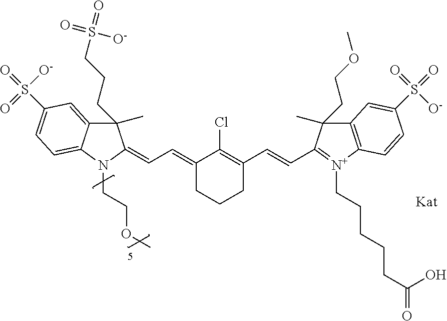

One non-limiting example is a substituted polymethine form of 755 Compound 6/2, shown below:

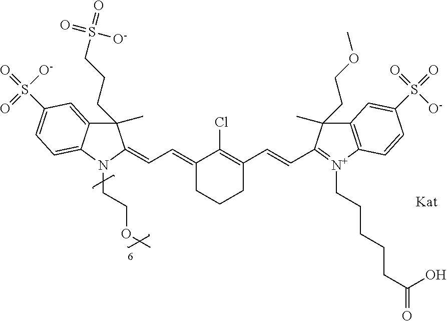

One non-limiting example is a substituted polymethine form of 755 Compound 0/1, shown below:

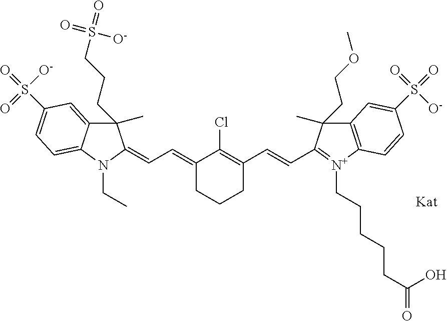 [SMILES: CCN1/C(=C/C=C2\CCCC(/C=C/C3=[N+](CCCCCC(=O)O)c4ccc(S(=O)(=O)[O-])cc4C3(C)CCOC)=C2Cl)C(C)(CCCS(=O)(=O)[O-])c2cc(S(=O)(=O)[O-])ccc21]

One non-limiting example is a substituted polymethine form of 755 Compound 0/1, shown below:

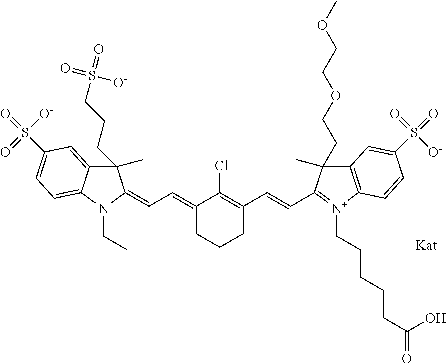 [SMILES: CCN1/C(=C/C=C2\CCCC(/C=C/C3=[N+](CCCCCC(=O)O)c4ccc(S(=O)(=O)[O-])cc4C3(C)CCOCCOC)=C2Cl)C(C)(CCCS(=O)(=O)[O-])c2cc(S(=O)(=O)[O-])ccc21]

One non-limiting example is a substituted polymethine form of 755 Compound 0/1, shown below:

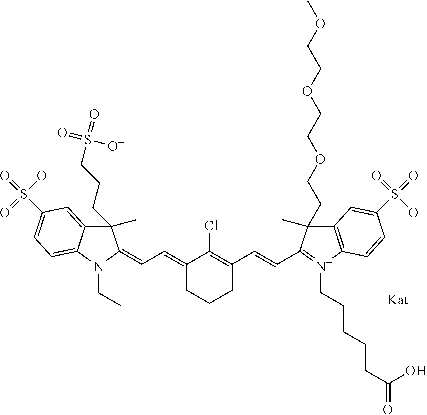 [SMILES: CCN1/C(=C/C=C2\CCCC(/C=C/C3=[N+](CCCCCC(=O)O)c4ccc(S(=O)(=O)[O-])cc4C3(C)CCOCCOCCOC)=C2Cl)C(C)(CCCS(=O)(=O)[O-])c2cc(S(=O)(=O)[O-])ccc21]

One non-limiting example is a substituted polymethine form of 755 Compound 0/1, shown below:

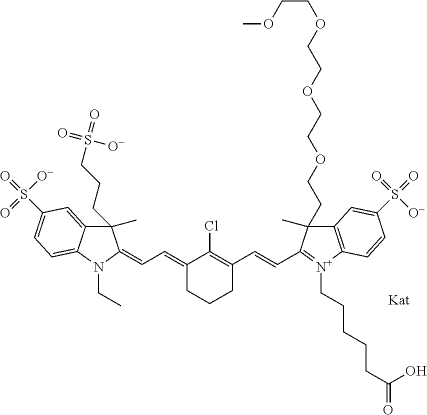 [SMILES: CCN1/C(=C/C=C2\CCCC(/C=C/C3=[N+](CCCCCC(=O)O)c4ccc(S(=O)(=O)[O-])cc4C3(C)CCOCCOCCOCCOC)=C2Cl)C(C)(CCCS(=O)(=O)[O-])c2cc(S(=O)(=O)[O-])ccc21]

One non-limiting example is a substituted polymethine form of 755 Compound 0/1, shown below:

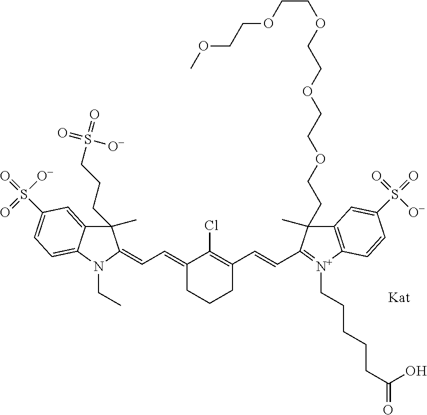 [SMILES: CCN1/C(=C/C=C2\CCCC(/C=C/C3=[N+](CCCCCC(=O)O)c4ccc(S(=O)(=O)[O-])cc4C3(C)CCOCCOCCOCCOCCOC)=C2Cl)C(C)(CCCS(=O)(=O)[O-])c2cc(S(=O)(=O)[O-])ccc21]

One non-limiting example is a substituted polymethine form of 755 Compound 0/1, shown below:

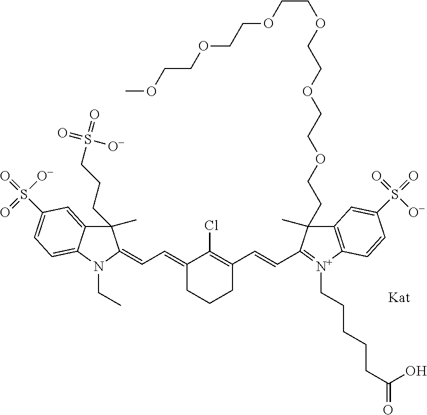 [SMILES: CCN1/C(=C/C=C2\CCCC(/C=C/C3=[N+](CCCCCC(=O)O)c4ccc(S(=O)(=O)[O-])cc4C3(C)CCOCCOCCOCCOCCOCCOC)=C2Cl)C(C)(CCCS(=O)(=O)[O-])c2cc(S(=O)(=O)[O-])ccc21]

One non-limiting example is a substituted polymethine form of 755 Compound 1/3 having an ethylene glycol, diethylene glycol, or (poly)ethylene glycol as described for general formula VI, such as the compound shown below:

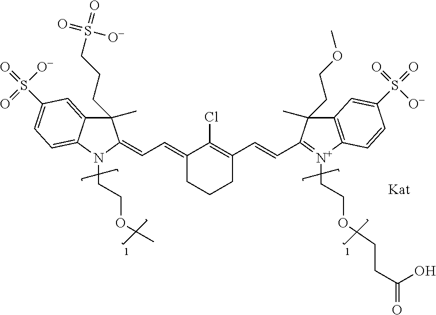

One non-limiting example is a substituted polymethine form of 755 Compound 4/4 having an ethylene glycol, diethylene glycol, or (poly)ethylene glycol as described for general formula VI, such as the compound shown below:

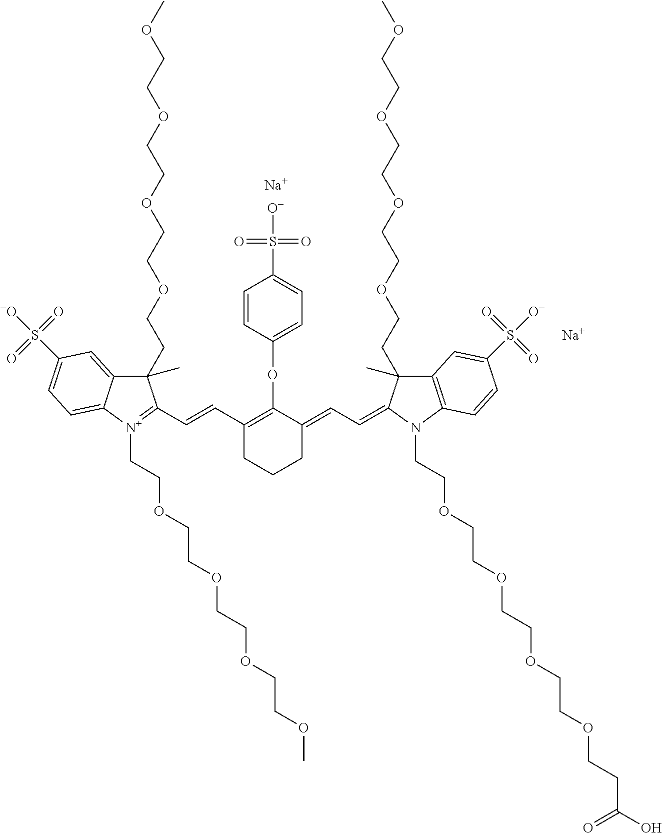

In one embodiment the compound is 755 Compound 0/1

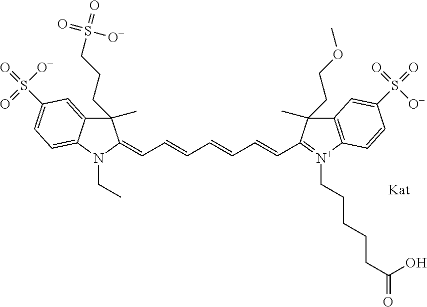 [SMILES: CCN1/C(=C/C=C/C=C/C=C/C2=[N+](CCCCCC(=O)O)c3ccc(S(=O)(=O)[O-])cc3C2(C)CCOC)C(C)(CCCS(=O)(=O)[O-])c2cc(S(=O)(=O)[O-])ccc21]

755 Compound 0/1 (1-(5-carboxypentyl)-2-((1E,3E,5E,7E)-7-(1-ethyl-3-methyl-5-sulfonato-3-(3-sulfonatopropyl)indolin-2-ylidene)hepta-1,3,5-trienyl)-3-(2-methoxyethyl)-3-methyl-3H-indolium-5-sulfonate) contains ethylene glycol at R2, sulfoalkyl at R1, ethyl at R9, and carboxyalkyl at R10.

One non-limiting example of an activated compound is the NHS-ester of 755 Compound 0/1, shown below:

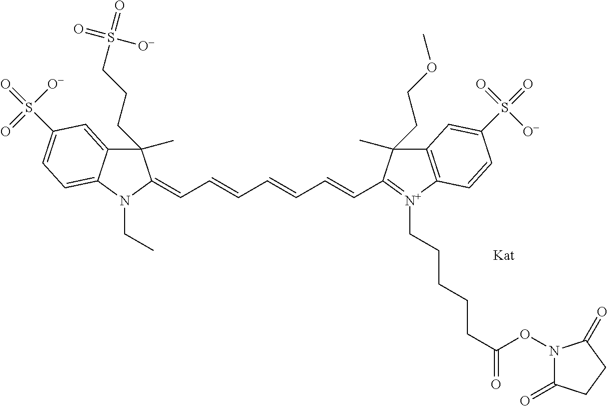 [SMILES: CCN1/C(=C/C=C/C=C/C=C/C2=[N+](CCCCCC(=O)ON3C(=O)CCC3=O)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 755 Compound 0/1 is a tetrafluorophenyl (TFP)-ester form, shown below:

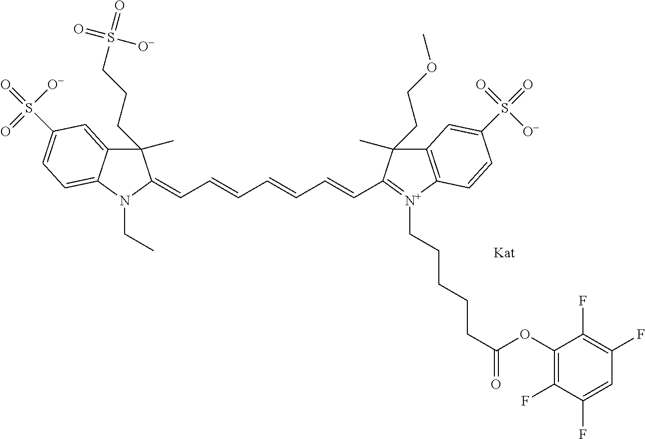 [SMILES: CCN1/C(=C/C=C/C=C/C=C/C2=[N+](CCCCCC(=O)Oc3c(F)c(F)cc(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 755 Compound 0/1 is a sulfotetrafluorophenyl (STP)-ester form, shown below:

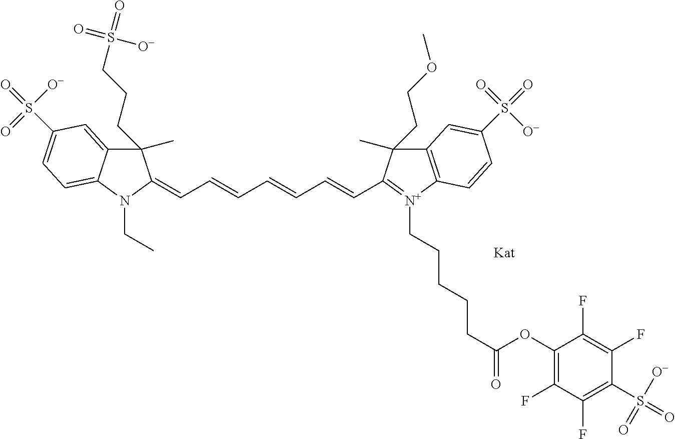 [SMILES: CCN1/C(=C/C=C/C=C/C=C/C2=[N+](CCCCCC(=O)Oc3c(F)c(F)c(S(=O)(=O)[O-])c(F)c3F)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 755 Compound 0/1 is a hydrazide form, shown below:

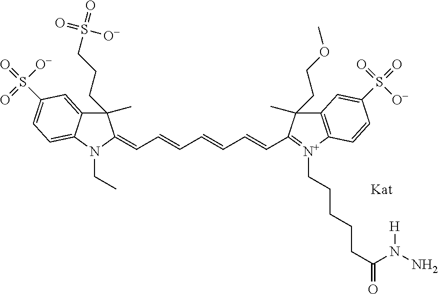 [SMILES: CCN1/C(=C/C=C/C=C/C=C/C2=[N+](CCCCCC(=O)NN)c3ccc(S(=O)(=O)[O-])cc3C2(C)CCOC)C(C)(CCCS(=O)(=O)[O-])c2cc(S(=O)(=O)[O-])ccc21]

One non-limiting example of an activated 755 Compound 0/1 is a maleimide form, shown below:

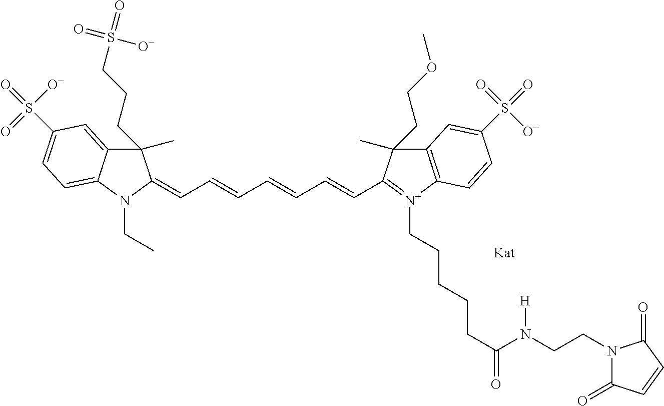 [SMILES: CCN1/C(=C/C=C/C=C/C=C/C2=[N+](CCCCCC(=O)NCCN3C(=O)C=CC3=O)c3ccc(S(=O)(=O)[O-])cc3C2(C)CCOC)C(C)(CCCS(=O)(=O)[O-])c2cc(S(=O)(=O)[O-])ccc21]

In one embodiment the compound is 755 Compound 0/1

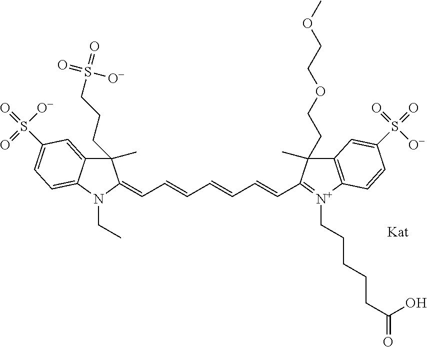 [SMILES: CCN1/C(=C/C=C/C=C/C=C/C2=[N+](CCCCCC(=O)O)c3ccc(S(=O)(=O)[O-])cc3C2(C)CCOCCOC)C(C)(CCCS(=O)(=O)[O-])c2cc(S(=O)(=O)[O-])ccc21]

755 Compound 0/1 (1-(5-carboxypentyl)-2-((1E,3E,5E,7E)-7-(1-ethyl-3-methyl-5-sulfonato-3-(3-sulfonatopropyl)indolin-2-ylidene)hepta-1,3,5-trienyl)-3-(2-(2-methoxyethoxy)ethyl)-3-methyl-3H-indolium-5-sulfonate) contains a diethylene glycol at R2, sulfoalkyl at R1, ethyl at R9, and carboxyalkyl at R10.

In one embodiment the compound is 755 Compound 0/1

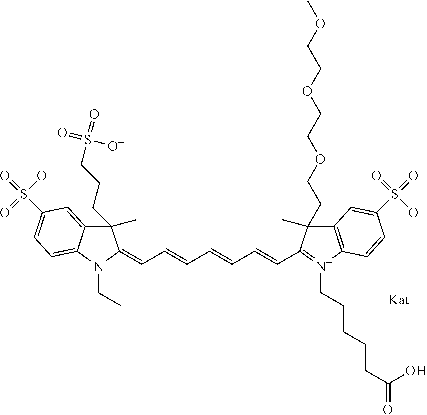 [SMILES: CCN1/C(=C/C=C/C=C/C=C/C2=[N+](CCCCCC(=O)O)c3ccc(S(=O)(=O)[O-])cc3C2(C)CCOCCOCCOC)C(C)(CCCS(=O)(=O)[O-])c2cc(S(=O)(=O)[O-])ccc21]

755 Compound 0/1 (1-(5-carboxypentyl)-2-((1E,3E,5E,7E)-7-(1-ethyl-3-methyl-5-sulfonato-3-(3-sulfonatopropyl)indolin-2-yl idene)hepta-1,3,5-trienyl)-3-(2-(2-(2-methoxyethoxy)ethoxy)ethyl)-3-methyl-3H-indolium-5-sulfonate) contains a (poly)ethylene glycol (PEG₃) at R2, sulfoalkyl at R1, ethyl at R9, and carboxyalkyl at R10.

In one embodiment the compound is 755 Compound 0/1

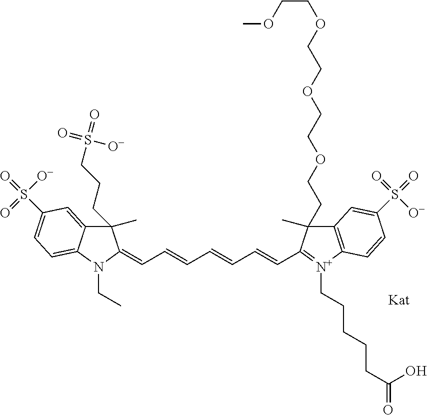 [SMILES: CCN1/C(=C/C=C/C=C/C=C/C2=[N+](CCCCCC(=O)O)c3ccc(S(=O)(=O)[O-])cc3C2(C)CCOCCOCCOCCOC)C(C)(CCCS(=O)(=O)[O-])c2cc(S(=O)(=O)[O-])ccc21]

755 Compound 0/1 (1-(5-carboxypentyl)-2-((1E,3E,5E,7E)-7-(1-ethyl-3-methyl-5-sulfonato-3-(3-sulfonatopropyl)indolin-2-ylidene)hepta-1,3,5-trienyl)-3-methyl-3-(2,5,8,11-tetraoxatridecan-13-yl)-3H-indolium-5-sulfonate) contains a (poly)ethylene glycol (PEG₄) at R2, sulfoalkyl at R1, ethyl at R9, and carboxyalkyl at R10.

In one embodiment the compound is 755 Compound 0/1

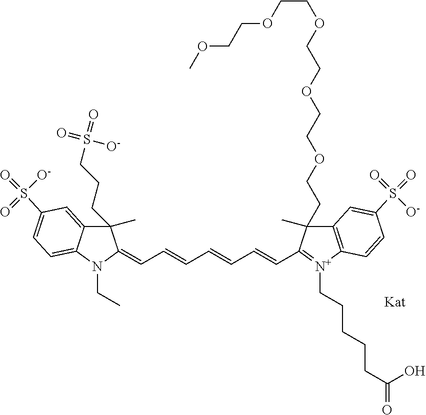 [SMILES: CCN1/C(=C/C=C/C=C/C=C/C2=[N+](CCCCCC(=O)O)c3ccc(S(=O)(=O)[O-])cc3C2(C)CCOCCOCCOCCOCCOC)C(C)(CCCS(=O)(=O)[O-])c2cc(S(=O)(=O)[O-])ccc21]

755 Compound 0/1 (1-(5-carboxypentyl)-2-((1E,3E,5E,7E)-7-(1-ethyl-3-methyl-5-sulfonato-3-(3-sulfonatopropyl)indolin-2-ylidene)hepta-1,3,5-trienyl)-3-(2,5,8,11,14-pentaoxahexadecan-16-yl)-3-methyl-3H-indolium-5-sulfonate) contains a (poly)ethylene glycol (PEG₅) at R2, sulfoalkyl at R1, ethyl at R9, and carboxyalkyl at R10.

In one embodiment the compound is 755 Compound 0/1

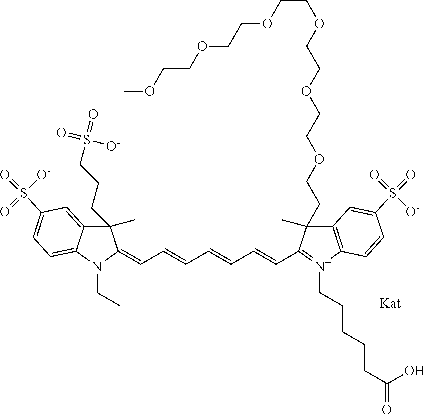 [SMILES: CCN1/C(=C/C=C/C=C/C=C/C2=[N+](CCCCCC(=O)O)c3ccc(S(=O)(=O)[O-])cc3C2(C)CCOCCOCCOCCOCCOCCOC)C(C)(CCCS(=O)(=O)[O-])c2cc(S(=O)(=O)[O-])ccc21]

755 Compound 0/1 (1-(5-carboxypentyl)-2-((1E,3E,5E,7E)-7-(1-ethyl-3-methyl-5-sulfonato-3-(3-sulfonatopropyl)indolin-2-ylidene)hepta-1,3,5-trienyl)-3-methyl-3-(2,5,8,11,14,17-hexaoxanonadecan-19-yl)-3H-indolium-5-sulfonate) contains a (poly)ethylene glycol (PEG₆) at R2, sulfoalkyl at R1, ethyl at R9, and carboxyalkyl at R10.

In one embodiment the compound is 755 Compound 0/1

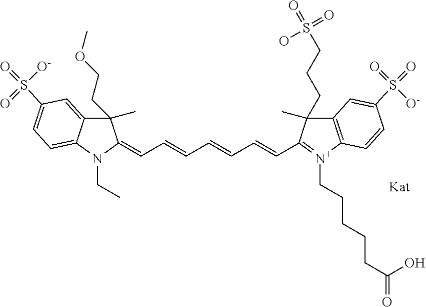 [SMILES: CCN1/C(=C/C=C/C=C/C=C/C2=[N+](CCCCCC(=O)O)c3ccc(S(=O)(=O)[O-])cc3C2(C)CCCS(=O)(=O)O)C(C)(CCOC)c2cc(S(=O)(=O)[O-])ccc21]

755 Compound 0/1 (1-(5-carboxypentyl)-2-((1E,3E,5E,7E)-7-(1-ethyl-3-(2-methoxyethyl)-3-methyl-5-sulfonatoindolin-2-ylidene)hepta-1,3,5-trienyl)-3-methyl-3-(3-sulfopropyl)-3H-indolium-5-sulfonate) contains ethylene glycol at R1, sulfoalkyl at R2, ethyl at R9, and carboxyalkyl at R10.

One non-limiting example of an activated compound is the NHS-ester of 755 Compound 0/1, shown below:

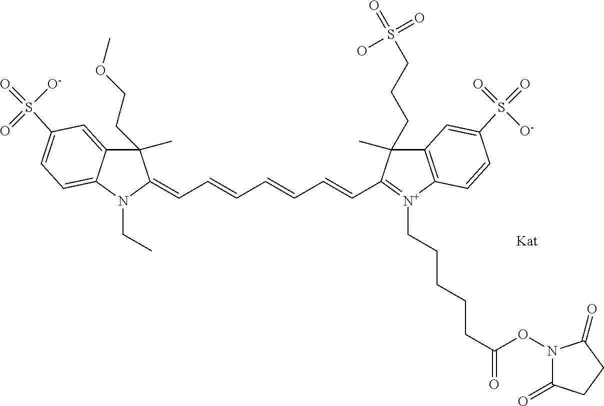 [SMILES: CCN1/C(=C/C=C/C=C/C=C/C2=[N+](CCCCCC(=O)ON3C(=O)CCC3=O)c3ccc(S(=O)(=O)[O-])cc3C2(C)CCCS(=O)(=O)O)C(C)(CCOC)c2cc(S(=O)(=O)[O-])ccc21]

In one embodiment the compound is 755 Compound 0/1

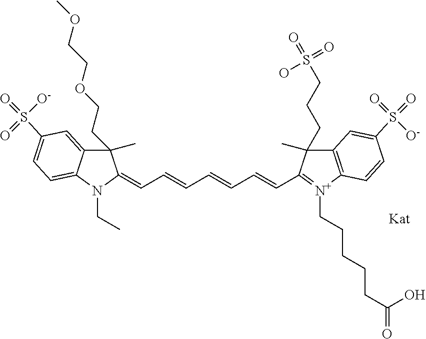 [SMILES: CCN1/C(=C/C=C/C=C/C=C/C2=[N+](CCCCCC(=O)O)c3ccc(S(=O)(=O)[O-])cc3C2(C)CCCS(=O)(=O)O)C(C)(CCOCCOC)c2cc(S(=O)(=O)[O-])ccc21]

755 Compound 0/1 (1-(5-carboxypentyl)-2-((1E,3E,5E,7E)-7-(1-ethyl-3-(2-(2-(2-methoxyethoxy)ethyl)-3-methyl-5-sulfonatoindolin-2-ylidene)hepta-1,3,5-trienyl)-3-methyl-3-(3-sulfopropyl)-3H-indolium-5-sulfonate) contains a diethylene glycol at R1, sulfoalkyl at R2, ethyl at R9, and carboxyalkyl at R10.

In one embodiment the compound is 755 Compound 0/1

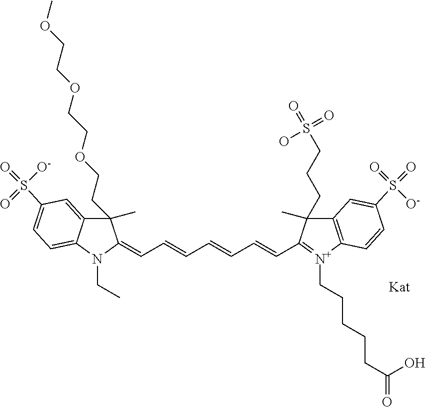 [SMILES: CCN1/C(=C/C=C/C=C/C=C/C2=[N+](CCCCCC(=O)O)c3ccc(S(=O)(=O)[O-])cc3C2(C)CCCS(=O)(=O)O)C(C)(CCOCCOCCOC)c2cc(S(=O)(=O)[O-])ccc21]

755 Compound 0/1 (1-(5-carboxypentyl)-2-((1E,3E,5E,7E)-7-(1-ethyl-3-(2-(2-(2-methoxyethoxy)ethoxy)ethyl)-3-methyl-5-sulfonatoindolin-2-ylidene)hepta-1,3,5-trienyl)-3-methyl-3-(3-sulfopropyl)-3H-indolium-5-sulfonate) contains a (poly)ethylene glycol (PEG₃) at R1, sulfoalkyl at R2, ethyl at R9, and carboxyalkyl at R10.

In one embodiment the compound is 755 Compound 0/1

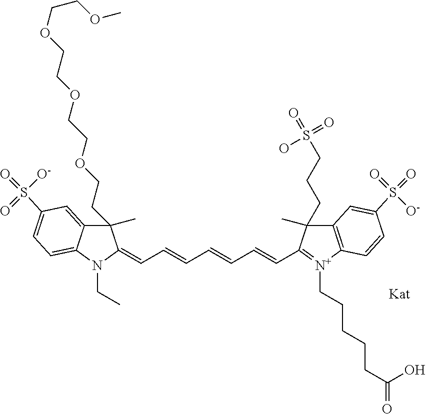 [SMILES: CCN1/C(=C/C=C/C=C/C=C/C2=[N+](CCCCCC(=O)O)c3ccc(S(=O)(=O)[O-])cc3C2(C)CCCS(=O)(=O)O)C(C)(CCOCCOCCOCCOC)c2cc(S(=O)(=O)[O-])ccc21]

755 Compound 0/1 (1-(5-carboxypentyl)-2-((1E,3E,5E,7E)-7-(1-ethyl-3-methyl-5-sulfonato-3-(2,5,8,11-tetraoxatridecan-13-yl)indolin-2-ylidene)hepta-1,3,5-trienyl)-3-methyl-3-(3-sulfopropyl)-3H-indolium-5-sulfonate) contains a (poly)ethylene glycol (PEG₄) at R1, sulfoalkyl at R2, ethyl at R9, and carboxyalkyl at R10.

In one embodiment the compound is 755 Compound 0/1

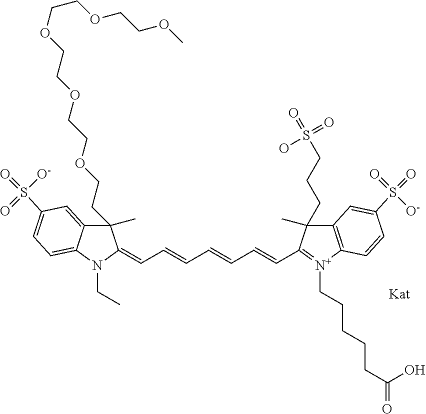 [SMILES: CCN1/C(=C/C=C/C=C/C=C/C2=[N+](CCCCCC(=O)O)c3ccc(S(=O)(=O)[O-])cc3C2(C)CCCS(=O)(=O)O)C(C)(CCOCCOCCOCCOCCOC)c2cc(S(=O)(=O)[O-])ccc21]

755 Compound 0/1 (1-(5-carboxypentyl)-2-((1E,3E,5E,7E)-7-(1-ethyl-3-(2,5,8,11,14-pentaoxahexadecan-16-yl)-3-methyl-5-sulfonatoindolin-2-ylidene)hepta-1,3,5-trienyl)-3-methyl-3-(3-sulfopropyl)-3H-indolium-5-sulfonate) contains a (poly)ethylene glycol (PEG₅) at R1, sulfoalkyl at R2, ethyl at R9, and carboxyalkyl at R10.

In one embodiment the compound is 755 Compound 0/1

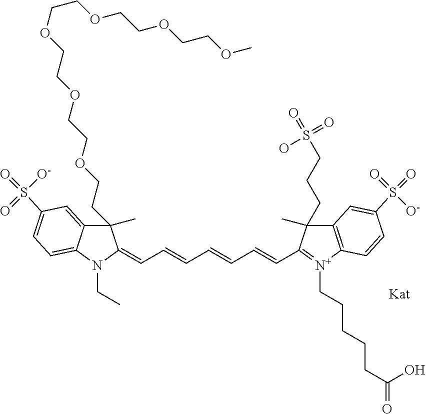 [SMILES: CCN1/C(=C/C=C/C=C/C=C/C2=[N+](CCCCCC(=O)O)c3ccc(S(=O)(=O)[O-])cc3C2(C)CCCS(=O)(=O)O)C(C)(CCOCCOCCOCCOCCOCCOC)c2cc(S(=O)(=O)[O-])ccc21]

755 Compound 0/1 (1-(5-carboxypentyl)-2-((1E,3E,5E,7E)-7-(1-ethyl-3-methyl-3-(2,5,8,11,14,17-hexaoxanonadecan-19-yl)-5-sulfonatoindolin-2-ylidene)hepta-1,3,5-trienyl)-3-methyl-3-(3-sulfopropyl)-3H-indolium-5-sulfonate) contains a (poly)ethylene glycol (PEG₆) at R1, sulfoalkyl at R2, ethyl at R9, and carboxyalkyl at R10.

In one embodiment the compound is 755 Compound 0/2

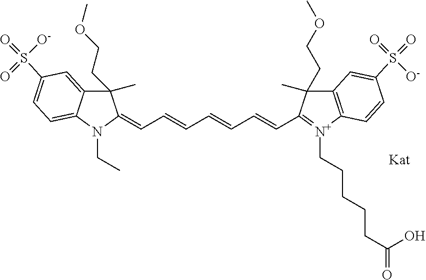 [SMILES: CCN1/C(=C/C=C/C=C/C=C/C2=[N+](CCCCCC(=O)O)c3ccc(S(=O)(=O)[O-])cc3C2(C)CCOC)C(C)(CCOC)c2cc(S(=O)(=O)[O-])ccc21]

755 Compound 0/2 (1-(5-carboxypentyl)-2-((1E,3E,5E,7E)-7-(1-ethyl-3-(2-methoxyethyl)-3-methyl-5-sulfonatoindolin-2-ylidene)hepta-1,3,5-trienyl)-3-(2-methoxyethyl)-3-methyl-3H-indolium-5-sulfonate) contains ethylene glycol at R1 and R2, ethyl at R9, and carboxyalkyl at R10.

One non-limiting example of an activated compound is the NHS-ester of 755 Compound 0/2, shown below:

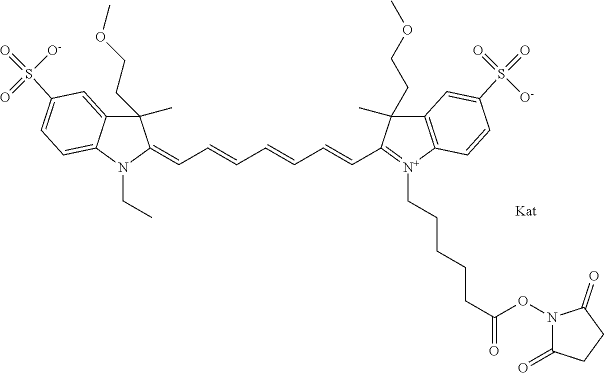 [SMILES: CCN1/C(=C/C=C/C=C/C=C/C2=[N+](CCCCCC(=O)ON3C(=O)CCC3=O)c3ccc(S(=O)(=O)[O-])cc3C2(C)CCOC)C(C)(CCOC)c2cc(S(=O)(=O)[O-])ccc21]

In one embodiment the compound is 755 Compound 0/2

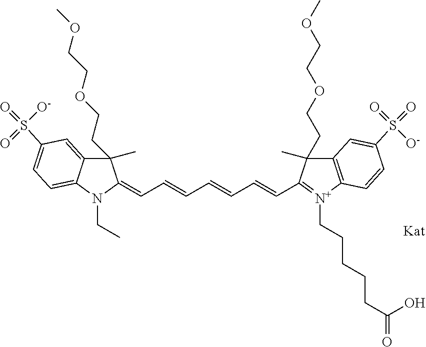 [SMILES: CCN1/C(=C/C=C/C=C/C=C/C2=[N+](CCCCCC(=O)O)c3ccc(S(=O)(=O)[O-])cc3C2(C)CCOCCOC)C(C)(CCOCCOC)c2cc(S(=O)(=O)[O-])ccc21]

755 Compound 0/2 (1-(5-carboxypentyl)-2-((1E,3E,5E,7E)-7-(1-ethyl-3-(2-(2-methoxyethoxy)ethyl)-3-methyl-5-sulfonatoindolin-2-ylidene)hepta-1,3,5-trienyl)-3-(2-(2-methoxyethoxy)ethyl)-3-methyl-3H-indolium-5-sulfonate) contains a diethylene glycol at R1 and R2, ethyl at R9, and carboxyalkyl at R10.

In one embodiment the compound is 755 Compound 0/2

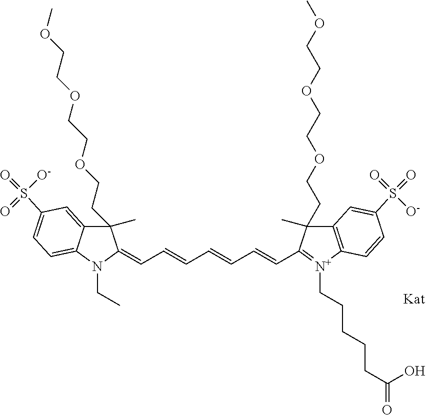 [SMILES: CCN1/C(=C/C=C/C=C/C=C/C2=[N+](CCCCCC(=O)O)c3ccc(S(=O)(=O)[O-])cc3C2(C)CCOCCOCCOC)C(C)(CCOCCOCCOC)c2cc(S(=O)(=O)[O-])ccc21]

755 Compound 0/2 (1-(5-carboxypentyl)-2-((1E,3E,5E,7E)-7-(1-ethyl-3-(2-(2-(2-methoxyethoxy)ethoxy)ethyl)-3-methyl-5-sulfonatoindolin-2-ylidene)hepta-1,3,5-trienyl)-3-(2-(2-(2-methoxyethoxy)ethoxy)ethyl)-3-methyl-3H-indolium-5-sulfonate) contains a (poly)ethylene glycol (PEG₃) at R1 and R2, ethyl at R9, and carboxyalkyl at R10.

In one embodiment the compound is 755 Compound 0/2

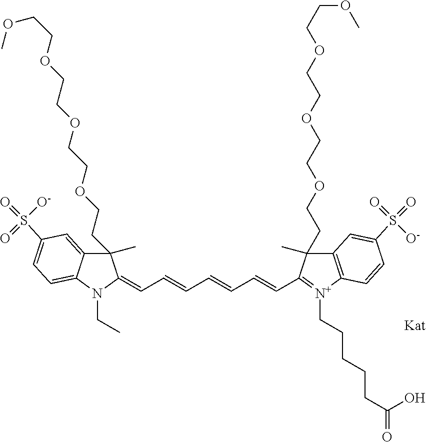 [SMILES: CCN1/C(=C/C=C/C=C/C=C/C2=[N+](CCCCCC(=O)O)c3ccc(S(=O)(=O)[O-])cc3C2(C)CCOCCOCCOCCOC)C(C)(CCOCCOCCOCCOC)c2cc(S(=O)(=O)[O-])ccc21]

755 Compound 0/2 (1-(5-carboxypentyl)-2-((1E,3E,5E,7E)-7-(1-ethyl-3-methyl-5-sulfonato-3-(2,5,8,11-tetraoxatridecan-13-yl)indolin-2-ylidene)hepta-1,3,5-trienyl)-3-methyl-3-(2,5,8,11-tetraoxatridecan-13-yl)-3H-indolium-5-sulfonate) contains a (poly)ethylene glycol (PEG₄) at R1 and R2, ethyl at R9, and carboxyalkyl at R10.

In one embodiment the compound is 755 Compound 0/2

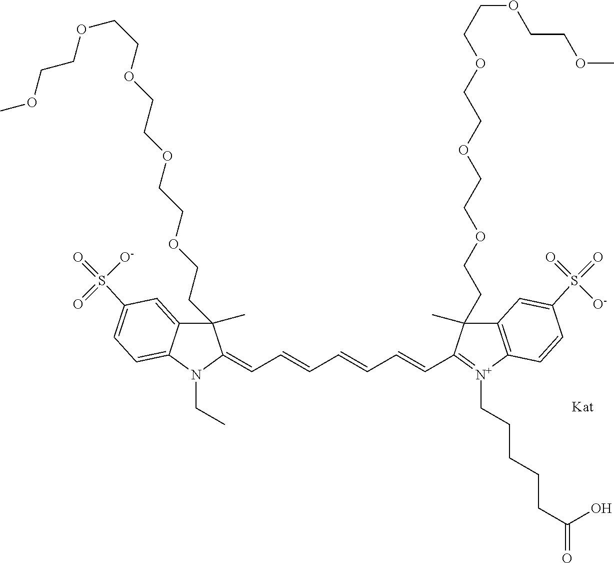

755 Compound 0/2 (1-(5-carboxypentyl)-2-((1E,3E,5E,7E)-7-(1-ethyl-3-(2,5,8,11,14-pentaoxahexadecan-16-yl)-3-methyl-5-sulfonatoindolin-2-ylidene)hepta-1,3,5-trienyl)-3-(2,5,8,11,14-pentaoxahexadecan-16-yl)-3-methyl-3H-indolium-5-sulfonate) contains a (poly)ethylene glycol (PEG₅) at R1 and R2, ethyl at R9, and carboxyalkyl at R10.

In one embodiment the compound is 755 Compound 0/2

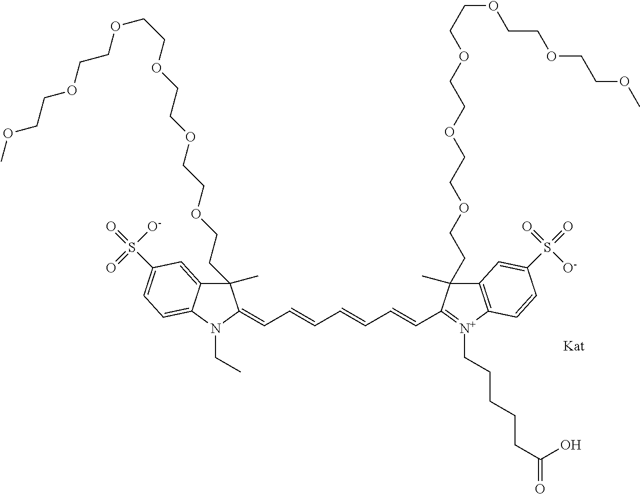

755 Compound 0/2 (1-(5-carboxypentyl)-2-((1E,3E,5E,7E)-7-(1-ethyl-3-methyl-3-(2,5,8,11,14,17-hexaoxanonadecan-19-yl)-3H-indolium-5-sulfonate) contains a (poly)ethylene glycol (PEG₆) at R1 and R2, ethyl at R9, and carboxyalkyl at R10.

In one embodiment the compound is 755 Compound 0/1

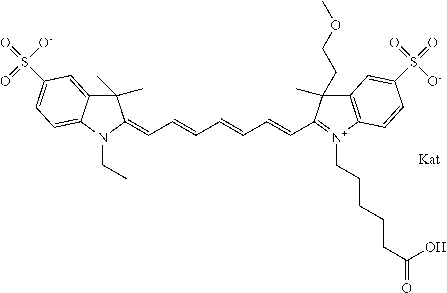 [SMILES: CCN1/C(=C/C=C/C=C/C=C/C2=[N+](CCCCCC(=O)O)c3ccc(S(=O)(=O)[O-])cc3C2(C)CCOC)C(C)(C)c2cc(S(=O)(=O)[O-])ccc21]

755 Compound 0/1 (1-(5-carboxypentyl)-2-((1E,3E,5E,7E)-7-(1-ethyl-3,3-dimethyl-5-sulfonatoindolin-2-ylidene)hepta-1,3,5-trienyl)-3-(2-methoxyethyl)-3-methyl-3H-indolium-5-sulfonate) contains ethylene glycol at R2, methyl at R1, ethyl at R9, and carboxyalkyl at R10.

One non-limiting example of an activated compound is the NHS-ester of 755 Compound 0/1, shown below:

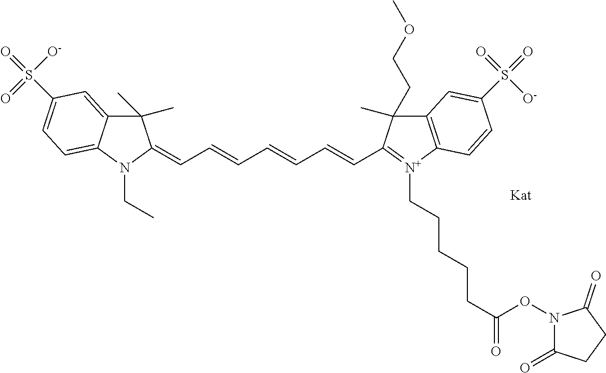 [SMILES: CCN1/C(=C/C=C/C=C/C=C/C2=[N+](CCCCCC(=O)ON3C(=O)CCC3=O)c3ccc(S(=O)(=O)[O-])cc3C2(C)CCOC)C(C)(C)c2cc(S(=O)(=O)[O-])ccc21]

In one embodiment the compound is 755 Compound 0/1

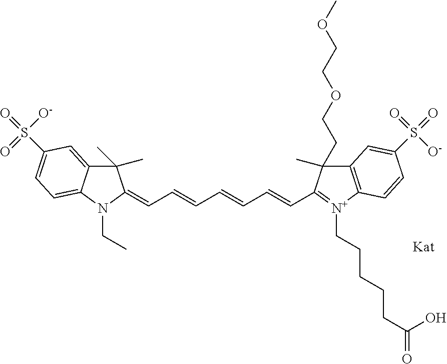 [SMILES: CCN1/C(=C/C=C/C=C/C=C/C2=[N+](CCCCCC(=O)O)c3ccc(S(=O)(=O)[O-])cc3C2(C)CCOCCOC)C(C)(C)c2cc(S(=O)(=O)[O-])ccc21]

755 Compound 0/1 (1-(5-carboxypentyl)-2-((1E,3E,5E,7E)-7-(1-ethyl-3,3-dimethyl-5-sulfonate) contains a diethylene glycol at R2, methyl at R1, ethyl at R9, and carboxyalkyl at R10.

In one embodiment the compound is 755 Compound 0/1

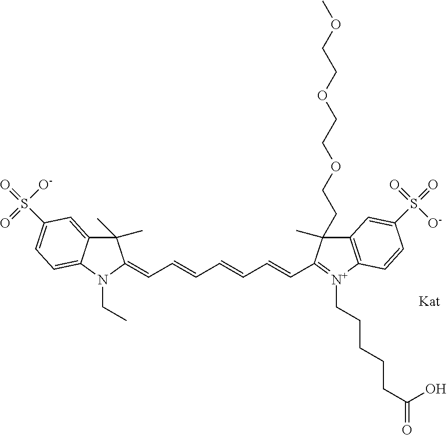 [SMILES: CCN1/C(=C/C=C/C=C/C=C/C2=[N+](CCCCCC(=O)O)c3ccc(S(=O)(=O)[O-])cc3C2(C)CCOCCOCCOC)C(C)(C)c2cc(S(=O)(=O)[O-])ccc21]

755 Compound 0/1 (1-(5-carboxypentyl)-2-((1E,3E,5E,7E)-7-(1-ethyl-3,3-dimethyl-5-sulfonatoindolin-2-ylidene)hepta-1,3,5-trienyl)-3-(2-(2-(2-methoxyethoxy)ethoxy)ethyl)-3-methyl-3H-indolium-5-sulfonate) contains a (poly)ethylene glycol (PEG₃) at R2, methyl at R1, ethyl at R9, and carboxyalkyl at R10.

In one embodiment the compound is 755 Compound 0/1

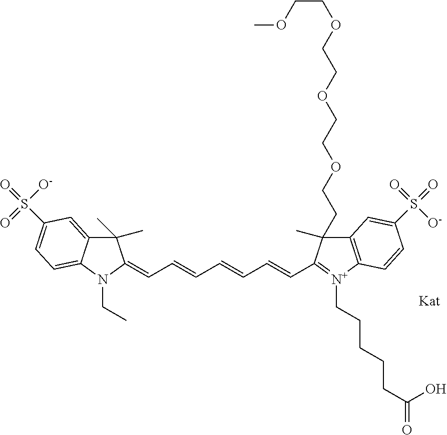 [SMILES: CCN1/C(=C/C=C/C=C/C=C/C2=[N+](CCCCCC(=O)O)c3ccc(S(=O)(=O)[O-])cc3C2(C)CCOCCOCCOCCOC)C(C)(C)c2cc(S(=O)(=O)[O-])ccc21]

755 Compound 0/1 (1-(5-carboxypentyl)-2-((1E,3E,5E,7E)-7-(1-ethyl-3,3-dimethyl-5-sulfonatoindolin-2-ylidene)hepta-1,3,5-trienyl)-3-methyl-3-(2,5,8,11-tetraoxatridecan-13-yl)-3H-indolium-5-sulfonate) contains a (poly)ethylene glycol (PEG₄) at R2, methyl at R1, ethyl at R9, and carboxyalkyl at R10.

In one embodiment the compound is 755 Compound 0/1

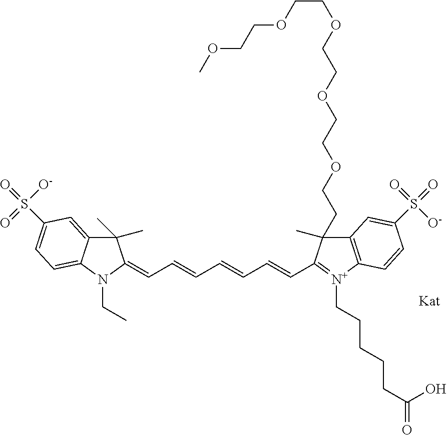 [SMILES: CCN1/C(=C/C=C/C=C/C=C/C2=[N+](CCCCCC(=O)O)c3ccc(S(=O)(=O)[O-])cc3C2(C)CCOCCOCCOCCOCCOC)C(C)(C)c2cc(S(=O)(=O)[O-])ccc21]

755 Compound 0/1 (1-(5-carboxypentyl)-2-((1E,3E,5E,7E)-7-(1-ethyl-3,3-dimethyl-5-sulfonatoindolin-2-ylidene)hepta-1,3,5-trienyl)-3-(2,5,8,11,14-pentaoxahexadecan-16-yl)-3-methyl-3H-indolium-5-sulfonate) contains a (poly)ethylene glycol (PEG₅) at R2, methyl at R1, ethyl at R9, and carboxyalkyl at R10.

In one embodiment the compound is 755 Compound 0/1

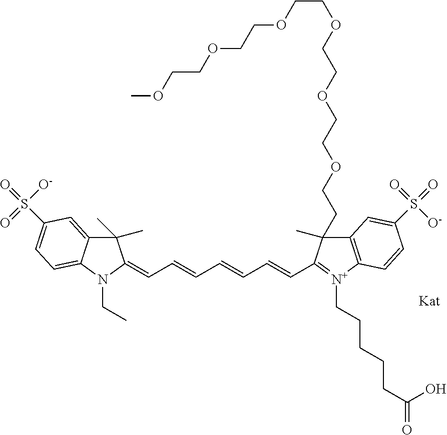 [SMILES: CCN1/C(=C/C=C/C=C/C=C/C2=[N+](CCCCCC(=O)O)c3ccc(S(=O)(=O)[O-])cc3C2(C)CCOCCOCCOCCOCCOCCOC)C(C)(C)c2cc(S(=O)(=O)[O-])ccc21]

755 Compound 0/1 (1-(5-carboxypentyl)-2-((1E,3E,5E,7E)-7-(1-ethyl-3,3-dimethyl-5-sulfonatoindolin-2-ylidene)hepta-1,3,5-trienyl)-3-methyl-3-(2,5,8,11,14,17-hexaoxanonadecan-19-yl)-3H-indolium-5-sulfonate) contains a (poly)ethylene glycol (PEG₆) at R2, methyl at R1, ethyl at R9, and carboxyalkyl at R10.

In one embodiment the compound is 755 Compound 0/1

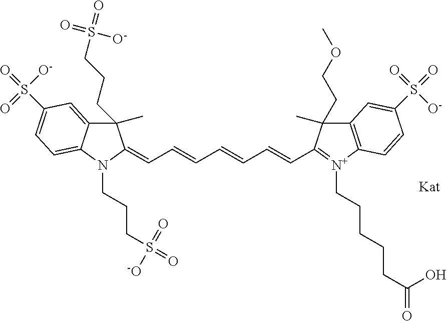

755 Compound 0/1 (3,3′-((E)-2-((2E,4E,6E)-7-(1-(5-carboxypentyl)-3-(2-methoxyethyl)-3-methyl-5-sulfonato-3H-indolium-2-yl)hepta-2,4,6-trienylidene)-3-methyl-5-sulfonatoindoline-1,3-diyl)dipropane-1-sulfonate) contains ethylene glycol at R2, sulfoalkyl at R1, sulfoalkyl at R9, and carboxyalkyl at R10.

One non-limiting example of an activated compound is the NHS-ester of 755 Compound 0/1, shown below:

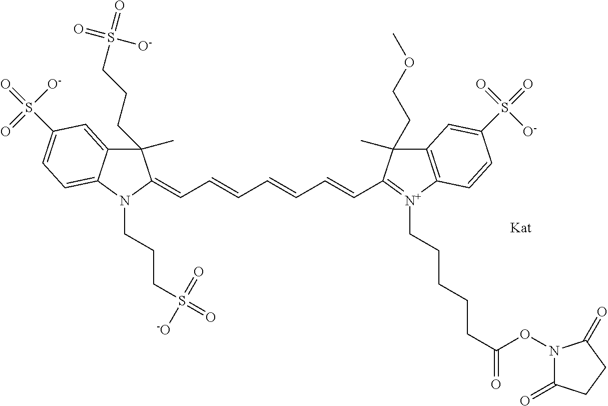

In one embodiment the compound is 755 Compound 0/1

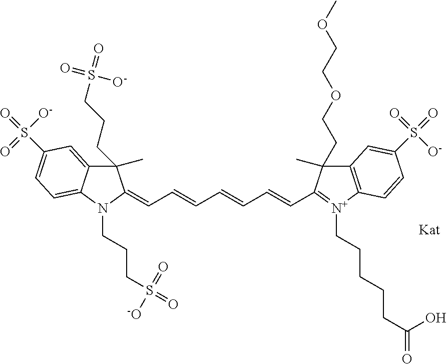

755 Compound 0/1 (3,3′-((E)-2-((2E,4E,6E)-7-(1-(5-carboxypentyl)-3-(2-(2-methoxyethoxy)ethyl)-3-methyl-5-sulfonato-3H-indolium-2-yl)hepta-2,4,6-trienylidene)-3-methyl-5-sulfonatoindoline-1,3-diyl)dipropane-1-sulfonate) contains a diethylene glycol at R2, a sulfoalkyl at R1, a sulfoalkyl at R9, and carboxyalkyl at R10.

In one embodiment the compound is 755 Compound 0/1

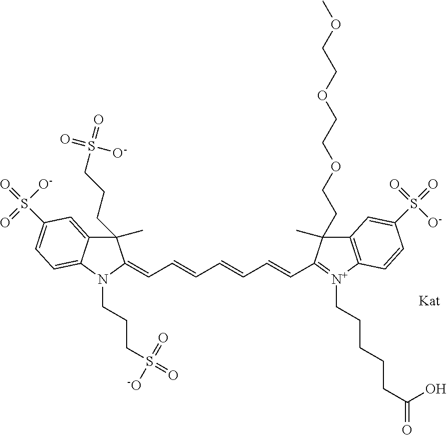

755 Compound 0/1 (3,3′-((E)-2-((2E,4E,6E)-7-(1-(5-carboxypentyl)-3-(2-(2-(2-methoxyethoxy)ethoxy)ethyl)-3-methyl-5-sulfonato-3H-indolium-2-yl)hepta-2,4,6-trienylidene)-3-methyl-5-sulfonatoindoline-1,3-diyl)dipropane-1-sulfonate) contains a (poly)ethylene glycol (PEG₃) at R2, sulfoalkyl at R1, sulfoalkyl at R9, and carboxyalkyl at R10.

In one embodiment the compound is 755 Compound 0/1

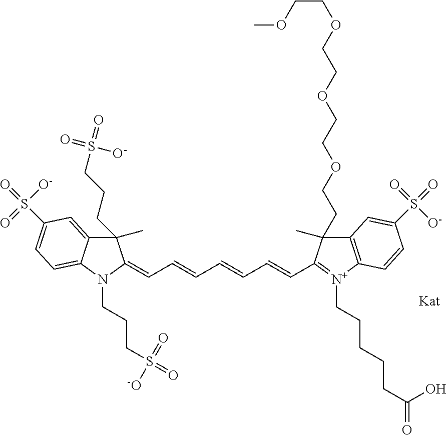

755 Compound 0/1 (3,3′-((E)-2-((2E,4E,6E)-7-(1-(5-carboxypentyl)-3-methyl-5-sulfonato-3-(2,5,8,11-tetraoxatridecan-13-yl)-3H-indolium-2-yl)hepta-2,4,6-trienylidene)-3-methyl-5-sulfonatoindoline-1,3-diyl)dipropane-1-sulfonate) contains a (poly)ethylene glycol (PEG₄) at R2, sulfoalkyl at R1, sulfoalkyl at R9, and carboxyalkyl at R10.

In one embodiment the compound is 755 Compound 0/1

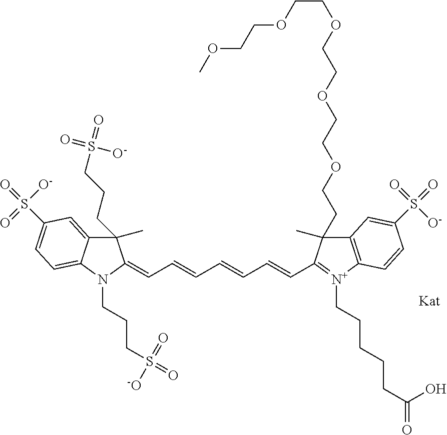

755 Compound 0/1 (3,3′-((E)-2-((2E,4E,6E)-7-(1-(5-carboxypentyl)-3-(2,5,8,11,14-sulfonatoindoline-1,3-diyl)dipropane-1-sulfonate) contains a (poly)ethylene glycol (PEG₅) at R2, sulfoalkyl at R1, ethyl at R9, and carboxyalkyl at R10.

In one embodiment the compound is 755 Compound 0/1

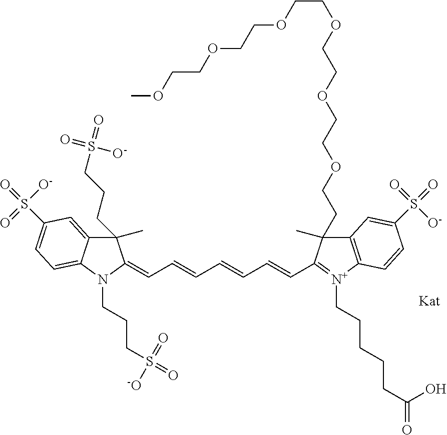

755 Compound 0/1 (3,3′-((E)-2-((2E,4E,6E)-7-(1-(5-carboxypentyl)-3-methyl-3-(2,5,8,11,14,17-sulfonatoindoline-1,3-diyl)dipropane-1-sulfonate) contains a (poly)ethylene glycol (PEG₆) at R2, sulfoalkyl at R1, ethyl at R9, and carboxyalkyl at R10.

In one embodiment the compound is 755 Compound 0/2

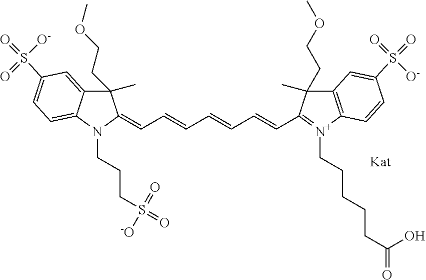

755 Compound 0/2 (1-(5-carboxypentyl)-3-(2-methoxyethyl)-2-((1E,3E,5E,7E)-7-(3-(2-methoxyethyl)-3-methyl-5-sulfonato-1-(3-sulfonatopropyl)indolin-2-ylidene)hepta-1,3,5-trienyl)-3-methyl-3H-indolium-5-sulfonate) contains ethylene glycol at R1 and R2, sulfoalkyl at R9, and carboxyalkyl at R10.

One non-limiting example of an activated compound is the NHS-ester of 755 Compound 0/2, shown below:

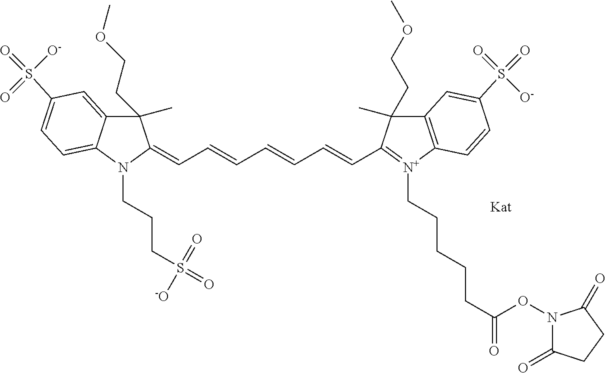

In one embodiment the compound is 755 Compound 0/2

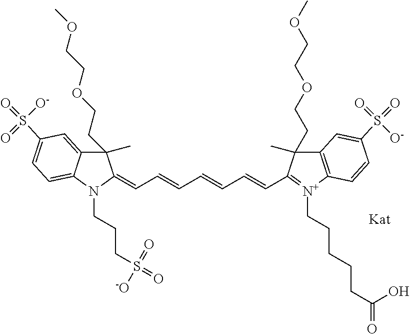

755 Compound 0/2 (1-(5-carboxypentyl)-3-(2-(2-methoxyethoxy)ethyl)-2-((1E,3E,5E,7E)-7-(3-(2-(2-methoxyethoxy)ethyl)-3-methyl-5-sulfonato-1-(3-sulfonatopropyl)indolin-2-ylidene)hepta-1,3,5-trienyl)-3-methyl-3H-indolium-5-sulfonate) contains a diethylene glycol at R1 and R2, sulfoalkyl at R9, and carboxyalkyl at R10.

In one embodiment the compound is 755 Compound 0/2

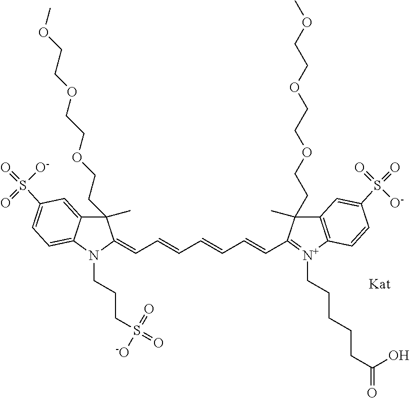

755 Compound 0/2 (1-(5-carboxypentyl)-3-(2-(2-(2-methoxyethoxy)ethoxy)ethyl)-2-((1E,3E,5E,7E)-7-(3-(2-(2-(2-methoxyethoxy)ethoxy)ethyl)-3-methyl-5-sulfonato-1-(3-sulfonatopropyl)indolin-2-ylidene)hepta-1,3,5-trienyl)-3-methyl-3H-indolium-5-sulfonate) contains a (poly)ethylene glycol (PEG₃) at R1 And R2, sulfoalkyl at R9, and carboxyalkyl at R10.

In one embodiment the compound is 755 Compound 0/2

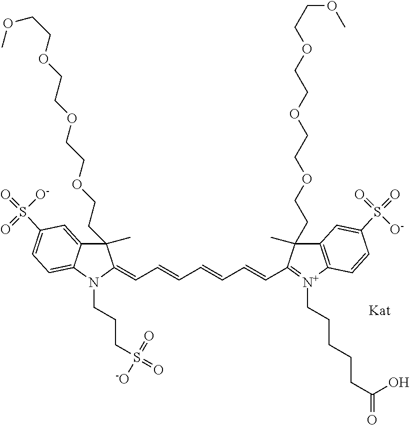

755 Compound 0/2 (1-(5-carboxypentyl)-3-methyl-2-((1E,3E,5E,7E)-7-(3-methyl-5-sulfonato-1-(3-sulfonatopropyl)-3-(2,5,8,11-tetraoxatridecan-13-yl)indolin-2-ylidene)hepta-1,3,5-trienyl)-3-(2,5,8,11-tetraoxatridecan-13-yl)-3H-indolium-5-sulfonate) contains a (poly)ethylene glycol (PEG₄) at R1 and R2, sulfoalkyl at R9, and carboxyalkyl at R10.

In one embodiment the compound is 755 Compound 0/2

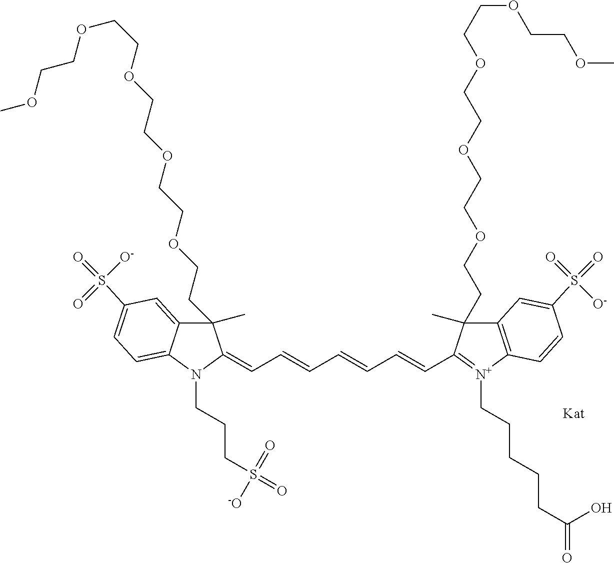

755 Compound 0/2 (2-((1E,3E,5E,7E)-7-(3-(2,5,8,11,14-pentaoxahexadecan-16-yl)-3-methyl-5-sulfonato-1-(3-sulfonatopropyl)indolin-2-ylidene)hepta-1,3,5-trienyl)-1-(5-carboxypentyl)-3-(2,5,8,11,14-pentaoxahexadecan-16-yl)-3-methyl-3H-indolium-5-sulfonate) contains a (poly)ethylene glycol (PEG₅) at R1 and R2, ethyl at R9, and carboxyalkyl at R10.

In one embodiment the compound is 755 Compound 0/2

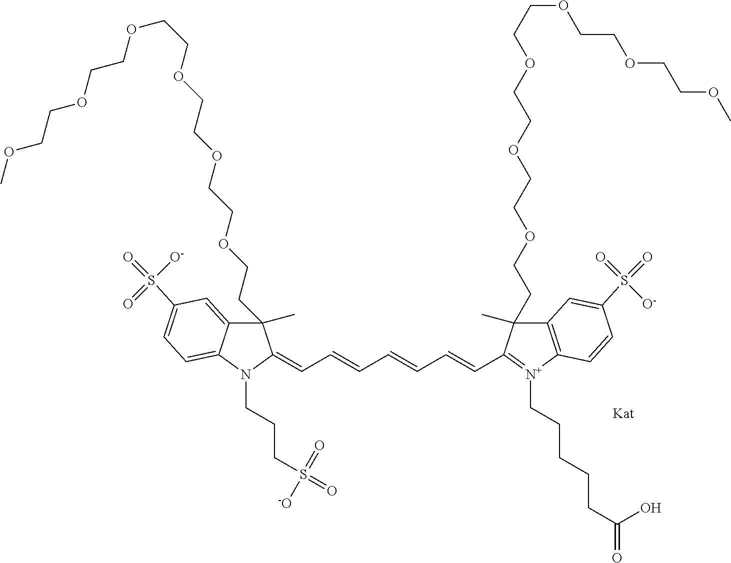

755 Compound 0/2 (1-(5-carboxypentyl)-3-methyl-2-((1E,3E,5E,7E)-7-(3-methyl-3-(2,5,8,11,14,17-hexaoxanonadecan-19-yl)-5-sulfonato-1-(3-sulfonatopropyl)indolin-2-ylidene)hepta-1,3,5-trienyl)-3-(2,5,8,11,14,17-hexaoxanonadecan-19-yl)-3H-indolium-5-sulfonate) contains a (poly)ethylene glycol (PEG₆) at R1 and R2, ethyl at R9, and carboxyalkyl at R10.

In embodiments, the degree of sulfonation is varied to, e.g., vary the compound's degree of hydrophilicity or hydrophobicity. One non-limiting example is a monosulfonate form of 755 Compound 1/2, shown below, but it is understood that the single sulfo group can be at any of the described positions:

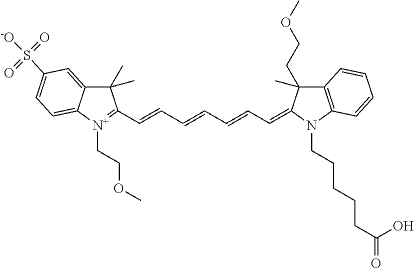

One non-limiting example is a disulfonate form of 755 Compound 1/2, shown below, but it is understood that the each of the two sulfo groups can be at any of the described positions:

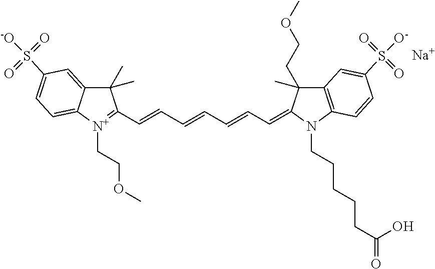

One non-limiting example is a trisulfonate form of 755 Compound 1/2, shown below, but it is understood that the each of the three sulfo groups can be at any of the described positions:

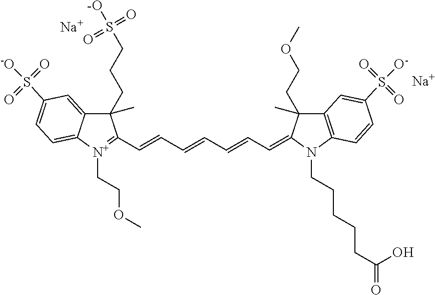

One non-limiting example is a tetrasulfonate form of 755 Compound 1, shown below, but it is understood that the each of the four sulfo groups can be at any of the described positions:

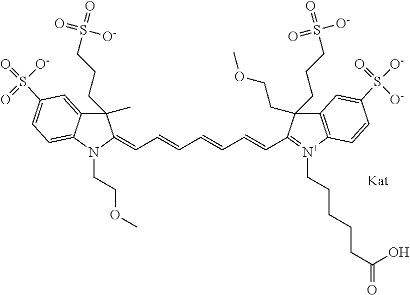

One non-limiting example is a monosulfonate form of 755 Compound 0/1, shown below, but it is understood that the single sulfo group can be at any of the described positions:

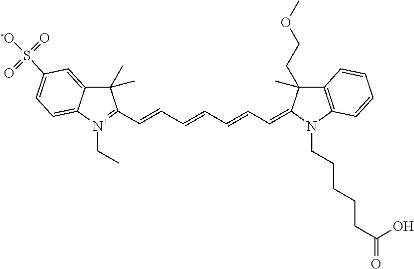

One non-limiting example is a disulfonate form of 755 Compound 0/1, shown below, but it is understood that the each of the two sulfo groups can be at any of the described positions:

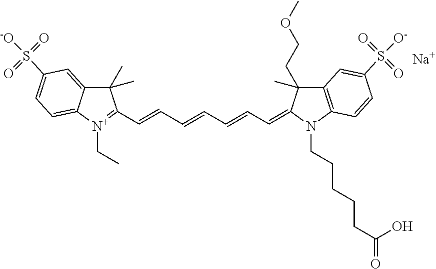

One non-limiting example is a trisulfonate form of 755 Compound 0/1, shown below, but it is understood that the each of the two sulfo groups can be at any of the described positions:

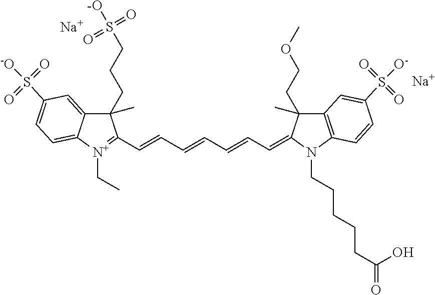

One non-limiting example is a tetrasulfonate form of 755 Compound 0/1, shown below, but it is understood that the each of the four sulfo groups can be at any of the described positions:

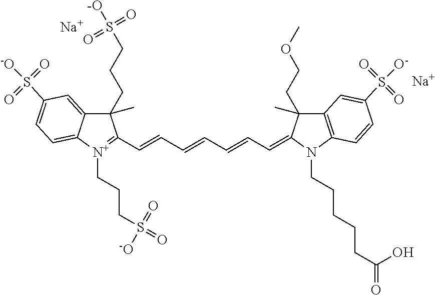

In various embodiments, an ethylene glycol group, diethylene glycol group, and/or a (poly)ethylene glycol group, which will collectively be referred to as a PEG group, unless specifically defined, may be present at position(s) in addition to presence on the N atom(s) of the indole.

One non-limiting example of an additionally PEG-substituted compound is a 755 Compound 1/2 according to general formula II where R1 is an ethylene glycol group terminating with a methyl group, shown below:

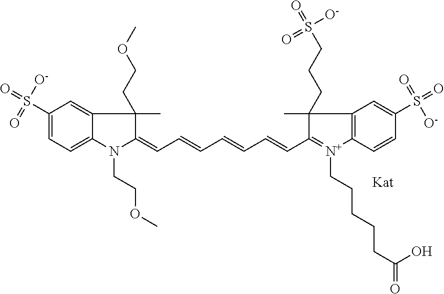

One non-limiting example of an additionally PEG-substituted compound is a 755 Compound 1/2 according to general formula II where R1 is a diethylene glycol group terminating with a methyl group, shown below:

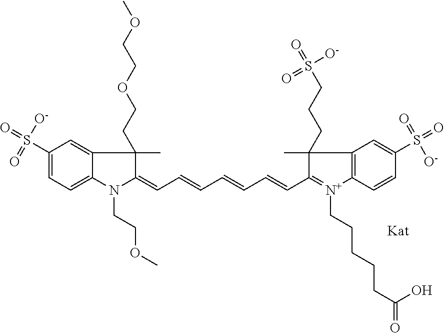

One non-limiting example of an additionally PEG-substituted compound is a 755 Compound 1/2 according to general formula II where R1 is a (poly)ethylene glycol (3) group terminating with a methyl group, shown below:

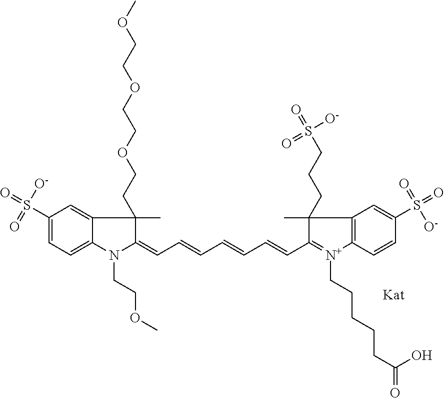

One non-limiting example of an additionally PEG-substituted compound is a 755 Compound 1/2 according to general formula II where R1 is a (poly)ethylene glycol (4) group terminating with a methyl group, shown below:

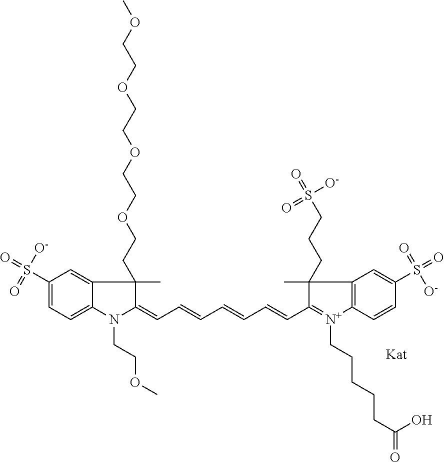

One non-limiting example of an additionally PEG-substituted compound is a 755 Compound 1/2 according to general formula II where R1 is a (poly)ethylene glycol (5) group terminating with a methyl group, shown below:

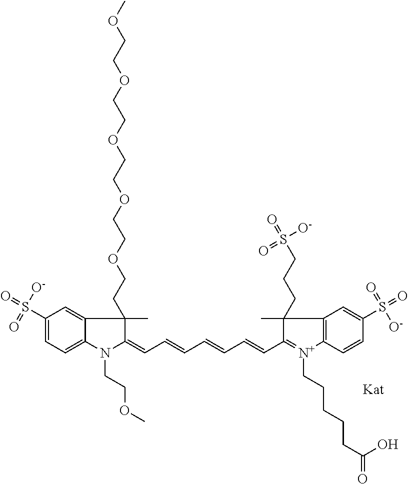

One non-limiting example of an additionally PEG-substituted compound is a 755 Compound 1/2 according to general formula II where R1 is a (poly)ethylene glycol (6) group terminating with a methyl group, shown below:

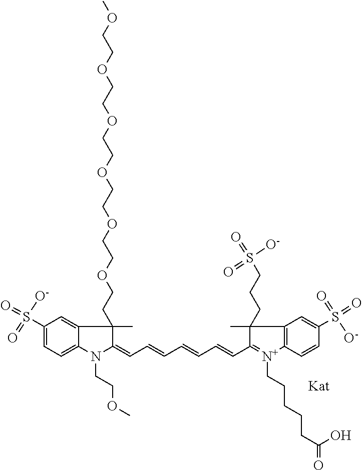

One non-limiting example of an additionally PEG-substituted compound is a 755 Compound 1/2 according to general formula II where R1 is a sulfonamide group -L-SO₂NH—P—Z where Z is a methyl group, shown below:

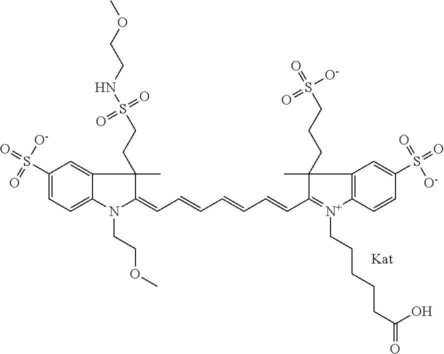

One non-limiting example of an additionally PEG-substituted compound is a 755 Compound 1/2 according to general formula II where R1 is a carboxamide group -L-CONH—P—Z where Z is a methyl group, shown below:

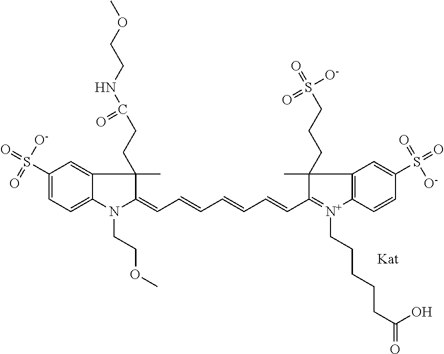

One non-limiting example of an additionally PEG-substituted compound is a 755 Compound 1/2 according to general formula II where R2 is an ethylene glycol group terminating with a methyl group, shown below:

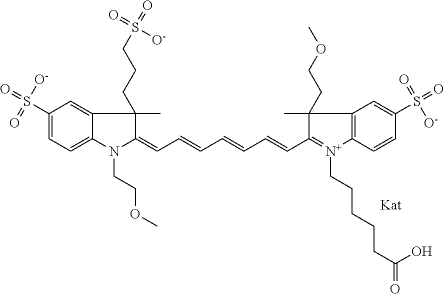

One non-limiting example of an additionally PEG-substituted compound is a 755 Compound 1/2 according to general formula II where R2 is a diethylene glycol group terminating with a methyl group, shown below:

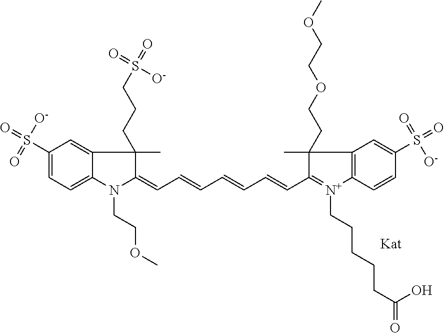

One non-limiting example of an additionally PEG-substituted compound is a 755 Compound 1/2 according to general formula II where R2 is a (poly)ethylene glycol (3) group terminating with a methyl group, shown below:

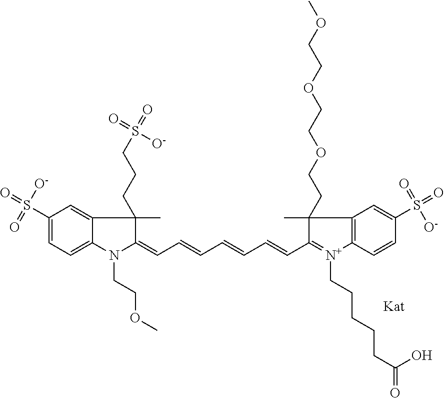

One non-limiting example of an additionally PEG-substituted compound is a 755 Compound 1/2 according to general formula II where R2 is a (poly)ethylene glycol (4) group terminating with a methyl group, shown below:

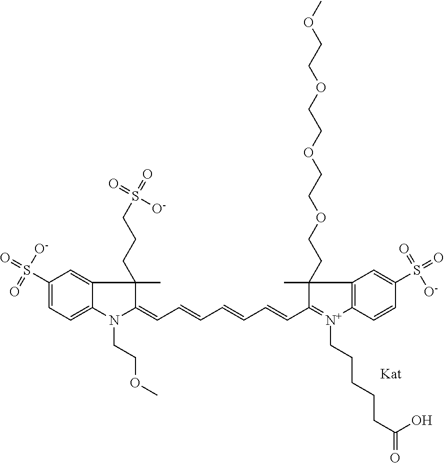

One non-limiting example of an additionally PEG-substituted compound is a 755 Compound 1/2 according to general formula II where R2 is a (poly)ethylene glycol (5) group terminating with a methyl group, shown below:

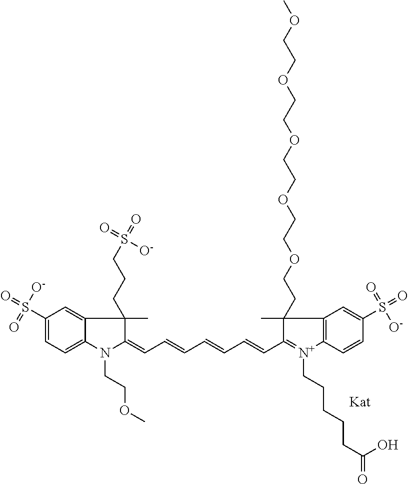

One non-limiting example of an additionally PEG-substituted compound is a 755 Compound 1/2 according to general formula II where R2 is a (poly)ethylene glycol (6) group terminating with a methyl group, shown below:

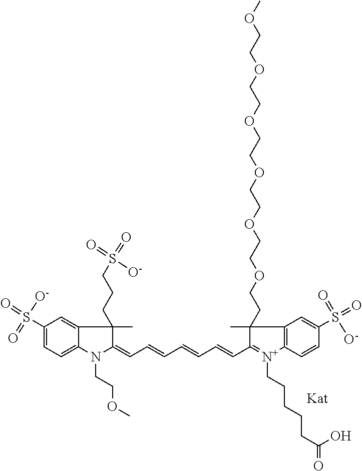

One non-limiting example of an additionally PEG-substituted compound is a 755 Compound 1/2 according to general formula II where R2 is a sulfonamide group -L-SO₂NH—P—Z where Z is a methyl group, shown below:

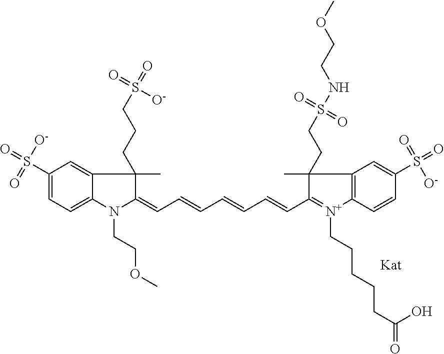

One non-limiting example of an additionally PEG-substituted compound is a 755 Compound 1/2 according to general formula II where R2 is a carboxamide group -L-CONH—P—Z where Z is a methyl group, shown below:

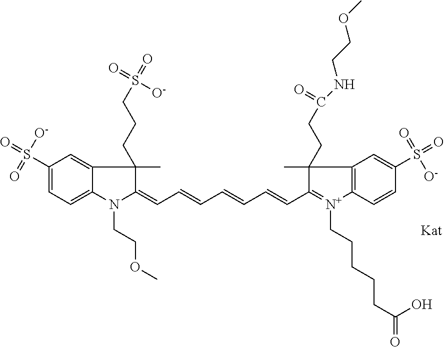

One non-limiting example of an additionally PEG-substituted compound is a 755 Compound 1/3 according to general formula II where both R1 and R2 are an ethylene glycol group terminating with a methyl group, shown below:

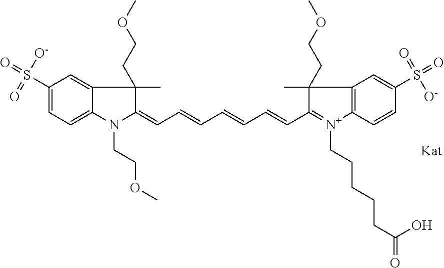

One non-limiting example of an additionally PEG-substituted compound is a 755 Compound 1/3 according to general formula II where both R1 and R2 are a diethylene glycol group terminating with a methyl group, shown below:

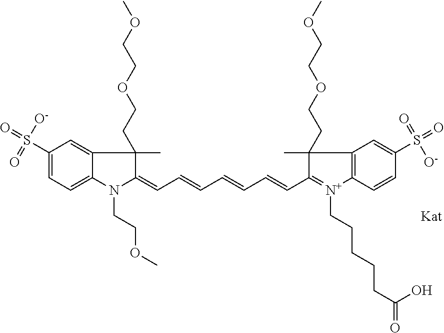

One non-limiting example of an additionally PEG-substituted compound is a 755 Compound 1/3 according to general formula II where both R1 and R2 are a (poly)ethylene glycol (3) group terminating with a methyl group, shown below:

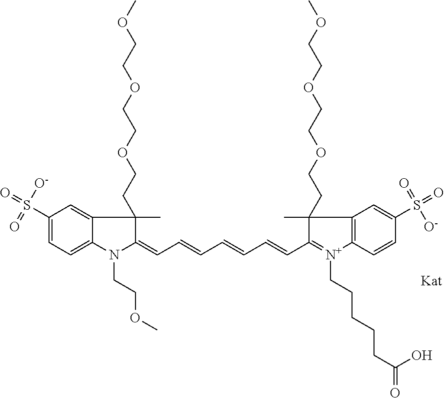

One non-limiting example of an additionally PEG-substituted compound is a 755 Compound 1/3 according to general formula II where both R1 and R2 are a (poly)ethylene glycol (4) group terminating with a methyl group, shown below:

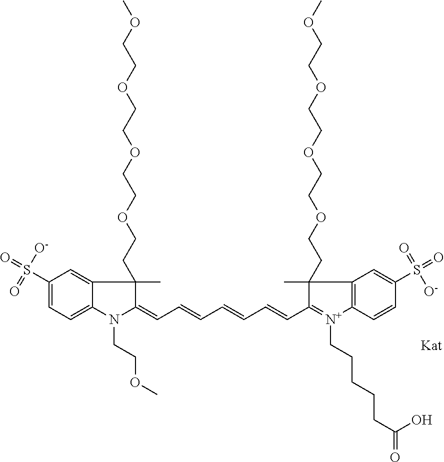

One non-limiting example of an additionally PEG-substituted compound is a 755 Compound 4/4 according to general formula II where both R1 and R2 are a (poly)ethylene glycol (4) group terminating with a methyl group, and R7 and R8 are sulfo, shown below (V17-03136):

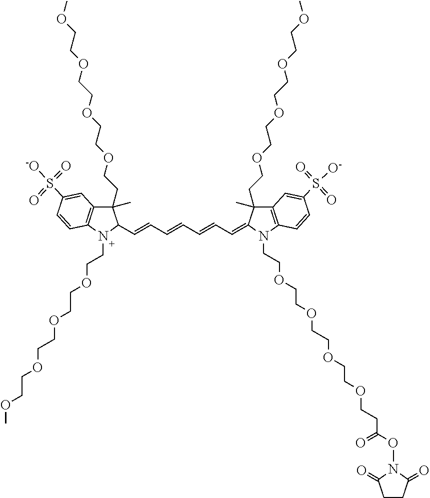

One non-limiting example of an additionally PEG-substituted compound is a 755 Compound 4/4 according to general formula II where both R1 and R2 are a (poly)ethylene glycol (4) group terminating with a methyl group, and R7 and R8 are H, shown below:

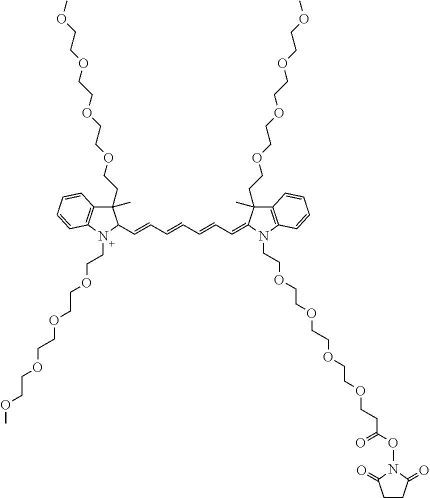

One non-limiting example of an additionally PEG-substituted compound is a 755 Compound 1/3 according to general formula II where both R1 and R2 are a (poly)ethylene glycol (5) group terminating with a methyl group, shown below:

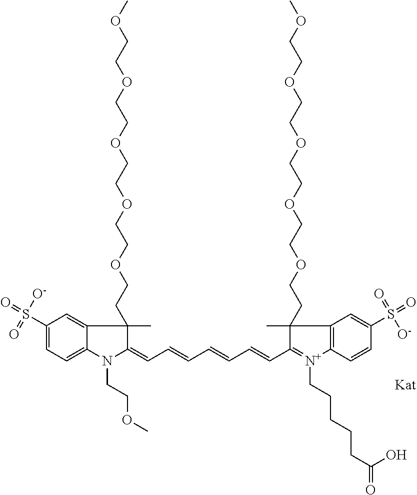

One non-limiting example of an additionally PEG-substituted compound is a 755 Compound 1/3 according to general formula II where both R1 and R2 are a (poly)ethylene glycol (6) group terminating with a methyl group, shown below:

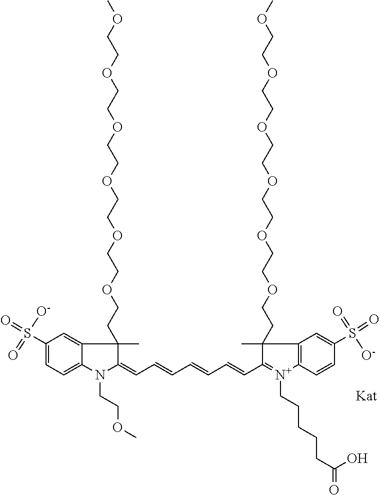

One non-limiting example of an additionally PEG-substituted compound is a 755 Compound 1/3 according to general formula II where both R1 and R2 are a sulfonamide group -L-SO₂NH—P—Z where Z is a methyl group, shown below:

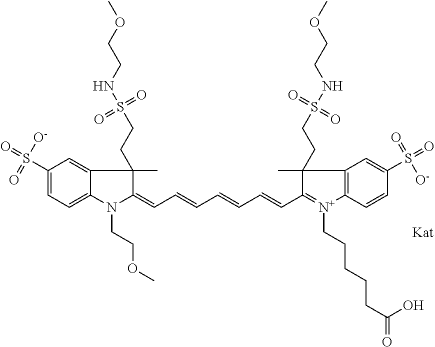

One non-limiting example of an additionally PEG-substituted compound is a 755 Compound 1/3 according to general formula II where both R1 and R2 are a carboxamide group -L-CONH—P—Z where Z is a methyl group, shown below:

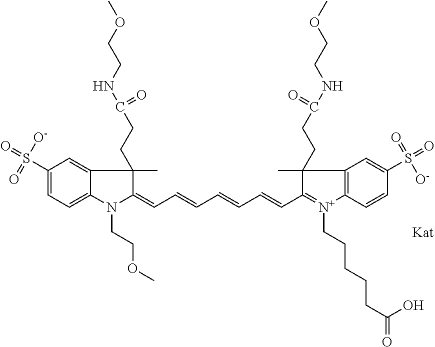

One non-limiting example of an additionally PEG-substituted compound is a 755 Compound 1/2 according to general formula II where R8 is an ethylene glycol group terminating with a methyl group, shown below:

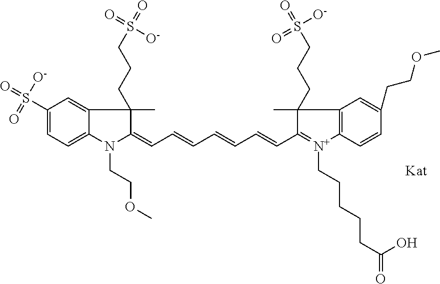

One non-limiting example of an additionally PEG-substituted compound is a 755 Compound 1/2 according to general formula II where R8 is a sulfonamide group —SO₂NH—P—Z where Z is a methyl group, shown below:

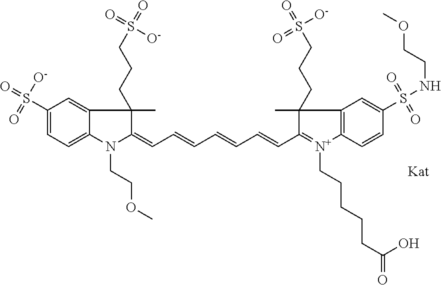

One non-limiting example of an additionally PEG-substituted compound is a 755 Compound 1/2 according to general formula II where R8 is a carboxamide group —CONH—P—Z where Z is a methyl group, shown below:

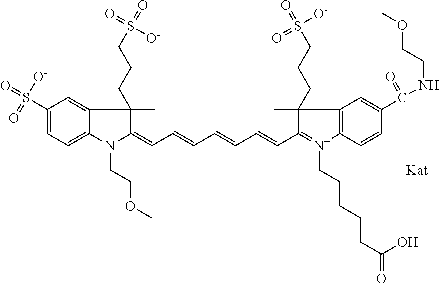

One non-limiting example of an additionally PEG-substituted compound is a 755 Compound 1/2 according to general formula II where R7 is an ethylene glycol group terminating with a methyl group, shown below:

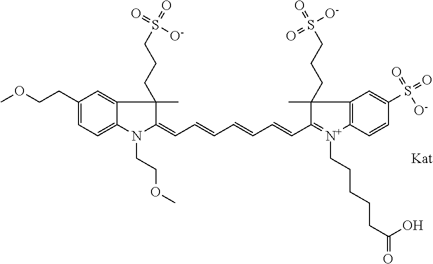

One non-limiting example of an additionally PEG-substituted compound is a 755 Compound 1/2 according to general formula II where R7 is a sulfonamide group —SO₂NH—P—Z where Z is a methyl group, shown below:

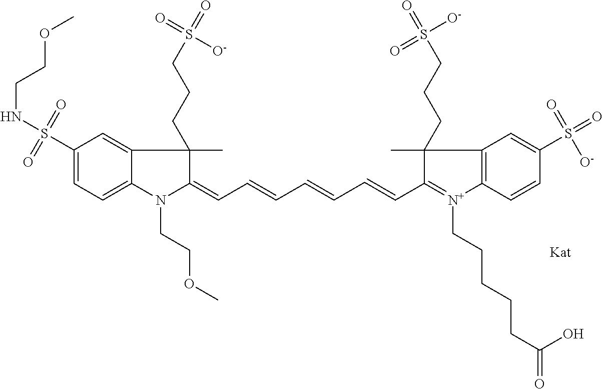

One non-limiting example of an additionally PEG-substituted compound is a 755 Compound 1/2 according to general formula II where R7 is a carboxamide group —CONH—P—Z where Z is a methyl group, shown below:

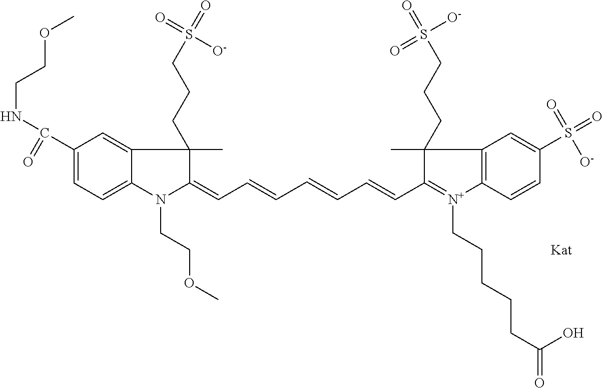

One non-limiting example of an additionally PEG-substituted compound is a 755 Compound 1/3 according to general formula II where both R7 and R8 are an ethylene glycol group terminating with a methyl group, shown below:

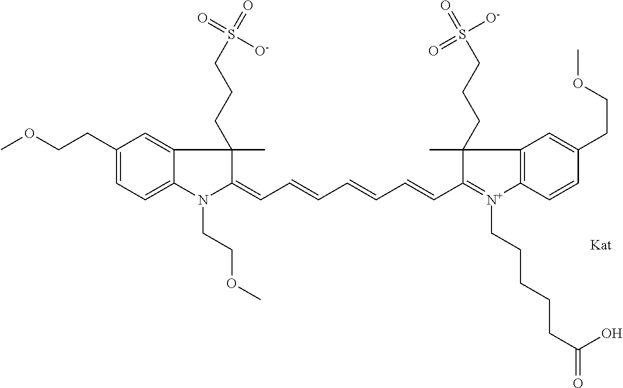

One non-limiting example of an additionally PEG-substituted compound is a 755 Compound 1/3 according to general formula II where both R7 and R8 are a sulfonamide group —SO₂NH—P—Z where Z is a methyl group, shown below:

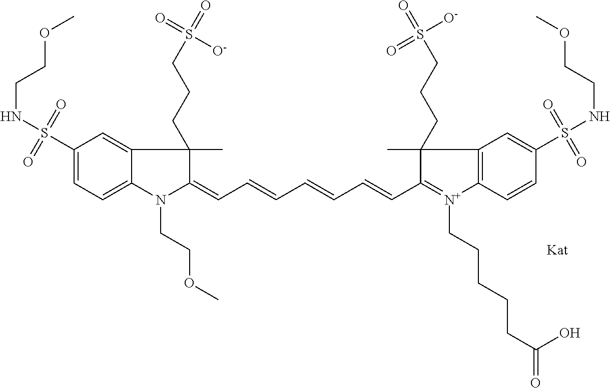

One non-limiting example of an additionally PEG-substituted compound is a 755 Compound 1/3 according to general formula II where both R7 and R8 are a carboxamide group —CONH—P—Z where Z is a methyl group, shown below:

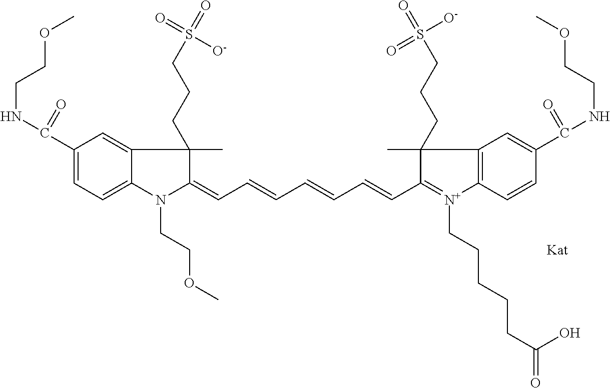

The disclosed compounds are used and are useful as chromophores and/or fluorophores. For example, they can be used for optical labelling and, therefore, for the qualitative and/or quantitative detection of proteins, nucleic acids, oligomers, DNA, RNA, biological cells, lipids, mono-, oligo- and polysaccharides, ligands, receptors, polymers, drugs, polymeric beads, etc.

The inventive compounds, containing the disclosed functionality or functionalities, may be synthesized using methods known in the art, e.g., as described as follows with all references expressly incorporated by reference herein in their entirety. The hydrophilicity or hydrophobicity of the inventive compounds is modified by the number and location of hydrophilic groups such as sulfo, carboxy, hydroxy, etc. In embodiments, the number and location of hydrophilic groups is symmetrical, such that the number and location of hydrophilic group(s) on one indole of the inventive cyanine compound is also found on the other indole. In embodiments, at least one hydrophilic group is on each indole of the inventive compound. Similarly, solubility, lack of aggregation, reactivity, lack of cross-reactivity, etc., are affected by the number and location of the disclosed functionality or functionalities on the compound.

In one embodiment short PEG groups are added on opposite sides and opposite ends of indole compounds to effectively surround the hydrophobic core structure of the molecule. In another embodiment sulfo groups are added to the outer phenyl rings of indole dyes along with the symmetrical placement of short PEG chains on opposite sides and opposite ends.

Even short PEG chain modifications, if at the appropriate positions to strategically surround the core dye structure, can have significant beneficial effects on the hydrophilicity and performance of these dyes in biological applications. Previous attempts to make dyes more hydrophilic and less “sticky” toward biomolecules included the addition of multiple sulfonates or much longer PEG chains to some locations on dye molecules. However, addition of too many sulfonates, while increasing the relative water solubility of dyes, can create undesirable nonspecific binding character due to negative charge interactions with positively charged biomolecules, particularly proteins. Previous attempts to make dyes more water soluble by adding long PEG chains to one or two sites on a dye has the detrimental effect of dramatically increasing the molecular weight of the dye, possibly preventing efficient access of dye-labeled antibodies and other dye-labeled targeting molecules to bind with inner cellular targets, while also not fully surrounding and masking the hydrophobic dye core structure. The inventors have discovered that by using short PEG chain modifications at critical sites on a dye structure that the total molecular size of labeled molecules can be limited, while nonspecificity is dramatically reduced by masking the hydrophobic dye core.

In one embodiment, short PEG groups are added on opposite sides and opposite ends of indole cyanine compounds to effectively surround the hydrophobic core structure of the molecule. In another embodiment, sulfo groups are added to the outer phenyl rings of indole cyanine dyes along with the symmetrical placement of short PEG chains on opposite sides and opposite ends.

The core indocyanine structure without additional functionalilities, along with its synthesis, was described by König in U.S. Pat. No. 1,524,791 and BP 434875, and included 3-, 5-, and 7-membered polymethine chains.

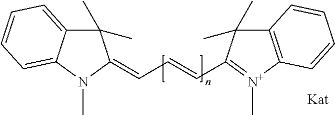

Synthesis of numerous modifications of the core indocyanine structure have been described. Such modifications provided various functionalilities, e.g., synthesis of N-isothiocyanato-alkyl- and aromatic-carboxyalkyl-functionalized indocyanines were described in U.S. Pat. Nos. 5,627,027; 6,048,982; 4,981,977; U.S. Publication No. 2006/0199949; Southwick, Anal. Chem. 67 (1995)1742-48).

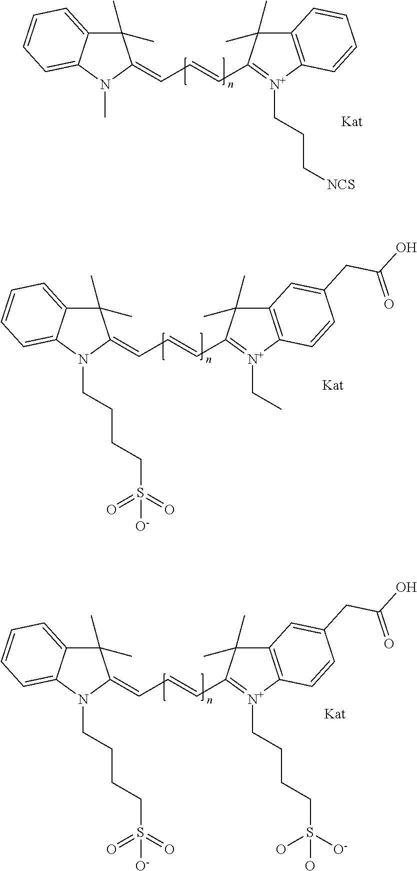

Synthesis of indocyanines with one or two N-carboxyalkyl functionalities were described in U.S. Pat. Nos. 5,268,486; 5,486,616; 5,569,587; 5,569,766; JP 03217837.

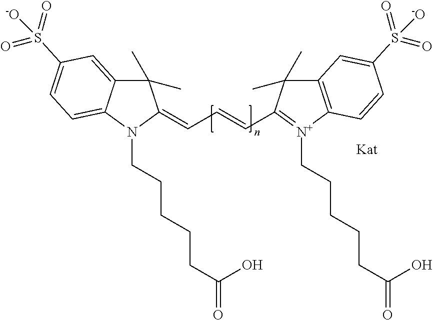

Synthesis of indocyanines containing C-carboxyalkyl were described in JP 05-313304; U.S. Publication Nos. 2006/0099638, 2006/0004188; 2002/0077487; 2002/0064794; U.S. Pat. Nos. 6,977,305 and 6,974,873.

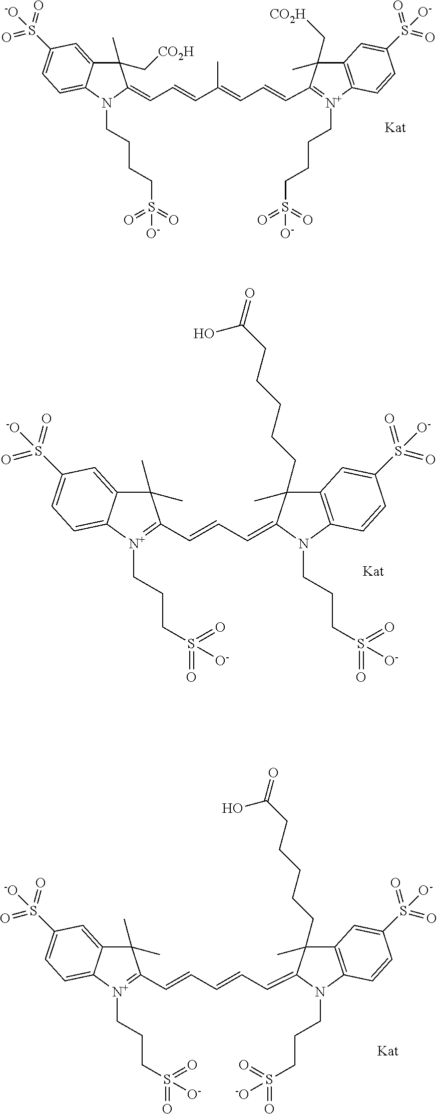

Synthesis of indocyanines with N- and C-sulfoalkyl were described in JP 05-313304; WO 2005/044923; U.S. Publication No. 2007/0203343.

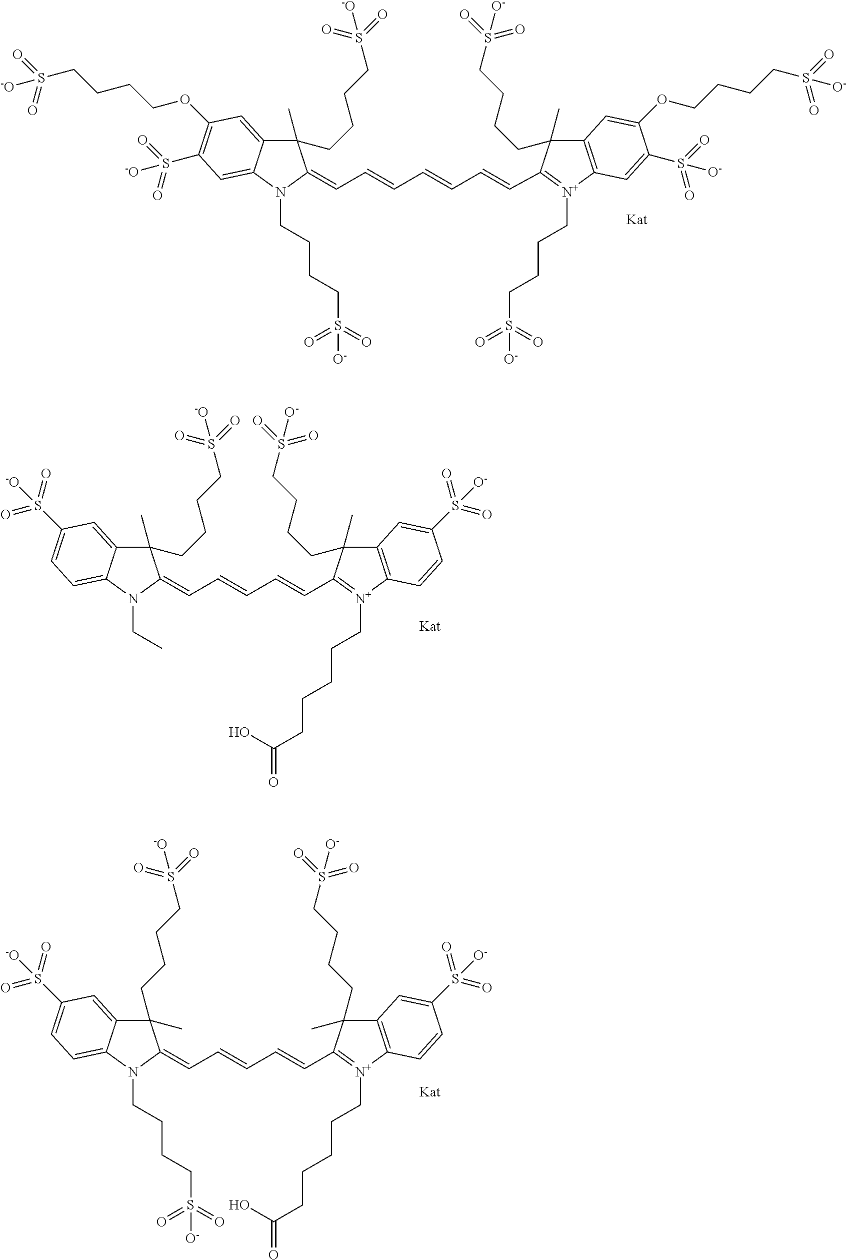

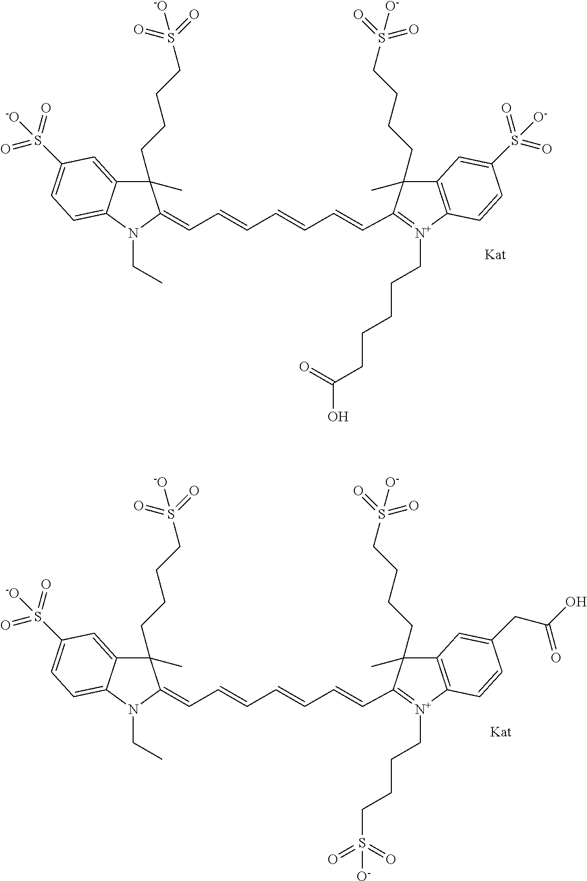

Synthesis of indocyanines with mixed C-carboxyalkyl and C-sulfoalkyl were described in EP 1792949 and U.S. Pat. No. 7,745,640.

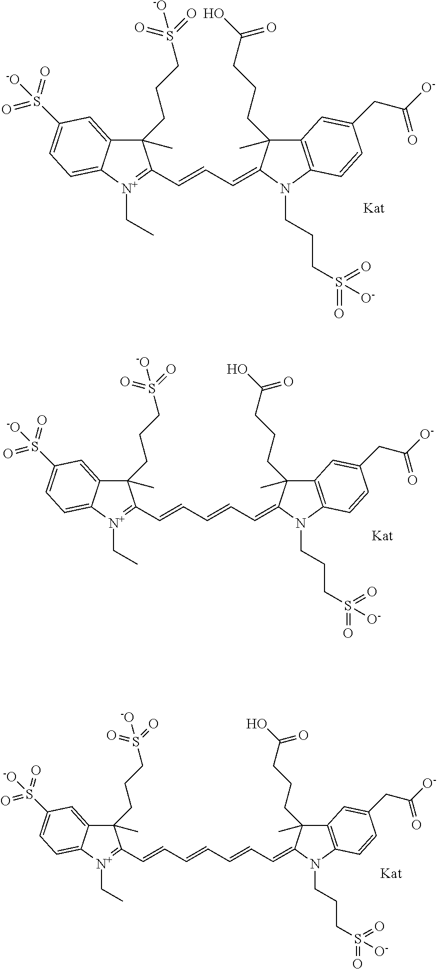

Synthesis of indocyanines having a PEG-containing, N-carboxyalkyl spacer were described in U.S. Pat. No. 6,939,532.

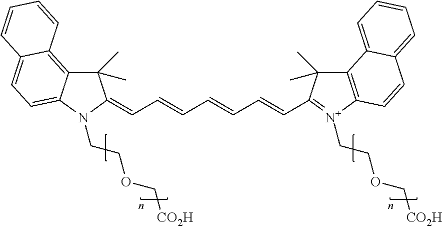

Functionalization of the N-carboxyalkyl with an amino-functionalized PEG-alkyl chain, and N- and C-substituted PEG-alkyl chains, were described in U.S. Publication No. 2009/0305410.

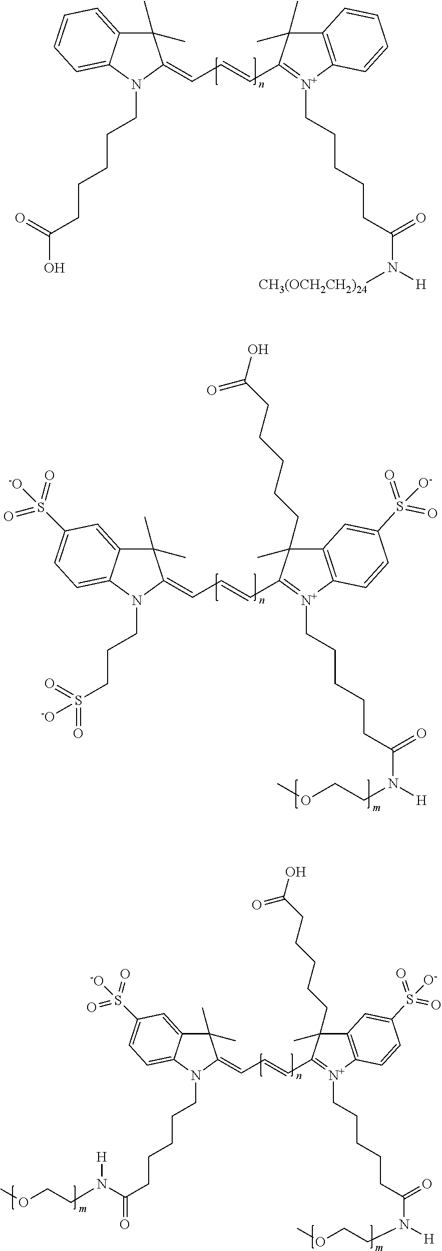

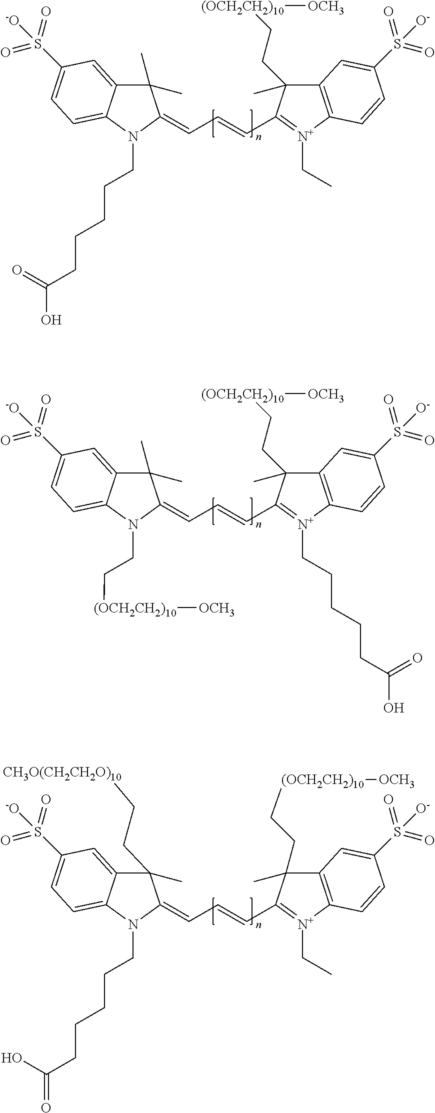

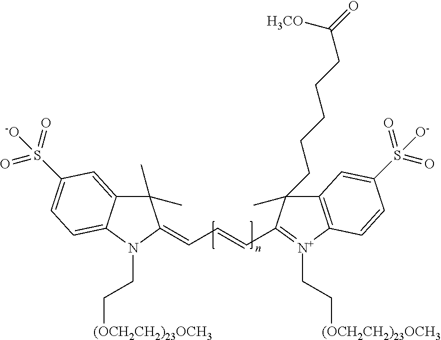

Synthesis of various polymethine bridge substitutions, and other functionalizations of indocyanines, were described in Strekowski, Heterocyclic Polymethine Dyes: Synthesis, Properties and Applications, (2008) Springer-Verlag, Berlin Heidelberg; Gragg, “Synthesis of Near-Infrared Heptamethine Cyanine Dyes” (2010). Chemistry Theses. Paper 28. http://digitalarchive.gsu.edu/chemistry_theses/28; Patonay et al. (2004) Noncovalent Labeling of Biomolecules with Red and Near-Infrared Dyes, Molecules 9 (2004) 40-49; and U.S. Pat. No. 7,172,907.

In one embodiment the compound is synthesized by a condensation reaction, known to one skilled in the art, of the two differently substituted indole heterocycles separated by a (poly)methine linker or bridge, e.g., C1, C3, or C5. Other synthesis methods are possible. As only one example, one of the indole heterocycles is first reacted with the C1, C3, or C5 linker. The 1:1 condensation product is isolated, and then condensed with the second indole heterocycle to result in the cyanine compound. The sequence of reacting the indole heterocycles is irrelevant. Thus, a plurality of differently functionalized, strongly hydrophilic, diastereomeric compounds that differ in total charge and specificity/reactivity of the active groups used for their immobilization, were prepared.

Conjugates of the compounds were prepared by covalently coupling the compounds to a biomolecule using the functional substituent on the N-position of the indole ring. This functional substituent was activated by routine protein chemistry reaction methods known to one skilled in the art. The activated compound may be converted to, e.g., and without limitation, aN-hydroxysuccinimide (NHS)-ester, an acid fluoride, a tetrafluorophenyl (TFP)- or sulfotetrafluorophenyl (STP)-ester, an iodoacetyl group, a maleimide, a hydrazide, a sulfonyl chloride, or a phenylazide. Methods for preparing such compounds are known to one skilled in the art. In one embodiment the activated substituent was then reacted with an amino group on the biomolecule under conditions to conjugate the desired biomolecule.

In one embodiment a non-activated carboxyl group on the N-position of the indole in the compound was coupled to an amine using a carbodimide.

In one embodiment a N-hydroxysuccinimidyl ester (X=—NHS) of a compound was formed as follows: 20 μmol dye with X=OH (carboxyalkyl group), 8 mg (40 μmol) dicyclohexylcarbodiimide, and 5 mg (40 μmol) N-hydroxysuccinimide were dissolved in 2 ml DMF and 100 μl water. Six μl (40 μmol) triethylamine was added. The reaction mixture was stirred at room temperature (about 20° C. to about 22° C.) for 24 hours and then filtered. The solvent was removed and the residue was washed with diethylether. The reaction proceeded quantitatively.

In one embodiment a maleimide (X=—NH—CH₂CH₂-maleimide) of a compound is formed as follows: 20 μmol dye with X=—NHS (N-hydroxysuccinimid-ester) was dissolved in 2 ml DMF and 100 μl water and mixed with 7.6 mg (30 μmol) 2-maleimidoethylamine-trifluoroacetate and 5 μl (30 μmol) N-ethyldiisopropyl-amine. The reaction mixture is stirred for 3 h at room temperature (about 20° C. to about 22° C.). The solvent was evaporated under reduced pressure. The residue is washed with diethylether and acetone and dried in vacuum. The reaction proceeds quantitatively.

In one embodiment a iodoacetamide (X=—NH—CH₂CH₂—NH—CO—CH₂—I) of a compound is formed as follows: 20 μmol dye with X=—NHS (N-hydroxysuccinimid-ester) was dissolved in 2 ml DMF and 100 μl water, followed by addition of 40 mg (300 μmol) ethylendiamindihydrochloride and 26 μl (150 μmol) N-ethyldiisopropyl-amine. The reaction mixture is stirred for 3 h at room temperature (about 20° C. to about 22° C.). The solvent is then evaporated under reduced pressure, the residue was dissolved in methanol, and the ethylendiamindihydrochlorid was removed by filtration. The methanol is evaporated under reduced pressure. The residue is dissolved in 2 ml dry DMF, followed by addition of 7 mg (25 μmol) N-succinimidyl iodoacetate and 4 μl (25 μmol) N-ethyldiisopropylamine. The reaction mixture is stirred for 3 h at room temperature. The solvent was evaporated under reduced pressure and the residue was purified by reverse phase HPLC.

In one embodiment a hydroxyl group, such as a terminal hydroxyl group, can be subsequently activated to a reactive derivative able to link with, for example, proteins and other molecules. Examples of activating groups include tosyl chloride (TsCl), tresyl chloride (TrCl), disuccinimidyl carbonate (DSC), divinyl sulfone, bis-epoxy compounds, carbonyl diimidazole (CDI), 2-fluoro-1-methylpyridinium (FMP), and trichloro-s-triazine (TsT). In one embodiment the hydroxyl group is activated to a succinimidyl carbonate, which is reactive with amines.

Coupling between the compound and the biomolecule may be performed as follows. The compound was reacted with the biomolecule in an organic or aqueous solution at pH between pH 5-pH 12 inclusive. The compound need not be dissolved in an organic solvent, such as dimethyl formamide (DMF) or dimethyl sulfoxide (DMSO) prior to adding the biomolecule. In one embodiment coupling reaction may be performed in a 100% aqueous solution. In one embodiment the coupling reaction occurs at room temperature (about 20° C. to about 22° C.).

To form a composition (dye), at least one biocompatible excipient was added to the compound(s), as known to one of ordinary skill in the art. Excipients include, but are not limited to, buffers, solubility enhancing agents, stabilizing agents, etc.

In one embodiment a kit for performing an assay method comprises a disclosed compound, and instructions for performing the method using the compound.

The disclosed activated compounds (i.e., the compound modified with a reactive group) are useful to label macromolecules (e.g., antibodies, streptavidin, etc.) using methods known to one skilled in the art, e.g., Hermanson, Bioconjugate Techniques, 2nd Ed., London, Elsevier Inc. 2008. The reaction was carried out for 1-2 h at room temperature (about 20° C. to about 22° C.), and then desalted by dialyzing against several changes of phosphate buffered saline (pH 7.2) or purified by gel filtration to remove the unreacted fluorescent dye. The resulting compound-biomolecule conjugate was used to detect, e.g., specific proteins in immunoassays, sugars in glycoproteins with lectins, protein-protein interactions, oligonucleotides in nucleic acid, hybridization, and in electrophoretic mobility shift assays (EMSA).

The resulting compound-biomolecule conjugates exhibited fluorescent properties. In this embodiment, they were used in optical methods including fluorescence optical qualitative and quantitative determination methods. Examples of such methods include, but are not limited to, microscopy, immunoassays, hybridization methods, chromatographic and electrophoretic methods, fluorescence resonance energy transfer (FRET) systems, bioluminescence reasonance energy transfer (BRET) systems, high throughput screenings, analysis of receptor-ligand interactions on a microarray, etc.

Compounds in any embodiment were used as dyes for optical labelling of organic or inorganic biomolecules, referred to as recognition units. Recognition units are molecules having specificity and/or affinity for a specific group of molecules. Examples include, but are not limited to, antibodies that have affinity for antigens, enzymes that bind and/or react with a specific bond or bonds within a sequence of amino acids in a peptide or react with a substrate, cofactors such as metals that enhance or inhibit specific interactions, lectins that bind specific sugars or sugar sequences (e.g., oligosaccharides, polysaccharides, dextrans, etc.), biotin binding proteins such as avidin and streptavidin that bind biotin and biotinylated molecules, antibody binding proteins such as Protein A, Protein G, Protein A/G and Protein L, sequences of amino acids or metals that have affinity for each other (e.g., histidine sequences that bind nickel or copper, phosphate containing proteins that bind gallium, aluminium, etc.), specific sequences of nucleic acids such as DNA and/or RNA oligonucleotides that have affinity for proteins, specific sequences of amino acids that have affinity for DNA and/or RNA, haptens, carotenoids, hormones (e.g., neurohormones), neurotransmitters, growth factors, toxins, biological cells, lipids, receptor binding drugs or organic or inorganic carrier materials, fluorescent proteins such as phycobilliproteins (e.g., phycoethrin, allophycocyanin), etc. Ionic interactions between recognition units and the disclosed compounds results in labeling of the recognition units. The recognition unit and compound can be covalently bound. The result is a conjugate for qualitative or quantitative determination of various biomaterials or other organic or inorganic materials using optical methods.

The inventive compounds and/or conjugates are used in optical, including fluorescence optical, qualitative and/or quantitative determination methods to diagnose properties of cells (molecular imaging), in biosensors (point of care measurements), for investigation of the genome, and in miniaturizing technologies. Microscopy, cytometry, cell sorting, fluorescence correlation spectroscopy (FCS), ultra high throughput screening (uHTS), multicolor fluorescence in situ hybridisation (mc-FISH), FRET-systems, BRET-systems, and microarrays (DNA- and protein-chips) are exemplary application fields. As known to one skilled in the art, a microarray is a grid-like arrangement where more than two different molecules are immobilized in a known predefined region on at least one surface, and is useful to evaluate receptor ligand interactions. As known to one skilled in the art, a receptor is a naturally occurring or synthetic molecule that exhibits an affinity to a given ligand. Receptors can be used in a pure form or bound to another specie. Receptors can be coupled covalently or noncovalently to a binding partner either directly or indirectly (e.g., through a coupling mediator). Receptor examples include, but are not limited to, agonists and antagonists for cell membrane receptors, toxins and other poisons, viral epitopes, hormones (e.g., opiates, steroids), hormone receptors, peptides, enzymes, enzyme substrates, drugs acting as cofactors, lectins, sugars, oligonucleotides, nucleic acids, oligosaccharides, cells, cell fragments, tissue fragments, proteins, antibodies, etc. As known to one skilled in the art, a ligand is a molecule that is recognized by a certain receptor. Ligand examples include, but are not limited to, agonists and antagonists for cell membrane receptors, toxins and other poisons, viral epitopes, hormones (e.g., opiates, steroids), hormone receptors, peptides, enzymes, enzyme substrates, drugs acting as cofactors, lectins, sugars, oligonucleotides, nucleic acids, oligosaccharides, proteins, antibodies, etc.

The following non-limiting examples further describe the compounds, methods, compositions, uses, and embodiments.

### EXAMPLE 1

### Synthesis of 4-methyl-5-oxohexane sulfonic acid used to synthesize Example 2 compound 2,3-dimethyl-3-(3-sulfopropyl)-3H-indole-5-sulfonic acid di-potassium salt and Example 8 compound 1,2-dimethyl-1-(3-sulfopropyl)-1H-benzo[e]indole-6,8-disulfonic acid tripotassium salt

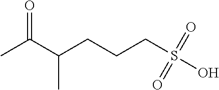

Sodium hydride (2.1 g, 80 wt %=69 mmol) was slurried in 10 ml of dry THF. The suspension was cooled to 0° C. and a solution of ethyl-2-methylacetoacetate (10 g, 69 mmol) in 10 ml of dry THF was added dropwise. The solution was stirred at room temperature for 1 h. A solution of 1,3-propanesultone (8.42 g, 69 mmol) in 10 ml of dry THF was added dropwise. Once the addition was complete, the solution was stirred for 2 h at 40° C. The solution was evaporated to dryness. The residue was dissolved in 100 ml water. The aqueous solution was extracted twice with ethylacetate, then 100 ml concentrated HCl was added and the solution was refluxed for 2 h. The solvent was evaporated in vacuum. The residue was purified by column chromatography (silica, methanol/dichloromethane) to give 4-methyl-5-oxohexane sulfonic acid. Yield 10 g; MS (ESI−): 193.2 [M]⁻

### EXAMPLE 2

### Synthesis of 2,3-dimethyl-3-(3-sulfopropyl)-3H-indole-5-sulfonic acid di-potassium salt used to synthesize Example 3 compound 1-(2-methoxy-ethyl)-2,3-dimethyl-5-sulfo-3-(3-sulfo-propyl)-3H-indolium and Example 4 compound 1-[2-(2-methoxy-ethoxy)-ethyl]-2,3-dimethyl-5-sulfo-3-(3-sulfo-propyl)-3H-indolium and Example 5 compound 1-{2-[2-(2-methoxy-ethoxy)-ethoxy]-ethyl}dimethyl-5-sulfo-3-(3-sulfo-propyl)-3H-indolium and Example 6 compound 1-(5-carboxypentyl)-2,3-dimethyl-5-sulfo-3-(3-sulfopropyl)-3H-indolium

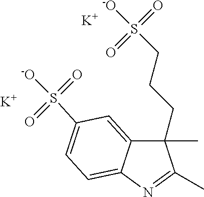

Ten g (51 mmol) 4-hydrazino-benzene sulfonic acid and 9.85 g (51 mmol) 4-methyl-5-oxohexane sulfonic acid were dissolved in 50 ml acetic acid. The solution was heated at 140° C. for 4 h. The solvent was evaporated in vacuum. The oily residue was dissolved in 20 ml methanol, then 50 ml of a saturated solution of KOH in 2-propanol was added to yield a yellow precipitate. The solid was filtered off and dried in vacuum. Yield 11 g, MS (ESI−): 172.5 [M]²⁻

### EXAMPLE 3

### Synthesis of 1-(2-methoxy-ethyl)-2,3-dimethyl-5-sulfo-3-(3-sulfo-propyl)-3H-indolium used to synthesize 550, 650, 755 Compound 1

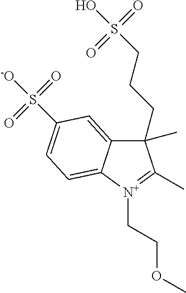

A mixture of 5 g (12.4 mmol) 2,3-dimethyl-3-(3-sulfopropyl)-3H-indole-5-sulfonic acid dipotassium salt and 5.89 g (25.6 mmol) 2-methoxyethyl-p-toluene sulfonate was heated under argon for 24 h. The residue was purified by column chromatography (reversed phase silica, methanol/water, TFA).

Yield 2.3 g, MS (ESI−): 404.1 [M-H]⁻

### EXAMPLE 4

### Synthesis of 1-[2-(2-methoxy-ethoxy)-ethyl]-2,3-dimethyl-5-sulfo-3-(3-sulfo-propyl)-3H-indolium used to synthesize 550, 650, 755 Compound 2

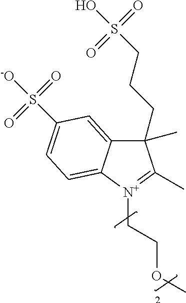

A mixture of 5 g (12.4 mmol) 2,3-dimethyl-3-(3-sulfopropyl)-3H-indole-5-sulfonic acid dipotassium salt and 7.1 g (25.6 mmol) [2-(2-methoxyethoxy)ethoxy]-p-toluene sulfonate was heated under argon for 24 h. The residue was purified by column chromatography (reversed phase silica, methanol/water, TFA). Yield 2.0 g. MS (ESI−): 448.2 [M-H]⁻

### EXAMPLE 5

### Synthesis of 1-{2-[2-(2-methoxy-ethoxy)-ethoxy]-ethyl}-2,3-dimethyl-5-sulfo-3-(3-sulfo-propyl)-3H-indolium used to synthesize 550, 650, 755 Compound 3

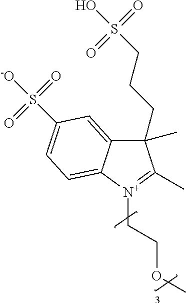

A mixture of 5 g (12.8 mmol) 2,3-dimethyl-3-(3-sulfopropyl)-3H-indole-5-sulfonic acid dipotassium salt and 8.14 g (25.6 mmol) [2-[2-(2-methoxyethoxy)ethoxy]ethoxy]p-toluene sulfonate was heated under argon for 24 h. The residue was purified by column chromatography (reversed phase silica, methanol/water, TFA). Yield 1.9 g, MS (ESI−): 492.1 [M-H]⁻

### EXAMPLE 6

### Synthesis of 1-(5-carboxypentyl)-2,3-dimethyl-5-sulfo-3-(3-sulfopropyl)-3H-indolium used to synthesize Example 7 compound 1-(5-carboxypentyl)-3-methyl-2-((E)-2-phenylamino-vinyl)-5-sulfo-3-(3-sulfo-propyl)-3H-indolium and Example 8 compound 1-(5-carboxypentyl)-3-methyl-2-((1E,3E)-4-phenylamino-buta-1,3-dienyl)-5-sulfo-3-(3-sulfopropyl)-3H-indolium and Example 9 compound 1-(5-carboxypentyl)-3-methyl-2-((1E,3E,5E)-6-phenylamino-hexa-1,3,5-trienyl)-5-sulfo-3-(3-sulfopropyl)-3H-indolium

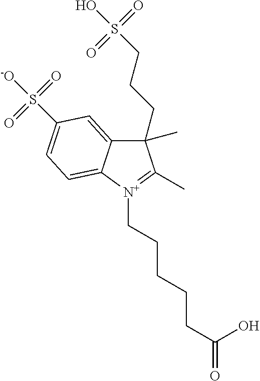

Both 5 g (15.7 mmol) 6-hydrazino-naphthalene-1,3-disulfonic acid and 4.93 g (25 mmol) 4-methyl-5-oxohexane sulfonic acid were dissolved in 50 ml acetic acid. The solution was heated at 140° C. for 4 h. The solvent was evaporated in a vacuum. The oily residue was dissolved in 20 ml methanol, then 50 ml of a saturated solution of KOH in 2-propanol was added to yield a yellow precipitate. The solid was filtered off and dried in vacuum. Yield 4.1 g, MS (ESI−): 158.2 [M]³⁻

### EXAMPLE 7

### Synthesis of 1-(5-carboxypentyl)-3-methyl-2-((E)-2-phenylamino-vinyl)-5-sulfo-3-(3-sulfo-propyl)-3H-indolium used to synthesize 550 Compounds

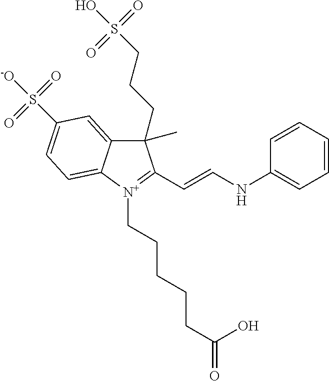

A combination of 0.92 g (2 mmol) 1-(5-carboxypentyl)-2,3-dimethyl-5-sulfo-3-(3-sulfopropyl)-3H-indolium and 0.43 g (2.2 mmol) N,N′-diphenylformamidine was dissolved in 20 ml methanol and stirred for 4 h under reflux. The solvent was removed under vacuum. The residue was washed carefully with ethyl acetate. A dark yellow solid was obtained which was processed without further purification.

MS (ESI−): 563.1 [M-H]⁻

### EXAMPLE 8

### Synthesis of 1-(5-carboxypentyl)-3-methyl-2-((1E,3E)-4-phenylamino-buta-1,3-dienyl)-5-sulfo-3-(3-sulfopropyl)-3H-indolium used to synthesize 650 Compounds

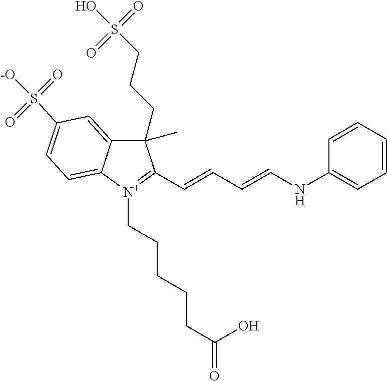

A combination of 0.92 g (2 mmol) 1-(5-carboxypentyl)-2,3-dimethyl-5-sulfo-3-(3-sulfopropyl)-3H-indolium and 0.57 g (2.2 mmol) malonaldehyde-bisphenylimine-hydrochloride were dissolved in 10 ml acetic acid and 10 ml acetic anhydride and stirred for 4 h at 120° C. The solvent was removed under vacuum. The residue was washed carefully with ethyl acetate. A dark brown solid was obtained which was processed without further purification. MS (ESI−): 589.2 [M-H]⁻

### EXAMPLE 9

### Synthesis of 1-(5-carboxypentyl)-3-methyl-2-((1E,3E,5E)-6-phenylamino-hexa-1,3,5-trienyl)-5-sulfo-3-(3-sulfopropyl)-3H-indolium used to synthesize 755 Compounds

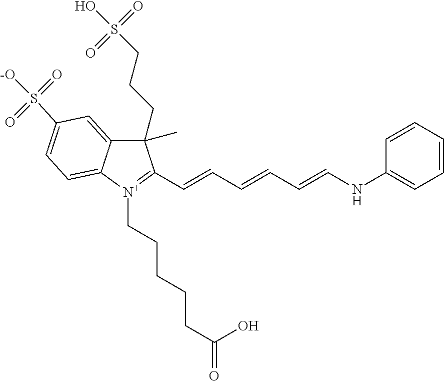

A combination of 0.92 g (2 mmol) 1-(5-carboxypentyl)-2,3-dimethyl-5-sulfo-3-(3-sulfopropyl)-3H-indolium and 0.63 g (2.2 mmol) glutacondianil-hydrochloride were dissolved in 10 ml acetic acid and 10 ml acetic anhydride and stirred for 4 h at 120° C. The solvent was removed under vacuum. The residue was washed carefully with ethyl acetate. A dark solid was obtained which was processed without further purification. MS (ESI−): 615.2 [M-H]⁻

### EXAMPLE 10

### Synthesis of 550 Compound 1 2-{(E)-3-[1-(5-carboxypentyl)-3-methyl-5-sulfo-3-(3-sulfopropyl)-1,3-dihydro-indol-(2E)-ylidene]-propenyl}-1-(2-methoxy-ethyl)-3-methyl-5-sulfo-3-(3-sulfo-propyl)-3H-indolium tri sodium salt

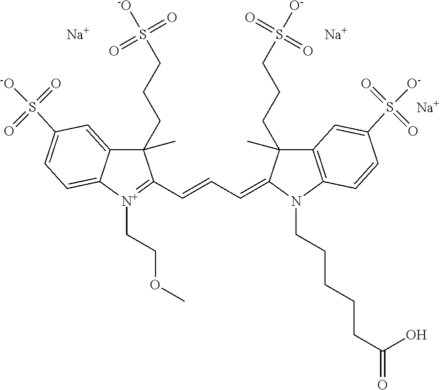

Five hundred sixty-four mg (1 mmol) 1-(5-carboxypentyl)-3-methyl-2-((E)-2-phenylamino-vinyl)-5-sulfo-3-(3-sulfopropyl)-3H-indolium and 404 mg (1 mmol) 1-(2-methoxy-ethyl)-2,3-dimethyl-5-sulfo-3-(3-sulfopropyl)-3H-indolium were dissolved in 20 ml of acetic acid/acetic anhydride (1/1), followed by 200 mg sodium acetate. The solution was stirred under reflux for 15 min. After cooling to room temperature, 20 ml diethylether was added. The resulting precipitate (mixture of the diastereomers 550 Compound 1 (isomer 1) and 550 Compound 1 (isomer 2)) was extracted by suction, washed with ether, and dried.

The residue was purified by column chromatography (RP-18, acetonitrile/water and concentrated HCl) to separate the diastereomers from each other. The diastereomer that first eluted from the column was termed diastereomer 1 (550 Compound 1 (isomer 1)). The diastereomer that eluted second from the column was termed diastereomer 2 (550 Compound 1 (isomer 2)). The diastereomers were separated, followed by neutralization and evaporation. Purification of the single diastereomeric compound was completed on a RP-18 column, acetonitrile/water. The corresponding fractions were pooled and the solvent was removed by distillation. The two products (diastereomers 550 Compound 1 (isomer 1) and 550 Compound 1 (isomer 2)) were dried in high vacuum.

550 Compound 1 (isomer 1):

yield: 12%

UV-vis (PBS): λmax=557 nm, λem=572 nm

MS (ESI−) [M/z]: 291.2 [M]³⁻; 448.3 [M+Na]²⁻

550 Compound 1 (isomer 2):

yield: 23%

UV-vis (PBS): λmax=557 nm, λem=572 nm

MS (ESI−) [M/z]: 291.1 [M]³⁻; 448.2 [M+Na]²⁻

### EXAMPLE 11

### Synthesis of 550 Compound 2 2-{(E)-3-[1-(5-carboxypentyl)-3-methyl-5-sulfo-3-(3-sulfopropyl)-1,3-dihydro-indol-(2E)-ylidene]-propenyl}-1-[2-(2-methoxy-ethoxy)-ethyl]-3-methyl-5-sulfo-3-(3-sulfo-propyl)-3H-indolium tri sodium salt

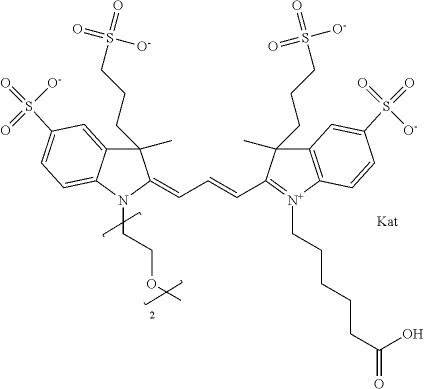

Both 1 mmol 1-(5-carboxypentyl)-3-methyl-2-((E)-2-phenylamino-vinyl)-5-sulfo-3-(3-sulfopropyl)-3H-indolium and 1 mmol 1-[2-(2-methoxy-ethoxy)-ethyl]-2,3-dimethyl-5-sulfo-3-(3-sulfo-propyl)-3H-indolium were dissolved in 20 ml acetic acid/acetic anhydride (1/1) followed by the addition of 200 mg sodium acetate. The solution was stirred under reflux for 15 min. After cooling to room temperature, 20 ml diethylether was added. The resulting precipitate (mixture of the diastereomers 550-1 compound 2 and 550-2 compound 2) was extracted by suction, washed with ether and dried.

The residue was purified by column chromatography (RP-18, acetonitrile/water and concentrated HCl), thereby separating the diastereomers from each other, as described in Example 10.

### EXAMPLE 12

### Synthesis of 550 Compound 3 2-{(E)-3-[1-(5-carboxypentyl)-3-methyl-5-sulfo-3-(3-sulfopropyl)-1,3-dihydro-indol-(2E)-ylidene]-propenyl}-1-{2-[2-(2-methoxy-ethoxy)-ethoxy]-ethyl}-3-methyl-5-sulfo-3-(3-sulfo-propyl)-3H-indolium tri sodium salt

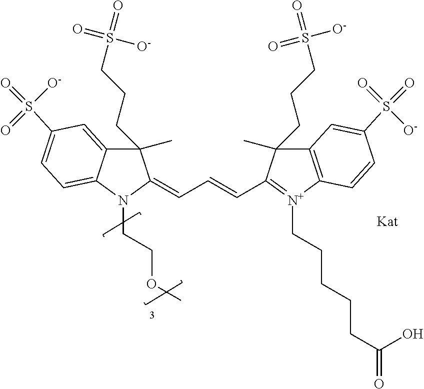

One mmol 1-(5-carboxypentyl)-3-methyl-2-((E)-2-phenylamino-vinyl)-5-sulfo-3-(3-sulfopropyl)-3H-indolium and 1 mmol 1-{2-[2-(2-methoxy-ethoxy)-ethoxy]-ethyl}-2,3-dimethyl-5-sulfo-3-(3-sulfo-propyl)-3H-indolium were dissolved in 20 ml acetic acid/acetic anhydride (1/1) followed by the addition of 200 mg sodium acetate. The solution was stirred under reflux for 15 min. After cooling to room temperature, 20 ml diethylether was added. The resulting precipitate (mixture of the diastereomers 550-1 compound 2 and 550-2 compound 2) was extracted by suction, washed with ether and dried.

The residue was purified by column chromatography (RP-18, acetonitrile/water and concentrated HCl), thereby separating the diastereomers from each other, as described in Example 10.

### EXAMPLE 13

### 650 Compound 1

### Synthesis of 2-{(1E,3E)-5-[1-(5-carboxypentyl)-3-methyl-5-sulfo-3-(3-sulfopropyl)-1,3-dihydro-indol-(2E)-ylidene]-penta-1,3-dienyl}-1-(2-methoxy-ethyl)-3-methyl-5-sulfo-3-(3-sulfopropyl)-3H-indolium tri sodium salt

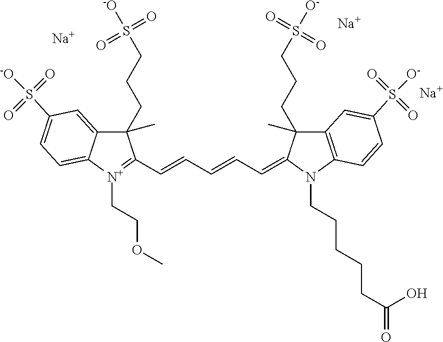

Both 90 mg (1 mmol) 1-(5-carboxypentyl)-3-methyl-2-((1E,3E)-4-phenylamino-buta-1,3-dienyl)-5-sulfo-3-(3-sulfopropyl)-3H-indolium and 404 mg (1 mmol) 1-(2-methoxy-ethyl)-2,3-dimethyl-5-sulfo-3-(3-sulfopropyl)-3H-indolium were dissolved in 20 ml of acetic acid/acetic anhydride (1/1) followed by the addition of 200 mg of sodium acetate. The solution was stirred under reflux for 15 min. After cooling to room temperature, 20 ml diethylether was added. The resulting precipitate (mixture of the diastereomers 650 Compound 1 (isomer 1) and 650 Compound 1 (isomer 2)) was extracted by suction, washed with ether, and dried.

The residue was purified by column chromatography (RP-18, acetonitrile/water and concentrated HCl) to separate the diastereomers from each other. The diastereomer that first eluted from the column was termed diastereomer 1 (650 Compound 1 (isomer 1)). The diastereomer that eluted second from the column was termed diastereomer 2 (650 Compound 1 (isomer 2)). The diastereomers were separated, followed by neutralization and evaporation. Purification of the single diastereomeric compound was completed on a RP-18 column, acetonitrile/water. The corresponding fractions were pooled and the solvent was removed by distillation. The two products (diastereomers 650 Compound 1 (isomer 1) and 650 Compound 1 (isomer 2)) were dried in high vacuum.

650 Compound 1 (isomer 1):

yield: 11%

UV-vis (PBS): λmax=654 nm, λem=672 nm

MS (ESI−) [M/z]: 299.7 [M]3; 461.0 [M+Na]²⁻

650 Compound 1 (isomer 2):

yield: 24%

UV-vis (PBS): λmax=654 nm, λem=672 nm

MS (ESI−) [M/z]: 299.6 [M]³⁻; 461.1 [M+Na]²⁻

### EXAMPLE 14

### 650 Compound 2

### Synthesis of 2-{(1E,3E)-5-[1-(5-carboxypentyl)-3-methyl-5-sulfo-3-(3-sulfopropyl)-1,3-dihydro-indol-(2E)-ylidene]-penta-1,3-dienyl}-1-[2-(2-methoxy-ethoxy)-ethyl]-3-methyl-5-sulfo-3-(3-sulfopropyl)-3H-indolium tri sodium salt

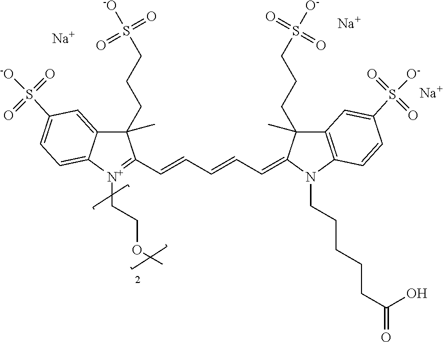

Both 564 mg (1 mmol) 1-(5-carboxypentyl)-3-methyl-2-((1E,3E)-4-phenylamino-buta-1,3-dienyl)-5-sulfo-3-(3-sulfopropyl)-3H-indolium and 449 mg (1 mmol) 1-[2-(2-methoxy-ethoxy)-ethyl]-2,3-dimethyl-5-sulfo-3-(3-sulfopropyl)-3H-indolium were dissolved in 20 ml of acetic acid/acetic anhydride (1/1) followed by the addition of 200 mg of sodium acetate. The synthesis and work-up were carried out according to Example 13.

650-1 compound 2:

yield: 11%

UV-vis (PBS): λmax=654 nm, λem=672 nm

MS (ESI−) [M/z]: 314.4 [M]³⁻; 483.0 [M+Na]²⁻

650-2 compound 2:

yield: 16%

UV-vis (PBS): λmax=654 nm, λem=672 nm

MS (ESI−) [M/z]: 314.5 [M]³⁻; 483.1 [M+Na]²⁻

### EXAMPLE 15

### 650 Compound 3

### Synthesis of 2-{(1E,3E)-5-[1-(5-carboxypentyl)-3-methyl-5-sulfo-3-(3-sulfopropyl)-1,3-dihydro-indol-(2E)-ylidene]-penta-1,3-dienyl}-1-{2-[2-(2-methoxy-ethoxy)-ethoxy]-ethyl}-3-methyl-5-sulfo-3-(3-sulfopropyl)-3H-indolium tri sodium salt—650 compound 3

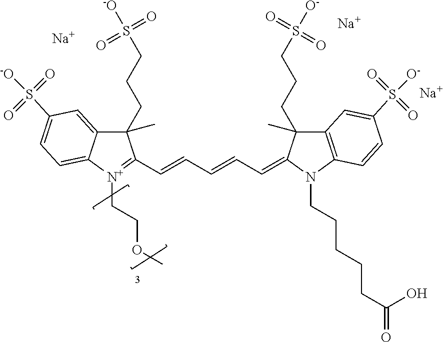

Both 564 mg (1 mmol) 1-(5-carboxypentyl)-3-methyl-2-((1E,3E)-4-phenylamino-buta-1,3-dienyl)-5-sulfo-3-(3-sulfopropyl)-3H-indolium and 493 mg (1 mmol) 1-{2-[2-(2-methoxy-ethoxy)-ethoxy]-ethyl}-2,3-dimethyl-5-sulfo-3-(3-sulfopropyl)-3H-indolium were dissolved in 20 ml acetic acid/acetic anhydride (1/1) followed by the addition of 200 mg sodium acetate. The synthesis and work-up were carried out according to Example 13.

650-1 compound 3:

yield: 10%

UV-vis (PBS): λmax=654 nm, λem=672 nm

MS (ESI−) [M/z]: 329.2 [M]³⁻; 505.0 [M+Na]²⁻

650-2 compound 3:

yield: 23%

UV-vis (PBS): λmax=654 nm, λem=672 nm

MS (ESI−) [M/z]: 329.1 [M]³⁻; 505.1 [M+Na]²⁻

### EXAMPLE 16

### Synthesis of 755 Compound 1 2-{(1E,3E,5E)-7-[1-(5-Carboxypentyl)-3-methyl-5-sulfo-3-(3-sulfopropyl)-1,3-dihydro-indol-(2E)-ylidene]-hepta-1,3,5-trienyl}-1-(2-methoxy-ethyl)-3-methyl-5-sulfo-3-(3-sulfopropyl)-3H-indolium tri sodium salt

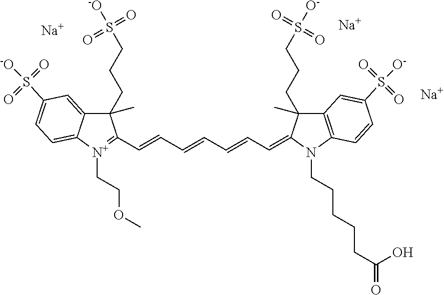

Six hundred and sixteen mg (1 mmol) 1-(5-Carboxypentyl)-3-methyl-2-((1E,3E,5E)-6-phenylamino-hexa-1,3,5-trienyl)-5-sulfo-3-(3-sulfopropyl)-3H-indolium and 404 mg (1 mmol) 1-(2-methoxy-ethyl)-2,3-dimethyl-5-sulfo-3-(3-sulfopropyl)-3H-indolium were dissolved in 20 ml of acetic acid/acetic anhydride (1/1) followed by the addition of 200 mg of sodium acetate. The solution was stirred under reflux for 15 min. After cooling to room temperature, 20 ml diethylether was added. The resulting precipitate (mixture of the diastereomers 755 Compound 1 (isomer 1) and 755 Compound 1 (isomer 2)) was extracted by suction, washed with ether, and dried.

The residue was purified by column chromatography (RP-18, acetonitrile/water and concentrated HCl) to separate the diastereomers from each other. The diastereomer that first eluted from the column was termed diastereomer 1 (755 Compound 1 (isomer 1)). The diastereomer that eluted second from the column was termed diastereomer 2 (755 Compound 1 (isomer 2)). The diastereomers were separated, followed by neutralization and evaporation. Purification of the single diastereomeric compound was completed on a RP-18 column, acetonitrile/water. The corresponding fractions were pooled and the solvent was removed by distillation. The two products (diastereomers 755 Compound 1 (isomer 1) and 755 Compound 1 (isomer 2)) were dried in high vacuum.

755 Compound 1 (isomer 1):

yield: 8%

UV-vis (PBS): λmax=752 nm; λem=778 nm

MS (ESI−) [M/z]: 308.4 [M]³⁻; 474.2 [M+Na]²⁻

755 Compound 1 (isomer 2):

yield: 16%

UV-vis (PBS): λmax=752 nm; λem=778 nm

MS (ESI−) [M/z]: 308.4 [M]³⁻; 474.2 [M+Na]²⁻.

### EXAMPLE 17

### Synthesis of 755 Compound 2 2-{(1E,3E,5E)-7-[1-(5-Carboxypentyl)-3-methyl-5-sulfo-3-(3-sulfopropyl)-1,3-dihydro-indol-(2E)-ylidene]-hepta-1,3,5-trienyl}-1-(2-methoxy-ethoxy)-3-methyl-5-sulfo-3-(3-sulfopropyl)-3H-indolium

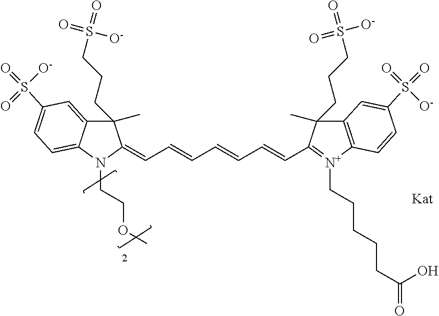

Both 1 mmol 1-(5-Carboxypentyl)-3-methyl-2-((1E,3E,5E)-6-phenylamino-hexa-1,3,5-trienyl)-5-sulfo-3-(3-sulfopropyl)-3H-indolium and 1 mmol 1-[2-(2-methoxy-ethoxy)-ethyl]-2,3-dimethyl-5-sulfo-3-(3-sulfo-propyl)-3H-indolium were dissolved in 20 ml acetic acid/acetic anhydride (1/1) followed by the addition of 200 mg sodium acetate. The solution was stirred under reflux for 15 min. After cooling to room temperature, 20 ml diethylether was added. The resulting precipitate (mixture of the diastereomers 755 compound 2 (isomer 1) and 755 compound 2 (isomer 2)) was extracted by suction, washed with ether and dried. The residue is purified by column chromatography (RP-18, acetonitrile/water and concentrated HCl), thereby separating the diastereomers from each other, as described in Example 16.

### EXAMPLE 18

### Synthesis of 755 Compound 3 2-{(1E,3E,5E)-7-[1-(5-Carboxypentyl)-3-methyl-5-sulfo-3-(3-sulfopropyl)-1,3-dihydro-indol-(2E)-ylidene]-hepta-1,3,5-trienyl}-1-{2-[2-(2-methoxy-ethoxy)-ethoxy]-ethyl}-3-methyl-5-sulfo-3-(3-sulfopropyl)-3H-indolium

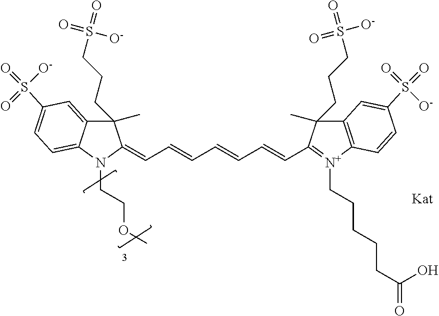

Both 1 mmol 1-(5-carboxypentyl)-3-methyl-2-((1E,3E,5E)-6-phenylamino-hexa-1,3,5-trienyl)-5-sulfo-3-(3-sulfopropyl)-3H-indolium and 1 mmol 1-{2-[2-(2-methoxy-ethoxy)-ethoxy]-ethyl}-2,3-dimethyl-5-sulfo-3-(3-sulfo-propyl)-3H-indolium were dissolved in 20 ml acetic acid/acetic anhydride (1/1) followed by the addition of 200 mg sodium acetate. The solution was stirred under reflux for 15 min. After cooling to room temperature, 20 ml diethylether was added. The resulting precipitate (mixture of the diastereomers 755 compound 3 (isomer 1) and 755 compound 3 (isomer 2)) was extracted by suction, washed with ether and dried. The residue is purified by column chromatography (RP-18, acetonitrile/water and concentrated HCl), thereby separating the diastereomers from each other, as described in Example 16.

### EXAMPLE 19

Properties of 550 Compounds 1-NHS were compared with commercially available dyes, as shown below.

| | | | | Alexa | Alexa |
|---|---|---|---|---|---|
| | 550 | 550 | | Fluor | Fluor |
| | 1/1-NHS | 0/2-NHS | CF555-SE | 555-NHS | 546- NHS |
| MW (g/mol) | 1040 | 1102.3 | 900 | ~1250 | 1260 |
| Ex (nm) | 562 | 557 | 555 | 554 | 555 |
| Em (nm) | 576 | 571 | 565 | 570 | 572 |
| ε (M⁻¹cm⁻¹) | 150,000 | 150,000 | 150,000 | 155,000 | 112,000 |
| (theoretical) | | | | | |
| PEG | 1/1 | 2/4 | N/A | 0 | N/A |
| (length/# | | | | | |
| of chain) | | | | | |
| Sulfonate | 4 | 2 | N/A | 4 | N/A |

### EXAMPLE 20

Inventive and commercial compounds, each as the NHS ester, were conjugated to goat anti-mouse (GAM) and goat anti-rabbit (GAR) antibodies. GAM and GAR at 10 mg/ml in phosphate buffered saline (PBS) were dialyzed against 50 mM borate buffer pH 8.5. The compounds were reconstituted in dimethylformamide (DMF) at 10 mg/ml and combined at 2.5×, 5×, 10×, 12.5×, or 20× molar excess with GAM or GAR for about two hours at room temperature to label the antibodies.

Labeled compounds, also termed dyes or labels, were subjected to PDDR to remove the unlabeled (free) compound; 100-200 μl of the packed resin was used per mg protein purified. The purified antibody-labeled dyes were then diluted 1:50 in PBS and scanned for absorbance from 700 nm to 230 nm to determine the protein concentration, and to determine the mole dye to mole protein ratio. Each conjugate was diluted 1:10 in 50% glycerol and heated in the presence of 10 mM dithiothreitol (DTT) for 5 min at 95° C., then separated by electrophoresis on polyacrylamide gels in the presence of sodium dodecyl sulfate (SDS-PAGE). The gels were scanned using the Typhoon 9400 Imager to verify removal of the unconjugated compound. Labeling efficiency was compared, with results showing degree of labeling below.

| | 2.5X | 5X | 10X | 12.5X | 20X |
|---|---|---|---|---|---|
| GAR | | | | | |
| 550 0/2-NHS | 1.6 | 3 | 5.6 | 7.6 | 11.8 |
| 550 1/1-NHS | 1.7 | 3.1 | 5.7 | 6.9 | 10 |
| CF-555-NHS | 1.5 | 2.7 | 4.8 | 5.6 | 8.5 |
| Alexa Fluor-555 NHS | 2 | 3.7 | 6.2 | 7.7 | 10.3 |
| Alexa Fluor-546 NHS | 3.1 | 5.4 | 4.7 | 4.5 | 3.6 |
| GAM | | | | | |
| 550 0/2-NHS | 1.7 | 3.2 | 6 | 7.1 | 11 |
| 550 1/1-NHS | 1.6 | 3 | 5.5 | 6.5 | 8.8 |
| CF-555-NHS | 1.4 | 3.3 | 5.8 | 6.9 | 8.5 |
| Alexa Fluor-555 NHS | 2 | 3.6 | 7 | 8.4 | 11.4 |
| Alexa Fluor-546 NHS | 3.1 | 5.8 | 7.3 | 5.7 | 6.4 |

Labeling efficiency was similar between 550 0/2 conjugates, and 550 1/1 and CF 555. Alexa Fluor dyes showed about 20-80% better labeling efficiency at the lowest molar excesses, but the Alexa Fluor conjugates appeared to aggregate at molar excess greater than 5×. Compound 550 0/2 was readily soluble in DMF.

### EXAMPLE 21

Performance of dye labeled-GAM conjugates and dye labeled-GAR conjugates was evaluated in a functional assay. Wells of a 96 white opaque plate were coated with target proteins mouse IgG immunoglobulin or rabbit IgG immunoglobulin. One hundred μl mouse or rabbit IgG at a concentration of 10 μg/ml was applied to the corresponding wells in columns 1 and 2. The target proteins were serially diluted 1:1 from the wells in columns 2 to 11 using 100 μl PBS. One hundred μl of samples from wells in column 11 were discarded. One hundred μl PBS was added to wells in column 12. The plates were incubated overnight at 4° C. and then blocked 2×200 μl with Thermo Scientific SuperBlock® Blocking Buffer. The coated plates were washed 2×200 μl with PBS-Tween and 1×200 μl with PBS. Based on the calculated concentrations, GAM and GAR conjugates were diluted 1:250 from a 1 mg/ml starting solution in PBS buffer. Conjugates diluted in PBS to 4 μg/ml were added to the corresponding plates (100 μl/well) and then incubated for one h in the dark. The plates were washed with 2×200 μl with PBS-Tween and 1×200 μl with PBS and filled with PBS buffer (100 μl/well) prior to scanning on Tecan Safire using 562 nmₑₓ/576 nmₑₘ to detect fluorescence intensity.

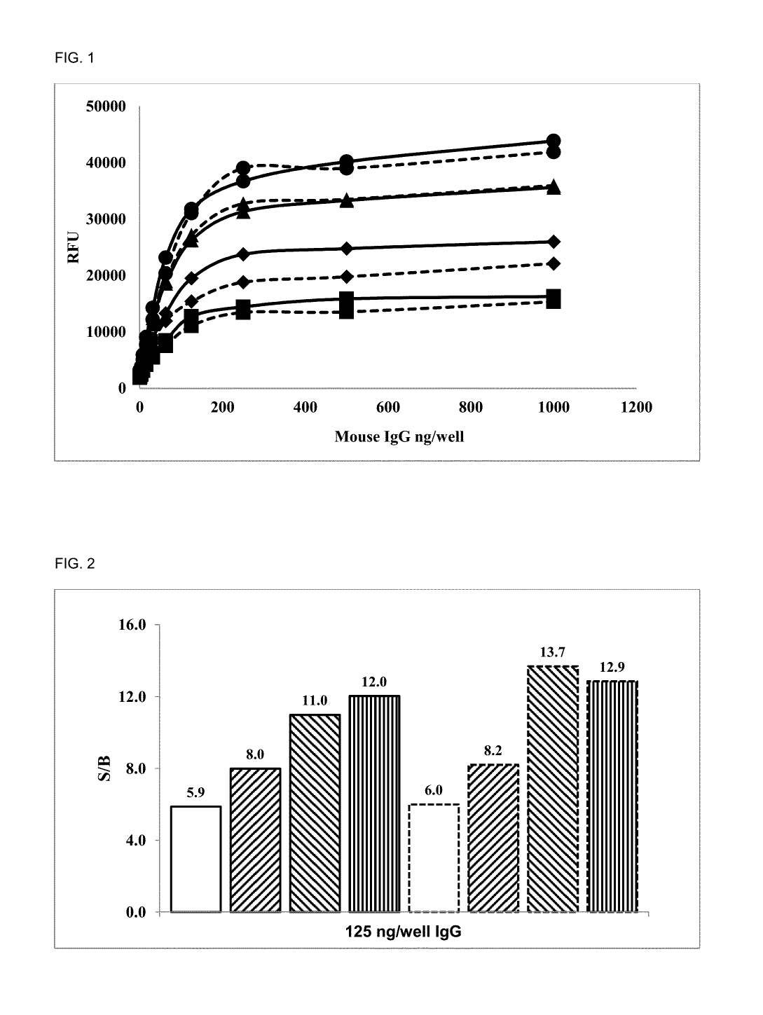

As shown in FIGS. 1-8, relative fluorescence units (RFU) or signal-to-background ratio (S/B) of the dyes were compared at various concentrations using the indicated conjugation conditions.

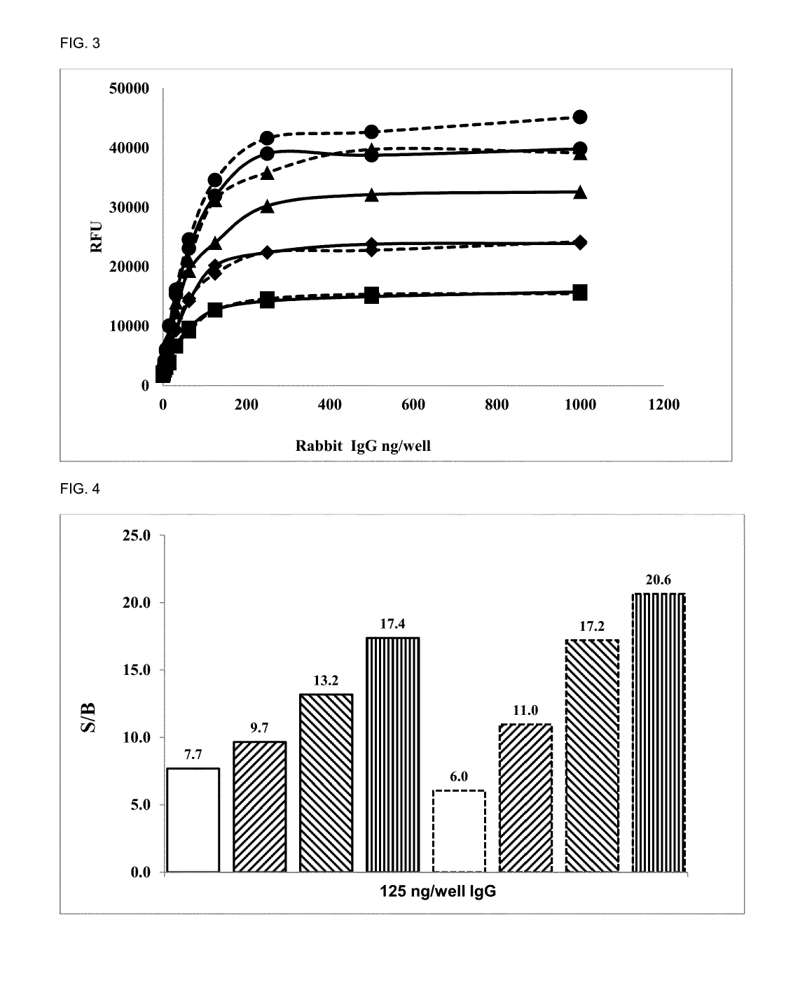

FIG. 1 shows results of a functional assay using GAM conjugated with either 550 0/2 (solid lines) or 550 1/1 (dashed lines) at a 2.5× molar excess of the dyes (squares), 5× molar excess (diamonds), 12.5× molar excess (triangles), or 20× molar excess (circles), showing relative fluorescence units (RFU) versus amount of target anybody per well (ng/well). FIG. 2 shows signal-to-background ratio (S/B) at the 125 ng mouse IgG condition of the functional assay of FIG. 1, showing either 550 0/2 (solid outlined bars) or 550 1/1 (dashed outlined bars) at a 2.5× molar excess of the dyes (open), 5× molar excess (upward diagonal), 12.5× molar excess (downward diagonal), or 20× molar excess (vertical).

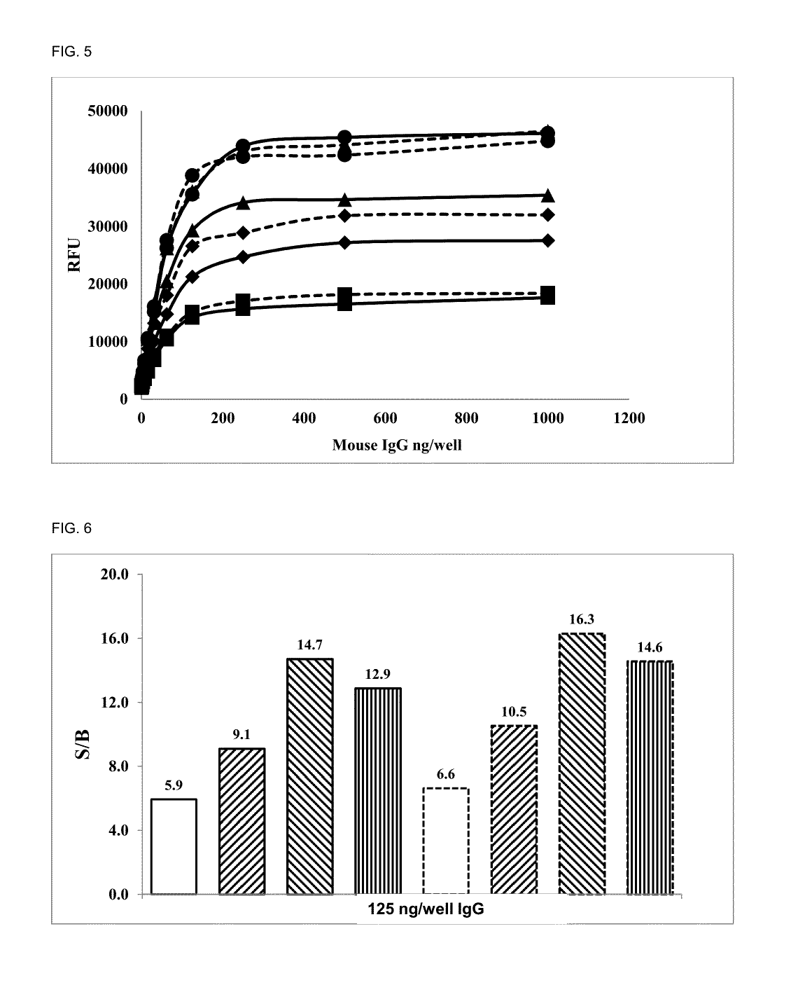

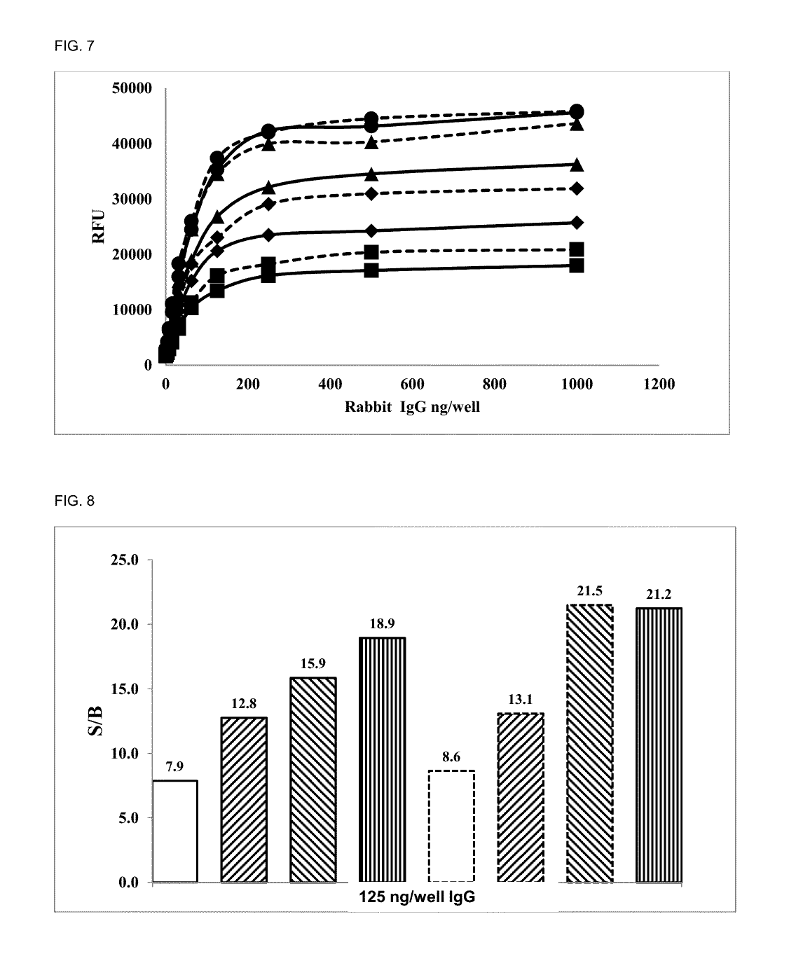

FIG. 3 shows results of a functional assay using GAR conjugated with either 550 0/2 (solid lines) or 550 1/1 (dashed lines) at a 2.5× molar excess of the dyes (squares), 5× molar excess (diamonds), 12.5× molar excess (triangles), or 20× molar excess (circles), showing relative fluorescence units (RFU) versus amount of target antibody per well (ng/well). FIG. 4 shows signal-to-background ratio (S/B) at the 125 ng mouse IgG condition of the functional assay of FIG. 3, showing either 550 0/2 (solid outlined bars) or 550 1/1 (dashed outlined bars) at a 2.5× molar excess of the dyes (open), 5× molar excess (upward diagonal), 12.5× molar excess (downward diagonal), or 20× molar excess (vertical). The binding efficiency of 550 0/2 conjugates on mouse or rabbit IgG coated plates were similar to 550 1/1 conjugates, and showed no quenching at high dye molar excesses.

FIG. 5 shows results of a functional assay using GAM conjugated with either 550 0/2 (solid lines) or CF 555 (dashed lines) at a 2.5× molar excess of the dyes (squares), 5× molar excess (diamonds), 12.5× molar excess (triangles), or 20× molar excess (circles), showing relative fluorescence units (RFU) versus amount of target antibody per well (ng/well). FIG. 6 shows signal-to-background ratio (S/B) at the 125 ng mouse IgG condition of the functional assay of FIG. 5, showing either 550 0/2 (solid outlined bars) or 550 1/1 (dashed outlined bars) at a 2.5× molar excess of the dyes (open), 5× molar excess (upward diagonal), 12.5× molar excess (downward diagonal), or 20× molar excess (vertical).

FIG. 7 shows results of a functional assay using GAR conjugated with either 550 0/2 (solid lines) or CF 555 (dashed lines) at a 2.5× molar excess of the dyes (squares), 5× molar excess (diamonds), 12.5× molar excess (triangles), or 20× molar excess (circles), showing relative fluorescence units (RFU) versus amount of target antibody per well (ng/well). FIG. 8 shows signal-to-background ratio (S/B) at the 125 ng mouse IgG condition of the functional assay of FIG. 7, showing either 550 0/2 (solid outlined bars) or 550 1/1 (dashed outlined bars) at a 2.5× molar excess of the dyes (open), 5× molar excess (upward diagonal), 12.5× molar excess (downward diagonal), or 20× molar excess (vertical). The binding efficiency of 550 0/2 conjugates on mouse or rabbit IgG coated plates were similar to CF 555 conjugates, and showed no quenching at high dye molar excesses.

### EXAMPLE 22

The inventive compounds and commercial dye were evaluated for immunofluorescence in cell based assays using the following protocol. Plates containing U2OS cells (human osteosarcoma cell line) were fixed in 4% paraformaldehyde in PBS/0.1% Triton X-100 for 15 min at room temperature. The cells were then permeabilized with 2% BSA in PBS/0.1% Triton X-100 for 15 min at room temperature. Negative controls contain only 2% BSA/PBS-0.1% Triton-X100 blocker. Diluted primary antibodies, mouse-anti-PDI, rabbit anti-calreticulin, or rabbit-anti-HDAC2, diluted in 2% BSA/PBS-0.1% Triton-X100 were added to the plates and incubated overnight at 4° C. Negative controls contained only 2% BSA/PBS-0.1% Triton-X100 blocker. The plates were washed 3×100 μl with PBS. Based on the calculated protein concentrations, the conjugates were diluted to 4 μg/ml (1:250 from 1 mg/ml stock) in PBS/0.1% Triton X-100 and added to the plates (50 μl/well) and incubated one h in the dark at room temperature. After incubation, the primary antibody solution was removed from the plates, and the plates were washed 3×100 μl with PBS. One hundred μl of 0.1 μg/ml Hoechst dye in PBS was added per well. The plates were then scanned on an ArrayScan® Plate Reader for imaging and quantitation.

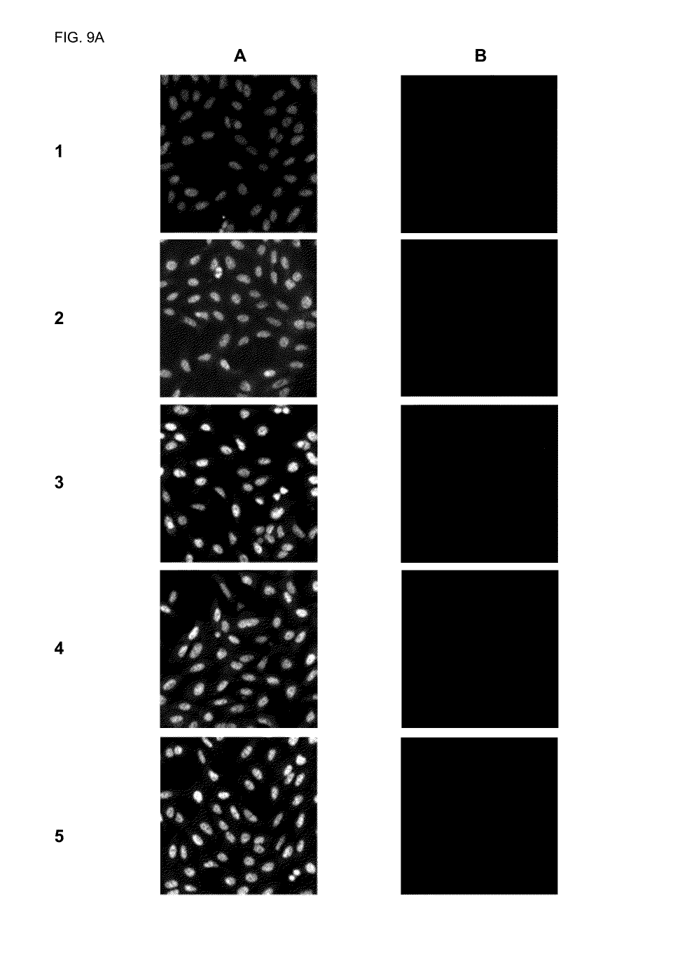

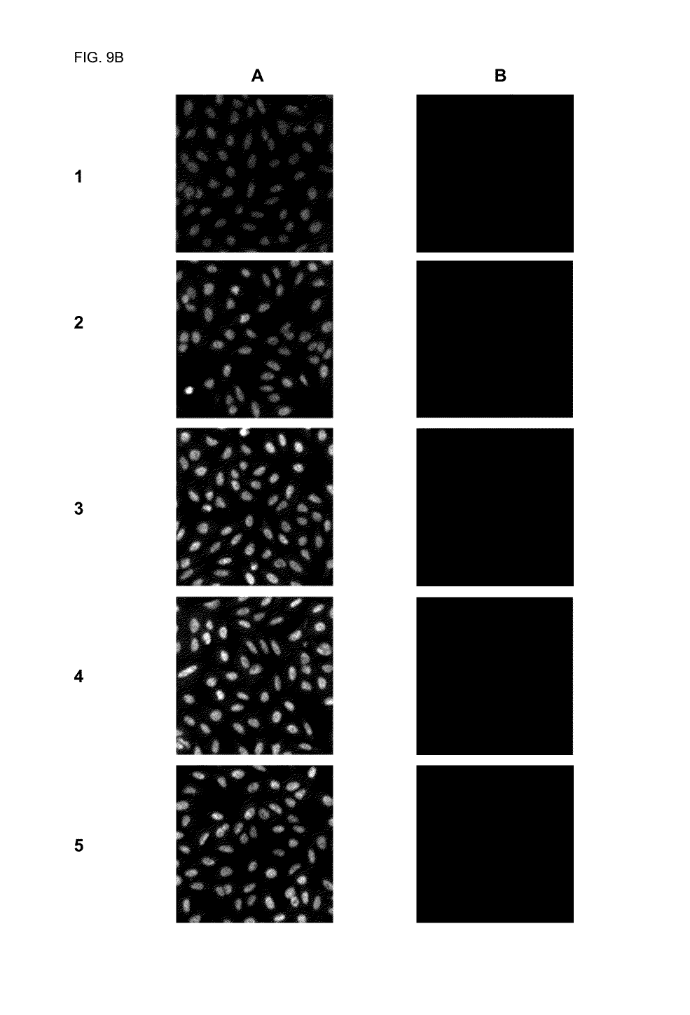

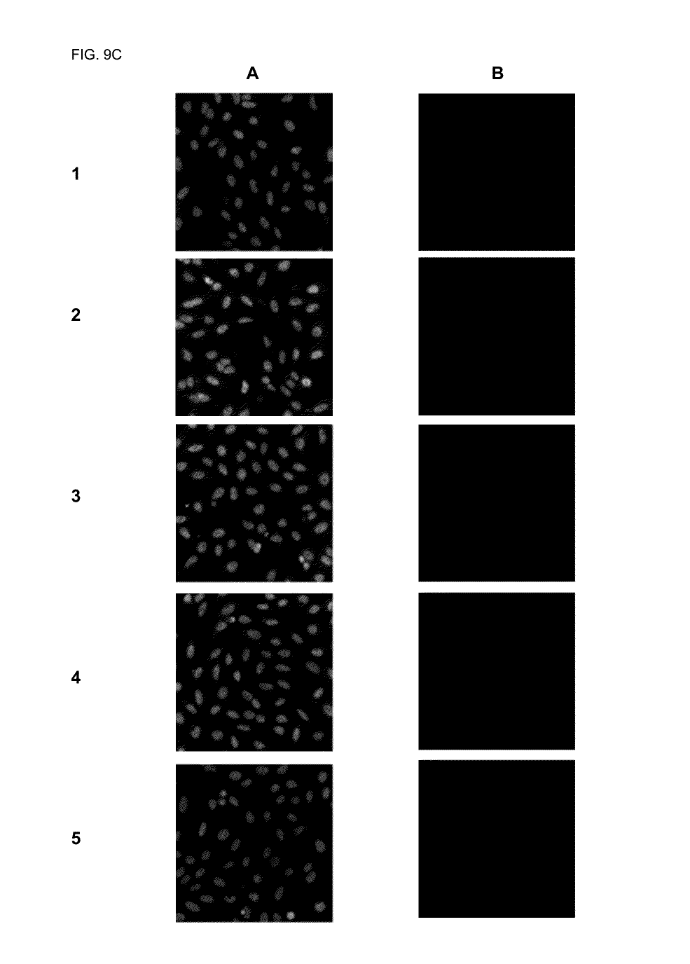

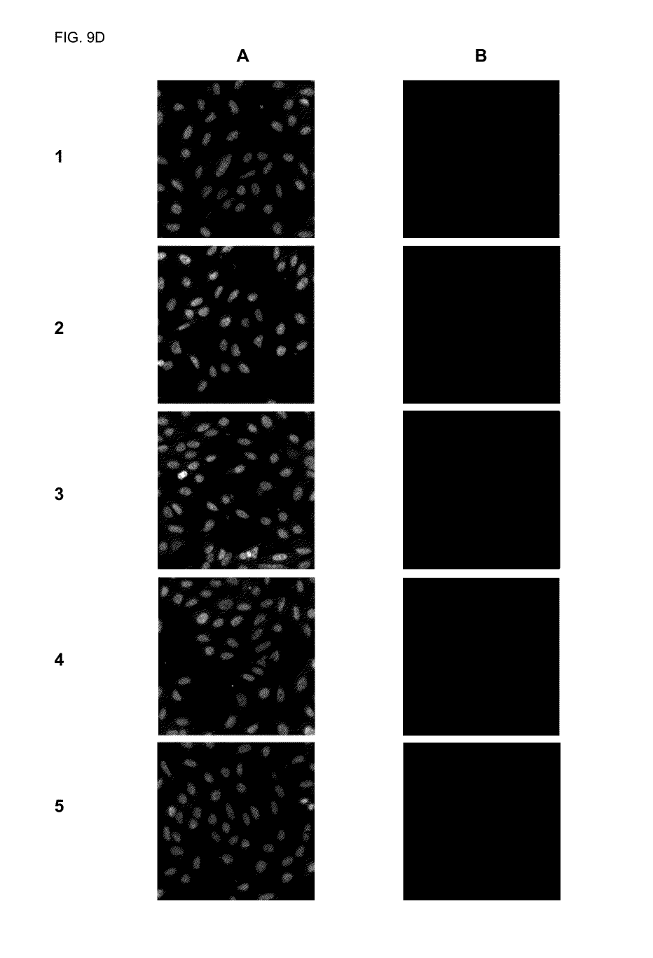

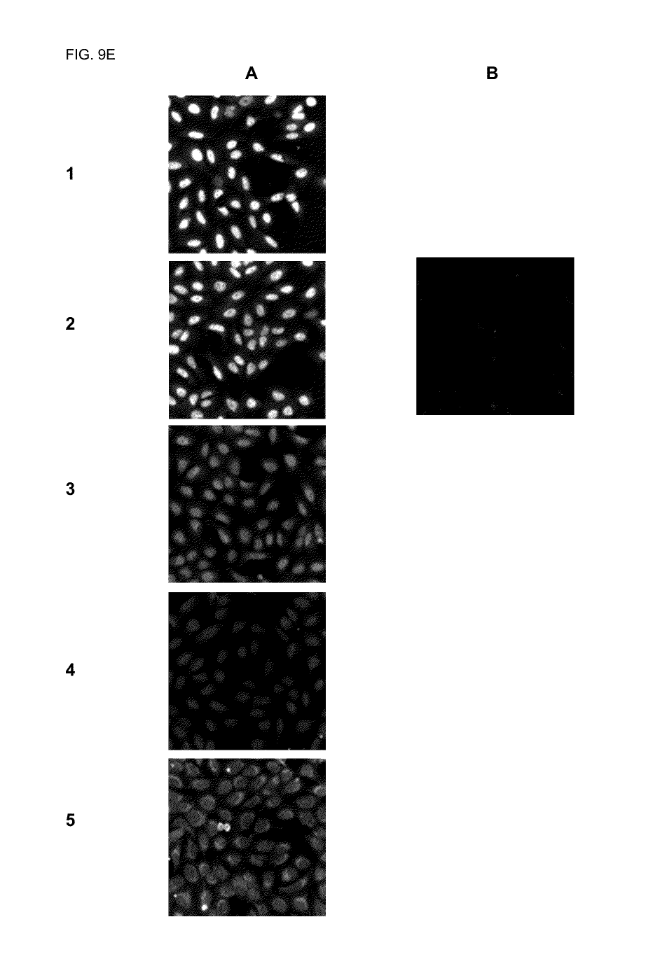

As shown in FIGS. 9-17 and in the following tables, 550 0/2 exhibited fluorescence that was similar or better than 550 1/1 and Alexa Fluor 555.

FIGS. 9A-E shows results of an immunofluorescence assay using rabbit-anti-HDAC2 as a primary antibody, and either 550 0/2-GAR (FIG. 9A; column A), 550 Compound 1/1-GAR (FIG. 9B; column A), CF 555-GAR (FIG. 9C; column A), Alexa Fluor 555-GAR (FIG. 9D; column A), or Alexa Fluor 546-GAR (FIG. 9E; column A) as secondary antibody, with negative controls shown in column B, where the compound was conjugated to GAR (secondary antibody) at 2.5× molar excess (row 1), 5× molar excess (row 2), 10× molar excess (row 3), 12.5× molar excess (row 4), or 20× molar excess (row 5). 550 0/2-GAR conjugates showed excellent staining specificity and increased fluorescence intensity with increasing dye molar excesses, and did not show sign of quenching. 550 0/2-GAR was brighter than 550 1/1-, CF 555-, and Alexa Fluor 555-GAR conjugates. Alexa Fluor 546-GAR showed the highest fluorescence staining at the lowest molar excess, but dramatically decreased in both intensity and specificity at higher molar excesses.

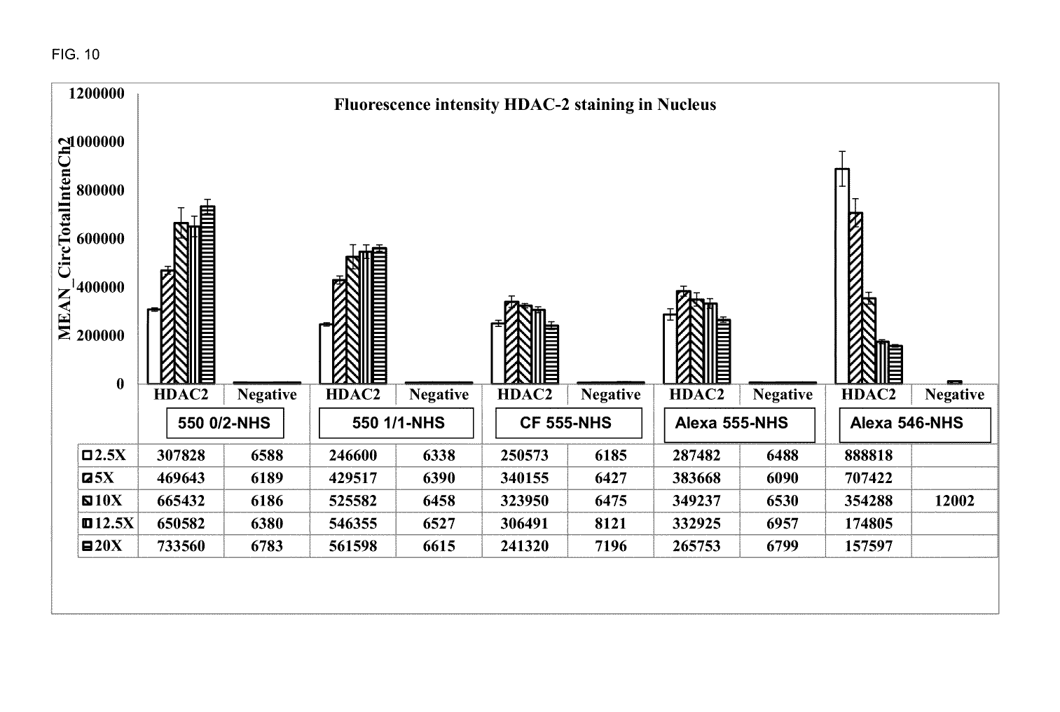

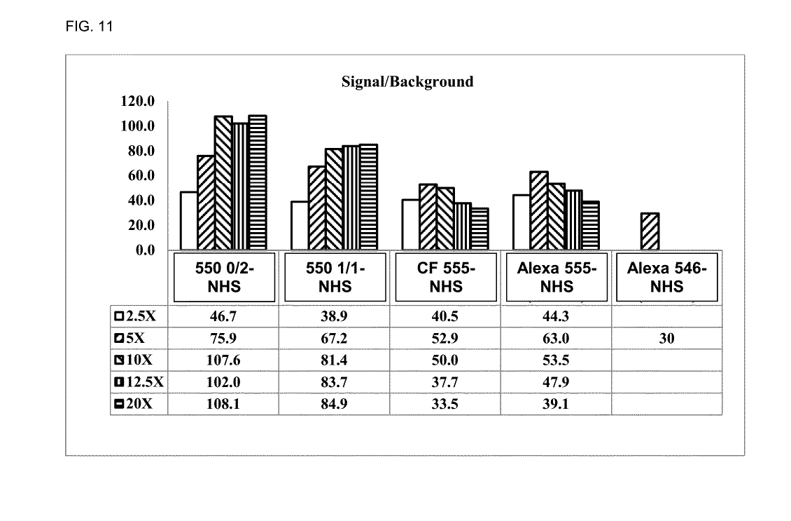

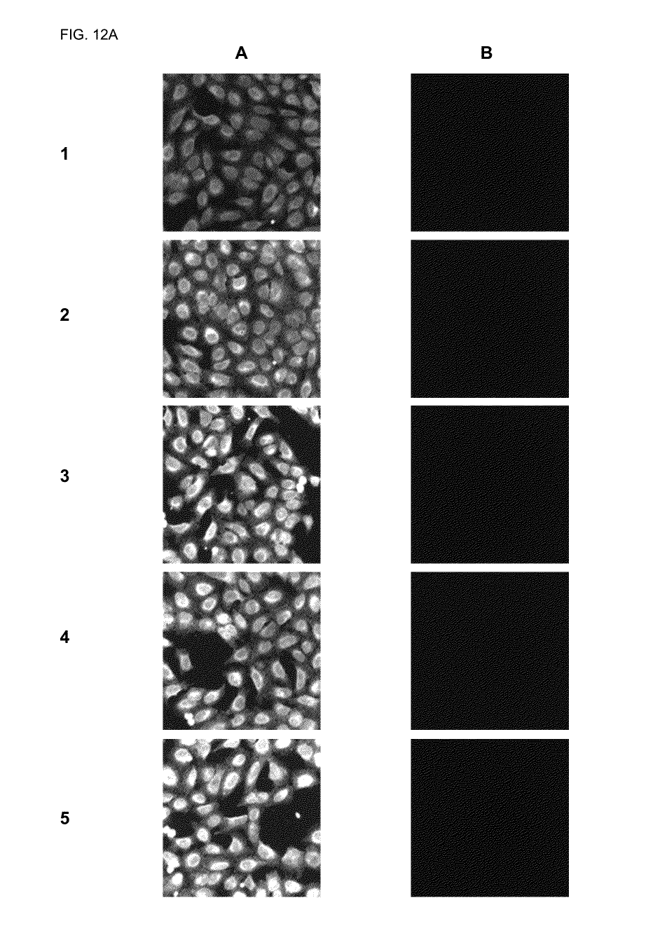

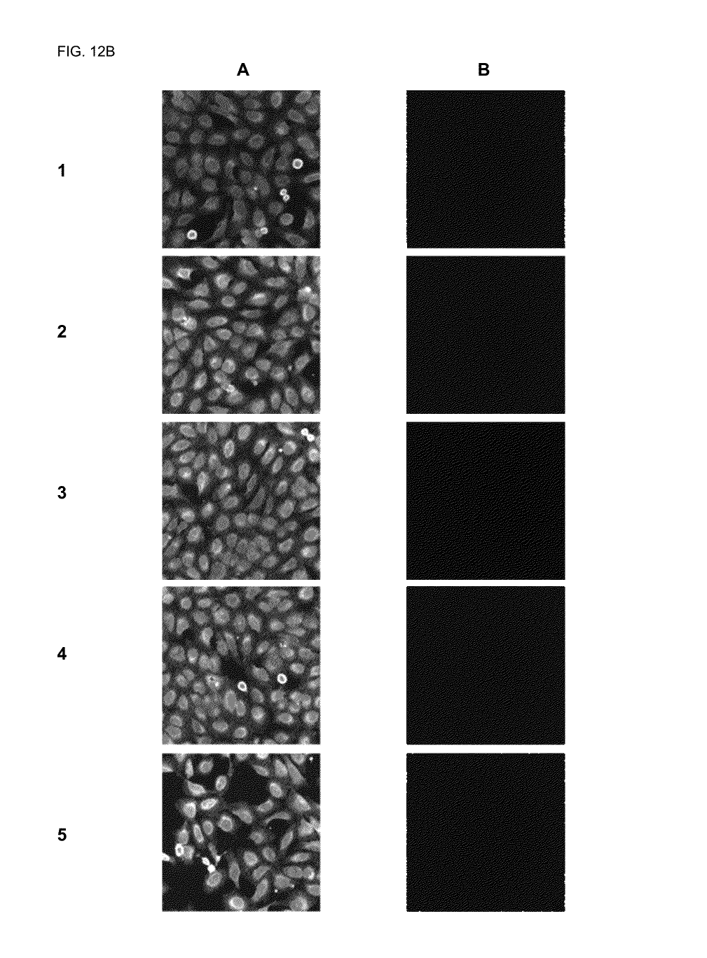

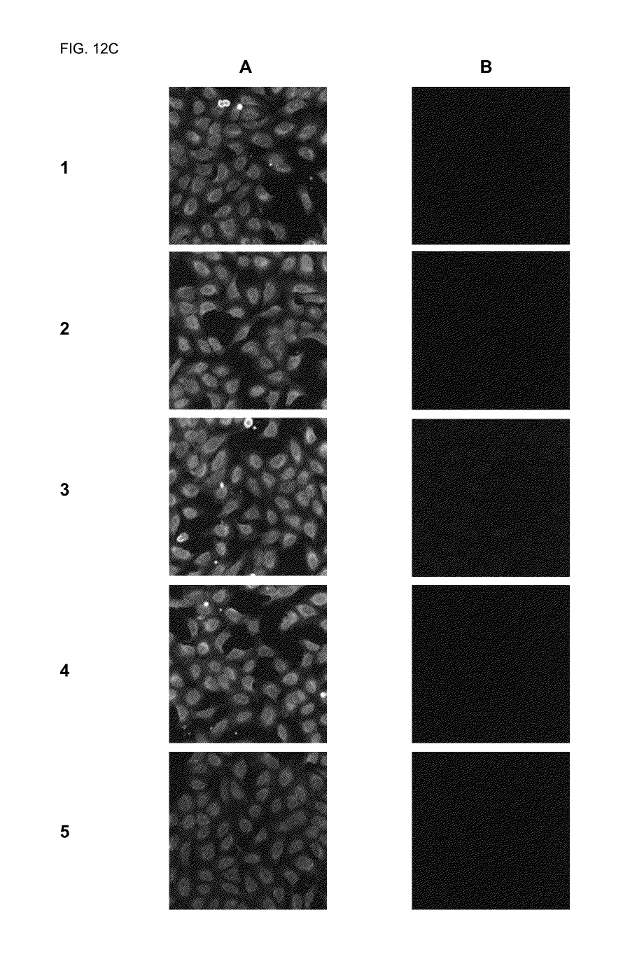

Quantitative analysis of the data of FIGS. 9A-9E, expressed as Mean Total Intensity, which is the average total intensity of all pixels within a defined area or defined primary object such as a nucleus, is shown in FIG. 10 and signal to background ratio (S/B) is shown in FIG. 11. 550 0/2-GAR conjugates showed increasing fluorescence intensity with increasing dye molar excesses, and did not show quenching. 550 0/2-GAR showed approximately 25% higher intensity than 550 1/1-GAR and about 40% higher intensity than CF 555-GAR and Alexa Fluor 555-GAR. Alexa Fluor 546-GAR showed the highest fluorescence intensity at the lowest molar excess but dramatically decreases at higher molar excesses. High staining specificity was observed with 550 0/2 and 550 1/1 conjugates. Alexa Fluor 555 and 546 showed dramatic loss of specificity at molar excesses greater than 10 OX.

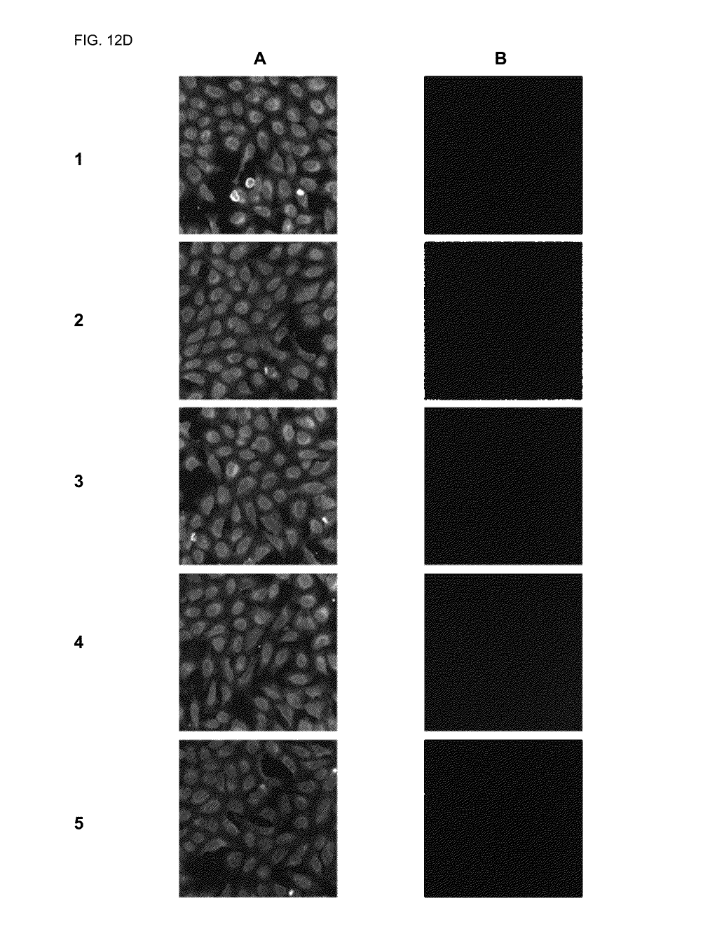

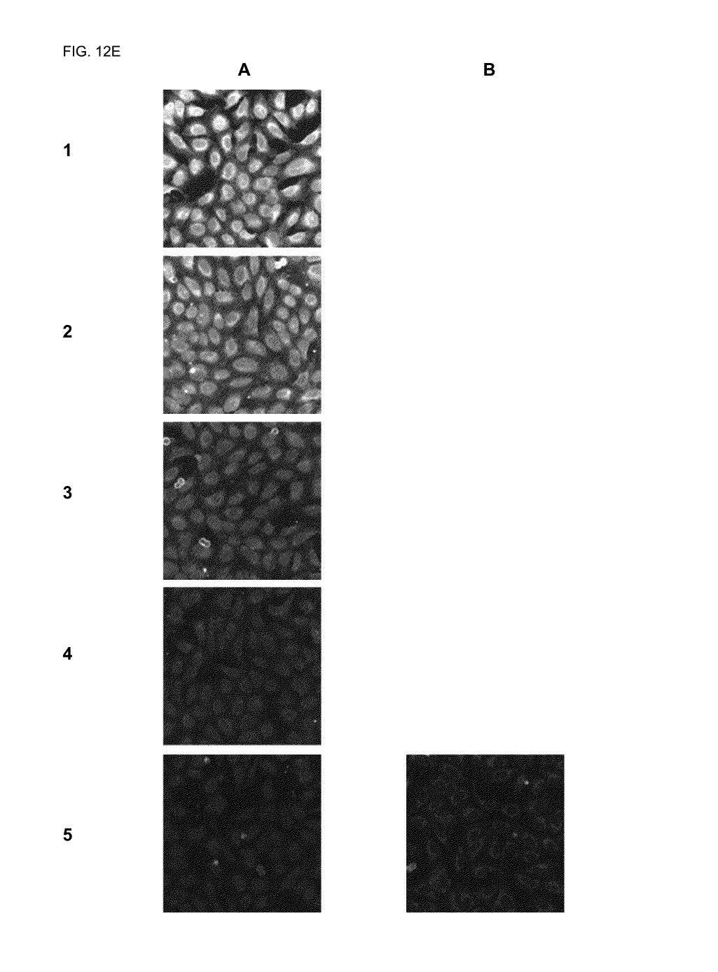

FIGS. 12A-12E shows results of an immunofluorescence assay using rabbit anti-calreticulin as a primary antibody, and either 550 0/2-GAR (FIG. 12A; column A), 550 Compound 1/1-GAR (FIG. 12B; column A), CF 555-GAR (FIG. 12C; column A), Alexa Fluor 555-GAR (FIG. 12D; column A), or Alexa Fluor 546-GAR (FIG. 12E; column A) as secondary antibody, with negative controls shown in column B, where the compound was conjugated to GAR (secondary antibody) at 2.5× molar excess (row 1), 5× molar excess (row 2), 10× molar excess (row 3), 12.5× molar excess (row 4), or 20× molar excess (row 5). 550 0/2-GAR conjugates showed excellent staining specificity and increased fluorescence intensity with increasing dye molar excesses, and did not show sign of quenching. 550 0/2-GAR was brighter than 550 1/1-, CF 555-, and Alexa Fluor 555-GAR conjugates. Alexa Fluor 546-GAR showed the highest fluorescence staining at the lowest molar excess, but dramatically decreased in both intensity and specificity at higher molar excesses.

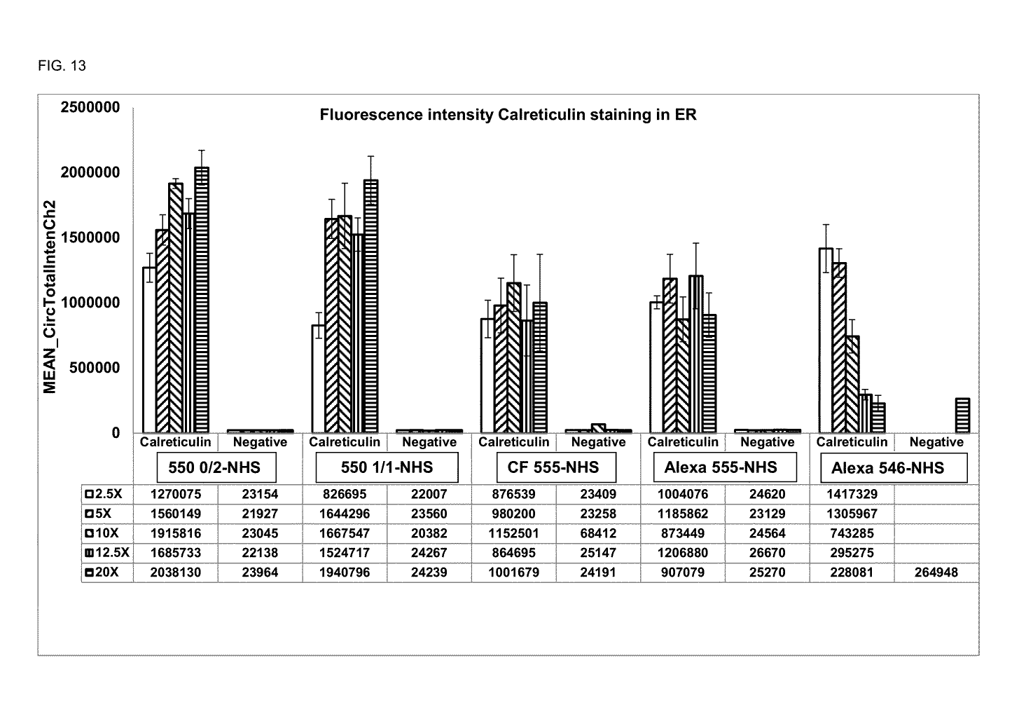

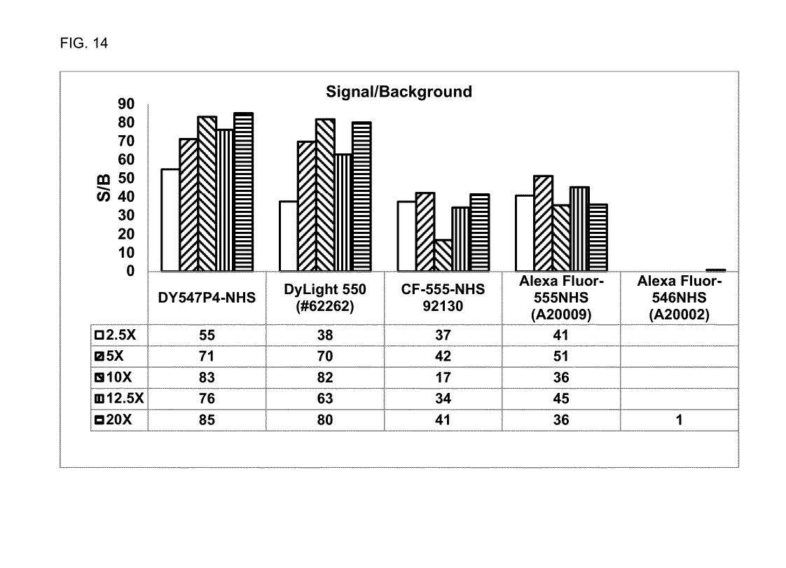

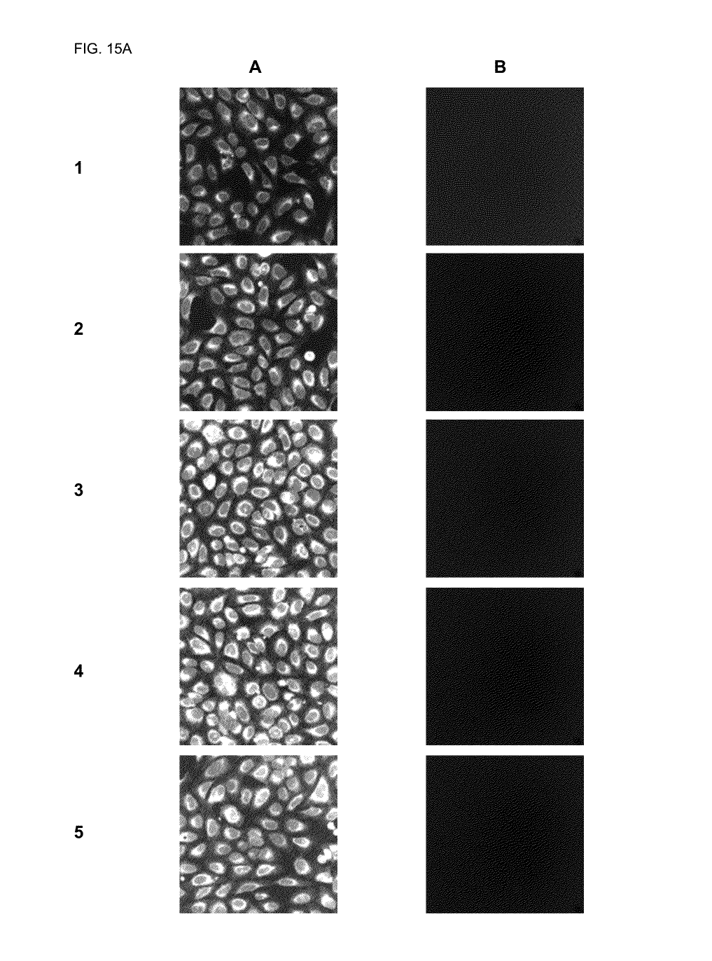

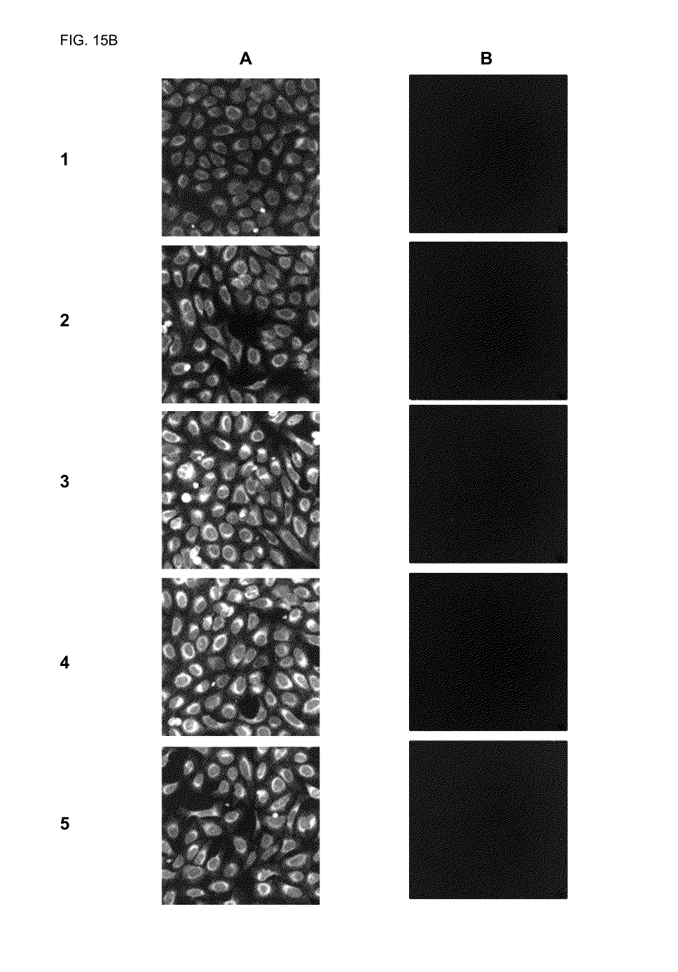

Quantitative analysis of the data of FIGS. 12A-12E, expressed as Mean Total Intensity, which is the average total intensity of all pixels within a defined area or defined primary object such as a nucleus, is shown in FIG. 13 and signal to background ratio (S/B) is shown in FIG. 14. 550 0/2-GAR conjugates showed increasing fluorescence intensity with increasing dye molar excesses, and did not show quenching. 550 0/2-GAR showed approximately 30% higher intensity than 550 1/1-GAR and about 40% higher intensity than CF 555 and Alexa Fluor 555 at low molar excess (2.5×). Alexa Fluor 546-GAR showed the highest fluorescence intensity at the dye lowest molar excess but dramatically decreased at higher molar excesses.

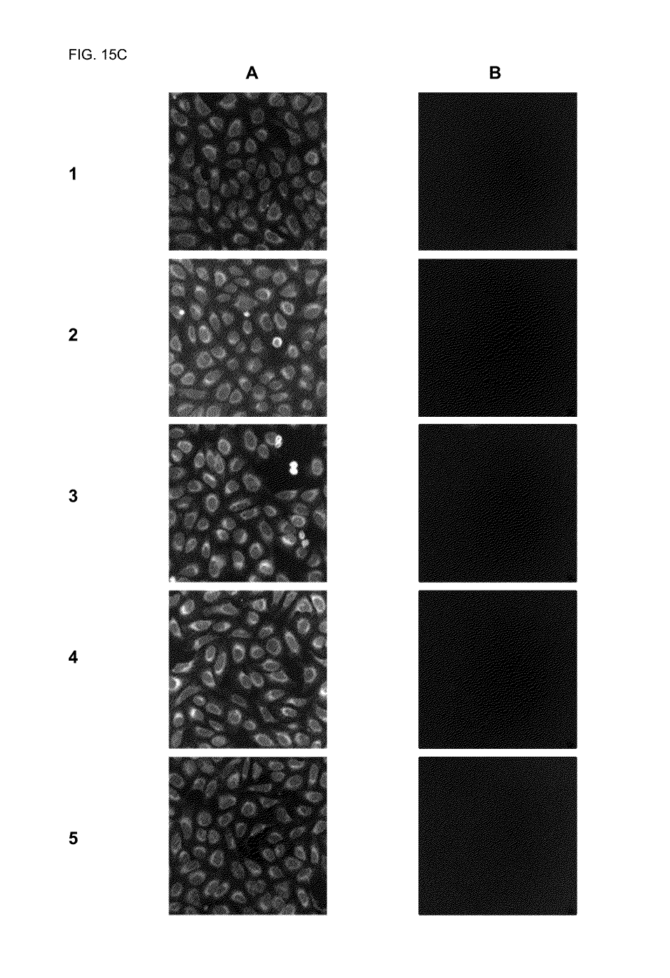

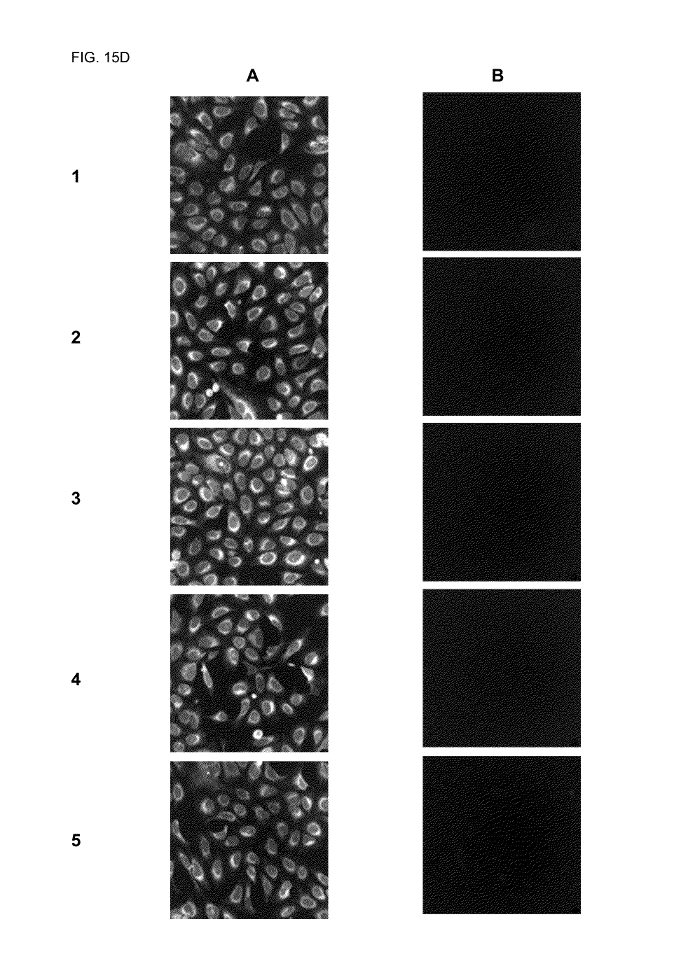

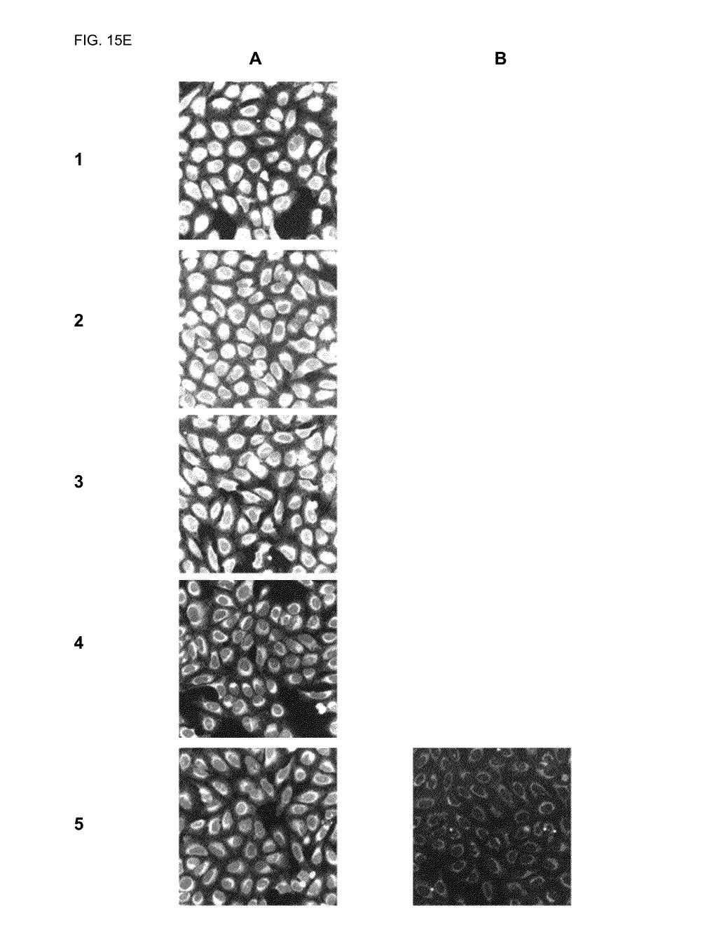

FIGS. 15A-15E shows results of an immunofluorescence assay using mouse anti-PDI as a primary antibody, and either 550 0/2-GAM (FIG. 15A; column A), 550 Compound 1/1-GAM (FIG. 15B; column A), CF 555-GAM (FIG. 15C; column A), Alexa Fluor 555-GAM (FIG. 15D; column A), or Alexa Fluor 546-GAM (FIG. 15E; column A) as secondary antibody, with negative controls shown in column B, where the compound was conjugated to GAR (secondary antibody) at 2.5× molar excess (row 1), 5× molar excess (row 2), 10× molar excess (row 3), 12.5× molar excess (row 4), or 20× molar excess (row 5). 550 0/2-GAM conjugates showed excellent staining specificity and increased fluorescence intensity with increasing dye molar excesses, and did not show quenching. 550 0/2-GAM was brighter than 550 1/1-, CF 555-, and Alexa Fluor 555-GAM conjugates. Alexa Fluor 546-GAM showed the highest fluorescence staining at the lowest molar excess but dramatically in decreased both intensity and specificity at higher molar excesses.

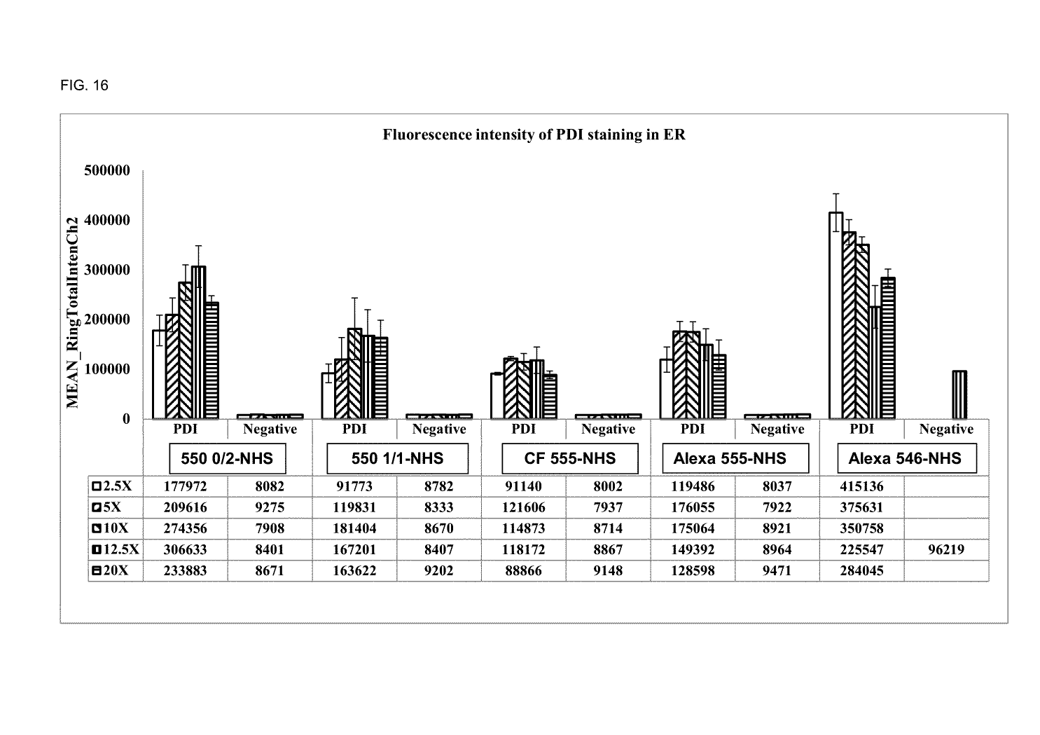

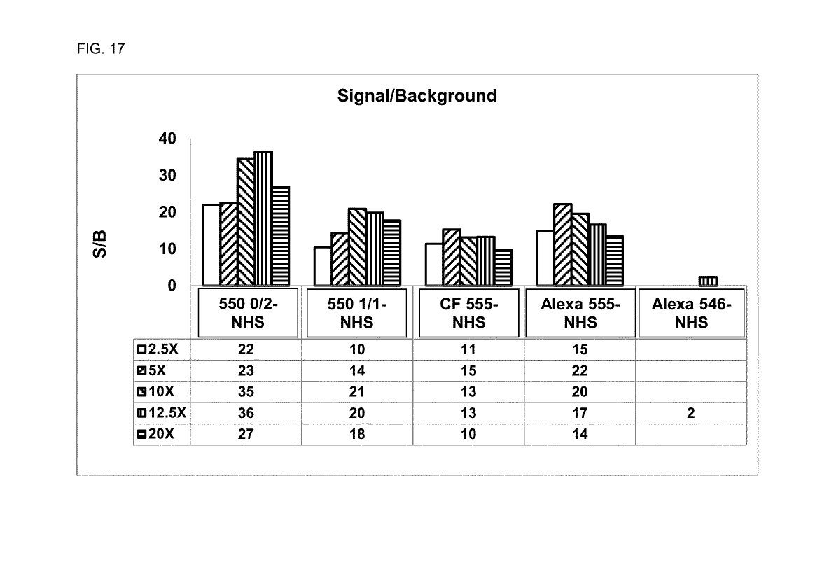

Quantitative analysis of the data of FIGS. 15A-15E, expressed as Mean Total Intensity, which is the average total intensity of all pixels within a defined area or defined primary object such as a nucleus, is shown in FIG. 16 and signal to background ratio (S/B) is shown in FIG. 17. 550 0/2-GAM conjugates showed increased fluorescence intensity with increasing dye molar excesses, and did not show quenching. 550 0/2-GAM showed approximately 30% higher intensity than 550 1/1-GAM and about 40% higher intensity than CF 555-GAM and Alexa Fluor 555-GAM at low molar excess (2.5×). Alexa Fluor 546-GAM showed the highest fluorescence intensity but dramatically decreased with increasing molar excesses.

### EXAMPLE 23

The inventive compounds are evaluated for stability. All compounds are packed under argon in plastic vials. The vials are sealed with a drying pad in an aluminium coated pouch, and then stored at 50° C. for seven days.

### EXAMPLE 24

The inventive compounds are evaluated in direct fluorescence labeling of cell surface proteins using methods known in the art. For example, suitable cell plates, such as IMR90 cells (human lung embryonic fibroblast), are washed and then incubated with the conjugates. The cell plates are then washed and imaged using an appropriate instrument, such as a Thermo Scientific ArrayScan VTI HCS Reader.

### EXAMPLE 25

The inventive compounds are used for in vivo imaging to obtain information about biological tissues that are not readily accessible. The compounds are responsive to light in the near infrared (NIR) region of the spectrum, which is a part of the spectrum that has minimal interference from the absorbance of biological materials. In one embodiment the compounds are used for fluorescent imaging of targets within animals. For example, in vivo imaging information can be obtained using methods such as X-ray, magnetic resonance imaging, positron emission tomography, ultrasound imaging and probing, and other non-invasive methods used for diagnosing and treating disease. Light in the NIR region, from about 650 nm to about 1000 nm wavelength, can permeate through several centimeters of tissue and therefore, can be used for in vivo imaging. Fluorescent dyes, such as the inventive compounds that are responsive to light in these longer wavelengths, can be used as conjugates with targeting molecules such as antibodies to bind and accumulate in, e.g., diseased tissue such as tumors, and may be used to distinguish healthy from diseased tissue. In some methods, the inventive compound may be attached to a biomolecule, such as a protein, peptide, or a drug, which is localized or retained in the desired tissue environment. Fluorescent in vivo imaging using NIR dyes such as the inventive compounds are diagnostic agents to discretely target disease tissue directly within animals or humans.

For in vivo imaging, the compound or a conjugate of the compound with a targeting agent, is administered to a tissue (e.g., intravenously), permitted to accumulate with excess compound removed by the circulatory system, then the tissue is irradiated with light at an appropriate wavelength. The NIR fluorescent light is recorded and/or an image is generated from the data obtained to specifically detect and visualize the targeted cells or tissues. The dose of compound administered can differ depending upon the specific tissue, application, etc., as long as the method achieves a detectable concentration of the compound in the tissue to be assessed.

### EXAMPLE 26

The inventive dyes are evaluated in biodistribution and bioclearance studies. In such an application 1 mg of NHS-DyLight 550 0/2 and NHS-DyLight 550 1/1 are reconstituted to 10 mg/ml and diluted to 1 mg/ml in PBS. The dyes are incubated from 30 minutes and then quenched by adding one-tenth the volume of 3M N-ethanolamine. One hundred μL of 1 mg/mL of each hydrolyzed dye solution was IV injected by the retro orbital plexus of non-tumored nude mice. One mouse is used for each dye. The animals are imaged on a Carestream MSFX at 0 h, 3 h, 6 h, 12 h, and 24 h post injection. After the final time point, animals are sacrificed and tissues collected for ex vivo imaging. The heart, liver, spleen, lungs and kidney tissues are obtained from one mouse from each cohort, and fixed and stained using hematoxylin and eosin (H&E). Colorimetric images are acquired at 20× on a Nikon 90i microscope. The results show that both the hydrolyzed NHS-DyLight 550 0/1 and NHS-DyLight 550 1/1 are removed from the circulatory system.

### EXAMPLE 27

### In Vivo Imaging Using 650 Compound 4/4 Conjugated to Anti-HER2 Antibody

650 Compound 4/4-NHS is conjugated to a rabbit anti-HER2 antibody (Genscript USA, Piscataway N.J.) by reconstituting the compound in dimethylformamide (DMF) at 10 mg/ml, then incubating at 10× molar excess with rabbit anti-HER2 antibody (0.1 mg) for 1 h at room temperature. The sample is then subjected to PDDR to remove unlabeled (free) 650 Compound 4/4. Ten microgram of the conjugate is injected intravenously (IV) to athymic mice bearing BT474 tumors. The animals are imaged over time at 1, 24, 48, 72, 96, and 120 hours post-injection using Pearl Impulse Imager from LI-COR Biosciences (LI-COR Instruments, Lincoln Nebr.).

Upon whole body imaging, fluorescence intensity is observed to be distributed over the whole animal during the first hour imaging and diminishing significantly at 72 hours. After 96 hours, the signal is localized and specific to the tumor.

### EXAMPLE 28

### In Vivo Imaging Using Either Monosulfonated or Disulfonated 650 Compound 4/4

The compound may be rendered less hydrophilic, i.e., more hydrophobic or less negatively charged, by altering the number of sulfonates. Fewer sulfonates render the compound more hydrophobic and less negatively charged. In this embodiment, the compound may be more readily retained in a desired tissue or location if the appropriate number of sulfonates is determined. For example, compound penetration into cells is more efficient if fewer sulfonates are on the compound. The compound may contain one, two, three, or four sulfonates. Hydrophobic compounds are also known to more efficiently cross the cell membrane, and therefore are more desirable when the target of interest is located within the cell.

Alendronate, a compound that binds to, and is retained in, LNCap prostate cancer cells, is conjugated with disulfonated and monosulfonated 650 Compound 4/4 by incubating a solution containing 1 mM disulfonated and/or monosulfonated 650 Compound 4/4-NHS in 1 ml of PBS and 0.5 ml tetrahydrofuran (THF) with 0.1 mM alendronate and 0.2 mM diisopropylethylamine at room temperature overnight. The conjugate is purified using reverse phase HPLC with 0-50% methanol against a 0.1 M ammonium acetate buffer, and is then lyophilized.

LNCap cells are grown orthotopically in nude mice. 650 Compound 1 (isomer 1)-alendronate (5 nmole) is injected into the tumor. Control mice are injected with non-reactive 650 Compound 4/4 containing a carboxylic acid residue instead of the reactive NHS ester. X-ray and near infra-red fluorescence images are captured.

Upon imaging the whole mouse, both the monosulfonated and disulfonated 650 Compound 4/4-alendroneate conjugate is retained in mouse tissue but the free dyes did not; the conjugates are retained in the LNCap cell-induced tumor for at least 18 hrs.

### EXAMPLE 29

### In Vivo Imaging Using Either Monosulfonated or Disulfonated 650 Compound 414

A drug delivery nanoparticle system conjugated with disulfonated and monosulfonated 650 Compound 4/4 is prepared as followed. A solution containing 1 mM disulfonated or monosulfonated 650 Compound 4/4-NHS in 1 ml of PBS is incubated overnight at room temperature with 0.1 mM of an anti-cancer drug conjugated with transferrin in the form of a nanoparticle. The resulting 650 Compound 4/4-nanoparticle conjugate is purified by centrifugation, and then lyophilized.

The 650 Compound 4/4-nanoparticle conjugate (1 nmole) is injected intravenously into the tail vein of different mice. Control mice are injected with non-reactive 650 Compound 4/4 dye containing carboxylic acid residue instead of reactive NHS ester. X-ray and near infra-red fluorescence images of mouse brain are captured.

Both 650 Compound 4/4-nanoparticle conjugate are found to localize in the mouse brain for greater than about 24 hours after injection. Tumor size progressively decreases after injection of 650 Compound 4/4-nanoparticle conjugate, compared to 650 Compound 4/4-nanoparticle without the anti-cancer drug.

### EXAMPLE 30

The mono-sulfonated derivative could be on any one of six possible positions on the 650 compound, accounting for the stereochemistry around the carbon positions on the rings as well as the non-symmetrical nature of the two ends of each dye. Similarly, the di- and tri-substituted sulfonates could be on multiple possible positions on the inventive compounds.

### EXAMPLE 31

Log P (partition coefficient) and log D (distribution coefficient) of inventive and commercial compounds were determined to assess compound hydrophilicity. The log P value of a compound is the logarithm of a compound's partition coefficient between n-octanol and water log(C_{octanol}/C_{water}), and is a well established measure of a compound's hydrophilicity. Log P is a constant for the molecule under its neutral form. Low hydrophilicity, and thus high log P, causes poor absorption or permeation. For compounds to have a reasonable probability of being well absorbed, their log P is generally <5.0. Lipophilicity is not determined by the partitioning of the neutral species in octanol/water, but by the distribution of both the neutral and positively charged forms of the molecule. Log D is related to hydrophilicity of a compound. The distribution coefficient, given by log D, takes into account all neutral and charged forms of the molecule. Because the charged forms generally do not enter the octanol phase, this distribution varies with pH. In the pH range where the molecule is mostly un-ionized, log D=log P. In the pH range where a significant fraction is ionized, log D becomes a function of log P, pH, and pKa. If one assumes that charged molecules do not enter the octanol at all, log D can be expressed as log D=log P−log(1+10**(pH−pKa)).

The following table provides theoretical calculated log D and log P values for 550 Compound 0/2 and 550 Compound 1/1, measured by the ChemAxon program.

550 Compound 0/2 (containing two
PEG₄ substituents, two sulfo substituents)

| pH | LogD |
|---|---|
| 1.50 | 1.48 |
| 5.00 | 0.68 |
| 6.50 | 0.68 |
| 7.40 | 0.68 |

LogP ionic species = 0.7
LogP nonionic species = 1.0
LogD at pI = 2

550 Compound 1/1 (containing one
PEG₁ substituent, two sulfo substituents)

| pH | LogD |
|---|---|
| 1.50 | 2.24 |
| 5.00 | 1.26 |
| 6.50 | 1.25 |
| 7.40 | 1.25 |

LogP ionic species = 1.3
LogP nonionic species = 1.6
LogD at pI = 2.6

The log P and log D calculations comparing 550 Compound 0/2 with two PEG₄ substituents, and 550 Compound 1/1 with one PEG₁ substituent, illustrates the unexpected benefits that even short PEG₄ chains have on the hydrophilicity of cyanine-type dyes. Although the literature teaches the hydrophilicity benefits of longer PEG polymer modifications on small dye molecules, it does not suggest that such benefits occur with short PEG chains. The 550 Compound 1/1 dye calculation of log D indicated that the molecule had a hydrophilicity index of 1.25 around neutral pH. By contrast, 550 Compound 0/2 displayed better hydrophilicity with a log D determination of 0.68 at the same neutral pH values. These observations emphasized the unexpected benefits the invention provided for cyanine dye compounds modified with the relatively short PEG chains described herein.

The embodiments shown and described in the specification are only specific embodiments of inventors who are skilled in the art and are not limiting in any way. Therefore, various changes, modifications, or alterations to those embodiments may be made without departing from the spirit of the invention in the scope of the following claims. The references cited are expressly incorporated by reference herein in their entirety.

What is claimed is:

1. A compound of general formula IIc 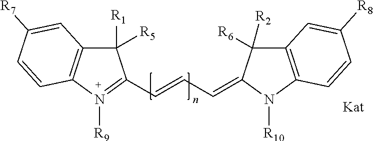
wherein each of R¹, R², R⁵, and R⁶ is the same or different and is independently selected from the group consisting of an aliphatic, heteroaliphatic, sulfoalkyl group, heteroaliphatic with terminal SO₃, a PEG group P-L-Z where P is selected from an ethylene glycol group, a diethylene glycol group, and a (poly)ethylene glycol group where the (poly)ethylene glycol group is (CH₂CH₂O)ₛ where s is an integer from 3-6 inclusive, a sulfonamide group -L-SO₂NH—P-L-Z, and a caboxamide group -L-CONH—P-L-Z;
each of R⁷ and R⁸ is the same or different and is independently selected from the group consisting of H, SO₃, a PEG group P-L-Z where P is selected from an ethylene glycol group, a diethylene glycol group, and a (poly)ethylene glycol group where the (poly)ethylene glycol group is (CH₂CH₂O)ₛ where s is an integer from 3-6 inclusive, a sulfonamide group —SO₂NH—P-L-Z, and a caboxamide group —CONH—P-L-Z;
each of R⁹ and R¹⁰ is the same or different and is independently selected from the group consisting of alkyl, sulfoalkyl, L^{a}-Z, and L^{a}-X;
where L^{a} is selected from the group consisting of a divalent linear (—(CH₂)ₒ—, o=0 to 15), crossed, or cyclic alkane group that can be substituted by at least one atom selected from substituted nitrogen and/or sulfur;
where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur;
where Z is selected from the group consisting of H, CH₃, an alkyl, a heteroalkyl, NH₂, —COO, —COOH, —COSH, CO—NH—NH₂, —COF, —COCl, —COBr, —COI, —COO-Su (succinimidyl/sulfosuccinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-O-phosphoramidite, —CONR′-L-CHO, —CONR′-L-maleimide, and —CONR′-L-NH—CO—CH₂—I; each of R′ and R″ is independently selected from the group consisting of H, aliphatic group, and heteroaliphatic group, and the biomolecule is a protein, antibody, nucleotide, oligonucleotide, biotin, or hapten;
X is selected from the group consisting of —SH, —NH₂, —NH—NH₂, —F, —Cl, —Br, I, —NHS (hydroxysuccinimidyl/sulfosuccinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group;
Kat is a number of Na⁺, K⁺, Ca²⁺, ammonia, or other cation(s) needed to compensate the negative charge brought by the cyanine; and n is an integer from 1 to 3 inclusive; with the proviso that at least one of R¹, R², R⁵, R⁶, R⁷, and R⁸ contains a PEG group.

2. A compound selected from the group consisting of 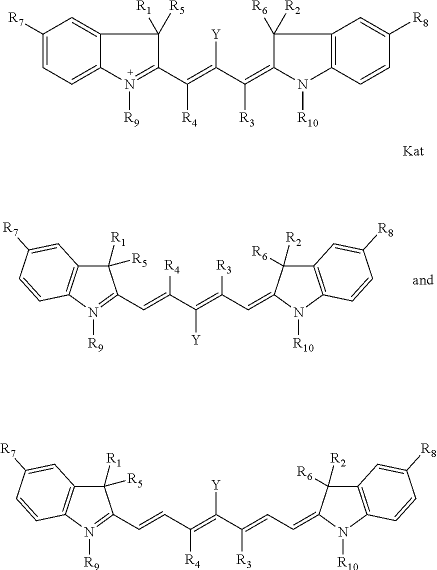
wherein each of R¹, R², R⁵, and R⁶ is the same or different and is independently selected from the group consisting of an aliphatic, heteroaliphatic, sulfoalkyl group, heteroaliphatic with terminal SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group -L-SO₂NH—P-L-Z, and a caboxamide group -L-CONH—P-L-Z;
each of R⁷ and R⁸ is the same or different and is independently selected from either H, SO₃, a PEG group P-L-Z where P is selected from an ethylene glycol group, a diethylene glycol group, and a (poly)ethylene glycol group where the (poly)ethylene glycol group is (CH₂CH₂O)ₛ, where s is an integer from 3-6 inclusive, a sulfonamide group —SO₂NH—P-L-Z, or a caboxamide group —CONH—P-L-Z;
each of R⁹ and R¹⁰ is the same or different and is independently selected from the group consisting of an alkyl, a sulfoalkyl, L^{a}-Z, and L^{a}-X;
where L^{a} is selected from the group consisting of a divalent linear (—(CH₂)ₒ—, o=0 to 15), crossed, or cyclic alkane group that can be substituted by at least one atom selected from substituted nitrogen and/or sulfur;
where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur;
where Z is selected from the group consisting of H, CH₃, alkyl, heteroalkyl, NH₂, —COO⁻, —COOH, —COSH, CO—NH—NH₂, —COF, —COCl, —COBr, —COI, —COO-Su (succinimidyl/sulfosuccinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO⁻, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-O-phosphoramidite, —CONR′-L-CHO, —CONR′-L-maleimide, and —CONR′-L-NH—CO—CH₂—I; R′ and R″ is selected from the group consisting of H, aliphatic group, and heteroaliphatic group, and the biomolecule is a protein, antibody, nucleotide, oligonucleotide, biotin, or hapten;
X is selected from the group consisting of —SH, —NH₂, —NH—NH₂, —F, —Cl, —Br, I, —NHS (hydroxysuccinimidyl/sulfosuccinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group;
Kat is a number of Na⁺, K⁺, Ca²⁺, ammonia, or other cation(s) needed to compensate the negative charge brought by the cyanine;
each of R3 and R4 is the same or different and is independently hydrogen, an aliphatic group, a heteroaliphatic group, or a PEG group P-L-Z where P is selected from an ethylene glycol group, a diethylene glycol group, and a (poly)ethylene glycol group where the (poly)ethylene glycol group is (CH₂CH₂O)ₛ where s is an integer from 3-6 inclusive; or R3 and R4 together form a cyclic structure where R3 and R4 are joined using a divalent structural element selected from the group consisting of —(CH₂)_{q}—, —(CH₂)_{q}O(CH₂)_{q′}—, —(CH₂)_{q}S(CH₂)_{q′}—, —(CH₂)_{q}CH═CH—, —OCH═CH— where each of q and q′ is the same or different and is a integer from 2 to 6 inclusive; and
Y is selected from the group consisting of hydrogen, alkyl, sulfoalkyl, fluorine, chlorine, bromine, a PEG group P-L-Z where P is selected from an ethylene glycol group, a diethylene glycol group, and a (poly)ethylene glycol group where the (poly)ethylene glycol group is (CH₂CH₂O)ₛ where s is an integer from 3-6 inclusive, and an oxygen-containing group OR^{PM}, where R^{PM} is selected from the group consisting of hydrogen, a substituted or unsubstituted alkyl group, a substituted or unsubstituted heteroalkyl group, a substituted or unsubstituted cyclic alkyl group, a substituted or unsubstituted heterocyclic alkyl group, a substituted or unsubstituted aryl group, and a substituted or unsubstituted heteroaryl group, where the group can be substituted one or more times with at least one of hydroxyl, sulfo, carboxy, and/or amino; with the proviso that at least one of R¹, R², R³, R⁴, R⁵, R⁶, R⁷, and R⁸ contains a PEG group.

3. A compound selected from the group consisting of

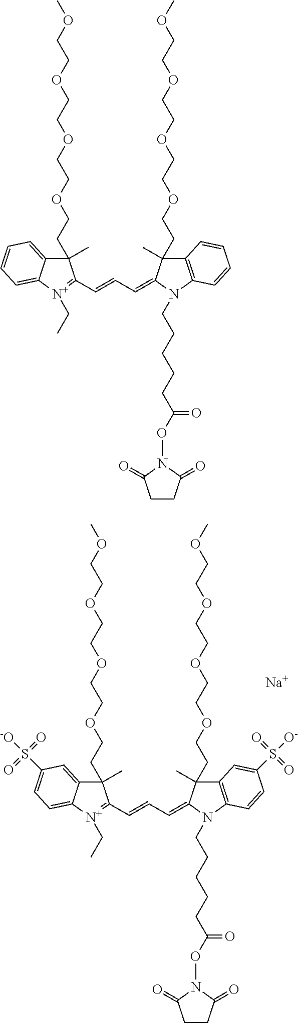

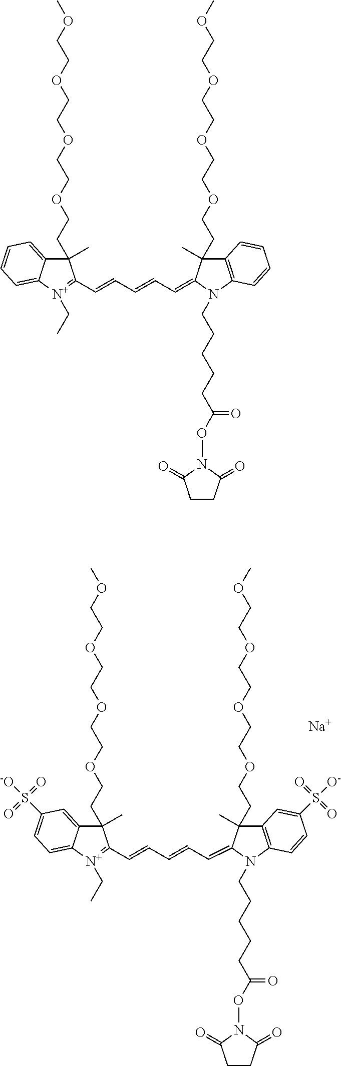

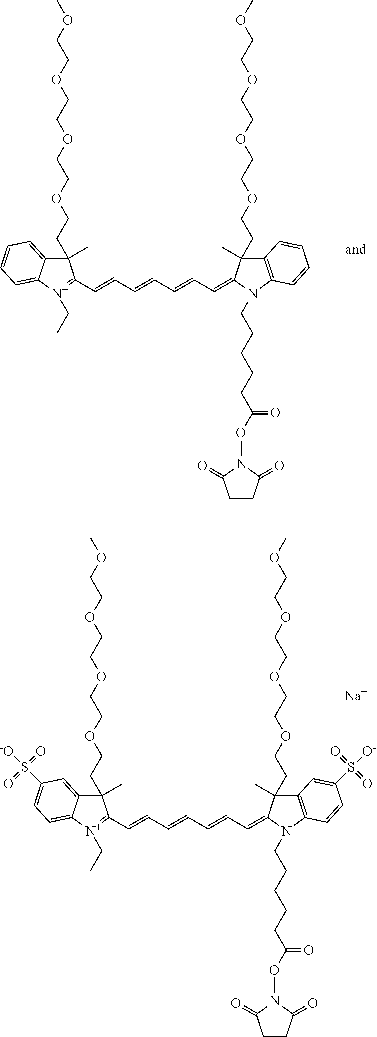

4. A method of labeling at least one biomolecule, the method comprising providing a composition comprising at least one excipient and the compound of claim 1 in an effective concentration to label at least one biomolecule under conditions sufficient for labeling the biomolecule with the compound.

5. The method of claim 4 wherein the biomolecule is selected from the group consisting of a protein, antibody, enzyme, nucleoside triphosphate, oligonucleotide, biotin, hapten, cofactor, lectin, antibody binding protein, carotenoid, carbohydrate, hormone, neurotransmitter, growth factors, toxin, biological cell, lipid, receptor binding drug, fluorescent proteins, organic carrier material, inorganic carrier material, and combinations thereof.

6. A method of detecting at least one biomolecule, the method comprising providing a composition comprising at least one excipient and the compound of claim 2 in an effective concentration to label at least one biomolecule under conditions sufficient for binding the compound to the biomolecule, and detecting the biomolecule-bound compound.

7. The method of claim 6 wherein the biomolecule is selected from a protein, antibody, enzyme, nucleoside triphosphate, oligonucleotide, biotin, hapten, cofactor, lectin, antibody binding protein, carotenoid, carbohydrate, hormone, neurotransmitter, growth factors, toxin, biological cell, lipid, receptor binding drug, fluorescent proteins, organic polymer carrier material, inorganic polymeric carrier material, and combinations thereof.

8. The method of claim 6 wherein the at least one biomolecule is detected in an assay selected from fluorescence microscopy, flow cytometry, in vivo imaging, immunoassay, hybridization, chromatographic assay, electrophoretic assay, microwell plate based assay, fluorescence resonance energy transfer (FRET) system, bioluminescence reasonance energy transfer (BRET) system, high throughput screening, or microarray.

9. The method of claim 6 wherein the biomolecule is detected by in vivo imaging comprising providing the biomolecule-bound compound to at least one of a biological sample, tissue, or organism, and detecting the biomolecule within the at least one of a biological sample, tissue, or organism.

10. A kit for detecting at least one biomolecule in a sample, the kit comprising the compound of claim 2 and at least one excipient, and instructions for use of the compound to detect a biomolecule in a sample.

11. A compound of general formula IIc 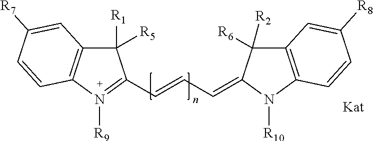
wherein each of R¹, R², R⁵, and R⁶ is the same or different and is independently selected from the group consisting of an aliphatic, heteroaliphatic, sulfoalkyl group, heteroaliphatic with terminal SO₃, a PEG group P-L-Z where P is selected from an ethylene glycol group, a diethylene glycol group, and a (poly)ethylene glycol group where the (poly)ethylene glycol group is (CH₂CH₂O)ₛ where s is an integer from 3-6 inclusive, a sulfonamide group -L-SO₂NH—P-L-Z, and a caboxamide group -L-CONH—P-L-Z;
each of R⁷ and R⁸ is the same or different and is independently selected from the group consisting of H, SO₃, a PEG group P-L-Z where P is selected from an ethylene glycol group, a diethylene glycol group, and a (poly)ethylene glycol group where the (poly)ethylene glycol group is (CH₂CH₂O)ₛ where s is an integer from 3-6 inclusive, a sulfonamide group —SO₂NH—P-L-Z, and a caboxamide group —CONH—P-L-Z;
each of R⁹ and R¹⁰ is the same or different and is independently selected from the group consisting of a PEG group P-L-Z where P is selected from an ethylene glycol group, a diethylene glycol group, and a (poly)ethylene glycol group where the (poly)ethylene glycol group is (CH₂CH₂O)ₛ where s is an integer from 3-6 inclusive, a PEG group P-L-X, L-Z, and L-X;
where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur;
where Z is selected from the group consisting of H, CH₃, an alkyl, a heteroalkyl, NH₂, —COO⁻, —COOH, —COSH, CO—NH—NH₂, —COF, —COCl, —COBr, —COI, —COO-Su (succinimidyl/sulfosuccinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-O-phosphoramidite, —CONR′-L-CHO, —CONR′-L-maleimide, and —CONR′-L-NH—CO—CH₂—I; each of R′ and R″ is independently selected from the group consisting of H, aliphatic group, and heteroaliphatic group, and the biomolecule is a protein, antibody, nucleotide, oligonucleotide, biotin, or hapten;
X is selected from the group consisting of —SH, —NH₂, —NH—NH₂, —F, —Cl, —Br, I, —NHS (hydroxysuccinimidyl/sulfosuccinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group;
Kat is a number of Na⁺, K⁺, Ca²⁺, ammonia, or other cation(s) needed to compensate the negative charge brought by the cyanine; and n is an integer from 1 to 3 inclusive; with the proviso that at least one of R¹, R², R⁵, R⁶, R⁷, and R⁸ contains a PEG group.

12. A compound selected from the group consisting of 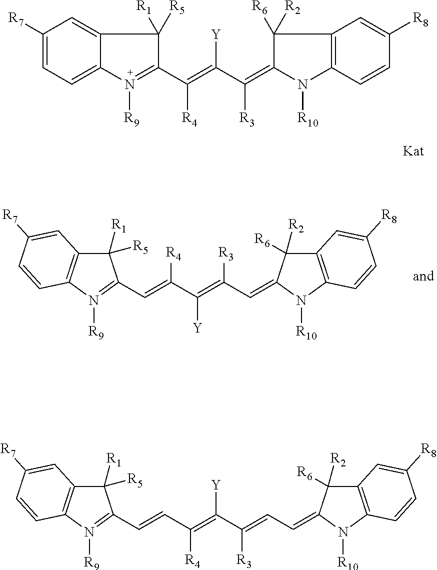
wherein each of R¹, R², R⁵, and R⁶ is the same or different and is independently selected from the group consisting of an aliphatic, heteroaliphatic, sulfoalkyl group, heteroaliphatic with terminal SO₃, a PEG group P-L-Z where P is selected from an ethylene glycol group, a diethylene glycol group, and a (poly)ethylene glycol group, where the (poly)ethylene glycol group is (CH₂CH₂O)ₛ, where s is an integer from 3-6 inclusive, a sulfonamide group -L-SO₂NH—P-L-Z, and a caboxamide group -L-CONH—P-L-Z;
each of R⁷ and R⁸ is the same or different and is independently selected from either H, SO₃, a PEG group P-L-Z where P is selected from an ethylene glycol group, a diethylene glycol group, and a (poly)ethylene glycol group where the (poly)ethylene glycol group is (CH₂CH₂O)ₛ, where s is an integer from 3-6 inclusive, a sulfonamide group —SO₂NH—P-L-Z, or a caboxamide group —CONH—P-L-Z;
each of R⁹ and R¹⁰ is the same or different and is independently selected from the group consisting of a PEG group P-L-Z where P is selected from an ethylene glycol group, a diethylene glycol group, and a (poly)ethylene glycol group where the (poly)ethylene glycol group is (CH₂CH₂O)ₛ where s is an integer from 3-6 inclusive, a PEG group P-L-X, L-Z, and L-X;
where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur;
where Z is selected from the group consisting of H, CH₃, alkyl, heteroalkyl, NH₂, —COO⁻, —COOH, —COSH, CO—NH—NH₂, —COF, —COCl, —COBr, —COI, —COO-Su (succinimidyl/sulfosuccinimidyl), —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO⁻, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-O-phosphoramidite, —CONR′-L-CHO, —CONR′-L-maleimide, and —CONR′-L-NH—CO—CH₂—I; R′ and R″ is selected from the group consisting of H, aliphatic group, and heteroaliphatic group, and the biomolecule is a protein, antibody, nucleotide, oligonucleotide, biotin, or hapten;
X is selected from the group consisting of —SH, —NH₂, —NH—NH₂, —F, —Cl, —Br, I, —NHS (hydroxysuccinimidyl/sulfosuccinimidyl), —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-O-phosphoramidite, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH₂—I, where R is —H or an aliphatic or heteroaliphatic group;
Kat is a number of Na⁺, K⁺, Ca²⁺, ammonia, or other cation(s) needed to compensate the negative charge brought by the cyanine;
each of R3 and R4 is the same or different and is independently hydrogen, an aliphatic group, a heteroaliphatic group, or a PEG group P-L-Z where P is selected from an ethylene glycol group, a diethylene glycol group, and a (poly)ethylene glycol group where the (poly)ethylene glycol group is (CH₂CH₂O)ₛ where s is an integer from 3-6 inclusive; or R3 and R4 together form a cyclic structure where R3 and R4 are joined using a divalent structural element selected from the group consisting of —(CH₂)_{q}—, —(CH₂)_{q}O(CH₂)_{q′}—, —(CH₂)_{q}S(CH₂)_{q′}—, —(CH₂)_{q}CH═CH—, —OCH═CH— where each of q and q′ is the same or different and is a integer from 2 to 6 inclusive; and
Y is selected from the group consisting of hydrogen, alkyl, sulfoalkyl, fluorine, chlorine, bromine, a PEG group P-L-Z where P is selected from an ethylene glycol group, a diethylene glycol group, and a (poly)ethylene glycol group where the (poly)ethylene glycol group is (CH₂CH₂O)ₛ where s is an integer from 3-6 inclusive, and an oxygen-containing group OR^{PM}, where R^{PM} is selected from the group consisting of hydrogen, a substituted or unsubstituted alkyl group, a substituted or unsubstituted heteroalkyl group, a substituted or unsubstituted cyclic alkyl group, a substituted or unsubstituted heterocyclic alkyl group, a substituted or unsubstituted aryl group, and a substituted or unsubstituted heteroaryl group, where the group can be substituted one or more times with at least one of hydroxyl, sulfo, carboxy, and/or amino; with the proviso that at least one of R¹, R², R³, R⁴, R⁵, R⁶, R⁷, and R⁸ contains a PEG group.
